(19) United States
(12) Patent Application Publication
(10) Pub. No.: US 2010/0222262 A1
(43) Pub. Date: Sep. 2, 2010

(54) SUBSTITUTED 1, 3-CYCLOPENTADIONE ATTENUATED ENDOTHELIAL INFLAMMATION AND ENDOTHELIAL-MONOCYTE INTERACTIONS

(75) Inventors: Veera Konda, Gig Harbor, WA (US); Anu Desai, Gig Harbor, WA (US); Matthew L. Tripp, Gig Harbor, WA (US); Gary K. Darland, Gig Harbor, WA (US); Joseph Lamb, Gig Harbor, WA (US); Jeffrey S. Bland, Fox Island, WA (US); Dennis A. Emma, Gig Harbor, WA (US)

Correspondence Address: McDermott Will & Emery, 600 13th Street, NW, Washington, DC, 20005-3096, US

(73) Assignee: METAPROTEOMICS, LLC, San Clemente, CA (US)

(21) Appl. No.: 12/417,205

(22) Filed: Apr. 2, 2009

Related U.S. Application Data
(60) Provisional application No. 61/041,631, filed on Apr. 2, 2008.

(51) Int. Cl. A61K 38/16; A61K 31/12; A61K 31/70; A61P 9/12; A61P 3/06; A61P 3/10; A61P 3/04; A61P 29/00; A61P 25/22; A61P 25/34

(52) U.S. Cl. 514/12; 514/690; 514/23

(57) ABSTRACT

Compositions and methods for reducing cardiovascular risk utilizing substituted 1,3-cyclopentadione compounds are described.

### CROSS-REFERENCE TO RELATED APPLICATIONS

This patent application claims priority to U.S. provisional application Ser. No. 61/041,631 filed on Apr. 2, 2008, the contents of which are incorporated herein in its entirety by reference.

### BACKGROUND OF THE INVENTION

1. Field of the Invention

The present invention relates generally to methods and compositions that can be used to modulate inflammatory pathways associated with endothelial function and cardiovascular complications, including (1) TNFa mediated VCAM-1, E selectin and MCP-1 in endothelial cells (HAEC) and (2) TNFa mediated Monocyte-Endothelial interactions via protein kinase modulation. More specifically, the invention relates to methods and compositions that utilize substituted 1,3-cyclopentadione compounds.

2. Description of the Related Art

Monocyte activation and adhesion to the endothelium play critical roles in inflammatory and cardiovascular diseases. The process is further complicated by hyperglycemia leading to the complications in diabetes. Both TNFa and hyperglycemia activate many genes involved in the inflammatory responses (Shanmugam N, Gae Gonzalo I T, Natarajan R. Molecular mechanisms of high glucose-induced cyclooxygenase-2 expression in monocytes. Diabetes. 53(3):795-802, 2004.).

MCP-1 is a potent chemoattractant for monocytes and plays pivotal role in early atherogenesis by promoting monocyte infiltration and adherence to the endothelium, leading to the formation atherosclerotic plaque (Charo I F, Taubman M B. Chemokines in the pathogenesis of vascular disease. Circ Res. 29; 95(9):858-66, 2004.).

Matrix metalloproteinases (MMPs) are the primary proteolytic enzymes in the extracellular space, contributing to weakening of the plaque cap via their ability to cleave the extracellular matrix (ECM) (Nagase H, Visse R, Murphy G. Structure and function of matrix metalloproteinases and TIMPs. Cardiovasc Res 2006; 69:562-73.). Atherosclerotic plaque rupture, causally related to the majority of acute coronary syndromes, commonly occurs at sites of continuous inflammation and collagen degradation (Virmani R, Kolodgie F D, Burke A P, Farb A, Schwartz S M. Lessons from sudden coronary death: a comprehensive morphological classification scheme for atherosclerotic lesions. Arterioscler Thromb Vasc Biol 2000; 20:1262-75.).

Clinical and experimental studies have implicated MMP-9 (gelatinase B) as a key determinant of atherosclerotic plaque stability (Gough P J, Gomez I G, Wille P T, Raines E W. Macrophage expression of active MMP-9 induces acute plaque disruption in apoE-deficient mice. J Clin Invest 2006; 116:59-69.; Fukuda D, Shimada K, Tanaka A, Kusuyama T, Yamashita H, Ehara S, et al. Comparison of levels of serum matrix metalloproteinase-9 in patients with acute myocardial infarction versus unstable angina pectoris versus stable angina pectoris. Am J Cardiol 2006; 97:175-80.; Blankenberg S, Rupprecht H J, Poirier O, Bickel C, Smieja M, Hafner G, et al. Plasma concentrations and genetic variation of matrix metalloproteinase 9 and prognosis of patients with cardiovascular disease. Circulation 2003; 107:1579-85.). MMP-9 principally derives from monocytes/macrophages (Chase A J, Bond M, Crook M F, Newby A C. Role of nuclear factor kappa B activation in metalloproteinase-1, -3, and -9 secretion by human macrophages in vitro and rabbit foam cells produced in vivo. Arterioscler Thromb Vasc Biol 2002; 22:765-71.; Stawowy P, Meyborg H, Stibenz D, Borges Pereira Stawowy N, Roser M, Thanabalasingam U, et al. Furin-like proprotein convertases are central regulators of the membrane type matrix metalloproteinase-promatrix metalloproteinase-2 proteolytic cascade in atherosclerosis. Circulation 2005; 111:2820-7.), the major cell type involved in the initiation, progression and complications of atherosclerosis. In MNCs MMP-9 is strongly inducible by a number of inflammatory mediators, including TNF-α (Stawowy P, Meyborg H, Stibenz D, Borges Pereira Stawowy N, Roser M, Thanabalasingam U, et al. Furin-like proprotein convertases are central regulators of the membrane type matrix metalloproteinase-promatrix metalloproteinase-2 proteolytic cascade in atherosclerosis. Circulation 2005; 111:2820-7.).

It has previously been shown that the anti-inflammatory compounds such as aspirin, glucocorticoids, and curcumin exert their effects through the inhibition of NF-κB signaling pathways (Kopp E and Ghosh S. Inhibition of NF-kappa B by sodium salicylate and aspirin. Science. 22: 270 (5244): 2017-9,1994.; De Bosscher K, Schmitz M L, Vanden Berghe W, Plaisance S, Fiers W, Haegeman G. Glucocorticoid-mediated repression of nuclear factor-kappaB-dependent transcription involves direct interference with transactivation. Proc Natl Acad Sci USA. 9; 94(25):13504-9, 1997.; Pan M H, Lin-Shiau S Y, Ho C T, Lin J H, Lin J K. Suppression of lipopolysaccharide-induced nuclear factor-kappaB activity by theaflavin-3,3′-digallate from black tea and other polyphenols through down-regulation of IkappaB kinase activity in macrophages. Biochem Pharmacol. 15; 59(4):357-67, 2000.).

Inflammatory mediators, such as TNFa activate NFkB, which regulate the expression of many genes involved in the inflammatory responses such as proinflammatory cytokines, adhesion molecules, chemokines including monocyte chemotactic protein-1 (MCP-1) (Ueda A, Ishigatsubo Y, Okubo T, Yoshimura T. Transcriptional regulation of the human monocyte chemoattractant protein-1 gene. Cooperation of two NF-kappaB sites and NF-kappaB/Rel subunit specificity. J Biol Chem. 5; 272(49):31092-9, 1997.). It has been shown that hyperglycemia activate inflammation through the activation of PKC and NF-kB signaling pathways in monocytes (Devaraj S, Venugopal S K, Singh U, Jialal I. Hyperglycemia induces monocytic release of interleukin-6 via induction of protein kinase c-{alpha} and -{beta}.Diabetes.; 54(1):85-91, 2005.; Shanmugam N, Gae Gonzalo I T, Natarajan R. Molecular mechanisms of high glucose-induced cyclooxygenase-2 expression in monocytes. Diabetes. 53(3):795-802, 2004.)

Reversible phosphorylation of proteins regulates nearly all aspects of cell physiology. Chronic dysregulation of specific kinases and phosphatases exert their effects by altering the normal phosphorylation state of intracellular proteins, giving rise to a number of disorders including cancers and inflammatory diseases. Hence, protein kinases are a major therapeutic target.

The over-activation of specific kinases is particularly associated with many diseases and as expected, certain, targeted kinase inhibitors have been shown to be efficacious in normalizing cellular physiology and bringing about remission. For example, imatinib (tyrosine kinase inhibitor) is effective in the treatment of leukemia [Savage D G, N Eng J Med 2002]; erlotinib (Epidermal Growth Factor Receptor inhibitor) for lung cancer (Reviewed in Expert Opin Pharmacother, Gridelli, 2007); and ruboxistaurin (PKC-beta II inhibitor) for reducing microvascular complications for diabetic patients (Joy S V, et al. Ruboxistaurin, a protein kinase C beta inhibitor, as an emerging treatment for diabetes microvascular complications. Ann Pharmacother. 39(10): 1693-9, 2005).

Endothelial derived Nitric Oxide (NO) is a key determinant of cardiovascular homeostasis modulating vascular endothelial responsiveness and thus regulating systemic blood pressure, vascular remodeling and angiogenesis (Moncada S, Higgs A: The L-Arginine-Nitric Oxide pathway. NEJM 1993; 329:2002-12). An important stimulus for the continuous production of NO is viscous drag related to blood flow across the endothelium. Endothelial NO synthase (eNOS) is under direct regulation by the protein kinase Akt. Shear stress and hyperglycemia through a series of mediating kinases directly activation Akt. (Dimmeler S, Fleming I, Fisslthaler B, Hermann C, Busse R, Zeiher A M. Activation of nitric oxide synthase in endothelial cells by Akt-dependent phosphorylation. Nature 1999; 399:601-5). One of these kinases which actually inhibit Akt is PKCβ. PKCβ is activated by hyperglycemia. Hyperglycemia has been directly shown to inhibit endothelial dependent vasodilation (Rammos G, Peppes V, Zakopoulos N. Transient Insulin Resistance in Normal Subjects: Acute Hyperglycemia inhibits Endothelial-Dependent Vasodilation in Normal Subjects. Metabolic Syndrome and Related Disorders 2008; 6(3):159-170.). Roboxistaurin inhibits PKCβ and thus normalizes endothelial function as measured by Flow Mediated Vasodilation (FMD) (Mehta N N, Sheetz M, Price K, Comiskey L, Amrutia S, Iqbal N, Mohler E R, Reilly M P. Selective PKC beta inhibition with roboxistaurin and endothelial function in type-2 diabetes mellitus. Cardiovasc Drugs Ther 2009; 23(1):17-24). FMD is a non-invasive ultrasonographic technique for measuring brachial artery flow physiology after an induced hypoxemia (Corretti M C et al. Guidelines for the Ultrasound Assessment of Endothelial-Dependent Flow-Mediated Vasodilation of the Brachial Artery—A Report of the International Brachial Artery Reactive Task Force. J Am Coll Cardiol 2002; 39(2): 257-65).

Previously, we reported that several compounds derived from hop cones, including humulones, lupulones, isohumulones, and reduced isohumulones (modified hop extract used as flavorings) potently inhibit lipopolysaccharide (LPS) stimulated PGE2 formation in RAW 264.7 cells (Tripp M, Darland G, Lerman R, Lukaczer D, Bland J, Babish J: Hop and modified hop extracts have potent in vitro anti-inflammatory properties. Acta Hort (ISHS) 2005, 668:217-228). Among the most active are the substituted 1,3-cyclopentadione compounds derived from hops are the tetrahydro-isoalpha acids, collectively denoted herein as denoted Meta-060 or “THIAA. Meta-060 is a modified hop extract comprised of a mixture of three related analogs, tetrahydro-iso-humulone, -cohumulone, and -adhumulone at a ratio of 49:42:9. META-060 inhibits LPS induced PGE2 production and COX-2 expression by inhibiting the NFkB signaling pathway in RAW 264.7 cells (Desai A, Konda V R, Darland G, Austin M, Prabhu K S, Bland J S, et al. META060 inhibits multiple kinases in the NF-kB pathway and suppresses LPS-mediated inflammation in vitro and ex vivo. Inflamm Res 2009).

Macrophage/monocyte activation participates pivotally in the pathophysiology of many chronic inflammatory diseases. The role of inflammation at all stages of the atherosclerotic process has become an active area of investigation, and there is a need for novel and innovative therapeutics to treat atherosclerosis. As such, the inventors have ascertained the inhibitory effects of Meta-060 on the NFkB pathway acting via kinase inhibition against key kinases involved in NFkB activation. Further, to assess specificity, the efficacy of META-060 was assessed against 85 different kinases.

Additionally, the inventors report on META-060′s ability to inhibit other inflammatory markers under NFkB regulation and associated with cardiovascular complications, including (1). TNFa mediated VCAM-1, E selectin and MCP-1 in endothelial cells (HAEC) and (2). TNFa mediated Monocyte-Endothelial interactions.

### SUMMARY OF THE INVENTION

The present invention relates generally to methods and compositions that can be used to modulate inflammatory pathways associated with endothelial function and cardiovascular complications, including (1) TNFa mediated VCAM-1, E selectin and MCP-1 in endothelial cells (HAEC) and (2) TNFa mediated Monocyte-Endothelial interactions via protein kinase modulation. More specifically, the invention relates to methods and compositions that utilize substituted 1,3-cyclopentadione compounds.

A first embodiment of the invention describes methods for improving a cardiovascular risk factor in a subject in need. The methods comprise treating the subject with a therapeutically effective amount of a substituted 1,3-cyclopentadione compound where said amount modulates the expression of cardiovascular risk factor associated marker gene expression.

A second embodiment of the invention describes methods for promoting vascular elasticity in a subject in need, where the method comprises treating the subject with a therapeutically effective amount of a substituted 1,3-cyclopentadione compound where said amount increases vascular elasticity or dilation.

A third embodiment of the invention describes compositions promoting cardiovascular health in a subject. In this embodiment the compositions comprise a therapeutically effective amount of a substituted 1,3-cyclopentadione compound wherein such amount (a) modulates the expression of cardiovascular risk associated marker gene expression; or (b) increases vascular elasticity or dilation.

### BRIEF DESCRIPTION OF THE DRAWINGS

FIG. 1 graphically depicts the effects of substituted 1,3-cyclopentadione compound s on kinase activity y for kinases regulating inflammation and provides a showing of the specificity of the substituted 1,3-cyclopentadione compounds via Gini coefficient expression.

FIG. 2 graphically depicts the effects of substituted 1,3-cyclopentadione compounds on the inhibition of selected endothelial inflammatory biomarkers.

FIG. 3 graphically depicts the inhibitory effects of substituted 1,3-cyclopentadione compounds on endothelial-monocyte interactions.

FIG. 4 depicts META-060 inhibition of THP-1 cell interactions to HAEC cells.

FIG. 5 depicts META-060 inhibition of MCP-1 expression in THP-1 cells.

FIG. 6 displays the inhibitory effect of META-060 on TNFa and LPS mediated MMP-9 levels in THP-1 cells. Panels A & B depict the respective effects of TNFa and LPS on MMP-9 levels while Panel C provides a zymograph displaying the effects on MMP-9 expression.

FIG. 7 graphically displays the inhibitory effects of META-060 on LPS mediated NFkB binding in THP-1 cells.

FIG. 8 displays the inhibitory effects of META-060 on TNFa activated genes in THP-1 cells.

### DETAILED DESCRIPTION OF THE INVENTION

The present invention relates generally to methods and compositions that can be used to modulate inflammatory pathways associated with endothelial function and cardiovascular complications, including (1) TNFa mediated VCAM-1, E selectin and MCP-1 in endothelial cells (HAEC) and (2) TNFa mediated Monocyte-Endothelial interactions via protein kinase modulation. More specifically, the invention relates to methods and compositions that utilize substituted 1,3-cyclopentadione compounds.

The patents, published applications, and scientific literature referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of recombinant DNA technology include Sambrook et al., Molecular Cloning: A Laboratory Manual, 2nd Ed., Cold Spring Harbor Laboratory Press, New York (1989); Kaufman et al., Eds., Handbook of Molecular and Cellular Methods in Biology in Medicine, CRC Press, Boca Raton (1995); McPherson, Ed., Directed Mutagenesis: A Practical Approach, IRL Press, Oxford (1991). Standard reference works setting forth the general principles of pharmacology include Goodman and Gilman's The Pharmacological Basis of Therapeutics, 11th Ed., McGraw Hill Companies Inc., New York (2006).

In the specification and the appended claims, the singular forms include plural referents unless the context clearly dictates otherwise. As used in this specification, the singular forms “a,” “an” and “the” specifically also encompass the plural forms of the terms to which they refer, unless the content clearly dictates otherwise. Additionally, as used herein, unless specifically indicated otherwise, the word “or” is used in the “inclusive” sense of “and/or” and not the “exclusive” sense of “either/or.” The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, the recitation of a numerical range for a variable is intended to convey that the invention may be practiced with the variable equal to any of the values within that range. Thus, for a variable that is inherently discrete, the variable can be equal to any integer value of the numerical range, including the end-points of the range. Similarly, for a variable that is inherently continuous, the variable can be equal to any real value of the numerical range, including the end-points of the range. As an example, a variable that is described as having values between 0 and 2, can be 0, 1 or 2 for variables that are inherently discrete, and can be 0.0, 0.1, 0.01, 0.001, or any other real value for variables that are inherently continuous.

Reference is made hereinafter in detail to specific embodiments of the invention. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention as defined by the appended claims. In the following description, numerous specific details are set forth in order to provide a thorough understanding of the present invention. The present invention may be practiced without some or all of these specific details. In other instances, well known process operations have not been described in detail, in order not to unnecessarily obscure the present invention.

Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

A first embodiment of the invention describes methods for improving a cardiovascular risk factor in a subject in need. The methods comprise treating the subject with a therapeutically effective amount of a substituted 1,3-cyclopentadione compound where said amount modulates the expression of cardiovascular risk factor associated marker gene expression.

In some aspects of this embodiment, the substituted 1,3-cyclopentadione compound is selected from the group comprising (+)-(4R,5S)-3,4-dihydroxy-2-(3-methylbutanoyl)-5-(3-methylbutyl)-4-(4-methylpentanoyl)cyclopent-2-en-1-one, (−)-(4S,5S)-3,4-dihydroxy-2-(3-methylbutanoyl)-5-(3-methylbutyl)-4-(4-methylpentanoyl)cyclopent-2-en-1-one, (+)-(4R,5S)-3,4-dihydroxy-5-(3-methylbutyl)-4-(4-methylpentanoyl)-2-(3-methylpropanoyl)cyclopent-2-en-1-one, (−)-(4S,5S)-3,4-dihydroxy-5-(3-methylbutyl)-4-(4-methylpentanoyl)-2-(3-methylpropanoyl)cyclopent-2-en-1-one, (+)-(4R,5S)-3,4-dihydroxy-2-(2-methylbutanoyl)-5-(3-methylbutyl)-4-(4-methylpentanoyl)cyclopent-2-en-1-one and (+)-(4R,5S)-3,4-dihydroxy-5-(3-methylbutyl)-4-(4-methylpentanoyl)-2-petanoylcyclopent-2-en-1-one.

In other aspects the cardiovascular risk associated marker gene being modulated is selected from the group consisting of TNFa, MCP-1, VCAM-1, MMP-3, ICAM1 and SDF1.

In yet other aspects, the method further comprises lifestyle modification or pharmaceutical treatment wherein the lifestyle modification or pharmaceutical treatment is selected from the group consisting of blood pressure reduction, cholesterol level modulation, diabetes treatment, increased exercise, inflammation, obesity and weight reduction, prothrombotic factors treatment, reduction in serum homocysteine and lipoprotein (a), serum triglyceride reduction, smoking cessation, and stress reduction.

As used herein, “improving a cardiovascular risk factor” means stabilizing, reducing or eliminating an identified cardiovascular risk factor thereby promoting improved cardiovascular health. Representative, non-limiting examples of cardiovascular risk factors include tobacco usage, elevated blood pressure, elevated serum total (and LDL) cholesterol, diabetes, advancing age, obesity, physical inactivity, family history of premature cardiac heart disease, elevated serum triglycerides, small LDL particles, elevated serum homocysteine or lipoprotein (a), prothrombotic factors (e.g., fibrinogen), and inflammatory factors (e.g., C-reactive protein).

The phrase “modulates the expression of cardiovascular risk factor associated marker gene expression” means the up or down regulation of the expression of the gene, or it's associated gene product production or activity. Non-limiting representative examples of genes identified or associated with various aspects of cardiovascular risk factors include AQP1, B3GAT3, BCL3, BTG2, C1ORF106, C1ORF38, C1ORF38, CACNA1A, CCDC75, CCL2, CCL8, CCND1, CD40, CD44, CD86, CLIP2, DDX58, DKFZP434H1419, DSC3, EHD1, EIF2AK2, FAM105A, FGD2, FKBP5, G3BP1, GPR153, HTR4, ICAM1, ICOSLG, IFI44, IFI44L, IFIH1, IFIT1, IFIT2, IFIT3, IGKC, IGKV1-5, IKBKE, IKZF1, IL10RA, IL18RAP, IL1B, IL7R, IRF7, ISG15, KIAA1731, KRT17, LHX, LIMD2, LTB, MARCKSL1, MCP-1, MMP3, MMP14, MMP9, MX1, MX2, NA, NAB1, NAB2, NOD2, NPTX1, OAS1, OAS2, OAS3, OASL, PDE4B, PIP5K1B, PRKCH, PTGIR, PTPN14, RASA, RASA4, RASSF4, REC8, RIN3, RSAD2, SAFB2, SAMD9, SDF1, SEMA4C, SH3TC1, SH3TC, SIGLEC1, SLAMF8, SLC16A3, SLC1A3, SP110, SPI1, SPIB, SSPO, STAT1, TAP1, TAPBP, THBD, TNFα, TRAC, TRIM22, UBE2B, UBQLN4, UBQLN4P, UST, VCAM1, XAF1, ZFP2, ZMIZ2, and ZNF710.

In some aspects, the lifestyle modification or pharmaceutical treatment is selected from the group consisting of blood pressure reduction, cholesterol level modulation, diabetes treatment, increased exercise, inflammation, obesity and weight reduction, prothrombotic factors treatment, reduction in serum homocysteine and lipoprotein (a), serum triglyceride reduction, smoking cessation, and stress reduction.

As used herein, the phrase “lifestyle modification” means those activities which an individual may undertake to improve cardiovascular risk factors absent or in conjunction with a pharmaceutical treatment. Representative examples of lifestyle modification include, without limitation, diet modifications for weight reduction, blood pressure or cholesterol control; increased exercise for stress relief or weight reduction; or smoking cessation. “Pharmaceutical treatment”, as used herein, refers to those agents obtained or prescribed by a health care provider which may be used in lieu of, or in conjunction with, lifestyle modifications to promote improved cardiovascular risk factors.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or compounds, but may also include additional features or compounds.

As used herein, the terms “derivatives” or a matter “derived” refer to a chemical substance related structurally to another substance and theoretically obtainable from it, i.e. a substance that can be made from another substance. Derivatives can include compounds obtained via a chemical reaction.

The term “pharmaceutically acceptable” is used in the sense of being compatible with the other ingredients of the compositions and not deleterious to the recipient thereof.

As used herein, “tetrahydro-isohumulone” shall refer to the cis and trans forms of (+)-(4R,5S)-3,4-dihydroxy-2-(3-methylbutanoyl)-5-(3-methylbutyl)-4-(4-methylpentanoyl)cyclopent-2-en-1-one and (−)-(4S,5S)-3,4-dihydroxy-2-(3-methylbutanoyl)-5-(3-methylbutyl)-4-(4-methylpentanoyl)cyclopent-2-en-1-one respectively.

“Tetrahydro-isocohumulone”, as used herein refers to the cis and trans forms of (+)-(4R,5S)-3,4-dihydroxy-5-(3-methylbutyl)-4-(4-methylpentanoyl)-2-(3-methylpropanoyl)cyclopent-2-en-1-one and (−)-(4S,5S)-3,4-dihydroxy-5-(3-methylbutyl)-4-(4-methylpentanoyl)-2-(3-methylpropanoyl)cyclopent-2-en-1-one respectively.

“Tetrahydro-adhumulone” shall be used herein to refer to the cis and trans forms of (+)-(4R,5S)-3,4-dihydroxy-2-(2-methylbutanoyl)-5-(3-methylbutyl)-4-(4-methylpentanoyl)cyclopent-2-en-1-one and (+)-(4R,5S)-3,4-dihydroxy-5-(3-methylbutyl)-4-(4-methylpentanoyl)-2-petanoylcyclopent-2-en-1-one respectively.

As used herein, “tetrahydro-isoalpha acid” (see Table 1) or “Meta060” refers to any mixture of one or more of tetrahydro-adhumulone, tetrahydro-isocohumulone and tetrahydro-isohumulone.

TABLE 1
Tetrahydroisoalpha acids

| Chemical Name | Synonym | Structure |
|---|---|---|
| (4R,5S)-3,4-dihydroxy-2-(3- methylbutanoyl)-5-(3-methylbutyl)-4-(4- methylpentanoyl)cyclopent-2-en-1-one | tetrahydro cis n iso-alpha acid | 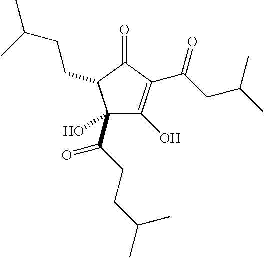 |
| (4S,5S)-3,4-dihydroxy-2-(3- methylbutanoyl)-5-(3-methylbutyl)-4-(4- methylpentanoyl)cyclopent-2-en-1-one | tetrahydro trans n iso-alpha acid | 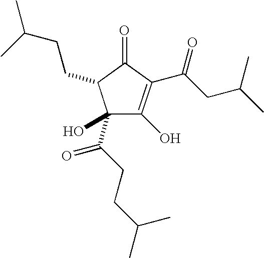 |
| (4S,5R)-3,4-dihydroxy-2-(3- methylbutanoyl)-5-(3-methylbutyl)-4-(4- methylpentanoyl)cyclopent-2-en-1-one | tetrahydro cis n iso-alpha acid | 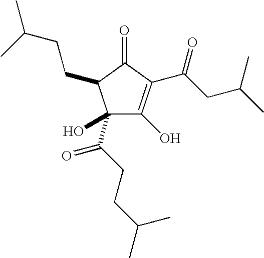 |
| (4R,5R)-3,4-dihydroxy-2-(3- methylbutanoyl)-5-(3-methylbutyl)-4-(4- methylpentanoyl)cyclopent-2-en-1-one | tetrahydro trans n iso-alpha acid | 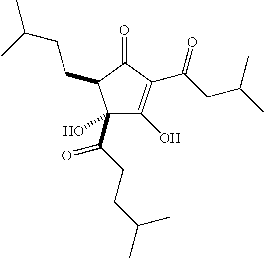 |
| (4R,5S)-3,4-dihydroxy-5-(3-methylbutyl)-4- (4-methylpentanoyl)-2-(3- methylpropanoyl)cyclopent-2-en-1-one | tetrahydro cis co iso-alpha acid | 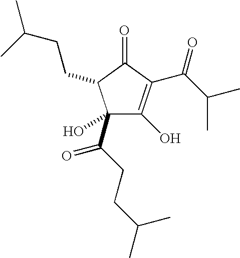 |
| (4S,5S)-3,4-dihydroxy-5-(3-methylbutyl)-4- (4-methylpentanoyl)-2-(3- methylpropanoyl)cyclopent-2-en-1-one | tetrahydro trans co iso-alpha acid | 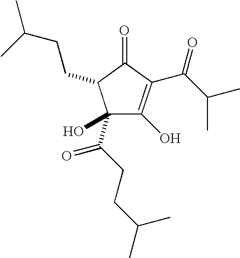 |
| (4S,5R)-3,4-dihydroxy-5-(3-methylbutyl)-4- (4-methylpentanoyl)-2-(3- methylpropanoyl)cyclopent-2-en-1-one | tetrahydro cis co iso-alpha acid | 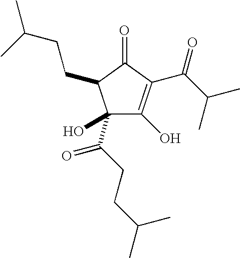 |
| (4R,5R)-3,4-dihydroxy-5-(3-methylbutyl)-4- (4-methylpentanoyl)-2-(3- methylpropanoyl)cyclopent-2-en-1-one | tetrahydro trans co iso-alpha acid | 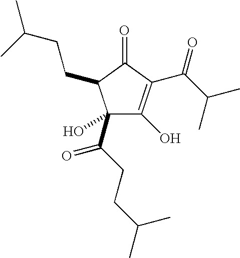 |
| (4R,5S)-3,4-dihydroxy-2-(2- methylbutanoyl)-5-(3-methylbutyl)-4-(4- methylpentanoyl)cyclopent-2-en-1-one | tetrahydro cis ad iso-alpha acid | 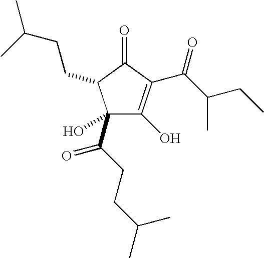 |
| (4S,5S)-3,4-dihydroxy-2-(2- methylbutanoyl)-5-(3-methylbutyl)-4-(4- methylpentanoyl)cyclopent-2-en-1-one | tetrahydro trans ad iso-alpha acid | 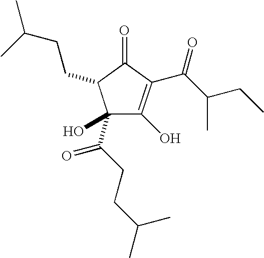 |
| (4S,5R)-3,4-dihydroxy-2-(2- methylbutanoyl)-5-(3-methylbutyl)-4-(4- methylpentanoyl)cyclopent-2-en-1-one | tetrahydro cis ad iso-alpha acid | 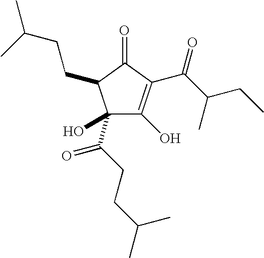 |
| (4R,5R)-3,4-dihydroxy-2-(2- methylbutanoyl)-5-(3-methylbutyl)-4-(4- methylpentanoyl)cyclopent-2-en-1-one | tetrahydro trans ad iso-alpha acid | 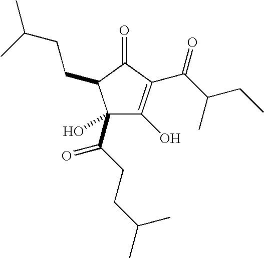 |

As used herein, “compounds” may be identified either by their chemical structure, chemical name, or common name. When the chemical structure and chemical or common name conflict, the chemical structure is determinative of the identity of the compound. The compounds described herein may contain one or more chiral centers and/or double bonds and therefore, may exist as stereoisomers, such as double-bond isomers (i.e., geometric isomers), enantiomers or diastereomers. Accordingly, the chemical structures depicted herein encompass all possible enantiomers and stereoisomers of the illustrated or identified compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof Accordingly, the chemical structures depicted herein encompass all possible tautomeric forms of the illustrated or identified compounds. The compounds described also encompass isotopically labeled compounds where one or more atoms have an atomic mass different from the atomic mass conventionally found in nature. Examples of isotopes that may be incorporated into the compounds of the invention include, but are not limited to, ²H, ³H, ¹³C, ¹⁴C, ¹⁵N, ¹⁸O, ¹⁷O, etc. Compounds may exist in unsolvated forms as well as solvated forms, including hydrated forms and as N-oxides. In general, compounds may be hydrated, solvated or N-oxides. Certain compounds may exist in multiple crystalline or amorphous forms. Also contemplated within the scope of the invention are congeners, analogs, hydrolysis products, metabolites and precursor or prodrugs of the compound. In general, unless otherwise indicated, all physical forms are equivalent for the uses contemplated herein and are intended to be within the scope of the present invention.

Compounds according to the invention may be present as salts. In particular, pharmaceutically acceptable salts of the compounds are contemplated. A “pharmaceutically acceptable salt” of the invention is a combination of a compound of the invention and either an acid or a base that forms a salt (such as, for example, the magnesium salt, denoted herein as “Mg” or “Mag”) with the compound and is tolerated by a subject under therapeutic conditions. In general, a pharmaceutically acceptable salt of a compound of the invention will have a therapeutic index (the ratio of the lowest toxic dose to the lowest therapeutically effective dose) of 1 or greater. The person skilled in the art will recognize that the lowest therapeutically effective dose will vary from subject to subject and from indication to indication, and will thus adjust accordingly.

A second embodiment of the invention describes methods for promoting vascular elasticity in a subject in need, the method comprising treating the subject with a therapeutically effective amount of a substituted 1,3-cyclopentadione compound wherein said amount increases vascular elasticity or dilation. In some aspects of this embodiment the substituted 1,3-cyclopentadione compound is selected from the group comprising (+)-(4R,5S)-3,4-dihydroxy-2-(3-methylbutanoyl)-5-(3-methylbutyl)-4-(4-methylpentanoyl)cyclopent-2-en-1-one, (−)-(4S,5S)-3,4-dihydroxy-2-(3-methylbutanoyl)-5-(3-methylbutyl)-4-(4-methylpentanoyl)cyclopent-2-en-1-one, (+)-(4R,5S)-3,4-dihydroxy-5-(3-methylbutyl)-4-(4-methylpentanoyl)-2-(3-methylpropanoyl)cyclopent-2-en-1-one, (−)-(4S,5S)-3,4-dihydroxy-5-(3-methylbutyl)-4-(4-methylpentanoyl)-2-(3-methylpropanoyl)cyclopent-2-en-1-one, (+)-(4R,5S)-3,4-dihydroxy-2-(2-methylbutanoyl)-5-(3-methylbutyl)-4-(4-methylpentanoyl)cyclopent-2-en-1-one and (+)-(4R,5S)-3,4-dihydroxy-5-(3-methylbutyl)-4-(4-methylpentanoyl)-2-petanoylcyclopent-2-en-1-one.

Another embodiment describes a composition for promoting cardiovascular health in a subject in need, where the composition comprises a therapeutically effective amount of a substituted 1,3-cyclopentadione compound wherein said amount (a) modulates the expression of cardiovascular risk associated marker gene expression; or (b) increases vascular elasticity or dilation.

In some aspects, the substituted 1,3-cyclopentadione compound is selected from the group comprising (+)-(4R,5S)-3,4-dihydroxy-2-(3-methylbutanoyl)-5-(3-methylbutyl)-4-(4-methylpentanoyl)cyclopent-2-en-1-one, (−)-(4S,5S)-3,4-dihydroxy-2-(3-methylbutanoyl)-5-(3-methylbutyl)-4-(4-methylpentanoyl)cyclopent-2-en-1-one, (+)-(4R,5S)-3,4-dihydroxy-5-(3-methylbutyl)-4-(4-methylpentanoyl)-2-(3-methylpropanoyl)cyclopent-2-en-1-one, (−)-(4S,5S)-3,4-dihydroxy-5-(3-methylbutyl)-4-(4-methylpentanoyl)-2-(3-methylpropanoyl)cyclopent-2-en-1-one, (+)-(4R,5S)-3,4-dihydroxy-2-(2-methylbutanoyl)-5-(3-methylbutyl)-4-(4-methylpentanoyl)cyclopent-2-en-1-one and (+)-(4R,5S)-3,4-dihydroxy-5-(3-methylbutyl)-4-(4-methylpentanoyl)-2-petanoylcyclopent-2-en-1-one.

In yet other aspects of this embodiment the composition further comprises a pharmaceutically acceptable excipient wherein the pharmaceutically acceptable excipient is selected from the group consisting of coatings, isotonic and absorption delaying agents, binders, adhesives, lubricants, disintergrants, coloring agents, flavoring agents, sweetening agents, absorbants, detergents, and emulsifying agents. Additionally, in other aspects the composition further comprises one or more members selected from the group consisting of antioxidants, vitamins, minerals, proteins, fats, and carbohydrates.

In other aspects of this embodiment, the substituted 1,3-cyclopentadione compound utilized in the composition is greater than 50% pure, preferably greater than 75% pure, more preferably greater than 90% pure and most preferably greater than 95% pure for the identified stereoisomer (the remaining amount being the alternative isomeric form). In some aspects the composition comprises from about 50 mg to 10,000 mg, preferably 100 mg to 8,000 mg, or most preferably 150 mg to 6,000 mg of the substituted 1,3-cyclopentadione compound per dosage.

The compounds according to the invention are optionally formulated in a pharmaceutically acceptable vehicle with any of the well known pharmaceutically acceptable carriers, including diluents and excipients [see Remington's Pharmaceutical Sciences, 18th Ed., Gennaro, Mack Publishing Co., Easton, Pa. 1990 and Remington: The Science and Practice of Pharmacy, Lippincott, Williams & Wilkins, 1995]. While the type of pharmaceutically acceptable carrier/vehicle employed in generating the compositions of the invention will vary depending upon the mode of administration of the composition to a mammal, generally pharmaceutically acceptable carriers are physiologically inert and non-toxic. Formulations of compositions according to the invention may contain more than one type of compound of the invention), as well as any other pharmacologically active ingredient useful for the treatment of the symptom/condition being treated.

The compounds of the present invention may be provided in a pharmaceutically acceptable vehicle using formulation methods known to those of ordinary skill in the art. The compositions of the invention can be administered by standard routes, though preferably administration is by inhalation routes. The compositions of the invention include those suitable for oral, inhalation, rectal, ophthalmic (including intravitreal or intracameral), nasal, topical (including buccal and sublingual), vaginal, or parenteral (including subcutaneous, intramuscular, intravenous, intradermal, and intratracheal). In addition, polymers may be added according to standard methodologies in the art for sustained release of a given compound.

Formulations suitable for administration by inhalation include formulations that can be dispensed by inhalation devices known to those in the art. Such formulations may include carriers such as powders and aerosols. The present invention encompasses liquid and powdered compositions suitable for nebulization and intrabronchial use, or aerosol compositions administered via an aerosol unit dispensing metered doses (“MDI”).The active ingredient may be formulated in an aqueous pharmaceutically acceptable inhalant vehicle, such as, for example, isotonic saline or bacteriostatic water and other types of vehicles that are well known in the art. The solutions are administered by means of a pump or squeeze-actuated nebulized spray dispenser, or by any other conventional means for causing or enabling the requisite dosage amount of the liquid composition to be inhaled into the patient's lungs. Powder compositions containing the anti-inflammatory compounds of the present invention include, by way of illustration, pharmaceutically acceptable powdered preparations of the active ingredient thoroughly intermixed with lactose or other inert powders acceptable for intrabronchial administration. The powder compositions can be administered via a dispenser, including, but not limited to, an aerosol dispenser or encased in a breakable capsule which may be inserted by the patient into a device that punctures the capsule and blows the powder out in a steady stream. Aerosol formulations for use in the subject method typically include propellants, surfactants, and co-solvents and may be filled into conventional aerosol containers that are closed by a suitable metering valve.

Formulations of compositions of the present invention suitable for nasal administration, wherein the carrier is a solid, include a coarse powder having a particle size, for example, in the range of 20 to 500 microns which is administered in the manner in which snuff is administered, i.e., by rapid inhalation through the nasal passage from a container of the powder held close up to the nose. Suitable formulations, wherein the carrier is a liquid, for administration, for example via a nasal spray, aerosol, or as nasal drops, include aqueous or oily solutions of the compound of the invention.

For oral administration, the compositions of the invention may be presented as discrete units such as capsules, caplets, gelcaps, cachets, pills, or tablets each containing a predetermined amount of the active ingredient as a powder or granules; as a solution or a suspension in an aqueous liquid or a non-aqueous liquid; or as an oil-in-water liquid emulsion or a water-in-oil emulsion and as a bolus, etc. Alternately, administration of a composition of all of the aspects of the present invention may be effected by liquid solutions, suspensions or elixirs, powders, lozenges, micronized particles and osmotic delivery systems.

Formulations of compositions according to the aspects of the present invention suitable for parenteral administration include aqueous and non-aqueous sterile injection solutions which may contain antioxidants, stabilizers, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) conditions requiring only the addition of the sterile liquid carrier, for example, water for injections, immediately prior to use. Extemporaneous injection solutions and suspensions may be prepared from sterile powders, granules and tablets of the kind previously described.

A tablet may be made by compression or molding, optionally with one or more accessory ingredients. Compressed tablets may be prepared by compressing, in a suitable machine, the active ingredient in a free-flowing form such as a powder or granules, optionally mixed with a binder, lubricant, inert diluent, preservative, surface active or dispersing agent. Molded tablets may be made by molding, in a suitable machine, a mixture of the powdered compound moistened with an inert liquid diluent. The tablets may be optionally coated or scored and may be formulated to provide a slow or controlled release of the active ingredient therein.

Formulations of compositions of the present invention for rectal administration may be prepared as a suppository with a suitable base comprising, such as, for example, cocoa butter.

Formulations of compositions of the present invention suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier. Formulations of compositions of the present invention suitable for topical administration to the skin may be presented as ointments, creams, gels, lotions and pastes comprising the ingredient to be administered in a pharmaceutical acceptable carrier. A topical delivery system contemplated is a transdermal patch containing the ingredient to be administered.

Formulations of compositions according to the aspects of the present invention suitable for vaginal administration may be presented as pessaries, suppositories, tampons, creams, gels, pastes, foams or spray formulations containing in addition to the compound of the invention such pharmaceutically acceptable carriers as are known in the art to be appropriate.

The term “modulate” or “modulation” is used herein to mean the up or down regulation of expression or activity of the gene by a compound, ingredient, etc., to which it refers.

As used herein, the term “protein kinase” represents transferase class enzymes that are able to transfer a phosphate group from a donor molecule to an amino acid residue of a protein. See Kostich, M., et al., Human Members of the Eukaryotic Protein Kinase Family, Genome Biology 3(9):research0043.1-0043.12, 2002 herein incorporated by reference in its entirety, for a detailed discussion of protein kinases and family/group nomenclature.

The methods and compositions of the present invention are intended for use with any mammal that may experience the benefits of the methods of the invention. Foremost among such mammals are humans, although the invention is not intended to be so limited, and is applicable to veterinary uses. Thus, in accordance with the invention, “mammals” or “mammal in need” include humans as well as non-human mammals, particularly domesticated animals including, without limitation, cats, dogs, and horses.

As used herein, by “treating” is meant reducing, preventing, and/or reversing the symptoms in the individual to which a compound of the invention has been administered, as compared to the symptoms of an individual not being treated according to the invention. A practitioner will appreciate that the compounds, compositions, and methods described herein are to be used in concomitance with continuous clinical evaluations by a skilled practitioner (physician or veterinarian) to determine subsequent therapy. Hence, following treatment the practitioners will evaluate any improvement in reducing cardiovascular risk factors or associated dyregularities according to standard methodologies. Such evaluation will aid and inform in evaluating whether to increase, reduce or continue a particular treatment dose, mode of administration, etc.

It will be understood that the subject to which a compound of the invention is administered need not suffer from a specific traumatic state. Indeed, the compounds of the invention may be administered prophylactically, prior to any development of symptoms. The term “therapeutic,” “therapeutically,” and permutations of these terms are used to encompass therapeutic, palliative as well as prophylactic uses. Hence, as used herein, by “treating or alleviating the symptoms” is meant reducing, preventing, and/or reversing the symptoms of the individual to which a compound of the invention has been administered, as compared to the symptoms of an individual receiving no such administration.

The term “therapeutically effective amount” is used to denote treatments at dosages effective to achieve the therapeutic result sought. Furthermore, one of skill will appreciate that the therapeutically effective amount of the compound of the invention may be lowered or increased by fine tuning and/or by administering more than one compound of the invention, or by administering a compound of the invention with another compound. See, for example, Meiner, C. L., “Clinical Trials: Design, Conduct, and Analysis,” Monographs in Epidemiology and Biostatistics, Vol. 8 Oxford University Press, USA (1986). The invention therefore provides a method to tailor the administration/treatment to the particular exigencies specific to a given mammal. As illustrated in the following examples, therapeutically effective amounts may be easily determined for example empirically by starting at relatively low amounts and by step-wise increments with concurrent evaluation of beneficial effect.

It will be appreciated by those of skill in the art that the number of administrations of the compounds according to the invention will vary from patient to patient based on the particular medical status of that patient at any given time including other clinical factors such as age, weight and condition of the mammal and the route of administration chosen.

As used herein, “symptom” denotes any sensation or change in bodily function that is experienced by a patient and is associated with a particular disease, i.e., anything that accompanies “X” and is regarded as an indication of “X”'s existence. It is recognized and understood that symptoms will vary from disease to disease or condition to condition. By way of non-limiting examples, symptoms associated with autoimmune disorders include fatigue, dizziness, malaise, increase in size of an organ or tissue (for example, thyroid enlargement in Grave's Disease), or destruction of an organ or tissue resulting in decreased functioning of an organ or tissue (for example, the islet cells of the pancreas are destroyed in diabetes).

“Inflammation” or “inflammatory condition” as used herein refers to a local response to cellular injury that is marked by capillary dilatation, leukocytic infiltration, redness, heat, pain, swelling, and often loss of function and that serves as a mechanism initiating the elimination of noxious agents and of damaged tissue. Representative symptoms of inflammation or an inflammatory condition include, if confined to a joint, redness, swollen joint that's warm to touch, joint pain and stiffness, and loss of joint function. Systemic inflammatory responses can produce “flu-like” symptoms, such as, for instance, fever, chills, fatigue/loss of energy, headaches, loss of appetite, and muscle stiffness.

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein.

### Examples

### Example 1

The purpose of this Example was to determine the effects of substituted 1,3-cyclopentadione compounds on protein kinase activity, especially that associated with the expression of selected cardiovascular risk associated markers and, additionally, on monocyte-endothelial cell interactions.

The inhibition of META-060 on in vitro kinase activity: In a final reaction volume of 25 μl the kinase of interest (5-10 mU) was incubated with the specific buffer and peptide substrate, 10 mM MgAcetate and [γ-33P-ATP]. The reaction was initiated by the addition of the MgATP mix. After incubation for 40 minutes at room temperature, the reaction was stopped by the addition of 5 μl of a 3% phosphoric acid solution. 10 μl of the reaction was then spotted onto a P30 filtermat and washed three times for 5 minutes in 50 mM phosphoric acid and once in methanol prior to drying and scintillation counting.

The inhibition of META-060 on TNFa induced VCAM1, E-selectin and MCP-1 in HAEC cells. HAEC cells were pre-incubated with various concentrations of META-060 (10, 5 and 1 mg/ml) for 1 hr and stimulated with TNFa (10 ng/ml) for 8 hrs. VCAM-/E-Selectin levels were measured by cell based ELISA method using antibodies for VCAM-1 and E-selectin and fold induction was calculated over unstimulated (control) cells. For MCP-1, the cell media were measured using human MCP-1 immunoassay kits (R&D Systems). Data represent Mean±SD of 8 individual samples.

The inhibition of META-060 on TNFa induced Monocyte-Endothelial interactions: The adhesion of fluorescently labeled human monocytic (THP-1) cells to confluent monolayers of HAECs was measured by using a microplate-based assay. HAEC or THP-1 cells were pre-incubated with META-060 (10 mg/ml) and Parthenolide (10 mM) for 1 hr and stimulated with TNFa (10 ng/ml) for 8 hrs. THP-1 cells were labeled with the fluorescent dye BCECF-AM (1 μmM final concentration; Sigma) in HBSS medium for 30 min at 4° C. Then cells were washed twice with (37° C.) HBSS medium and resupended in EGM2 medium. 100,000 THP-1 cells were added to each well (96 well plate) and incubated for 30 min. Unbound THP-1 cells were washed twice with PBS by centrifugation by putting plate upside down with seal. Standard curve for BCECF-AM labeled THP-1 cells were generated. 200 μl of PBS were added to each well, and fluorescence was measured using Vector2 (Perkin Elmer) fluorescent plate reader (excitation: 485 nm; emission: 535 nm). THP-1 stimulation (A). THP-1 cells without stimulation, (B). THP-1 cell with TNFa, (C) THP-1 cells with parthenolide (10 ug/ml) and TNFa (D) THP-1 cells with META-060 (10 ug/ml) and TNFa. Data represent Mean±SD of 6 individual samples.

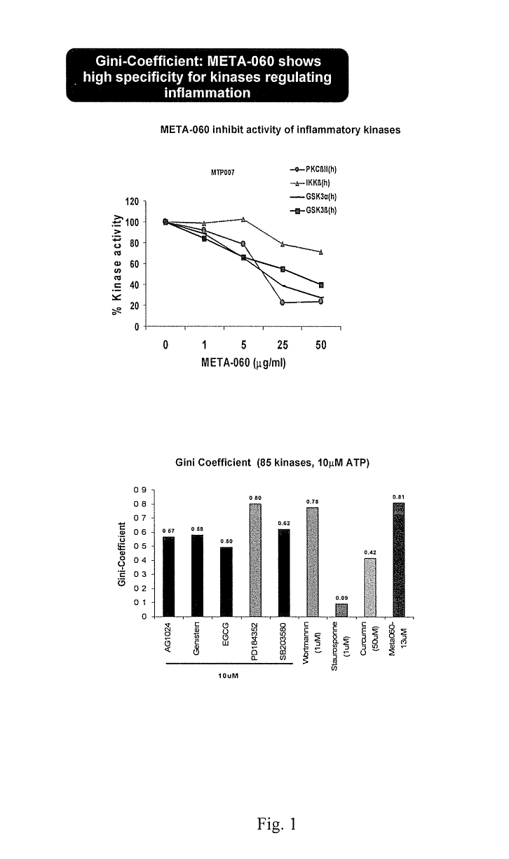

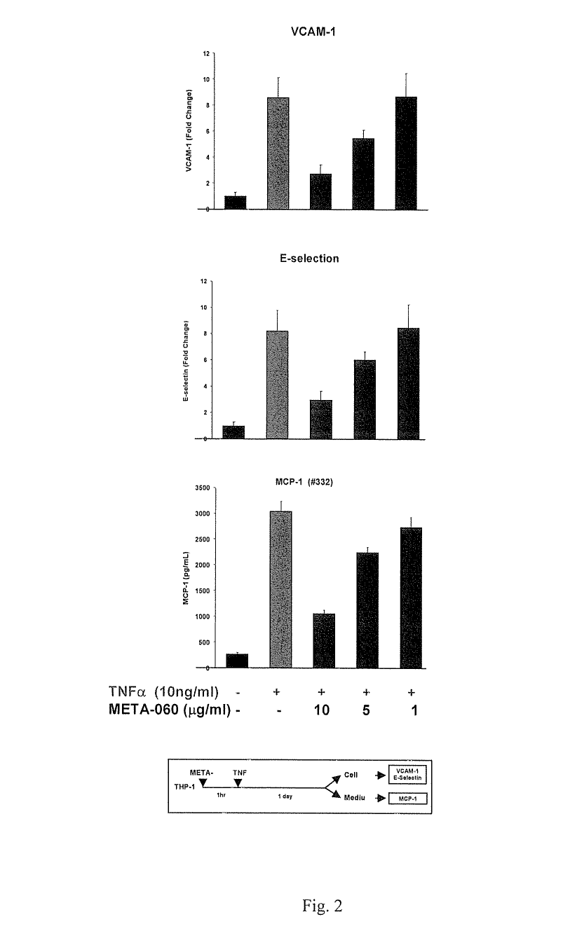

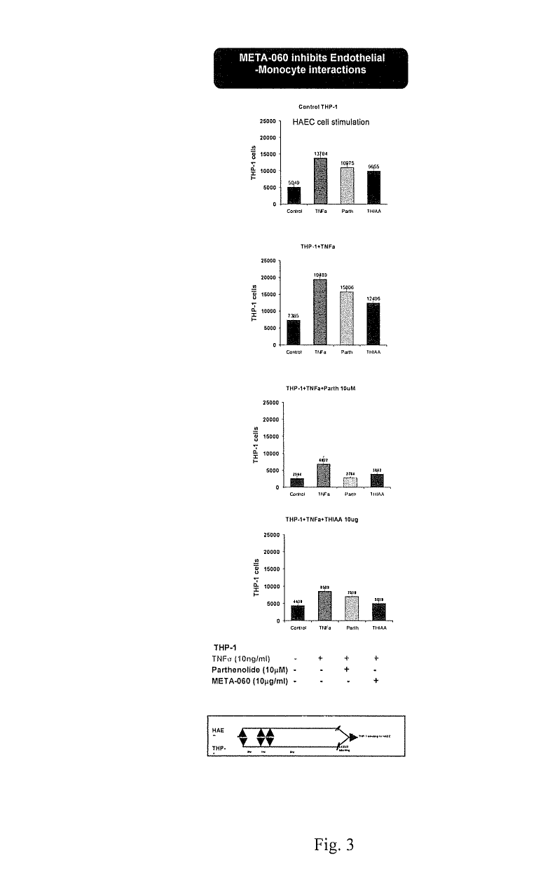

Results—The results are presented in FIGS. 1-3 and Table 2 below.

TABLE 2
Gini Coefficients and Hit Rate (>50% Inhibition) for 40 inhibitors against
85 Kinases (see Supporting Information) at 10 iM(G10)

| Inhibitor | Concn (uM) | G₁₀ | hit rate |
|---|---|---|---|
| AG1024 | 10 | 0.568 | 11 |
| AG1296 | 10 | 0.498 | 4 |
| AG1478 | 10 | 0.500 | 25 |
| AG18 | 10 | 0.680 | 1 |
| AG183 | 10 | 0.460 | 14 |
| AG538 | 10 | 0.417 | 17 |
| Alsterpaullone | 1 | 0.633 | 22 |
| Calphostin C | 10 | 0.606 | 2 |
| Cdk2/5 inh. | 10 | 0.555 | 7 |
| Curcumin | 50 | 0.417 | 38 |
| EGCG | 10 | 0.495 | 21 |
| Genistein | 10 | 0.582 | 0 |
| H89 | 10 | 0.442 | 34 |
| HA1077 | 10 | 0.650 | 19 |
| Herbimycin A | 10 | 0.534 | 0 |
| Hispidin | 10 | 0.790 | 2 |
| Indirubin | 10 | 0.291 | 52 |
| JNK inh II | 10 | 0.445 | 35 |
| K252c | 10 | 0.236 | 58 |
| KT5720 | 1 | 0.423 | 33 |
| Lavendustin A | 1 | 0.726 | 0 |
| Lavendustin B | 1 | 0.515 | 0 |
| LY294002 | 50 | 0.619 | 6 |
| Olomoucin | 50 | 0.556 | 13 |
| PD153035 | 0.001 | 0.616 | 0 |
| PD184352 | 10 | 0.802 | 1 |
| PP1 analogue | 1 | 0.758 | 15 |
| PP2 | 1 | 0.722 | 14 |
| PP3 | 1 | 0.634 | 0 |
| Ro31-8220 | 1 | 0.432 | 39 |
| Roscovitine | 10 | 0.744 | 10 |
| Rottlerin | 10 | 0.595 | 3 |
| SB202190 | 10 | 0.553 | 29 |
| SB203580 | 10 | 0.621 | 17 |
| ST638 | 10 | 0.568 | 14 |
| Staurosporine | 1 | 0.093 | 78 |
| SU6656 | 1 | 0.607 | 19 |
| Wortmannin | 1 | 0.775 | 0 |
| Y27632 | 10 | 0.628 | 13 |
| ZM336372 | 10 | 0.635 | 11 |

Graczyk PP, Journal of Medicinal Chemistry, 2007

The data presented show that Meta-060: a) inhibits TNFa mediated MCP-1, VCAM-1 and E-selectin expression in endothelial cells; b) inhibits TNFa mediated THP-1-HAEC cell interactions; c) inhibits kinases involved in inflammatory signaling pathways, such as, PKCbII, NF-kB, PI3K and GSK3; and d) Gini-coefficient value of 0.81 at 13 uM concentration, suggesting that META-060 shows high specificity for kinase inhibition.

The development of kinase inhibitors is complicated by their relative lack of specificity [Bain, Biochem J 2003, Graczek 2007] resulting in off-target side effects. In a review article, Graczek proposed the Gini-coefficient (0=lack specificity, 1=highest specificity), as a method to predict the specificity of kinase inhibitors against a panel of kinases. Forty commercially available inhibitors were tested for the inhibition of 85 kinases with Gini-coefficient ranging from 0.1 to 0.8. Here we add for comparison.

### Example 2

The purpose of this study was to evaluate the effect of substituted 1,3-cyclopentadione compounds on monocyte-endothelial interactions, expression of MCP-1 and MMP-9 levels in monocytic cells, THP-1. Additionally evaluated was the expression of various inflammatory genes in TNFa activated THP-1 cells and in vitro kinase screening of over 250 kinases in cell free enzyme assays

THP-1 and HAEC cell interactions (A). Human monocytic cell line, THP-1 were incubated with Low (5 mM) and High (25 mM) glucose for eight days. THP-1 cells were treated with THIAA for 8 hrs. B. THP-1 cells were activated with TNFa for 8 hrs in the presence and absence of test compound for 1 hr. Cells were labeled with BCECF for 30 min and added on monolayers of human endothelial cells (HAEC) for 30 min. Number of THP-1 cells bound to the wells were measured using standard curve from BCECF labeled THP-1 cells and fold induction was calculated from average of eight samples.

MCP-1 Expression: The inhibition of META-060 on TNFa (10 ng/ml), LPS (1 ug/ml) and cytokines (TNFa, Il-1b and IFNg, 10 ng/ml each) induced MCP-1 production in THP-1 cells. Cells were pre-incubated with various concentrations of META-060 for 1 hr and stimulated with TNFa (10 ng/ml) for 24 hrs. MCP-1 levels were measured using human MCP-1 immunoassay kits (R&D Systems). Data represent Mean±SD of 8 samples.

MMP-9 Expression: The inhibition of META-060 on TNFa (10 ng/ml) and LPS (1 ug/ml) induced MMP-9 production in THP-1 cells. Cells were pre-incubated with various concentrations of META-060 for 1 hr and stimulated with A. TNFa (10 ng/ml) or (B) LPS (1 ug/ml) for 24 hrs. MMP-9 levels in the medium were measured using human MMP-9 immunoassay kits (GE Healthcare). Data represent Mean±SD of a representative experiment. For MMP-9 activity (C) LPS conditioned medium was mixed 1:1 with Novex buffer (Invitrogen) and electrophoresed in 10% SDS-PAGE containing 0.1% gelatin. Gels were renaturated by exchanging SDS for Triton X-100 (2.5%), followed by 24 h incubation at 37° C. in activation buffer (50 mmol/L Tris, pH 7.6; 5 mmol/L CaCl2; 0.2 mol/L NaCl and 0.02% Brij). Gels were subsequently stained with Coomassie staining solution (0.5% Coomassie R250; 30% MeOH; 10% acetic acid) for 2 h, followed by destaining (50% MeOH and 10% acetic acid).

Electrophoretic mobility shift assays (EMSA): THP-1 cells were pre-incubated with test compounds for 1 hr and stimulated with LPS (1 μg/ml) for 2 hrs. Nuclear extracts were prepared essentially as described by Dignam, et al [Nucl. Acids. Res 11:1475-1489) and NF-kB binding to DNA was assessed using electrophoretic mobility shift assay with labeled NF-kB consensus DNA probe (5′AGTTGAGGGGACTTTCCCAGGC).

Regulation of protein phosphorylation in THP-1 cells: THP-1 cells were seeded in 6 well plate, pre-incubated in the presence and absence of META-060 (20 ug/ml)) for 1 hr and stimulated with TNFa (10 ng/ml) or LPS (10 ug/ml) for 1 hr. Cell lysates were prepared as per the instructions from user manual from MILLIPLEX MAP technology (Millipore, Billerica, Mass.). Multi-Pathway Signaling kit is used to detect changes in phosphorylated Erk/MAP kinase 1/2 (Thr185/Tyr187), STAT3 (Ser727), STAT5A/B (Tyr694/699), JNK (Thr183/Tyr185), p70 S6 kinase (Thr412) and p38 (Thr180/Tyr182) in cell lysates using the Luminex® system. Cell lysate (25 ug/well) were used and phosphoproteins were measured in the lysates as per the instructions and fold inductions were calculated using unstimulated samples as a control (Table 3 & 4).

TABLE 3
Effect of Meta060 on TNFa mediated protein
phosphorylation in THP-1 cells

| | | | Meta060 (ug/ml) + TNFa | | |
|---|---|---|---|---|---|
| | Control | TNFa | (10 ng/ml) | | |
| Phosphoproteins | (Unstimulated) | (10 ng/ml) | 20 ug | 10 ug | 5 ug |
| Erk1/2 | 1.0 | 1.5 | 1.4 | 1.6 | 1.6 |
| STAT3 | 1.0 | 1.4 | 1.2 | 1.2 | 1.4 |
| JNK | 1.0 | 1.3 | 1.0 | 0.8 | 1.1 |
| p70 S6K | 1.0 | 1.3 | 1.1 | 1.0 | 1.6 |
| STAT5A/B | 1.0 | 0.8 | 0.7 | 0.4 | 1.3 |
| p38 | 1.0 | 2.0 | 1.5 | 1.4 | 1.6 |

TABLE 4
Effect of Meta060 on LPS mediated protein
phosphorylation in THP-1 cells.

| | | | Meta060 (ug/ml) + LPS | | |
|---|---|---|---|---|---|
| | Control | LPS | (10 ug/ml) | | |
| Phosphoproteins | (Unstimulated) | (10 ug/ml) | 20 ug | 10 ug | 5 ug |
| JNK | 1.0 | 7.2 | 3.6 | 5.0 | 5.5 |
| p70 S6K | 1.0 | 1.5 | 0.7 | 0.9 | 1.5 |
| STAT5A/B | 1.0 | 1.5 | 1.4 | 1.1 | 1.0 |
| p38 | 1.0 | 7.0 | 5.4 | 4.8 | 6.5 |

Regulation of transcriptional factors in THP-1 cells: THP-1 cells were seeded in 100 mm dish, pre-incubated with META-060 (10 mg/ml)) for 1 hr and stimulated with TNFa (10 ng/ml) or LPS (10 ug/ml) for 2 hrs. Nuclear extracts were prepared as per the instructions from user manual (Panomics, Calif.). Transcriptional factors were measured using Procarta® Transcription Factor Plex panel 1 (Panomics, Calif.) according to the manufacturer instructions using Luminex 200. The fold induction was calculated from 3 measurements. TFIID was used as an internal control and unstimulated samples used to calculate fold induction. The data is presented in Tables 5 & 6.

TABLE 5
Effect of Meta060 on TNFa mediated transcriptional factors in THP-1
cells

| | Control | | Meta060 (20 ug/ml) + |
|---|---|---|---|
| | (unstimulated) | TNFa (10 ng/ml) | TNFa (10 ng/ml) |
| ELK-1 | 1.00 | 0.87 | 1.09 |
| NFkB | 1.00 | 1.86 | 1.40 |
| FAST-1 | 1.00 | 1.40 | 1.21 |
| OCT | 1.00 | 1.21 | 0.98 |
| P53 | 1.00 | 1.96 | 1.89 |
| PAX-3 | 1.00 | 1.20 | 0.92 |
| NF-E2 | 1.00 | 1.84 | 1.42 |
| AP-2 | 1.00 | 1.35 | 1.11 |
| NF-E1(YY1) | 1.00 | 2.38 | 2.71 |
| ATF-2 | 1.00 | 1.50 | 1.18 |
| NF-1 | 1.00 | 2.13 | 1.77 |
| ISRE | 1.00 | 1.93 | 1.61 |
| PAX-5 | 1.00 | 1.15 | 1.37 |
| Nkx-2.5 | 1.00 | 1.60 | 1.25 |
| AR | 1.00 | 1.71 | 1.32 |
| ETS/PEA | 1.00 | 1.42 | 1.17 |
| AP-1 | 1.00 | 1.91 | 1.18 |
| E2F1 | 1.00 | 1.86 | 1.70 |
| MyoD | 1.00 | 1.67 | 1.48 |
| PPAR | 1.00 | 2.01 | 1.19 |
| RUNX/AML | 1.00 | 1.40 | 0.90 |
| Brm3 | 1.00 | 0.44 | 0.75 |
| NF-Y | 1.00 | 2.44 | 1.87 |
| c-myb | 1.00 | 1.42 | 1.08 |
| CREB | 1.00 | 1.74 | 1.35 |
| ER | 1.00 | 1.60 | 1.30 |
| GR/PR | 1.00 | 1.43 | 1.25 |
| HIF-1 | 1.00 | 1.74 | 1.45 |
| FKHR | 1.00 | 1.31 | 0.96 |
| GATA | 1.00 | 1.44 | 1.10 |
| IRF | 1.00 | 1.74 | 1.43 |
| STAT-1 | 1.00 | 0.90 | 0.79 |
| HNF1 | 1.00 | 1.11 | 1.07 |
| STAT-4 | 1.00 | 1.32 | 1.09 |
| STAT-5 | 1.00 | 1.27 | 1.19 |
| MEF-2 | 1.00 | 1.24 | 0.97 |
| NFAT | 1.00 | 1.10 | 0.73 |

TABLE 6
Effect of Meta060 on LPS mediated transcriptional factors in THP-1 cells

| | | LPS | Meta060 (20 ug/ml) + |
|---|---|---|---|
| | Control (Unstimulated) | (10 ug/ml) | LPS (10 ug/ml) |
| ELK-1 | 1.00 | 0.38 | 0.57 |
| NFkB | 1.00 | 3.20 | 0.83 |
| FAST-1 | 1.00 | 1.24 | 1.45 |
| OCT | 1.00 | 0.72 | 0.69 |
| P53 | 1.00 | 1.19 | 2.60 |
| PAX-3 | 1.00 | 0.92 | 0.81 |
| NF-E2 | 1.00 | 2.71 | 2.11 |
| AP-2 | 1.00 | 0.91 | 1.05 |
| NF-E1(YY1) | 1.00 | 1.16 | 2.46 |
| ATF-2 | 1.00 | 1.31 | 1.10 |
| NF-1 | 1.00 | 1.46 | 1.86 |
| ISRE | 1.00 | 1.14 | 2.77 |
| PAX-5 | 1.00 | 1.42 | 0.75 |
| Nkx-2.5 | 1.00 | 0.97 | 1.47 |
| AR | 1.00 | 1.40 | 2.63 |
| ETS/PEA | 1.00 | 0.88 | 0.95 |
| AP-1 | 1.00 | 4.06 | 1.42 |
| E2F1 | 1.00 | 1.68 | 2.92 |
| MyoD | 1.00 | 0.88 | 1.65 |
| PPAR | 1.00 | 1.07 | 1.04 |
| RUNX/AML | 1.00 | 1.42 | 1.10 |
| Brm3 | 1.00 | 0.22 | 0.13 |
| NF-Y | 1.00 | 1.68 | 2.02 |
| c-myb | 1.00 | 1.03 | 1.92 |
| CREB | 1.00 | 1.71 | 1.77 |
| ER | 1.00 | 1.31 | 2.36 |
| GR/PR | 1.00 | 0.69 | 1.75 |
| HIF-1 | 1.00 | 1.54 | 2.45 |
| FKHR | 1.00 | 1.04 | 0.93 |
| GATA | 1.00 | 0.88 | 1.37 |
| IRF | 1.00 | 0.83 | 1.13 |
| STAT-1 | 1.00 | 0.71 | 0.53 |
| HNF1 | 1.00 | 0.77 | 1.42 |
| STAT-4 | 1.00 | 0.81 | 1.07 |
| STAT-5 | 1.00 | 1.09 | 0.94 |
| MEF-2 | 1.00 | 0.75 | 0.79 |
| NFAT | 1.00 | 0.70 | 0.53 |

Human Gene array analysis: THP-1 cells were pre-incubated with META-060 (10 mg/ml) or parthenolide (10 mM) for 1 hr and stimulated with TNFa (10 ng/ml) for 4 hrs. Genes were analyzed using Affymetrix human genome array U133A 2.0 and measured ˜22,000 transcripts (by Expression Analysis Inc, North Carolina). Genes which are known to activate endothelial and monocyte interactions were shown. Each value represent Mean of 2 independent measurements.

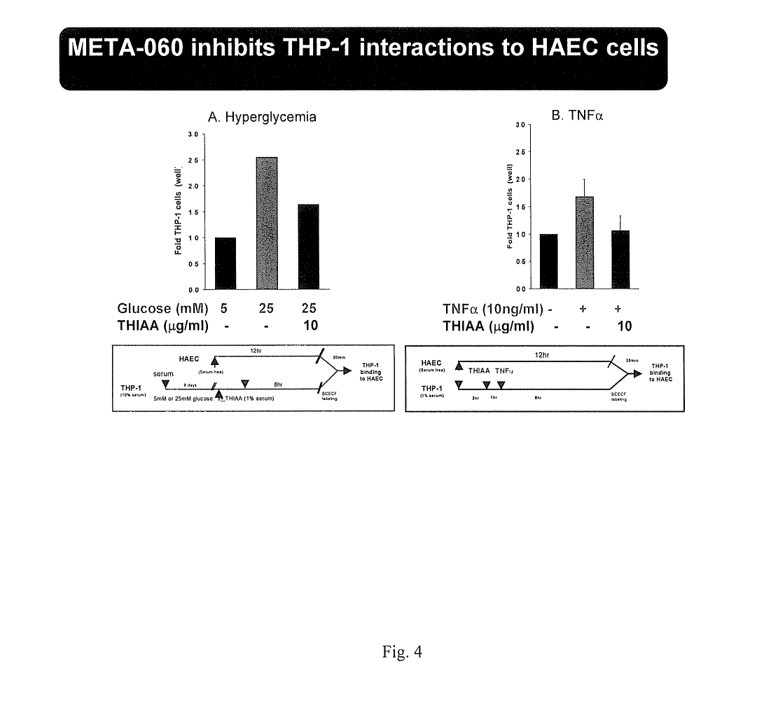

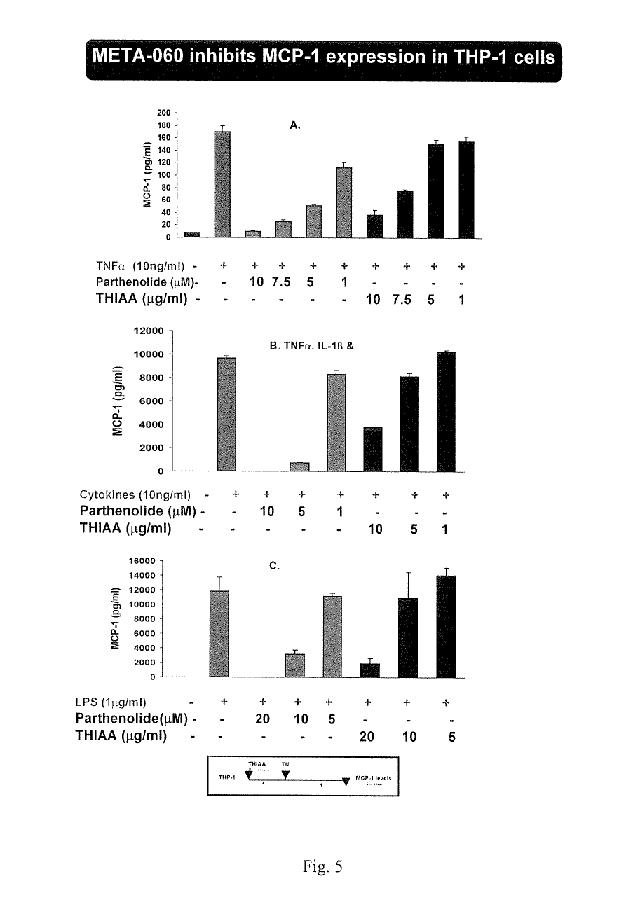

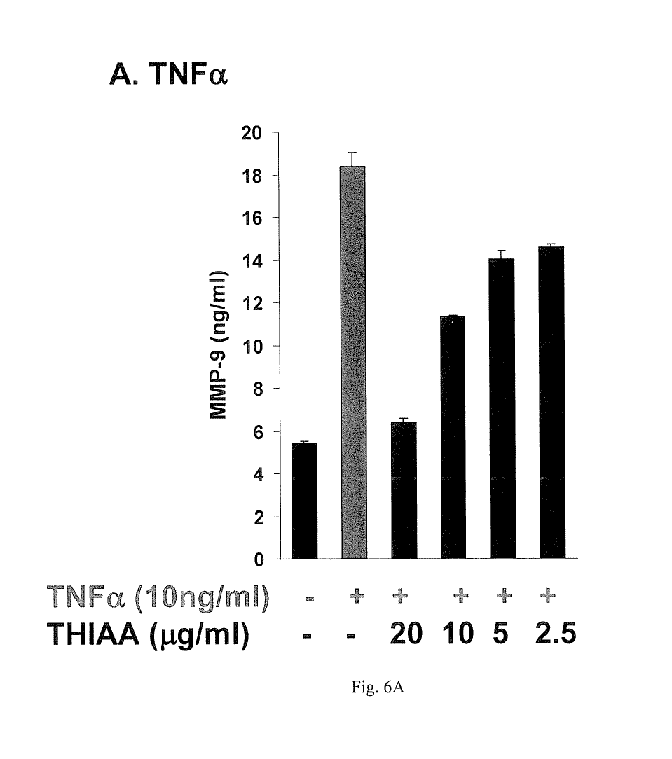

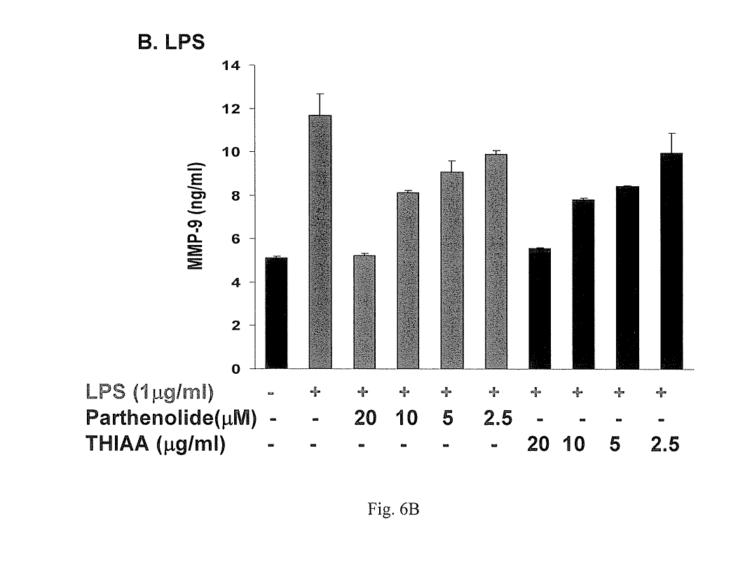

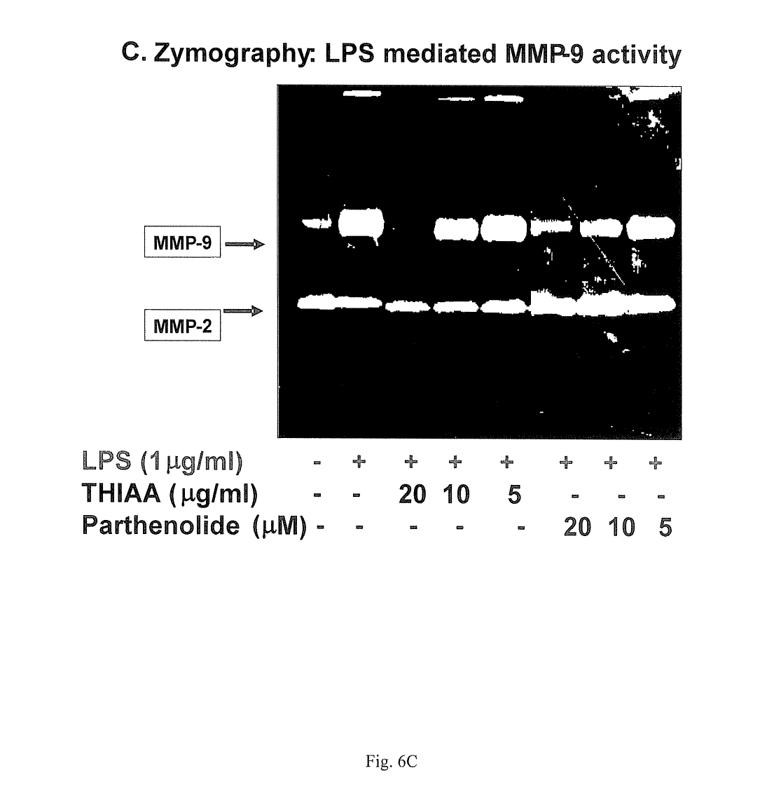

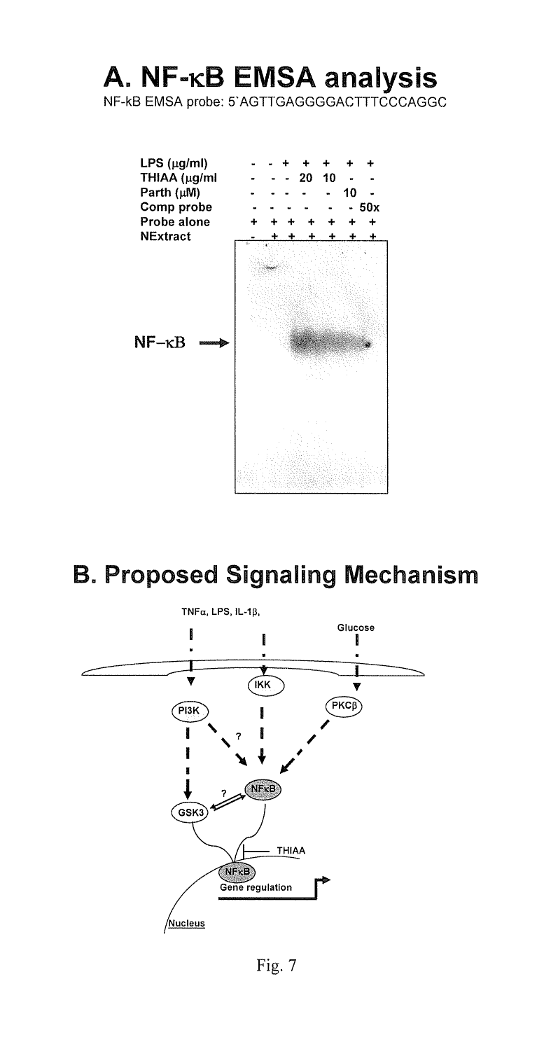

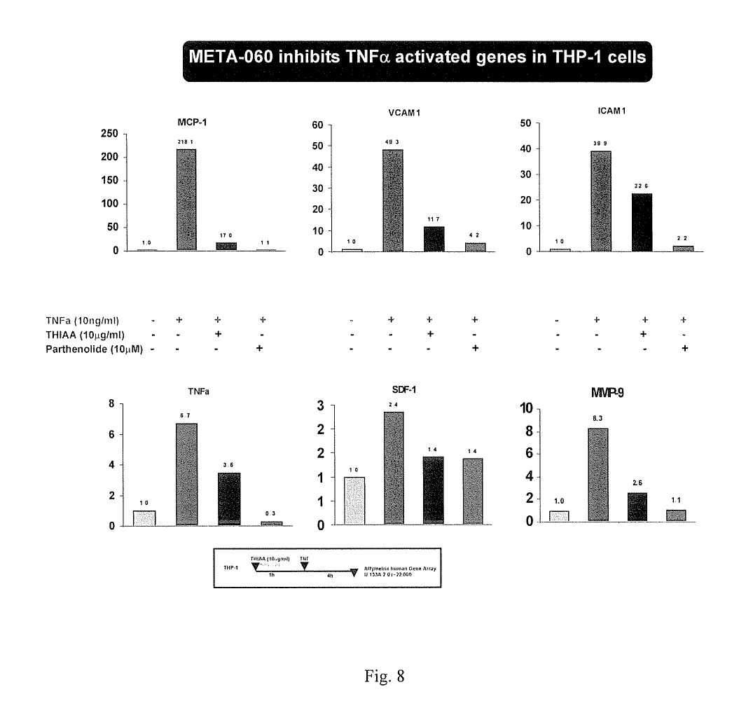

Results—The results for this Example are presented in FIGS. 4-8 and Table 7 and indicate that META-060 inhibits: a) Hyperglycemia and TNFa mediated, THP-1-HAEC cell interactions; b) Cytokines and LPS mediated MCP-1 secretion; c) TNFa and LPS activated MMP-9 levels; d) LPS mediated NF-kB binding; and e) TNFa mediated inflammatory genes including, MCP-1, VCAM, ICAM-1, MMP-9 and TNFa in THP-1 cells.

TABLE 7
EA 0732: 1 hr preincubation with test compounds, 4 hr stimulation with LPS (1 ug/ml) or TNFa (10 ng/ml) in THP-1 monocyttic cells
(Affymetrix Human Gene array U133A 2.0)

| | | | | | | | THIAA inhibition | | | | THIAA inhibition |
|---|---|---|---|---|---|---|---|---|---|---|---|
| | | | Seq | | | | (Fraction of | | | | (Fraction of |
| | Locus | Gene | Derived | Control | TNFa | THIAA | TNFa | Control | LPS | THIAA | LPS |
| Probe Set ID | Link | Symbol | From | (DMSO) | (10 ng/ml) | (10 ug/ml) + TNFa | stimulation) | (DMSO) | (1 ug/ml) | (10 ug) + LPS | stimulation) |
| 209387_s_at | 4071 | TM4SF1 | M90657 | 7.77 | 32.01 | 0.77 | 0.02 | 7.77 | 4.26 | 8.92 | |
| 216307_at | 1607 | DGKB | AB018261 | 1.77 | 22.51 | 0.71 | 0.03 | 1.77 | 2.84 | 5.19 | |
| 207837_at | 11030 | RBPMS | NM_006867 | 4.20 | 24.09 | 0.77 | 0.03 | 4.20 | 5.37 | 17.29 | |
| 208529_at | 690 | BTF3L1 | NM_001208 | 4.85 | 26.42 | 0.99 | 0.04 | 4.85 | 3.48 | 0.80 | |
| 216923_at | 6792 | CDKL5 | AL049684 | 8.08 | 34.36 | 1.35 | 0.04 | 8.08 | 20.57 | 33.50 | |
| 207679_at | 5077 | PAX3 | NM_000438 | 9.48 | 40.46 | 2.02 | 0.05 | 9.48 | 25.09 | 2.74 | 0.11 |
| 215523_at | 346157 | RPI- | AL031118 | 7.54 | 26.87 | 1.38 | 0.05 | 7.54 | 5.16 | 19.70 | |
| | | 153G14.3 | | | | | | | | | |
| 211525_s_at | 2814 | GP5 | L11238 | 6.37 | 18.86 | 0.98 | 0.05 | 6.37 | 3.45 | 15.65 | |
| 216787_at | 2281 | FKBP1B | AK026273 | 8.41 | 55.23 | 2.89 | 0.05 | 8.41 | 13.95 | 7.39 | 0.53 |
| 220820_at | | | NM_018539 | 21.18 | 39.60 | 2.19 | 0.06 | 21.18 | 14.35 | 31.81 | |
| 208045_at | 9981 | SPAR | NM_005129 | 16.48 | 25.57 | 1.44 | 0.06 | 16.48 | 16.54 | 4.07 | |
| 206446_s_at | 63036 | ELA2A | NM_001971 | 3.65 | 25.64 | 1.47 | 0.06 | 3.65 | 18.52 | 1.86 | 0.10 |
| 207527_at | 3765 | KCNJ9 | NM_004983 | 8.00 | 53.09 | 3.08 | 0.06 | 8.00 | 18.72 | 11.49 | 0.61 |
| 220474_at | 89874 | SLC25A21 | NM_030631 | 3.74 | 15.58 | 0.94 | 0.06 | 3.74 | 10.94 | 10.48 | |
| 211522_s_at | 2798 | GNRHR | L03380 | 0.71 | 22.66 | 1.41 | 0.06 | 0.71 | 1.92 | 3.48 | |
| 220187_at | 79689 | STEAP4 | NM_024636 | 2.65 | 31.68 | 2.01 | 0.06 | 2.65 | 5.09 | 5.01 | |
| 221322_at | 64111 | NPVF | NM_022150 | 0.99 | 20.28 | 1.35 | 0.07 | 0.99 | 1.71 | 5.69 | |
| 219136_s_at | 64788 | TMEM112 | NM_022773 | 7.70 | 35.86 | 2.40 | 0.07 | 7.70 | 3.74 | 4.74 | |
| 205886_at | 5968 | REG1B | NM_006507 | 10.51 | 33.27 | 2.25 | 0.07 | 10.51 | 4.49 | 9.15 | |
| 217265_at | 51090 | PLLP | AL020989 | 2.32 | 23.95 | 1.62 | 0.07 | 2.32 | 1.62 | 4.05 | |
| 217213_at | 6530 | SLC6A2 | AB022847 | 1.94 | 23.63 | 1.67 | 0.07 | 1.94 | 5.60 | 2.77 | |
| 209183_s_at | 11067 | C10orf10 | AL136653 | 17.77 | 130.43 | 9.25 | 0.07 | 17.77 | 34.77 | 15.72 | 0.45 |
| 207912_s_at | 1617 /// | DAZ1 | U21663 | 4.20 | 27.76 | 1.97 | 0.07 | 4.20 | 15.41 | 9.48 | 0.62 |
| | | /// | | | | | | | | | |
| | | DAZ3 | | | | | | | | | |
| | | /// | | | | | | | | | |
| | | DAZ2 | | | | | | | | | |
| | | /// DAZ | | | | | | | | | |
| 205430_at | 653 | BMP5 | NM_021073 | 2.72 | 17.89 | 1.30 | 0.07 | 2.72 | 1.41 | 0.99 | |
| 220222_at | 55472 | C8orf39 | NM_018608 | 3.66 | 30.66 | 2.30 | 0.07 | 3.66 | 4.12 | 4.62 | |
| 215117_at | 5897 | RAG2 | AW058148 | 8.35 | 18.61 | 1.41 | 0.08 | 8.35 | 4.91 | 3.71 | |
| 209988_s_at | 429 | ASCL1 | BC001638 | 8.11 | 14.39 | 1.09 | 0.08 | 8.11 | 1.55 | 9.42 | |
| 206941_x_at | 9723 | SEMA3E | NM_012431 | 4.04 | 19.31 | 1.49 | 0.08 | 4.04 | 1.01 | 3.56 | |
| 213197_at | 460 | ASTN1 | AB006627 | 1.54 | 20.39 | 1.59 | 0.08 | 1.54 | 8.60 | 3.80 | |
| 216598_s_at | 6347 | CCL2 | S69738 | 13.11 | 2860.66 | 222.78 | 0.08 | 13.11 | 430.87 | 121.62 | 0.28 |
| 216839_at | 3908 | LAMA2 | AK026829 | 2.73 | 14.41 | 1.12 | 0.08 | 2.73 | 16.92 | 14.17 | |
| 216134_at | 23150 | FRMD4B | AK000244 | 14.80 | 26.35 | 2.07 | 0.08 | 14.80 | 12.75 | 8.25 | |
| 206300_s_at | 5744 | PTHLH | NM_002820 | 10.21 | 15.70 | 1.24 | 0.08 | 10.21 | 21.67 | 3.62 | 0.17 |
| 202196_s_at | 27122 | DKK3 | NM_013253 | 2.32 | 18.77 | 1.50 | 0.08 | 2.32 | 3.00 | 6.94 | |
| 216986_s_at | 3662 | IRF4 | D78261 | 30.30 | 58.93 | 4.80 | 0.08 | 30.30 | 28.46 | 13.55 | |
| 222258_s_at | 23677 | SH3BP4 | AF015043 | 3.89 | 22.00 | 1.80 | 0.08 | 3.89 | 6.77 | 9.89 | |
| 210016_at | 23040 | MYT1L | BF223003 | 6.75 | 31.55 | 2.59 | 0.08 | 6.75 | 12.00 | 10.95 | |
| 210116_at | 4068 | SH2D1A | AF072930 | 1.34 | 52.10 | 4.35 | 0.08 | 1.34 | 5.61 | 13.15 | |
| 215795_at | 57644 | MYH7B | AK000947 | 5.19 | 18.50 | 1.56 | 0.08 | 5.19 | 4.94 | 2.39 | |
| 204612_at | 5569 | PKIA | NM_006823 | 8.28 | 23.53 | 2.00 | 0.09 | 8.28 | 14.37 | 2.71 | 0.19 |
| 219803_at | 27329 | ANGPTL3 | NM_014495 | 10.26 | 29.88 | 2.58 | 0.09 | 10.26 | 12.07 | 2.39 | |
| 206666_at | 3003 | GZMK | NM_002104 | 3.15 | 49.67 | 4.34 | 0.09 | 3.15 | 10.81 | 7.38 | 0.68 |
| 220807_at | 3049 | HBQ1 | NM_005331 | 10.08 | 32.36 | 2.89 | 0.09 | 10.08 | 2.86 | 11.50 | |
| 220880_at | | | NM_018601 | 3.53 | 27.80 | 2.50 | 0.09 | 3.53 | 7.47 | 7.17 | |
| 205460_at | 4862 | NPAS2 | NM_002518 | 14.34 | 30.55 | 2.75 | 0.09 | 14.34 | 38.35 | 16.29 | 0.42 |
| 219827_at | 7352 | UCP3 | NM_003356 | 27.43 | 67.56 | 6.24 | 0.09 | 27.43 | 19.79 | 31.19 | |
| 221347_at | 1133 | CHRM5 | NM_012125 | 3.09 | 21.42 | 1.98 | 0.09 | 3.09 | 15.25 | 4.10 | 0.27 |
| 208525_s_at | 135948 / | OR2F1 | NM_012369 | 1.30 | 28.88 | 2.68 | 0.09 | 1.30 | 3.15 | 4.84 | |
| | | /// | | | | | | | | | |
| | | OR2F2 | | | | | | | | | |
| 220314_at | 51402 | HSFX1 | NM_016153 | 4.81 | 35.95 | 3.35 | 0.09 | 4.81 | 6.22 | 7.54 | |
| | // | /// | | | | | | | | | |
| | | LOC728302 | | | | | | | | | |
| 220460_at | 53919 | SLCO1C1 | NM_017435 | 17.61 | 26.61 | 2.50 | 0.09 | 17.61 | 4.89 | 6.94 | |
| 216003_at | 374286 | CDRT1 | U43383 | 15.63 | 27.12 | 2.55 | 0.09 | 15.63 | 6.37 | 2.41 | |
| 207910_at | 10648 | SCGB1D1 | NM_006552 | 0.73 | 14.86 | 1.40 | 0.09 | 0.73 | 1.70 | 0.92 | |
| 217412_at | | | AE000659 | 5.42 | 22.08 | 2.08 | 0.09 | 5.42 | 3.44 | 6.72 | |
| 216641_s_at | 3898 | LAD1 | U58994 | 10.18 | 46.17 | 4.35 | 0.09 | 10.18 | 17.11 | 3.57 | 0.21 |
| 216939_s_at | 3360 | HTR4 | Y08756 | 7.77 | 26.90 | 2.55 | 0.09 | 7.77 | 11.29 | 3.96 | |
| 207909_x_at | 1617 /// | DAZ1 | U21663 | 3.69 | 12.24 | 1.17 | 0.10 | 3.69 | 4.26 | 4.16 | |
| | | /// | | | | | | | | | |
| | | DAZ3 | | | | | | | | | |
| | | /// | | | | | | | | | |
| | | DAZ2 | | | | | | | | | |
| | | /// DAZ | | | | | | | | | |
| 211044_at | 9830 | TRIM14 | BC006333 | 15.45 | 59.97 | 5.79 | 0.10 | 15.45 | 27.99 | 5.98 | 0.21 |
| 217350_at | 160313 | LOC160313 | AB041269 | 4.57 | 15.56 | 1.53 | 0.10 | 4.57 | 4.28 | 2.30 | |
| 204464_s_at | 1909 | EDNRA | NM_001957 | 14.40 | 26.56 | 2.64 | 0.10 | 14.40 | 6.32 | 7.03 | |
| 208497_x_at | 4762 | NEUROG1 | NM_006161 | 19.48 | 61.49 | 6.14 | 0.10 | 19.48 | 74.79 | 87.44 | |
| 210713_at | 6453 | ITSN1 | U61166 | 6.26 | 22.08 | 2.23 | 0.10 | 6.26 | 7.49 | 5.93 | |
| 207640_x_at | 4917 | NTN2L | NM_006181 | 11.60 | 58.01 | 5.88 | 0.10 | 11.60 | 13.16 | 10.71 | |
| 220010_at | 23630 | KCNE1L | NM_012282 | 29.49 | 48.71 | 4.94 | 0.10 | 29.49 | 26.70 | 28.21 | |
| 204878_s_at | 9344 | TAOK2 | NM_004783 | 13.61 | 80.91 | 8.23 | 0.10 | 13.61 | 35.30 | 46.05 | |
| 204424_s_at | 55885 | LMO3 | AL050152 | 19.24 | 31.57 | 3.22 | 0.10 | 19.24 | 24.51 | 7.04 | |
| 215784_at | 913 | CD1E | AA309511 | 6.86 | 43.27 | 4.45 | 0.10 | 6.86 | 9.32 | 1.24 | |
| 207070_at | 5995 | RGR | NM_002921 | 10.13 | 52.32 | 5.53 | 0.11 | 10.13 | 5.17 | 30.01 | |
| 203903_s_at | 9843 | HEPH | NM_014799 | 3.20 | 36.29 | 3.85 | 0.11 | 3.20 | 27.70 | 20.68 | 0.75 |
| 210850_s_at | 2002 | ELK1 | AF000672 | 22.89 | 40.28 | 4.28 | 0.11 | 22.89 | 4.24 | 11.26 | |
| 212278_x_at | 7337 | UBE3A | BF588511 | 18.75 | 41.53 | 4.42 | 0.11 | 18.75 | 3.29 | 11.11 | |
| 215796_at | | | BF976764 | 3.69 | 20.46 | 2.20 | 0.11 | 3.69 | 3.52 | 14.70 | |
| 207077_at | 51032 | ELA2B | NM_015849 | 23.57 | 40.66 | 4.40 | 0.11 | 23.57 | 10.79 | 25.59 | |
| 206842_at | 3750 | KCND1 | NM_004979 | 17.40 | 49.85 | 5.40 | 0.11 | 17.40 | 75.09 | 54.08 | 0.72 |
| AFFX-r2-Bs- | | | AFFX-r2- | 21.90 | 43.37 | 4.70 | 0.11 | 21.90 | 9.16 | 16.25 | |
| thr-M_s_at | | | Bs-thr-M | | | | | | | | |
| 215468_at | 647070 | LOC647070 | AK001442 | 18.02 | 32.66 | 3.57 | 0.11 | 18.02 | 17.14 | 8.13 | |
| 220450_at | 646593 | LOC646593 | NM_024914 | 4.35 | 12.48 | 1.37 | 0.11 | 4.35 | 10.12 | 4.30 | 0.42 |
| 220916_at | 80188 | FLJ13310 | NM_025118 | 12.10 | 33.61 | 3.69 | 0.11 | 12.10 | 16.15 | 19.97 | |
| 220506_at | 2974 | GUCY1B2 | NM_004129 | 3.63 | 15.14 | 1.67 | 0.11 | 3.63 | 4.63 | 3.26 | |
| 214410_at | 4308 | TRPM1 | N32151 | 10.51 | 18.87 | 2.09 | 0.11 | 10.51 | 6.91 | 4.05 | |
| 216927_at | | | AC003030 | 7.46 | 27.89 | 3.11 | 0.11 | 7.46 | 12.19 | 16.44 | |
| 216893_s_at | 1285 | COL4A3 | U02519 | 10.39 | 29.16 | 3.26 | 0.11 | 10.39 | 11.40 | 15.20 | |
| 219167_at | 51285 | RASL12 | NM_016563 | 44.41 | 69.87 | 7.86 | 0.11 | 44.41 | 6.60 | 25.12 | |
| 220619_at | 55636 | CHD7 | NM_017783 | 3.84 | 20.71 | 2.34 | 0.11 | 3.84 | 9.81 | 27.35 | |
| 210823_s_at | 5802 | PTPRS | U40317 | 8.22 | 32.00 | 3.63 | 0.11 | 8.22 | 29.63 | 25.27 | |
| 214451_at | 7021 | TFAP2B | NM_003221 | 6.56 | 10.50 | 1.20 | 0.11 | 6.56 | 4.13 | 2.26 | |
| 220552_at | 7224 | TRPC5 | NM_012471 | 2.04 | 25.60 | 2.92 | 0.11 | 2.04 | 5.31 | 3.71 | |
| 215225_s_at | 2840 | GPR17 | Z94154 | 5.57 | 18.41 | 2.12 | 0.11 | 5.57 | 6.78 | 21.51 | |
| 214008_at | 5756 | TWF1 | N25562 | 22.51 | 47.26 | 5.46 | 0.12 | 22.51 | 30.76 | 26.92 | |
| 221087_s_at | 80833 | APOL3 | NM_014349 | 17.01 | 35.33 | 4.08 | 0.12 | 17.01 | 11.31 | 14.20 | |
| 214145_s_at | 6710 | SPTB | BG223341 | 9.29 | 47.02 | 5.44 | 0.12 | 9.29 | 10.10 | 6.43 | |
| 217119_s_at | 2833 | CXCR3 | Z79783 | 61.47 | 134.49 | 15.64 | 0.12 | 61.47 | 13.05 | 12.26 | |
| 222174_at | 4149 | MAX | AU145025 | 11.80 | 31.26 | 3.64 | 0.12 | 11.80 | 6.18 | 20.16 | |
| 211843_x_at | 1551 | CYP3A7 | AF315325 | 4.01 | 28.96 | 3.39 | 0.12 | 4.01 | 21.66 | 7.47 | 0.35 |
| 207846_at | 5449 | POU1F1 | NM_000306 | 2.72 | 19.22 | 2.25 | 0.12 | 2.72 | 12.41 | 3.55 | 0.29 |
| 208059_at | 1237 | CCR8 | NM_005201 | 8.05 | 38.33 | 4.49 | 0.12 | 8.05 | 16.31 | 10.86 | 0.67 |
| 206982_at | 1411 | CRYBA1 | NM_005208 | 7.77 | 15.73 | 1.85 | 0.12 | 7.77 | 5.91 | 2.26 | |
| 216588_at | 120872 / | RPL7 | AL031577 | 10.70 | 16.25 | 1.92 | 0.12 | 10.70 | 21.61 | 7.10 | 0.33 |
| | | /// | | | | | | | | | |
| | | LOC120872 | | | | | | | | | |
| | | /// | | | | | | | | | |
| | | LOC1307 | | | | | | | | | |
| 210504_at | 10661 | KLF1 | U65404 | 5.69 | 38.42 | 4.57 | 0.12 | 5.69 | 35.03 | 1.70 | 0.05 |
| 220214_at | 7762 | ZNF215 | NM_013250 | 8.69 | 33.83 | 4.05 | 0.12 | 8.69 | 15.46 | 5.57 | 0.36 |
| 207255_at | 3953 | LEPR | NM_002303 | 15.52 | 35.32 | 4.24 | 0.12 | 15.52 | 9.79 | 10.04 | |
| 208139_s_at | | | NM_031269 | 3.66 | 31.29 | 3.79 | 0.12 | 3.66 | 1.55 | 5.67 | |
| 217409_at | 4644 | MYO5A | Z22957 | 5.67 | 10.30 | 1.25 | 0.12 | 5.67 | 2.68 | 1.50 | |
| 215835_at | 949 | SCARB1 | AV708130 | 3.43 | 29.06 | 3.53 | 0.12 | 3.43 | 12.10 | 6.25 | 0.52 |
| 209047_at | 358 | AQP1 | AL518391 | 129.69 | 1090.70 | 133.76 | 0.12 | 129.69 | 307.48 | 190.88 | 0.62 |
| 214502_at | 8970 | HIST1H2BJ | NM_021058 | 2.75 | 13.12 | 1.61 | 0.12 | 2.75 | 3.00 | 1.94 | |
| 216447_at | | | AK024525 | 1.80 | 19.69 | 2.43 | 0.12 | 1.80 | 3.39 | 14.05 | |
| 208533_at | 6656 | SOX1 | NM_005986 | 5.71 | 17.16 | 2.12 | 0.12 | 5.71 | 1.80 | 2.81 | |
| 216363_at | | | S73614 | 26.46 | 66.54 | 8.24 | 0.12 | 26.46 | 30.88 | 21.77 | |
| 218656_s_at | 10186 | LHFP | NM_005780 | 4.45 | 22.24 | 2.77 | 0.12 | 4.45 | 2.79 | 2.51 | |
| 211920_at | 629 | CFB | AF349679 | 3.25 | 36.01 | 4.50 | 0.12 | 3.25 | 32.91 | 3.67 | 0.11 |
| 202086_at | 4599 | MX1 | NM_002462 | 306.62 | 3062.73 | 389.68 | 0.13 | 306.62 | 4083.25 | 648.81 | 0.16 |
| 220228_at | 23431 | AP4E1 | AB030653 | 4.96 | 20.77 | 2.65 | 0.13 | 4.96 | 2.35 | 1.31 | |
| 211164_at | 2042 | EPHA3 | AF213460 | 1.05 | 18.86 | 2.44 | 0.13 | 1.05 | 2.42 | 2.73 | |
| 216687_x_at | 7366 | UGT2B15 | U06641 | 8.05 | 24.52 | 3.19 | 0.13 | 8.05 | 17.44 | 8.77 | 0.50 |
| 220226_at | 79054 | TRPM8 | NM_024080 | 20.22 | 56.93 | 7.45 | 0.13 | 20.22 | 14.18 | 23.50 | |
| 208143_s_at | 10876 | FAM12A | NM_006683 | 2.76 | 29.14 | 3.83 | 0.13 | 2.76 | 4.52 | 1.82 | |
| 214939_x_at | 4301 | MLLT4 | AB011399 | 10.19 | 16.08 | 2.12 | 0.13 | 10.19 | 9.34 | 14.66 | |
| 205106_at | 4515 | MTCP1 | NM_014221 | 37.46 | 59.04 | 7.79 | 0.13 | 37.46 | 24.53 | 33.09 | |
| 214966_at | 2901 | GRIK5 | S40369 | 3.18 | 25.09 | 3.33 | 0.13 | 3.18 | 9.21 | 9.25 | |
| 212224_at | 216 | ALDH1A1 | NM_000689 | 15.84 | 54.38 | 7.21 | 0.13 | 15.84 | 43.36 | 21.96 | 0.51 |
| 219456_s_at | 79890 | RIN3 | NM_024832 | 70.81 | 145.86 | 19.38 | 0.13 | 70.81 | 54.48 | 15.97 | |
| 217706_at | 220074 | LRRC51 | AV742010 | 7.83 | 42.90 | 5.71 | 0.13 | 7.83 | 14.82 | 10.49 | 0.71 |
| 211745_x_at | 3039 | HBA1 | BC005931 | 19.64 | 60.30 | 8.12 | 0.13 | 19.64 | 4.91 | 19.30 | |
| 221186_at | | | NM_025116 | 14.39 | 36.10 | 4.87 | 0.13 | 14.39 | 9.20 | 6.91 | |
| 213973_at | 6238 | RRBP1 | BE646396 | 36.16 | 80.15 | 10.89 | 0.14 | 36.16 | 27.42 | 29.48 | |
| 209763_at | 91851 | CHRDL1 | AL049176 | 6.88 | 29.60 | 4.05 | 0.14 | 6.88 | 18.04 | 25.39 | |
| 220556_at | 23439 | ATP1B4 | NM_012069 | 4.40 | 21.10 | 2.91 | 0.14 | 4.40 | 9.06 | 8.98 | |
| 203930_s_at | 4137 | MAPT | NM_016835 | 3.50 | 24.27 | 3.35 | 0.14 | 3.50 | 13.37 | 21.10 | |
| 210354_at | 3458 | IFNG | M29383 | 3.80 | 24.89 | 3.46 | 0.14 | 3.80 | 7.75 | 8.95 | |
| 217316_at | 390892 | OR7A10 | AC005255 | 4.05 | 35.52 | 4.95 | 0.14 | 4.05 | 8.15 | 2.83 | |
| 205772_s_at | 9465 | AKAP7 | NM_004842 | 20.57 | 38.43 | 5.41 | 0.14 | 20.57 | 23.89 | 14.88 | |
| 214837_at | 213 | ALB | M12523 | 9.90 | 33.25 | 4.69 | 0.14 | 9.90 | 19.48 | 7.07 | 0.36 |
| 207580_at | 4115 | MAGEB4 | NM_002367 | 5.24 | 15.23 | 2.15 | 0.14 | 5.24 | 10.97 | 14.43 | |
| 220491_at | 57817 | HAMP | NM_021175 | 26.85 | 58.18 | 8.26 | 0.14 | 26.85 | 44.03 | 38.80 | |
| 213722_at | 6657 | SOX2 | AW007161 | 2.54 | 17.20 | 2.45 | 0.14 | 2.54 | 6.29 | 8.45 | |
| 220568_at | | | NM_018582 | 13.19 | 20.61 | 2.93 | 0.14 | 13.19 | 4.49 | 2.79 | |
| 206071_s_at | 2042 | EPHA3 | AF213459 | 1.38 | 26.66 | 3.79 | 0.14 | 1.38 | 2.36 | 18.60 | |
| 210579_s_at | 10107 | TRIM10 | AF220122 | 8.29 | 54.23 | 7.75 | 0.14 | 8.29 | 48.81 | 49.49 | |
| 204439_at | 10964 | IFI44L | NM_006820 | 62.17 | 663.59 | 94.85 | 0.14 | 62.17 | 681.10 | 109.27 | 0.16 |
| 207242_s_at | 2897 | GRIK1 | NM_000830 | 3.57 | 36.81 | 5.26 | 0.14 | 3.57 | 8.45 | 7.77 | |
| 211472_at | 23654 | PLXNB2 | AF336795 | 4.02 | 17.27 | 2.47 | 0.14 | 4.02 | 5.62 | 1.89 | |
| 212750_at | 26051 | PPP1R16B | AB020630 | 6.69 | 28.07 | 4.05 | 0.14 | 6.69 | 19.11 | 10.07 | 0.53 |
| 209597_s_at | 10687 | PNMA2 | AB020690 | 21.05 | 41.90 | 6.05 | 0.14 | 21.05 | 15.98 | 6.85 | |
| 203838_s_at | 10188 | TNK2 | AI146308 | 37.41 | 91.12 | 13.18 | 0.14 | 37.41 | 33.55 | 15.37 | |
| 210086_at | 55806 | HR | AF039196 | 6.55 | 19.01 | 2.75 | 0.14 | 6.55 | 18.18 | 11.24 | 0.62 |
| 208514_at | 3753 | KCNE1 | NM_000219 | 3.98 | 16.90 | 2.45 | 0.15 | 3.98 | 3.92 | 12.77 | |
| 214772_at | 25758 | C11orf41 | H08993 | 6.82 | 27.01 | 3.95 | 0.15 | 6.82 | 12.77 | 31.78 | |
| 204667_at | 3169 | FOXA1 | NM_004496 | 3.55 | 46.41 | 6.86 | 0.15 | 3.55 | 26.09 | 19.78 | |
| 205388_at | 7125 | TNNC2 | NM_003279 | 11.77 | 41.53 | 6.15 | 0.15 | 11.77 | 15.33 | 9.57 | |
| 206749_at | 910 | CD1B | NM_001764 | 11.77 | 27.68 | 4.12 | 0.15 | 11.77 | 10.87 | 13.60 | |
| 221697_at | 440738 | MAP1LC3C | AF276659 | 6.23 | 22.08 | 3.29 | 0.15 | 6.23 | 8.08 | 16.92 | |
| 218644_at | 26499 | PLEK2 | NM_016445 | 63.35 | 140.84 | 21.03 | 0.15 | 63.35 | 100.88 | 95.53 | |
| 214067_at | 115939 | C16orf42 | AL031709 | 4.09 | 21.02 | 3.14 | 0.15 | 4.09 | 8.77 | 13.53 | |
| 207393_at | 3062 | HCRTR2 | NM_001526 | 17.49 | 33.30 | 5.03 | 0.15 | 17.49 | 4.39 | 6.84 | |
| 208422_at | 4481 | MSR1 | NM_002445 | 2.61 | 11.37 | 1.73 | 0.15 | 2.61 | 12.31 | 5.96 | 0.48 |
| 207587_at | 1418 | CRYGA | NM_014617 | 4.73 | 30.31 | 4.61 | 0.15 | 4.73 | 2.50 | 16.76 | |
| 219764_at | 11211 | FZD10 | NM_007197 | 7.21 | 14.71 | 2.24 | 0.15 | 7.21 | 8.61 | 5.14 | |
| 216193_at | 440366 | LOC440366 | X69637 | 7.02 | 25.01 | 3.82 | 0.15 | 7.02 | 22.40 | 27.45 | |
| 211227_s_at | 730420 / | PCDH11Y | AF332216 | 12.95 | 25.45 | 3.90 | 0.15 | 12.95 | 27.12 | 24.97 | |
| | | /// | | | | | | | | | |
| | | LOC730420 | | | | | | | | | |
| 216418_at | 215 /// | ABCD1 | AL133173 | 7.45 | 28.01 | 4.30 | 0.15 | 7.45 | 14.65 | 21.25 | |
| | | /// | | | | | | | | | |
| | | LOC642762 | | | | | | | | | |
| | | /// | | | | | | | | | |
| | | LOC648 | | | | | | | | | |
| 207293_s_at | 186 | AGTR2 | U16957 | 2.10 | 15.75 | 2.44 | 0.15 | 2.10 | 2.90 | 4.52 | |
| 206347_at | 5165 | PDK3 | NM_005391 | 40.62 | 80.76 | 12.54 | 0.16 | 40.62 | 35.91 | 44.38 | |
| 221420_at | | | NM_005179 | 5.63 | 10.20 | 1.59 | 0.16 | 5.63 | 6.22 | 1.92 | |
| 221140_s_at | 29933 | GPR132 | NM_013345 | 36.52 | 92.14 | 14.42 | 0.16 | 36.52 | 66.33 | 203.99 | |
| 208401_s_at | 2740 | GLP1R | U01157 | 7.03 | 29.03 | 4.55 | 0.16 | 7.03 | 5.22 | 7.44 | |
| 220854_at | | | NM_014123 | 8.49 | 19.50 | 3.06 | 0.16 | 8.49 | 1.44 | 2.51 | |
| 214453_s_at | 10561 | IFI44 | NM_006417 | 79.18 | 689.10 | 108.37 | 0.16 | 79.18 | 632.18 | 153.77 | 0.24 |
| 219285_s_at | 51199 | NIN | NM_016350 | 2.10 | 14.68 | 2.33 | 0.16 | 2.10 | 4.65 | 4.66 | |
| 204856_at | 10331 | B3GNT3 | NM_014256 | 13.44 | 40.89 | 6.50 | 0.16 | 13.44 | 24.93 | 13.30 | 0.53 |
| 216171_at | 1937 | EEF1G | AK025271 | 1.35 | 11.26 | 1.80 | 0.16 | 1.35 | 1.73 | 5.26 | |
| 209915_s_at | 9378 | NRXN1 | AB035356 | 3.74 | 20.28 | 3.24 | 0.16 | 3.74 | 2.16 | 5.72 | |
| 208226_x_at | 53616 | ADAM22 | NM_004194 | 13.66 | 24.39 | 3.90 | 0.16 | 13.66 | 21.56 | 11.11 | 0.52 |
| 208032_s_at | 2892 | GRIA3 | NM_000828 | 2.63 | 18.61 | 2.98 | 0.16 | 2.63 | 5.72 | 2.65 | |
| 203587_at | 379 | ARL4D | U25771 | 9.71 | 26.35 | 4.24 | 0.16 | 9.71 | 10.06 | 9.08 | |
| 205108_s_at | 338 | APOB | NM_000384 | 6.65 | 23.09 | 3.73 | 0.16 | 6.65 | 3.21 | 5.11 | |
| 206310_at | 6691 | SPINK2 | NM_021114 | 7.24 | 26.12 | 4.22 | 0.16 | 7.24 | 15.04 | 8.47 | 0.56 |
| 211480_s_at | 6579 | SLCO1A2 | AF085224 | 2.65 | 10.98 | 1.79 | 0.16 | 2.65 | 3.01 | 2.98 | |
| 210614_at | 7274 | TTPA | U21938 | 10.55 | 29.37 | 4.79 | 0.16 | 10.55 | 4.37 | 10.08 | |
| 208063_s_at | 10753 | CAPN9 | NM_006615 | 7.64 | 35.56 | 5.82 | 0.16 | 7.64 | 9.83 | 5.86 | |
| 210659_at | 1240 | CMKLR1 | U79526 | 7.99 | 59.40 | 9.72 | 0.16 | 7.99 | 18.35 | 17.88 | |
| 208392_x_at | 3431 | SP110 | NM_004510 | 83.11 | 293.94 | 48.41 | 0.16 | 83.11 | 280.34 | 21.95 | 0.08 |
| 206811_at | 114 | ADCY8 | NM_001115 | 2.79 | 26.34 | 4.36 | 0.17 | 2.79 | 26.63 | 12.96 | 0.49 |
| 222362_at | 3268 | HRBL | H07885 | 7.53 | 57.01 | 9.44 | 0.17 | 7.53 | 26.58 | 11.59 | 0.44 |
| 207206_s_at | 239 | ALOX12 | NM_000697 | 14.18 | 43.89 | 7.27 | 0.17 | 14.18 | 12.68 | 7.35 | |
| 201185_at | 5654 | HTRA1 | NM_002775 | 6.55 | 41.28 | 6.86 | 0.17 | 6.55 | 5.71 | 23.79 | |
| 220016_at | 79026 | AHNAK | NM_024060 | 11.01 | 52.73 | 8.79 | 0.17 | 11.01 | 20.63 | 18.55 | |
| 204698_at | 3669 | ISG20 | NM_002201 | 6.50 | 24.33 | 4.08 | 0.17 | 6.50 | 315.07 | 146.78 | 0.47 |
| 214977_at | 6503 | SLA | AK023852 | 4.77 | 24.45 | 4.10 | 0.17 | 4.77 | 32.81 | 31.55 | |
| 220636_at | 64446 | DNAI2 | NM_023036 | 8.55 | 46.43 | 7.80 | 0.17 | 8.55 | 14.24 | 5.23 | 0.37 |
| 207142_at | 3760 | KCNJ3 | NM_002239 | 1.07 | 14.81 | 2.50 | 0.17 | 1.07 | 7.90 | 2.26 | |
| 215028_at | 57556 | SEMA6A | AB002438 | 5.37 | 13.68 | 2.32 | 0.17 | 5.37 | 5.32 | 31.18 | |
| 215774_s_at | | | AV650470 | 1.20 | 11.66 | 1.98 | 0.17 | 1.20 | 22.70 | 4.33 | 0.19 |
| 210182_at | 1325 | CORT | AB000263 | 16.34 | 46.81 | 7.96 | 0.17 | 16.34 | 37.50 | 20.88 | 0.56 |
| 221151_at | 56979 | PRDM9 | NM_020227 | 4.44 | 27.14 | 4.63 | 0.17 | 4.44 | 4.60 | 12.30 | |
| 208606_s_at | 54361 | WNT4 | NM_030761 | 3.95 | 20.76 | 3.55 | 0.17 | 3.95 | 6.18 | 6.72 | |
| 208559_at | 3651 | PDX1 | NM_013311 | 7.16 | 17.20 | 2.94 | 0.17 | 7.16 | 12.64 | 5.51 | 0.44 |
| 207409_at | 3950 | LECT2 | NM_002302 | 7.49 | 15.66 | 2.68 | 0.17 | 7.49 | 6.38 | 1.43 | |
| 203475_at | 1588 | CYP19A1 | NM_000103 | 30.55 | 83.20 | 14.26 | 0.17 | 30.55 | 79.24 | 84.70 | |
| 216929_x_at | 28 | ABO | U15197 | 17.66 | 37.04 | 6.35 | 0.17 | 17.66 | 36.60 | 10.58 | 0.29 |
| 216857_at | | | L48728 | 36.05 | 57.75 | 9.93 | 0.17 | 36.05 | 11.23 | 35.47 | |
| 210915_x_at | 28568 | TRBV19 | M15564 | 16.84 | 34.84 | 6.00 | 0.17 | 16.84 | 14.86 | 11.93 | |
| | // | /// | | | | | | | | | |
| | | TRBC1 | | | | | | | | | |
| 214883_at | 7067 | THRA | X55005 | 4.36 | 19.94 | 3.46 | 0.17 | 4.36 | 4.15 | 7.25 | |
| 209670_at | 28755 | TRAC | M12959 | 51.55 | 212.86 | 36.99 | 0.17 | 51.55 | 68.90 | 29.65 | |
| 204972_at | 4939 | OAS2 | NM_016817 | 275.77 | 1007.17 | 175.54 | 0.17 | 275.77 | 955.50 | 240.13 | 0.25 |
| 213630_at | 23148 | KIAA0363 | AB002361 | 6.20 | 15.38 | 2.69 | 0.17 | 6.20 | 4.14 | 14.27 | |
| 215295_at | 1838 | DTNB | Y15718 | 3.32 | 16.46 | 2.88 | 0.17 | 3.32 | 7.02 | 4.89 | |
| 208391_s_at | 2740 | GLP1R | NM_002062 | 4.03 | 11.14 | 1.95 | 0.17 | 4.03 | 5.25 | 2.39 | |
| 217705_at | 5587 | PRKD1 | AW085172 | 1.71 | 18.82 | 3.31 | 0.18 | 1.71 | 17.69 | 0.89 | 0.05 |
| 209768_s_at | 2812 /// | GP1BB | AI860917 | 13.81 | 37.74 | 6.65 | 0.18 | 13.81 | 4.57 | 8.18 | |
| | | /// | | | | | | | | | |
| | | SEPT5 | | | | | | | | | |
| 206227_at | 8483 | CILP | NM_003613 | 17.85 | 49.71 | 8.80 | 0.18 | 17.85 | 22.56 | 28.21 | |
| 213004_at | 23452 | ANGPTL2 | AI074333 | 20.60 | 54.18 | 9.63 | 0.18 | 20.60 | 19.17 | 28.22 | |
| 207267_s_at | 53820 | DSCR6 | NM_018962 | 14.60 | 28.74 | 5.12 | 0.18 | 14.60 | 8.45 | 16.28 | |
| 215478_at | 9699 | RIMS2 | AF007156 | 9.55 | 45.67 | 8.16 | 0.18 | 9.55 | 13.17 | 16.35 | |
| 215361_at | | | AK022242 | 3.15 | 16.21 | 2.90 | 0.18 | 3.15 | 1.62 | 1.33 | |
| 200939_s_at | 473 | RERE | AI920976 | 3.12 | 14.35 | 2.57 | 0.18 | 3.12 | 5.87 | 2.95 | |
| 207209_at | 1068 | CETN1 | NM_004066 | 36.81 | 55.51 | 9.95 | 0.18 | 36.81 | 17.31 | 40.48 | |
| 206416_at | 7755 | ZNF205 | NM_003456 | 39.32 | 77.41 | 13.88 | 0.18 | 39.32 | 13.12 | 21.92 | |
| 220562_at | 54905 | CYP2W1 | NM_017781 | 37.04 | 73.39 | 13.16 | 0.18 | 37.04 | 29.51 | 31.98 | |
| 216651_s_at | 2572 | GAD2 | X69936 | 3.24 | 35.32 | 6.34 | 0.18 | 3.24 | 11.09 | 10.07 | |
| 211198_s_at | 23308 | ICOSLG | AF289028 | 12.92 | 114.47 | 20.57 | 0.18 | 12.92 | 147.71 | 172.98 | |
| 214393_at | 8153 | RND2 | AI884814 | 8.89 | 40.89 | 7.35 | 0.18 | 8.89 | 17.19 | 21.45 | |
| 215703_at | 1080 | CFTR | W60595 | 13.92 | 38.36 | 6.89 | 0.18 | 13.92 | 18.69 | 15.56 | |
| 210199_at | 1409 | CRYAA | U66584 | 12.28 | 34.91 | 6.28 | 0.18 | 12.28 | 24.91 | 20.91 | |
| 203084_at | 7040 | TGFB1 | NM_000660 | 41.41 | 91.16 | 16.41 | 0.18 | 41.41 | 38.49 | 11.22 | |
| 216790_at | 9154 | SLC28A1 | AK026465 | 6.42 | 23.05 | 4.15 | 0.18 | 6.42 | 16.15 | 10.52 | 0.65 |
| 209277_at | 7980 | TFPI2 | AL574096 | 5.96 | 13.68 | 2.47 | 0.18 | 5.96 | 164.41 | 445.38 | |
| 221292_at | 8643 | PTCH2 | NM_003738 | 21.02 | 36.76 | 6.63 | 0.18 | 21.02 | 7.94 | 18.39 | |
| 207542_s_at | 358 | AQP1 | NM_000385 | 78.24 | 562.65 | 101.82 | 0.18 | 78.24 | 138.56 | 121.71 | |
| 216805_at | 50807 | DDEF1 | AK027254 | 2.26 | 24.10 | 4.36 | 0.18 | 2.26 | 15.19 | 13.08 | |
| 210008_s_at | 6183 | MRPS12 | BC001617 | 38.78 | 84.69 | 15.34 | 0.18 | 38.78 | 73.25 | 59.12 | |
| 220559_at | 2019 | EN1 | NM_001426 | 12.44 | 20.59 | 3.73 | 0.18 | 12.44 | 10.77 | 13.77 | |
| 222328_x_at | 55384 | MEG3 | AI133721 | 7.82 | 30.86 | 5.63 | 0.18 | 7.82 | 4.12 | 10.67 | |
| 213537_at | 3113 | HLA- | AI128225 | 7.89 | 14.48 | 2.65 | 0.18 | 7.89 | 15.62 | 12.45 | |
| | | DPA1 | | | | | | | | | |
| 216314_at | 167 | CRISP1 | X95238 | 2.14 | 14.60 | 2.68 | 0.18 | 2.14 | 2.81 | 1.09 | |
| 203131_at | 5156 | PDGFRA | NM_006206 | 7.99 | 16.02 | 2.95 | 0.18 | 7.99 | 6.21 | 11.43 | |
| 210569_s_at | 27180 | SIGLEC9 | AF247180 | 20.09 | 32.10 | 5.92 | 0.18 | 20.09 | 4.65 | 12.82 | |
| 206596_s_at | 4901 | NRL | M81840 | 5.03 | 40.72 | 7.52 | 0.18 | 5.03 | 3.10 | 10.56 | |
| 203888_at | 7056 | THBD | NM_000361 | 399.76 | 963.86 | 179.21 | 0.19 | 399.76 | 1715.58 | 491.61 | 0.29 |
| 216821_at | 149501 / | KRT8 | AL137067 | 19.09 | 54.43 | 10.13 | 0.19 | 19.09 | 24.72 | 17.91 | |
| | | /// | | | | | | | | | |
| | | LOC149501 | | | | | | | | | |
| | | /// | | | | | | | | | |
| | | LOC3443 | | | | | | | | | |
| 205840_x_at | 2688 | GH1 | NM_000515 | 4.95 | 17.81 | 3.32 | 0.19 | 4.95 | 13.28 | 14.15 | |
| 215941_at | | | D16471 | 4.71 | 19.57 | 3.65 | 0.19 | 4.71 | 1.92 | 5.43 | |
| 206242_at | 9032 | TM4SF5 | NM_003963 | 8.09 | 19.69 | 3.69 | 0.19 | 8.09 | 11.08 | 7.52 | |
| 209752_at | 5967 | REG1A | AF172331 | 3.75 | 23.56 | 4.42 | 0.19 | 3.75 | 3.67 | 3.30 | |
| 206454_s_at | 6010 | RHO | AA058836 | 8.78 | 41.48 | 7.80 | 0.19 | 8.78 | 17.96 | 3.50 | 0.19 |
| 208007_at | | | NM_012122 | 15.22 | 23.18 | 4.37 | 0.19 | 15.22 | 4.61 | 16.67 | |
| 221438_s_at | 56158 | TEX12 | NM_031275 | 3.55 | 11.57 | 2.18 | 0.19 | 3.55 | 11.24 | 6.70 | 0.60 |
| 203887_s_at | 7056 | THBD | NM_000361 | 729.02 | 1651.91 | 312.45 | 0.19 | 729.02 | 3541.42 | 894.93 | 0.25 |
| 215226_at | 23086 | EXPH5 | AK000786 | 14.05 | 34.33 | 6.51 | 0.19 | 14.05 | 4.96 | 13.74 | |
| 221183_at | | | NM_025064 | 1.53 | 16.94 | 3.21 | 0.19 | 1.53 | 1.83 | 4.94 | |
| 214385_s_at | 4586 /// | MUC5AC | AI521646 | 2.20 | 22.52 | 4.28 | 0.19 | 2.20 | 9.30 | 2.79 | |
| | | /// | | | | | | | | | |
| | | LOC730855 | | | | | | | | | |
| 216690_at | 26664 | OR7C1 | AC005255 | 3.57 | 21.89 | 4.16 | 0.19 | 3.57 | 5.94 | 8.16 | |
| 222224_at | 342538 | NACAL | AJ278883 | 19.79 | 36.46 | 6.93 | 0.19 | 19.79 | 9.71 | 4.58 | |
| 203009_at | 4059 | BCAM | NM_005581 | 18.07 | 55.85 | 10.62 | 0.19 | 18.07 | 29.07 | 15.04 | 0.52 |
| 207578_s_at | 3360 | HTR4 | NM_000870 | 29.50 | 172.70 | 32.87 | 0.19 | 29.50 | 47.16 | 57.22 | |
| 220373_at | 54798 | DCHS2 | NM_017639 | 3.99 | 14.50 | 2.77 | 0.19 | 3.99 | 5.26 | 3.45 | |
| 216708_x_at | 3535 | IGL@ | D84143 | 11.02 | 29.20 | 5.57 | 0.19 | 11.02 | 8.06 | 7.85 | |
| 219103_at | 55616 | DDEFL1 | NM_017707 | 14.17 | 78.77 | 15.03 | 0.19 | 14.17 | 27.58 | 5.68 | 0.21 |
| 205266_at | 3976 | LIF | NM_002309 | 14.44 | 39.25 | 7.51 | 0.19 | 14.44 | 61.96 | 121.11 | |
| 220535_at | 55138 | FAM90A1 | NM_018088 | 10.39 | 26.78 | 5.12 | 0.19 | 10.39 | 7.43 | 2.73 | |
| 205552_s_at | 4938 | OAS1 | NM_002534 | 137.96 | 744.19 | 142.55 | 0.19 | 137.96 | 363.07 | 107.94 | 0.30 |
| 216709_at | 400655 | LOC400655 | AL162040 | 9.34 | 36.70 | 7.03 | 0.19 | 9.34 | 7.20 | 7.33 | |
| 204684_at | 4884 | NPTX1 | NM_002522 | 64.95 | 318.61 | 61.26 | 0.19 | 64.95 | 1104.38 | 533.67 | 0.48 |
| 215534_at | | | AL117546 | 17.98 | 56.95 | 11.02 | 0.19 | 17.98 | 60.54 | 14.84 | 0.25 |
| 210454_s_at | 3763 | KCNJ6 | U24660 | 7.82 | 23.44 | 4.55 | 0.19 | 7.82 | 36.83 | 5.96 | 0.16 |
| 216722_at | 139538 | VENTXP1 | AF164963 | 3.43 | 17.55 | 3.41 | 0.19 | 3.43 | 25.00 | 5.14 | 0.21 |
| 216626_at | | | AL050026 | 6.50 | 22.14 | 4.31 | 0.19 | 6.50 | 9.22 | 6.38 | |
| 211602_s_at | 7220 | TRPC1 | U31110 | 21.05 | 39.13 | 7.65 | 0.20 | 21.05 | 5.40 | 24.02 | |
| 203325_s_at | 1289 | COL5A1 | AI130969 | 8.42 | 13.96 | 2.73 | 0.20 | 8.42 | 8.56 | 2.05 | |
| 202869_at | 4938 | OAS1 | NM_016816 | 165.01 | 762.84 | 149.48 | 0.20 | 165.01 | 722.61 | 133.48 | 0.18 |
| 217232_x_at | 3043 | HBB | AF059180 | 26.73 | 55.35 | 10.85 | 0.20 | 26.73 | 35.18 | 74.89 | |
| 207193_at | 181 | AGRP | NM_001138 | 10.75 | 46.71 | 9.17 | 0.20 | 10.75 | 17.91 | 16.59 | |
| 213541_s_at | 2078 | ERG | AI351043 | 4.58 | 33.06 | 6.49 | 0.20 | 4.58 | 23.23 | 30.75 | |
| 206155_at | 1244 | ABCC2 | NM_000392 | 2.30 | 10.16 | 2.00 | 0.20 | 2.30 | 10.86 | 6.30 | 0.58 |
| 209559_at | 9026 | HIP1R | AB013384 | 12.55 | 50.86 | 10.02 | 0.20 | 12.55 | 13.89 | 5.17 | |
| 207944_at | 4951 | OCM | NM_006188 | 10.46 | 32.47 | 6.40 | 0.20 | 10.46 | 5.00 | 5.29 | |
| 207368_at | 3352 | HTR1D | NM_000864 | 1.23 | 37.50 | 7.40 | 0.20 | 1.23 | 4.58 | 13.42 | |
| 216875_x_at | 55547 | HAB1 | X83412 | 18.50 | 33.24 | 6.57 | 0.20 | 18.50 | 20.61 | 24.45 | |
| 202458_at | 11098 | PRSS23 | NM_007173 | 15.23 | 45.10 | 8.91 | 0.20 | 15.23 | 157.75 | 161.56 | |
| 207460_at | 3004 | GZMM | NM_005317 | 15.65 | 38.99 | 7.71 | 0.20 | 15.65 | 12.47 | 8.42 | |
| 211608_at | | | U50073 | 3.99 | 26.51 | 5.26 | 0.20 | 3.99 | 20.38 | 4.28 | 0.21 |
| 204851_s_at | 1641 | DCX | BE551351 | 2.49 | 15.40 | 3.06 | 0.20 | 2.49 | 2.96 | 3.78 | |
| 214461_at | 3929 | LBP | NM_004139 | 4.72 | 13.83 | 2.75 | 0.20 | 4.72 | 2.55 | 4.53 | |
| 221409_at | 56656 | OR2S2 | NM_019897 | 16.57 | 44.94 | 8.94 | 0.20 | 16.57 | 44.62 | 17.53 | 0.39 |
| 204395_s_at | 2869 | GRK5 | NM_005308 | 4.85 | 39.76 | 7.93 | 0.20 | 4.85 | 13.18 | 3.49 | 0.26 |
| 206795_at | 2151 | F2RL2 | NM_004101 | 5.69 | 24.09 | 4.81 | 0.20 | 5.69 | 11.45 | 1.02 | 0.09 |
| 205495_s_at | 10578 | GNLY | NM_006433 | 20.35 | 49.27 | 9.84 | 0.20 | 20.35 | 3.70 | 5.10 | |
| 219514_at | 23452 | ANGPTL2 | NM_012098 | 5.02 | 35.13 | 7.02 | 0.20 | 5.02 | 13.05 | 4.40 | 0.34 |
| 220142_at | 60484 | HAPLN2 | NM_021817 | 25.08 | 49.29 | 9.85 | 0.20 | 25.08 | 14.99 | 21.25 | |
| 208181_at | 8365 | HIST1H4H | NM_003543 | 1.92 | 18.25 | 3.65 | 0.20 | 1.92 | 2.67 | 15.62 | |
| 203596_s_at | 24138 | IFIT5 | NM_012420 | 8.22 | 53.28 | 10.67 | 0.20 | 8.22 | 91.58 | 20.13 | 0.22 |
| 206883_x_at | 2815 | GP9 | NM_000174 | 44.47 | 74.63 | 14.96 | 0.20 | 44.47 | 20.41 | 8.43 | |
| 206856_at | 10990 | LILRB5 | NM_006840 | 6.75 | 16.31 | 3.28 | 0.20 | 6.75 | 7.61 | 12.82 | |
| 207349_s_at | 7352 | UCP3 | NM_022803 | 18.73 | 84.10 | 16.96 | 0.20 | 18.73 | 27.32 | 31.07 | |
| 207638_at | 5651 | PRSS7 | NM_002772 | 0.46 | 10.71 | 2.16 | 0.20 | 0.46 | 6.15 | 3.29 | |
| 208285_at | 26659 | OR7A5 | NM_017506 | 8.56 | 27.54 | 5.59 | 0.20 | 8.56 | 3.30 | 2.75 | |
| 214059_at | 10561 | IFI44 | BE049439 | 26.82 | 109.98 | 22.31 | 0.20 | 26.82 | 80.13 | 40.28 | 0.50 |
| 216854_at | 10220 | GDF11 | AF028333 | 6.73 | 10.80 | 2.19 | 0.20 | 6.73 | 11.00 | 4.64 | 0.42 |
| 208281_x_at | 1617 /// | DAZ1 | NM_020364 | 4.25 | 41.71 | 8.50 | 0.20 | 4.25 | 32.65 | 12.54 | 0.38 |
| | | /// | | | | | | | | | |
| | | DAZ3 | | | | | | | | | |
| | | /// | | | | | | | | | |
| | | DAZ2 | | | | | | | | | |
| | | /// DAZ | | | | | | | | | |
| 216487_at | 3709 | ITPR2 | AL049988 | 4.02 | 13.51 | 2.76 | 0.20 | 4.02 | 2.23 | 3.70 | |
| 221149_at | 27202 | GPR77 | NM_018485 | 2.75 | 25.88 | 5.30 | 0.20 | 2.75 | 51.40 | 37.04 | 0.72 |
| 207342_at | 1258 | CNGB1 | NM_001297 | 3.35 | 11.94 | 2.45 | 0.20 | 3.35 | 3.50 | 2.02 | |
| 218400_at | 4940 | OAS3 | NM_006187 | 193.77 | 989.10 | 203.04 | 0.21 | 193.77 | 715.99 | 284.58 | 0.40 |
| 220531_at | 79907 | FLJ14126 | NM_024849 | 3.50 | 13.61 | 2.80 | 0.21 | 3.50 | 4.45 | 3.32 | |
| 211103_at | 4647 | MYO7A | U55209 | 10.66 | 36.22 | 7.46 | 0.21 | 10.66 | 5.69 | 8.66 | |
| 207569_at | 6098 | ROS1 | NM_002944 | 13.54 | 40.21 | 8.28 | 0.21 | 13.54 | 10.51 | 13.64 | |
| 209992_at | 5208 | PFKFB2 | AB044805 | 6.22 | 31.87 | 6.57 | 0.21 | 6.22 | 6.47 | 14.75 | |
| 207774_at | 83734 | ATG10 | NM_025020 | 27.24 | 59.76 | 12.34 | 0.21 | 27.24 | 18.31 | 35.76 | |
| 211606_at | | | U43279 | 3.75 | 14.10 | 2.92 | 0.21 | 3.75 | 10.14 | 5.37 | 0.53 |
| 203842_s_at | 22924 | MAPRE3 | NM_012326 | 8.33 | 26.12 | 5.40 | 0.21 | 8.33 | 18.46 | 7.12 | 0.39 |
| 220534_at | 79097 | TRIM48 | NM_024114 | 22.18 | 44.07 | 9.13 | 0.21 | 22.18 | 33.60 | 12.66 | 0.38 |
| 214376_at | | | AI263044 | 6.65 | 12.21 | 2.53 | 0.21 | 6.65 | 11.63 | 13.39 | |
| 205506_at | 7429 | VIL1 | NM_007127 | 2.43 | 11.39 | 2.37 | 0.21 | 2.43 | 1.70 | 12.49 | |
| 219153_s_at | 79875 | THSD4 | BG163478 | 9.93 | 37.84 | 7.87 | 0.21 | 9.93 | 12.52 | 11.53 | |
| 208448_x_at | 3449 | IFNA16 | NM_002173 | 17.33 | 36.83 | 7.67 | 0.21 | 17.33 | 11.22 | 7.85 | |
| 206343_s_at | 3084 | NRG1 | NM_013959 | 3.46 | 11.68 | 2.45 | 0.21 | 3.46 | 10.32 | 98.74 | |
| 214987_at | | | AL049449 | 12.98 | 31.12 | 6.55 | 0.21 | 12.98 | 18.04 | 4.13 | |
| 215214_at | 3535 | IGL@ | H53689 | 15.76 | 26.23 | 5.52 | 0.21 | 15.76 | 36.35 | 46.53 | |
| 204874_x_at | 8938 | BAIAP3 | NM_003933 | 4.70 | 23.75 | 5.00 | 0.21 | 4.70 | 4.92 | 9.80 | |
| 220245_at | 51151 | SLC45A2 | NM_016180 | 34.04 | 72.99 | 15.37 | 0.21 | 34.04 | 30.18 | 31.16 | |
| 213816_s_at | 4233 | MET | AA005141 | 8.10 | 22.36 | 4.72 | 0.21 | 8.10 | 6.78 | 9.19 | |
| 213955_at | 91977 | MYOZ3 | BE673151 | 9.73 | 24.84 | 5.25 | 0.21 | 9.73 | 6.89 | 4.61 | |
| 210267_at | 57185 | NPAL3 | BC001265 | 5.35 | 35.89 | 7.59 | 0.21 | 5.35 | 14.82 | 15.15 | |
| 203990_s_at | 7403 | UTX | AI140752 | 3.30 | 10.49 | 2.23 | 0.21 | 3.30 | 9.39 | 5.38 | |
| 215010_s_at | 9024 | BRSK2 | AJ006701 | 10.54 | 37.26 | 7.92 | 0.21 | 10.54 | 2.94 | 3.94 | |
| 220622_at | 79782 | LRRC31 | NM_024727 | 3.02 | 10.62 | 2.26 | 0.21 | 3.02 | 13.15 | 10.31 | |
| 40524_at | 11099 | PTPN21 | X79510 | 4.73 | 22.60 | 4.81 | 0.21 | 4.73 | 25.76 | 35.61 | |
| 205539_at | 10677 | AVIL | NM_006576 | 33.26 | 52.80 | 11.24 | 0.21 | 33.26 | 22.28 | 22.23 | |
| 214498_at | 434 | ASIP | NM_001672 | 2.35 | 17.56 | 3.75 | 0.21 | 2.35 | 18.51 | 4.35 | 0.23 |
| 208168_s_at | 1118 | CHIT1 | NM_003465 | 5.09 | 15.71 | 3.36 | 0.21 | 5.09 | 3.62 | 2.89 | |
| 214200_s_at | 1291 | COL6A1 | AI193744 | 4.27 | 25.62 | 5.49 | 0.21 | 4.27 | 25.50 | 7.40 | 0.29 |
| 214947_at | | | AF052146 | 173.91 | 458.82 | 98.30 | 0.21 | 173.91 | 100.82 | 30.97 | |
| 220957_at | 64693 | CTAGE1 | NM_022663 | 10.02 | 46.85 | 10.10 | 0.22 | 10.02 | 15.60 | 18.19 | |
| 216472_at | 6453 | ITSN1 | AF003737 | 8.07 | 52.67 | 11.39 | 0.22 | 8.07 | 25.37 | 37.51 | |
| 213905_x_at | 10194 | BGN | AA845258 | 3.46 | 15.70 | 3.39 | 0.22 | 3.46 | 12.08 | 19.73 | |
| | // | /// | | | | | | | | | |
| | | TSHZ1 | | | | | | | | | |
| 204845_s_at | 2028 | ENPEP | NM_001977 | 3.08 | 12.61 | 2.73 | 0.22 | 3.08 | 2.76 | 1.73 | |
| 211442_x_at | 64816 | CYP3A43 | AF280111 | 6.49 | 24.68 | 5.34 | 0.22 | 6.49 | 6.58 | 5.42 | |
| 202997_s_at | 4017 | LOXL2 | BE251211 | 3.18 | 10.97 | 2.38 | 0.22 | 3.18 | 3.93 | 3.30 | |
| 216612_x_at | | | AK021988 | 8.07 | 18.70 | 4.07 | 0.22 | 8.07 | 12.79 | 21.33 | |
| 219211_at | 11274 | USP18 | NM_017414 | 108.73 | 392.94 | 85.42 | 0.22 | 108.73 | 273.28 | 66.66 | 0.24 |
| | // | /// | | | | | | | | | |
| | | LOC727996 | | | | | | | | | |
| | | /// | | | | | | | | | |
| | | LOC728 | | | | | | | | | |
| 222178_s_at | | | AL133262 | 3.73 | 10.55 | 2.30 | 0.22 | 3.73 | 1.64 | 3.70 | |
| 204870_s_at | 5126 | PCSK2 | NM_002594 | 12.30 | 33.16 | 7.22 | 0.22 | 12.30 | 13.17 | 5.12 | |
| 211832_s_at | 4193 | MDM2 | AF201370 | 11.58 | 20.16 | 4.40 | 0.22 | 11.58 | 33.23 | 25.55 | |
| 221342_at | 80739 | C6orf25 | NM_025260 | 8.88 | 28.62 | 6.26 | 0.22 | 8.88 | 4.78 | 13.74 | |
| 201438_at | 1293 | COL6A3 | NM_004369 | 7.30 | 11.62 | 2.55 | 0.22 | 7.30 | 6.17 | 5.79 | |
| 206455_s_at | 6010 | RHO | NM_000539 | 21.22 | 38.08 | 8.36 | 0.22 | 21.22 | 41.58 | 10.28 | 0.25 |
| 208020_s_at | 775 | CACNA1C | NM_000719 | 8.53 | 22.08 | 4.85 | 0.22 | 8.53 | 3.30 | 13.32 | |
| 222254_at | | | AL034429 | 4.11 | 16.86 | 3.70 | 0.22 | 4.11 | 2.65 | 5.24 | |
| 208219_at | 91 | ACVR1B | NM_020328 | 8.45 | 45.25 | 9.95 | 0.22 | 8.45 | 18.74 | 12.83 | 0.68 |
| 208208_at | 8735 | MYH13 | NM_003802 | 6.15 | 37.65 | 8.29 | 0.22 | 6.15 | 4.01 | 17.13 | |
| 209159_s_at | 65009 | NDRG4 | AV724216 | 8.20 | 70.63 | 15.63 | 0.22 | 8.20 | 10.61 | 9.35 | |
| 208545_x_at | 6874 | TAF4 | NM_003185 | 28.98 | 44.60 | 9.88 | 0.22 | 28.98 | 20.75 | 22.09 | |
| 217490_at | | | AL049301 | 11.98 | 47.84 | 10.61 | 0.22 | 11.98 | 21.29 | 23.32 | |
| 205914_s_at | 2902 /// | GRIN1 | D13515 | 4.39 | 11.05 | 2.47 | 0.22 | 4.39 | 9.71 | 9.90 | |
| | | /// | | | | | | | | | |
| | | LOC731701 | | | | | | | | | |
| 207034_s_at | 2736 | GLI2 | NM_030379 | 3.03 | 37.90 | 8.46 | 0.22 | 3.03 | 2.71 | 4.92 | |
| 205018_s_at | 10150 | MBNL2 | NM_005757 | 4.63 | 18.46 | 4.14 | 0.22 | 4.63 | 16.00 | 5.20 | 0.33 |
| 207420_at | 10584 | COLEC10 | NM_006438 | 8.48 | 14.72 | 3.30 | 0.22 | 8.48 | 11.84 | 3.45 | |
| 217236_x_at | 3500 | IGHG1 | S74639 | 4.47 | 60.17 | 13.48 | 0.22 | 4.47 | 25.60 | 30.56 | |
| 214492_at | 6444 | SGCD | NM_000337 | 3.20 | 23.26 | 5.24 | 0.23 | 3.20 | 9.28 | 22.52 | |
| 207151_at | 117 | ADCYAP1R1 | NM_001118 | 24.36 | 39.56 | 8.96 | 0.23 | 24.36 | 11.34 | 11.52 | |
| 202270_at | 2633 | GBP1 | NM_002053 | 3.58 | 17.81 | 4.03 | 0.23 | 3.58 | 7.29 | 7.94 | |
| 204802_at | 6236 | RRAD | NM_004165 | 1.53 | 77.18 | 17.53 | 0.23 | 1.53 | 249.55 | 639.77 | |
| 216245_at | 3557 | IL1RN | BE563442 | 4.56 | 23.85 | 5.42 | 0.23 | 4.56 | 6.81 | 25.37 | |
| 210159_s_at | 11074 | TRIM31 | AF230386 | 8.30 | 24.94 | 5.69 | 0.23 | 8.30 | 11.07 | 9.49 | |
| 209850_s_at | 10435 | CDC42EP2 | BC005406 | 22.00 | 71.03 | 16.22 | 0.23 | 22.00 | 32.79 | 21.64 | |
| 217428_s_at | 1300 | COL10A1 | X98568 | 6.56 | 20.03 | 4.59 | 0.23 | 6.56 | 4.20 | 5.81 | |
| 206975_at | 4049 | LTA | NM_000595 | 3.72 | 18.73 | 4.30 | 0.23 | 3.72 | 58.43 | 26.95 | 0.46 |
| 212821_at | 26030 | PLEKHG3 | AI738980 | 29.35 | 93.51 | 21.47 | 0.23 | 29.35 | 7.41 | 27.90 | |
| 215076_s_at | 1281 | COL3A1 | AU144167 | 5.45 | 11.08 | 2.55 | 0.23 | 5.45 | 29.13 | 8.76 | 0.30 |
| 219761_at | 51267 | CLEC1A | NM_016511 | 12.48 | 37.32 | 8.57 | 0.23 | 12.48 | 30.77 | 14.58 | 0.47 |
| 214324_at | 2813 | GP2 | BF222483 | 13.12 | 24.61 | 5.66 | 0.23 | 13.12 | 7.42 | 4.76 | |
| 218186_at | 57111 | RAB25 | NM_020387 | 4.04 | 10.27 | 2.36 | 0.23 | 4.04 | 5.81 | 5.61 | |
| 211169_s_at | 5506 | PPP1R3A | AF024579 | 4.63 | 16.00 | 3.69 | 0.23 | 4.63 | 15.83 | 13.96 | |
| 209013_x_at | 7204 | TRIO | AF091395 | 12.35 | 18.84 | 4.35 | 0.23 | 12.35 | 9.26 | 8.74 | |
| 217688_at | | | AA224446 | 3.53 | 29.19 | 6.75 | 0.23 | 3.53 | 2.54 | 5.29 | |
| 207737_at | | | NM_021981 | 6.02 | 20.00 | 4.63 | 0.23 | 6.02 | 16.27 | 2.37 | 0.15 |
| 206372_at | 4618 | MYF6 | NM_002469 | 7.85 | 29.22 | 6.77 | 0.23 | 7.85 | 6.07 | 3.02 | |
| 214604_at | 3237 | HOXD11 | NM_021192 | 3.35 | 19.53 | 4.53 | 0.23 | 3.35 | 19.69 | 11.06 | 0.56 |
| 207978_s_at | 8013 | NR4A3 | NM_006981 | 6.57 | 62.28 | 14.50 | 0.23 | 6.57 | 139.87 | 137.78 | |
| 216581_at | 441133 | LOC441133 | AL022068 | 3.70 | 33.06 | 7.71 | 0.23 | 3.70 | 8.64 | 4.16 | |
| 207656_s_at | 51 | ACOX1 | NM_004035 | 3.97 | 18.29 | 4.27 | 0.23 | 3.97 | 6.86 | 3.38 | |
| 221914_at | 6853 | SYN1 | H19843 | 4.35 | 24.00 | 5.60 | 0.23 | 4.35 | 7.60 | 6.39 | |
| 212489_at | 1289 | COL5A1 | AI983428 | 2.67 | 13.99 | 3.28 | 0.23 | 2.67 | 8.97 | 15.19 | |
| 209789_at | 10391 | CORO2B | BF939649 | 11.86 | 20.38 | 4.78 | 0.23 | 11.86 | 32.55 | 7.48 | 0.23 |
| 208253_at | 27181 | SIGLEC8 | NM_014442 | 7.55 | 25.65 | 6.04 | 0.24 | 7.55 | 2.42 | 24.75 | |
| 201235_s_at | 7832 | BTG2 | BG339064 | 110.45 | 1093.07 | 257.99 | 0.24 | 110.45 | 709.50 | 1467.85 | |
| 215901_at | 347344 | ZNF81 | X68011 | 5.87 | 34.73 | 8.20 | 0.24 | 5.87 | 3.30 | 10.64 | |
| 220289_s_at | 55057 | AIM1L | NM_017977 | 7.82 | 13.51 | 3.19 | 0.24 | 7.82 | 4.11 | 6.45 | |
| | // | /// | | | | | | | | | |
| | | FLJ38020 | | | | | | | | | |
| 217160_at | 7258 | TSPY1 | M94893 | 3.58 | 13.64 | 3.22 | 0.24 | 3.58 | 4.89 | 5.05 | |
| 207427_at | 49 | ACR | NM_001097 | 3.24 | 16.20 | 3.84 | 0.24 | 3.24 | 8.57 | 4.94 | |
| 219364_at | 79132 | LGP2 | NM_024119 | 12.91 | 56.98 | 13.50 | 0.24 | 12.91 | 36.47 | 9.26 | 0.25 |
| 210880_s_at | 10278 | EFS | AB001467 | 11.19 | 35.86 | 8.50 | 0.24 | 11.19 | 2.73 | 20.70 | |
| 214490_at | 416 | ARSF | NM_004042 | 17.84 | 35.68 | 8.48 | 0.24 | 17.84 | 17.39 | 10.11 | |
| 214799_at | 23114 | NFASC | AI821777 | 5.41 | 17.95 | 4.27 | 0.24 | 5.41 | 3.82 | 2.01 | |
| 207114_at | 80740 | LY6G6C | NM_025261 | 14.12 | 36.43 | 8.66 | 0.24 | 14.12 | 47.18 | 31.42 | 0.67 |
| 207900_at | 6361 | CCL17 | NM_002987 | 3.85 | 21.82 | 5.19 | 0.24 | 3.85 | 22.03 | 36.17 | |
| 207690_at | 257 | ALX3 | NM_006492 | 11.07 | 18.24 | 4.34 | 0.24 | 11.07 | 7.42 | 9.50 | |
| 209671_x_at | 28755 | TRA@ | M12423 | 24.38 | 104.03 | 24.78 | 0.24 | 24.38 | 17.84 | 13.99 | |
| | // | /// | | | | | | | | | |
| | | TRAC | | | | | | | | | |
| 207110_at | 3768 | KCNJ12 | NM_021012 | 6.09 | 12.02 | 2.87 | 0.24 | 6.09 | 3.06 | 2.22 | |
| 217275_at | | | L77565 | 13.24 | 40.54 | 9.67 | 0.24 | 13.24 | 10.39 | 10.70 | |
| 205261_at | 5225 | PGC | NM_002630 | 33.37 | 62.29 | 14.92 | 0.24 | 33.37 | 61.82 | 39.44 | 0.64 |
| 206123_at | 3996 | LLGL1 | D50550 | 17.03 | 44.07 | 10.59 | 0.24 | 17.03 | 8.65 | 22.49 | |
| 215647_at | 64062 | RBM26 | AK021576 | 0.35 | 16.36 | 3.94 | 0.24 | 0.35 | 8.09 | 2.65 | |
| 203868_s_at | 7412 | VCAM1 | NM_001078 | 8.54 | 412.52 | 99.55 | 0.24 | 8.54 | 498.26 | 82.10 | 0.16 |
| 217660_at | 79784 | MYH14 | AW188214 | 3.59 | 17.44 | 4.22 | 0.24 | 3.59 | 11.95 | 20.39 | |
| 205998_x_at | 1576 | CYP3A4 | NM_017460 | 81.17 | 152.61 | 37.01 | 0.24 | 81.17 | 99.79 | 134.07 | |
| 220158_at | 56891 | LGALS14 | NM_020129 | 23.45 | 37.04 | 8.99 | 0.24 | 23.45 | 43.75 | 15.17 | 0.35 |
| 216312_at | 492 | ATP2B3 | AW615612 | 12.39 | 26.54 | 6.45 | 0.24 | 12.39 | 34.49 | 5.68 | 0.16 |
| 217334_at | 442186 | OR2J3 | AL022727 | 7.41 | 13.17 | 3.20 | 0.24 | 7.41 | 2.84 | 2.97 | |
| 208142_at | 10876 | FAM12A | NM_006683 | 6.33 | 10.44 | 2.54 | 0.24 | 6.33 | 4.73 | 2.79 | |
| 210684_s_at | 1742 | DLG4 | AF028825 | 9.47 | 40.37 | 9.83 | 0.24 | 9.47 | 9.74 | 17.94 | |
| 48031_r_at | 10826 | C5orf4 | H93077 | 12.87 | 28.02 | 6.84 | 0.24 | 12.87 | 15.35 | 17.71 | |
| 220639_at | 79853 | TM4SF20 | NM_024795 | 8.65 | 14.86 | 3.63 | 0.24 | 8.65 | 12.17 | 2.57 | |
| 204912_at | 3587 | IL10RA | NM_001558 | 460.98 | 2093.69 | 512.21 | 0.24 | 460.98 | 3175.53 | 1078.66 | 0.34 |
| 215063_x_at | 55631 | LRRC40 | AL390149 | 127.53 | 218.71 | 53.60 | 0.25 | 127.53 | 332.09 | 119.06 | 0.36 |
| 219416_at | 51435 | SCARA3 | NM_016240 | 20.83 | 44.03 | 10.82 | 0.25 | 20.83 | 29.92 | 31.71 | |
| 204018_x_at | 3039 /// | HBA1 | NM_000558 | 26.22 | 50.79 | 12.49 | 0.25 | 26.22 | 11.05 | 11.47 | |
| | | /// | | | | | | | | | |
| | | HBA2 | | | | | | | | | |
| 221170_at | 59340 | HRH4 | AF312230 | 8.58 | 15.44 | 3.80 | 0.25 | 8.58 | 7.70 | 6.49 | |
| 211640_x_at | 4917 | NTN2L | L23519 | 3.85 | 10.12 | 2.49 | 0.25 | 3.85 | 3.25 | 3.96 | |
| 207434_s_at | 4151 /// | FXYD2 | NM_021603 | 15.62 | 28.70 | 7.07 | 0.25 | 15.62 | 75.78 | 12.35 | 0.16 |
| | | /// | | | | | | | | | |
| | | MB | | | | | | | | | |
| 207967_at | 11311 | VPS45 | NM_007258 | 14.12 | 49.68 | 12.26 | 0.25 | 14.12 | 9.45 | 15.67 | |
| 214228_x_at | 7293 | TNFRSF4 | AJ277151 | 2.59 | 13.05 | 3.22 | 0.25 | 2.59 | 215.37 | 126.68 | 0.59 |
| 205483_s_at | 9636 | ISG15 | NM_005101 | 404.40 | 2028.75 | 501.75 | 0.25 | 404.40 | 1879.17 | 752.00 | 0.40 |
| 202335_s_at | 7320 | UBE2B | NM_003337 | 27.08 | 65.10 | 16.12 | 0.25 | 27.08 | 33.41 | 25.60 | |
| 214720_x_at | 151011 | 10-Sep | BF981643 | 5.14 | 16.24 | 4.02 | 0.25 | 5.14 | 1.41 | 5.50 | |
| 211046_at | 81033 | KCNH6 | BC006334 | 8.79 | 18.09 | 4.49 | 0.25 | 8.79 | 6.46 | 19.86 | |
| 204237_at | 51454 | GULP1 | NM_016315 | 5.54 | 11.53 | 2.86 | 0.25 | 5.54 | 66.23 | 15.16 | 0.23 |
| 203153_at | 3434 | IFIT1 | NM_001548 | 48.44 | 560.68 | 139.17 | 0.25 | 48.44 | 703.99 | 149.53 | 0.21 |
| 201374_x_at | 5516 | PPP2CB | AI379894 | 30.08 | 48.17 | 11.97 | 0.25 | 30.08 | 27.87 | 30.29 | |
| 215815_at | 8896 | BUD31 | AK001615 | 6.09 | 18.48 | 4.60 | 0.25 | 6.09 | 5.89 | 3.75 | |
| 215658_at | | | AK024897 | 2.77 | 12.42 | 3.09 | 0.25 | 2.77 | 4.54 | 7.99 | |
| 211157_at | | | AF119878 | 13.22 | 31.90 | 7.94 | 0.25 | 13.22 | 15.37 | 10.23 | |
| 217257_at | | | L37198 | 37.87 | 76.37 | 19.04 | 0.25 | 37.87 | 81.33 | 48.87 | 0.60 |
| 214400_at | 3640 | INSL3 | AI991694 | 8.68 | 19.24 | 4.80 | 0.25 | 8.68 | 14.49 | 10.55 | 0.73 |
| 206318_at | 57119 | SPINLW1 | NM_020398 | 11.11 | 35.45 | 8.85 | 0.25 | 11.11 | 14.15 | 21.68 | |
| 211670_x_at | 10214 | SSX3 | S82471 | 5.16 | 25.54 | 6.41 | 0.25 | 5.16 | 5.96 | 6.45 | |
| 221091_at | 10022 | INSL5 | NM_005478 | 17.80 | 41.97 | 10.54 | 0.25 | 17.80 | 15.50 | 12.30 | |
| 208111_at | 554 | AVPR2 | NM_000054 | 7.29 | 11.61 | 2.92 | 0.25 | 7.29 | 12.39 | 6.95 | 0.56 |
| 207144_s_at | 4435 | CITED1 | NM_004143 | 4.42 | 17.74 | 4.47 | 0.25 | 4.42 | 5.58 | 6.42 | |
| 205216_s_at | 350 | APOH | NM_000042 | 27.83 | 42.62 | 10.75 | 0.25 | 27.83 | 8.56 | 4.24 | |
| 208372_s_at | 3984 | LIMK1 | AF134379 | 31.95 | 67.95 | 17.21 | 0.25 | 31.95 | 9.65 | 11.98 | |
| 207562_at | 1609 | DGKQ | NM_001347 | 42.97 | 76.12 | 19.32 | 0.25 | 42.97 | 55.44 | 30.29 | |
| 217110_s_at | 4585 | MUC4 | AJ242547 | 8.32 | 17.23 | 4.37 | 0.25 | 8.32 | 9.78 | 3.27 | |
| 221331_x_at | 1493 | CTLA4 | NM_005214 | 6.30 | 33.90 | 8.63 | 0.25 | 6.30 | 37.21 | 27.95 | |
| 221033_s_at | 56163 | RNF17 | NM_031277 | 12.15 | 41.60 | 10.59 | 0.25 | 12.15 | 20.10 | 3.46 | 0.17 |
| 219962_at | 59272 | ACE2 | NM_021804 | 4.67 | 23.24 | 5.92 | 0.25 | 4.67 | 4.18 | 4.42 | |
| 216734_s_at | 643 | BLR1 | X68829 | 4.22 | 15.67 | 4.00 | 0.26 | 4.22 | 4.65 | 3.90 | |
| 206000_at | 4224 | MEP1A | NM_005588 | 4.05 | 11.34 | 2.90 | 0.26 | 4.05 | 58.09 | 8.08 | 0.14 |
| 215240_at | 3690 | ITGB3 | AI189839 | 5.55 | 10.32 | 2.64 | 0.26 | 5.55 | 4.29 | 2.77 | |
| 217545_at | 79784 | MYH14 | AW081820 | 3.56 | 17.60 | 4.50 | 0.26 | 3.56 | 5.91 | 4.04 | |
| 210745_at | 3175 | ONECUT1 | U96173 | 9.74 | 34.52 | 8.83 | 0.26 | 9.74 | 10.51 | 4.19 | |
| 215304_at | 79875 | THSD4 | U79293 | 11.88 | 45.27 | 11.60 | 0.26 | 11.88 | 13.82 | 3.30 | |
| 217004 s_at | 4168 | MCF2 | X13230 | 10.93 | 16.91 | 4.36 | 0.26 | 10.93 | 5.96 | 13.11 | |
| 220469_at | 11316 | COPE | NM_025088 | 42.07 | 70.79 | 18.26 | 0.26 | 42.07 | 19.49 | 46.10 | |
| 204626_s_at | 3690 | ITGB3 | NM_000212 | 9.87 | 32.67 | 8.44 | 0.26 | 9.87 | 17.27 | 15.12 | |
| 210085_s_at | 8416 | ANXA9 | AF230929 | 4.13 | 33.94 | 8.77 | 0.26 | 4.13 | 5.88 | 8.34 | |
| 220426_at | 79025 | C20orf195 | NM_024059 | 4.76 | 26.95 | 6.99 | 0.26 | 4.76 | 5.32 | 9.25 | |
| 209793_at | 2890 /// | GRIA1 | AL567302 | 8.25 | 12.69 | 3.29 | 0.26 | 8.25 | 8.24 | 6.30 | |
| | | /// | | | | | | | | | |
| | | RGS12 | | | | | | | | | |
| 204964_s_at | 8082 | SSPN | AL136756 | 21.60 | 41.58 | 10.80 | 0.26 | 21.60 | 13.29 | 9.02 | |
| 210855_at | 9687 | GREB1 | AF245390 | 10.75 | 36.31 | 9.44 | 0.26 | 10.75 | 31.19 | 26.24 | |
| 206602_s_at | 3232 | HOXD3 | NM_006898 | 1.77 | 26.94 | 7.01 | 0.26 | 1.77 | 7.43 | 13.58 | |
| 209855_s_at | 3817 | KLK2 | AF188747 | 6.04 | 17.55 | 4.57 | 0.26 | 6.04 | 4.04 | 6.82 | |
| 213230_at | 30850 | CDR2L | AI422335 | 6.04 | 39.05 | 10.22 | 0.26 | 6.04 | 8.31 | 18.48 | |
| 221852_at | 408050 | NOMO3 | N39536 | 5.97 | 13.72 | 3.59 | 0.26 | 5.97 | 3.18 | 7.85 | |
| 213345_at | 4776 | NFATC4 | AI624015 | 19.25 | 37.51 | 9.85 | 0.26 | 19.25 | 9.45 | 8.72 | |
| 206400_at | 3963 | LGALS7 | NM_002307 | 6.36 | 12.72 | 3.35 | 0.26 | 6.36 | 3.16 | 11.72 | |
| 205348_s_at | 1780 | DYNC1I1 | NM_004411 | 15.06 | 26.79 | 7.07 | 0.26 | 15.06 | 18.00 | 10.60 | |
| 206236_at | 2828 | GPR4 | NM_005282 | 19.98 | 33.35 | 8.80 | 0.26 | 19.98 | 25.16 | 11.68 | |
| 207208_at | 27288 | HNRNPG-T | NM_014469 | 5.85 | 22.13 | 5.86 | 0.26 | 5.85 | 6.03 | 5.70 | |
| 210194_at | 22925 | PLA2R1 | U17033 | 5.57 | 14.96 | 3.96 | 0.26 | 5.57 | 3.92 | 24.25 | |
| 212325_at | 22998 | DKFZP686A01247 | AK026815 | 5.01 | 23.44 | 6.22 | 0.27 | 5.01 | 9.76 | 7.99 | |
| 203784_s_at | 55794 | DDX28 | BG477502 | 33.90 | 72.78 | 19.33 | 0.27 | 33.90 | 6.27 | 38.39 | |
| 205867_at | 5781 | PTPN11 | NM_002834 | 47.50 | 72.17 | 19.18 | 0.27 | 47.50 | 40.31 | 33.13 | |
| 208387_s_at | 10893 | MMP24 | NM_006690 | 24.00 | 41.40 | 11.02 | 0.27 | 24.00 | 26.02 | 8.45 | |
| 207126_x_at | 54657 | UGT1A4 | NM_000463 | 15.67 | 39.52 | 10.53 | 0.27 | 15.67 | 52.15 | 34.28 | 0.66 |
| 210179_at | 3769 | KCNJ13 | AJ007557 | 4.20 | 20.78 | 5.55 | 0.27 | 4.20 | 20.00 | 3.32 | 0.17 |
| 220809_at | 79972 | FLJ14327 | NM_024912 | 20.37 | 34.48 | 9.22 | 0.27 | 20.37 | 20.89 | 24.91 | |
| 207398_at | 3239 | HOXD13 | NM_000523 | 6.42 | 22.14 | 5.92 | 0.27 | 6.42 | 3.22 | 6.10 | |
| 206271_at | 7098 | TLR3 | NM_003265 | 7.14 | 19.04 | 5.10 | 0.27 | 7.14 | 5.85 | 7.90 | |
| 206051_at | 1996 | ELAVL4 | NM_021952 | 7.51 | 57.30 | 15.37 | 0.27 | 7.51 | 4.90 | 13.20 | |
| 203824_at | 7103 | TSPAN8 | NM_004616 | 2.54 | 11.40 | 3.06 | 0.27 | 2.54 | 5.35 | 8.58 | |
| 221008_s_at | 64850 | AGXT2L1 | NM_031279 | 5.21 | 24.33 | 6.53 | 0.27 | 5.21 | 24.32 | 7.31 | 0.30 |
| 211008_s_at | 7329 | UBE2I | BC000744 | 35.29 | 82.06 | 22.05 | 0.27 | 35.29 | 25.84 | 32.59 | |
| 209761_s_at | 3431 | SP110 | AA969194 | 422.13 | 1021.62 | 274.72 | 0.27 | 422.13 | 445.52 | 193.77 | |
| 222346_at | 284217 | LAMA1 | AI633741 | 10.50 | 21.15 | 5.69 | 0.27 | 10.50 | 3.99 | 3.35 | |
| 213783_at | 4242 | MFNG | AI760053 | 26.55 | 53.00 | 14.29 | 0.27 | 26.55 | 40.72 | 27.17 | 0.67 |
| 221295_at | 1149 /// | CIDEA | NM_001279 | 5.34 | 47.52 | 12.82 | 0.27 | 5.34 | 11.70 | 6.95 | 0.59 |
| | | /// | | | | | | | | | |
| | | GGT1 | | | | | | | | | |
| 219915_s_at | 117247 | SLC16A10 | NM_018593 | 30.90 | 53.44 | 14.43 | 0.27 | 30.90 | 248.74 | 115.97 | 0.47 |
| 205883_at | 7704 | ZBTB16 | NM_006006 | 11.54 | 32.27 | 8.72 | 0.27 | 11.54 | 30.38 | 3.64 | 0.12 |
| 214023_x_at | 347733 | TUBB2B | AL533838 | 1.73 | 14.85 | 4.01 | 0.27 | 1.73 | 14.14 | 33.14 | |
| 214528_s_at | 7849 | PAX8 | NM_013951 | 9.12 | 34.34 | 9.29 | 0.27 | 9.12 | 12.86 | 26.81 | |
| 220626_at | 51156 | SERPINA10 | NM_016186 | 7.15 | 25.23 | 6.83 | 0.27 | 7.15 | 11.54 | 28.40 | |
| 220979_s_at | 81849 | ST6GALNAC5 | NM_030965 | 5.10 | 12.98 | 3.52 | 0.27 | 5.10 | 14.14 | 6.08 | 0.43 |
| 207602_at | 9407 | TMPRSS11D | NM_004262 | 32.35 | 56.30 | 15.25 | 0.27 | 32.35 | 46.04 | 25.46 | |
| 216618_at | | | AL117520 | 7.07 | 11.29 | 3.06 | 0.27 | 7.07 | 7.09 | 7.76 | |
| 216827_at | | | AL355379 | 16.94 | 32.06 | 8.69 | 0.27 | 16.94 | 10.81 | 2.25 | |
| 211422_at | 80036 | TRPM3 | AL136545 | 8.97 | 25.10 | 6.81 | 0.27 | 8.97 | 19.27 | 2.95 | 0.15 |
| 222085_at | 400451 | LOC400451 | AW452357 | 11.89 | 26.00 | 7.07 | 0.27 | 11.89 | 9.88 | 6.06 | |
| 208006_at | 2299 | FOXI1 | NM_012188 | 5.22 | 27.86 | 7.60 | 0.27 | 5.22 | 8.94 | 18.28 | |
| 220131_at | 53822 | FXYD7 | NM_022006 | 10.07 | 34.61 | 9.45 | 0.27 | 10.07 | 11.60 | 10.75 | |
| 217222_at | | | S74639 | 8.78 | 20.57 | 5.64 | 0.27 | 8.78 | 8.73 | 8.55 | |
| 214762_at | 534 | ATP6V1G2 | BF340635 | 25.32 | 44.89 | 12.33 | 0.27 | 25.32 | 18.58 | 14.67 | |
| 210323_at | 27285 | TEKT2 | AB033823 | 4.58 | 39.12 | 10.75 | 0.27 | 4.58 | 9.93 | 9.21 | |
| 205666_at | 2326 | FMO1 | NM_002021 | 8.79 | 28.09 | 7.73 | 0.28 | 8.79 | 17.62 | 27.75 | |
| 215247_at | 440895 | LOC440895 | AI561253 | 30.64 | 60.12 | 16.57 | 0.28 | 30.64 | 21.20 | 15.50 | |
| 213651_at | 27124 | PIB5PA | AI935720 | 5.97 | 17.74 | 4.90 | 0.28 | 5.97 | 7.26 | 23.83 | |
| 216946_at | 3111 | HLA- | M29335 | 6.56 | 19.83 | 5.49 | 0.28 | 6.56 | 33.45 | 9.10 | 0.27 |
| | | DOA | | | | | | | | | |
| 217479_at | 388336 | FLJ45455 | AL110201 | 3.67 | 25.71 | 7.16 | 0.28 | 3.67 | 13.97 | 11.27 | |
| 216611_s_at | 6530 | SLC6A2 | AB022847 | 8.07 | 14.18 | 3.95 | 0.28 | 8.07 | 4.91 | 3.85 | |
| 215630_at | 23180 | RFTN1 | AU147528 | 17.98 | 68.15 | 19.07 | 0.28 | 17.98 | 92.48 | 80.57 | |
| 208366_at | 27328 | PCDH11X | NM_014522 | 9.95 | 29.24 | 8.19 | 0.28 | 9.95 | 25.23 | 26.22 | |
| 214572_s_at | 3640 | INSL3 | NM_005543 | 9.53 | 18.33 | 5.14 | 0.28 | 9.53 | 8.07 | 4.02 | |
| 221241_s_at | 79370 | BCL2L14 | NM_030766 | 4.68 | 15.45 | 4.33 | 0.28 | 4.68 | 8.86 | 2.77 | |
| 204779_s_at | 3217 | HOXB7 | NM_004502 | 32.81 | 66.69 | 18.71 | 0.28 | 32.81 | 14.88 | 16.60 | |
| 213797_at | 91543 | RSAD2 | AI337069 | 131.05 | 296.80 | 83.36 | 0.28 | 131.05 | 197.36 | 72.89 | 0.37 |
| 216047_x_at | 23544 | SEZ6L | AL050253 | 27.19 | 44.58 | 12.53 | 0.28 | 27.19 | 21.70 | 28.99 | |
| 211830_s_at | 8911 | CACNA1I | AF211189 | 21.92 | 52.89 | 14.87 | 0.28 | 21.92 | 68.28 | 53.07 | |
| 220689_at | | | NM_018055 | 18.47 | 42.63 | 12.00 | 0.28 | 18.47 | 8.81 | 11.09 | |
| 202714_s_at | 9692 | KIAA0391 | NM_014672 | 34.87 | 65.48 | 18.46 | 0.28 | 34.87 | 48.35 | 9.91 | |
| 214088_s_at | 2525 | FUT3 | AW080549 | 31.31 | 47.46 | 13.39 | 0.28 | 31.31 | 14.12 | 22.92 | |
| 214371_at | 23617 | TSSK2 | AI652441 | 23.19 | 40.13 | 11.34 | 0.28 | 23.19 | 4.53 | 9.47 | |
| 216043_x_at | 9727 | RAB11FIP3 | AK024135 | 46.70 | 88.94 | 25.15 | 0.28 | 46.70 | 33.99 | 37.28 | |
| 208474_at | 9074 | CLDN6 | NM_021195 | 13.31 | 35.38 | 10.04 | 0.28 | 13.31 | 16.34 | 26.80 | |
| 207531_at | 1420 | CRYGC | NM_020989 | 10.18 | 25.54 | 7.26 | 0.28 | 10.18 | 29.22 | 12.39 | 0.42 |
| 202988_s_at | 5996 | RGS1 | NM_002922 | 5.07 | 16.67 | 4.74 | 0.28 | 5.07 | 22.11 | 9.52 | 0.43 |
| 210248_at | 7476 | WNT7A | D83175 | 5.65 | 22.72 | 6.47 | 0.28 | 5.65 | 7.33 | 25.92 | |
| 215542_at | | | AK023121 | 2.74 | 11.96 | 3.41 | 0.28 | 2.74 | 16.67 | 1.20 | 0.07 |
| 215857_at | 56926 | NCLN | AK022309 | 5.81 | 23.75 | 6.77 | 0.29 | 5.81 | 17.34 | 5.71 | 0.33 |
| 207660_at | 1756 | DMD | NM_004019 | 4.55 | 12.30 | 3.51 | 0.29 | 4.55 | 21.97 | 10.34 | 0.47 |
| 204834_at | 10875 | FGL2 | NM_006682 | 235.09 | 447.31 | 128.08 | 0.29 | 235.09 | 443.74 | 52.00 | 0.12 |
| 217351_at | 392479 | LOC392479 | AL024458 | 2.40 | 20.21 | 5.80 | 0.29 | 2.40 | 10.45 | 4.22 | 0.40 |
| 205226_at | 5157 | PDGFRL | NM_006207 | 9.81 | 39.25 | 11.28 | 0.29 | 9.81 | 137.33 | 59.71 | 0.43 |
| 207346_at | 2054 | STX2 | NM_001980 | 30.26 | 56.81 | 16.33 | 0.29 | 30.26 | 26.17 | 14.27 | |
| 220817_at | 7223 | TRPC4 | NM_016179 | 2.29 | 26.09 | 7.50 | 0.29 | 2.29 | 22.80 | 2.57 | 0.11 |
| 213592_at | 187 | AGTRL1 | X89271 | 5.58 | 19.76 | 5.69 | 0.29 | 5.58 | 5.95 | 2.23 | |
| 218523_at | 64077 | LHPP | NM_022126 | 6.80 | 36.10 | 10.41 | 0.29 | 6.80 | 15.36 | 17.14 | |
| 213335_s_at | 10402 | ST3GAL6 | AK001922 | 5.57 | 51.23 | 14.78 | 0.29 | 5.57 | 8.30 | 2.80 | |
| 214234_s_at | 1577 | CYP3A5 | X90579 | 7.84 | 22.21 | 6.43 | 0.29 | 7.84 | 59.75 | 17.90 | 0.30 |
| 216740_at | 55809 | TRERF1 | AK024851 | 12.83 | 21.32 | 6.18 | 0.29 | 12.83 | 22.35 | 10.56 | 0.47 |
| 220196_at | 94025 | MUC16 | NM_024690 | 1.84 | 10.24 | 2.97 | 0.29 | 1.84 | 2.45 | 2.97 | |
| 214255_at | 57194 | ATP10A | N35112 | 175.94 | 339.96 | 98.64 | 0.29 | 175.94 | 191.53 | 120.37 | |
| 217583_at | 5053 | PAH | BG433489 | 3.84 | 22.23 | 6.45 | 0.29 | 3.84 | 4.17 | 2.55 | |
| 221297_at | 55507 | GPRC5D | NM_018654 | 17.02 | 53.14 | 15.51 | 0.29 | 17.02 | 12.50 | 6.35 | |
| 206404_at | 2254 | FGF9 | NM_002010 | 9.63 | 42.36 | 12.37 | 0.29 | 9.63 | 8.19 | 6.64 | |
| 211844_s_at | 8828 | NRP2 | AF022859 | 2.87 | 16.43 | 4.81 | 0.29 | 2.87 | 15.81 | 41.73 | |
| 221105_at | | | NM_018395 | 7.33 | 18.28 | 5.35 | 0.29 | 7.33 | 9.13 | 1.94 | |
| 213849_s_at | 5521 | PPP2R2B | AA974416 | 27.68 | 76.12 | 22.27 | 0.29 | 27.68 | 29.98 | 23.79 | |
| 209684_at | 54453 | RIN2 | AL136924 | 4.45 | 31.01 | 9.08 | 0.29 | 4.45 | 25.64 | 11.26 | 0.44 |
| 206210_s_at | 107F | CETP | NM_000078 | 6.85 | 11.17 | 3.27 | 0.29 | 6.85 | 4.45 | 8.82 | |
| 215906_at | | | S65921 | 5.40 | 12.11 | 3.55 | 0.29 | 5.40 | 4.61 | 4.01 | |
| 217182_at | 4586 | MUC5AC | Z34282 | 15.36 | 29.14 | 8.54 | 0.29 | 15.36 | 65.91 | 21.97 | 0.33 |
| 205031_at | 1949 | EFNB3 | NM_001406 | 9.49 | 23.52 | 6.90 | 0.29 | 9.49 | 6.67 | 8.20 | |
| 208586_s_at | 548313 / | SSX4 | NM_005636 | 2.43 | 25.54 | 7.50 | 0.29 | 2.43 | 7.83 | 5.05 | |
| | | /// | | | | | | | | | |
| | | SSX4B | | | | | | | | | |
| 206237_s_at | 3084 | NRG1 | NM_013957 | 5.69 | 15.26 | 4.49 | 0.29 | 5.69 | 33.05 | 353.86 | |
| 215184_at | 23604 | DAPK2 | AK026801 | 8.52 | 18.96 | 5.59 | 0.29 | 8.52 | 17.70 | 7.18 | 0.41 |
| 206906_at | 7087 | ICAM5 | NM_003259 | 16.04 | 36.34 | 10.71 | 0.29 | 16.04 | 47.11 | 50.78 | |
| 221405_at | 51190 | LOC51190 | NM_016317 | 17.54 | 27.35 | 8.06 | 0.29 | 17.54 | 11.82 | 8.66 | |
| 210960_at | 146 | ADRA1D | M76446 | 7.97 | 14.80 | 4.36 | 0.29 | 7.97 | 6.61 | 13.19 | |
| 211193_at | 8626 | TP73L | AF061512 | 17.52 | 34.72 | 10.23 | 0.29 | 17.52 | 13.14 | 22.02 | |
| 213293_s_at | 10346 | TRIM22 | AA083478 | 141.18 | 578.88 | 170.81 | 0.30 | 141.18 | 722.64 | 140.68 | 0.19 |
| 205201_at | 2737 | GLI3 | NM_000168 | 39.46 | 86.24 | 25.46 | 0.30 | 39.46 | 52.78 | 75.97 | |
| 204678_s_at | 3775 | KCNK1 | U90065 | 1.57 | 12.69 | 3.75 | 0.30 | 1.57 | 6.93 | 14.94 | |
| 217095_x_at | 9437 | NCR1 | AJ006122 | 6.84 | 20.73 | 6.12 | 0.30 | 6.84 | 13.96 | 20.19 | |
| 216196_at | 440366 | LOC440366 | X69637 | 17.85 | 45.99 | 13.63 | 0.30 | 17.85 | 21.40 | 6.17 | |
| 216181_at | 8871 | SYNJ2 | AK026758 | 19.40 | 49.75 | 14.77 | 0.30 | 19.40 | 9.21 | 45.08 | |
| 208592_s_at | 913 | CD1E | NM_030893 | 3.05 | 22.83 | 6.78 | 0.30 | 3.05 | 10.77 | 11.82 | |
| 208260_at | 553 | AVPR1B | NM_000707 | 31.11 | 52.45 | 15.59 | 0.30 | 31.11 | 24.50 | 15.98 | |
| 221128_at | 8728 | ADAM19 | NM_023038 | 12.63 | 19.33 | 5.75 | 0.30 | 12.63 | 23.18 | 20.16 | |
| 202312_s_at | 1277 | COL1A1 | K01228 | 14.76 | 27.86 | 8.31 | 0.30 | 14.76 | 18.57 | 19.40 | |
| 215303_at | | | BE046461 | 1.69 | 14.59 | 4.36 | 0.30 | 1.69 | 2.69 | 9.78 | |
| 216344_at | 261734 | NPHP4 | AL117405 | 8.37 | 29.12 | 8.71 | 0.30 | 8.37 | 20.27 | 20.35 | |
| 216007_at | 64084 | CLSTN2 | AF052176 | 26.37 | 45.13 | 13.51 | 0.30 | 26.37 | 31.45 | 30.75 | |
| 214300_s_at | 7156 | TOP3A | AI676092 | 18.81 | 45.26 | 13.55 | 0.30 | 18.81 | 56.78 | 26.21 | 0.46 |
| 220901_at | 80045 | GPR157 | NM_024980 | 12.24 | 37.33 | 11.20 | 0.30 | 12.24 | 35.85 | 41.54 | |
| 218943_s_at | 23586 | DDX58 | NM_014314 | 142.59 | 612.93 | 183.90 | 0.30 | 142.59 | 569.23 | 191.35 | 0.34 |
| 205456_at | 916 | CD3E | NM_000733 | 8.46 | 31.35 | 9.42 | 0.30 | 8.46 | 28.21 | 15.42 | 0.55 |
| 217474_at | | | AL117652 | 20.76 | 33.63 | 10.10 | 0.30 | 20.76 | 15.31 | 41.40 | |
| 210521_s_at | 26998 | FETUB | AB017551 | 4.87 | 13.82 | 4.15 | 0.30 | 4.87 | 15.40 | 3.50 | 0.23 |
| 211896_s_at | 1634 | DCN | AF138302 | 16.50 | 61.98 | 18.66 | 0.30 | 16.50 | 48.16 | 30.59 | 0.64 |
| 220851_at | | | NM_014095 | 4.72 | 21.20 | 6.38 | 0.30 | 4.72 | 21.07 | 3.69 | 0.17 |
| AFFX- | 6772 | STAT1 | AFFX- | 163.93 | 393.76 | 118.62 | 0.30 | 163.93 | 188.44 | 124.21 | |
| HUMISGF3A/ | | | HUMISGF3A/ | | | | | | | | |
| M97935_5_at | | | M97935_5 | | | | | | | | |
| 222223_s_at | 26525 | IL1F5 | AF216693 | 12.17 | 35.41 | 10.67 | 0.30 | 12.17 | 22.90 | 54.22 | |
| 215651_at | | | AK026682 | 23.12 | 78.52 | 23.69 | 0.30 | 23.12 | 13.82 | 16.87 | |
| 207116_s_at | 26330 | GAPDHS | NM_014364 | 4.44 | 15.90 | 4.80 | 0.30 | 4.44 | 16.14 | 11.36 | 0.70 |
| 211095_at | 4763 | NF1 | D12625 | 12.13 | 24.22 | 7.32 | 0.30 | 12.13 | 25.34 | 19.07 | |
| 213948_x_at | 57863 | IGSF4B | AI564838 | 3.25 | 23.97 | 7.24 | 0.30 | 3.25 | 9.17 | 6.36 | |
| 207064_s_at | 314 | AOC2 | NM_009590 | 8.55 | 30.67 | 9.28 | 0.30 | 8.55 | 28.82 | 28.29 | |
| 213706_at | 2819 | GPD1 | AI368018 | 5.45 | 18.19 | 5.51 | 0.30 | 5.45 | 5.37 | 4.42 | |
| 220129_at | 54937 | SOHLH2 | NM_017826 | 5.02 | 38.90 | 11.79 | 0.30 | 5.02 | 9.76 | 15.42 | |
| 215956_at | 5868 | RAB5A | AK022065 | 24.75 | 48.08 | 14.60 | 0.30 | 24.75 | 22.56 | 37.72 | |
| 204562_at | 3662 | IRF4 | NM_002460 | 33.88 | 93.40 | 28.43 | 0.30 | 33.88 | 62.68 | 78.51 | |
| 219466_s_at | 336 | APOA2 | NM_001643 | 14.30 | 43.55 | 13.26 | 0.30 | 14.30 | 16.43 | 6.18 | |
| 207212_at | 6550 | SLC9A3 | NM_004174 | 5.47 | 10.51 | 3.20 | 0.30 | 5.47 | 2.01 | 2.34 | |
| 220582_at | | | NM_025071 | 17.72 | 36.69 | 11.18 | 0.30 | 17.72 | 7.72 | 32.19 | |
| 220687_at | | | NM_018175 | 9.22 | 40.97 | 12.50 | 0.31 | 9.22 | 33.20 | 32.79 | |
| 205864_at | 6545 | SLC7A4 | NM_004173 | 26.00 | 48.04 | 14.67 | 0.31 | 26.00 | 17.49 | 9.75 | |
| 207742_s_at | 2649 | NR6A1 | NM_001489 | 7.62 | 21.90 | 6.69 | 0.31 | 7.62 | 26.80 | 39.67 | |
| 219039_at | 54910 | SEMA4C | NM_017789 | 106.17 | 286.50 | 87.50 | 0.31 | 106.17 | 151.64 | 63.78 | |
| 208080_at | 6790 | AURKA | NM_003158 | 20.78 | 50.95 | 15.60 | 0.31 | 20.78 | 17.01 | 24.82 | |
| 208902_s_at | 256949 / | RPS28 | BF431363 | 13.06 | 81.23 | 24.89 | 0.31 | 13.06 | 9.13 | 14.40 | |
| | | /// | | | | | | | | | |
| | | ANKRD47 | | | | | | | | | |
| 218736_s_at | 54873 | PALMD | NM_017734 | 2.59 | 21.92 | 6.72 | 0.31 | 2.59 | 2.49 | 7.08 | |
| 221154_at | 283116 / | TRIM49 | NM_020358 | 3.58 | 10.71 | 3.28 | 0.31 | 3.58 | 9.62 | 2.42 | |
| | | /// | | | | | | | | | |
| | | LOC283116 | | | | | | | | | |
| | | /// | | | | | | | | | |
| | | LOC65 | | | | | | | | | |
| 207138_at | 5253 | PHF2 | NM_005392 | 2.00 | 10.77 | 3.30 | 0.31 | 2.00 | 9.64 | 6.66 | |
| 215258_at | 199731 | IGSF4C | AC005525 | 13.35 | 26.98 | 8.28 | 0.31 | 13.35 | 16.16 | 6.75 | |
| 207907_at | 8740 | TNFSF14 | NM_003807 | 244.50 | 469.67 | 144.24 | 0.31 | 244.50 | 9.70 | 98.35 | |
| 204931_at | 6943 | TCF21 | NM_003206 | 3.93 | 13.46 | 4.14 | 0.31 | 3.93 | 11.16 | 4.05 | 0.36 |
| 210781_x_at | 2902 | GRIN1 | AF015731 | 7.99 | 17.08 | 5.25 | 0.31 | 7.99 | 10.02 | 3.25 | |
| 203936_s_at | 4318 | MMP9 | NM_004994 | 152.92 | 1272.48 | 391.26 | 0.31 | 152.92 | 1661.16 | 453.20 | 0.27 |
| 209125_at | 140446 / | KRT6A | J00269 | 15.03 | 25.14 | 7.74 | 0.31 | 15.03 | 5.22 | 16.94 | |
| | | /// | | | | | | | | | |
| | | KRT6C | | | | | | | | | |
| | | /// | | | | | | | | | |
| | | KRT6E | | | | | | | | | |
| 208711_s_at | 595 | CCND1 | BC000076 | 17.91 | 87.06 | 26.85 | 0.31 | 17.91 | 76.97 | 43.26 | 0.56 |
| 215372_x_at | 9223 | MAGI1 | AU146794 | 20.71 | 77.29 | 23.88 | 0.31 | 20.71 | 28.71 | 3.47 | |
| 201565_s_at | 3398 | ID2 | NM_002166 | 2022.60 | 3332.02 | 1030.00 | 0.31 | 2022.60 | 5470.79 | 4962.34 | |
| 214538_x_at | 9628 | RGS6 | AF073921 | 3.00 | 14.82 | 4.58 | 0.31 | 3.00 | 4.90 | 3.30 | |
| 219685_at | 59353 | TMEM35 | NM_021637 | 1.62 | 22.42 | 6.93 | 0.31 | 1.62 | 4.58 | 7.64 | |
| 215466_at | | | AF035314 | 19.20 | 32.80 | 10.15 | 0.31 | 19.20 | 5.74 | 26.25 | |
| 210699_at | | | AF116679 | 10.24 | 45.32 | 14.04 | 0.31 | 10.24 | 46.75 | 6.85 | 0.15 |
| 220435_at | 55532 | SLC30A10 | NM_018713 | 7.48 | 16.27 | 5.04 | 0.31 | 7.48 | 6.72 | 4.82 | |
| 222069_s_at | | | BF058311 | 3.91 | 14.94 | 4.63 | 0.31 | 3.91 | 6.95 | 8.17 | |
| 208468_at | 11166 | SOX21 | NM_007084 | 8.43 | 19.47 | 6.04 | 0.31 | 8.43 | 9.45 | 26.33 | |
| 206996_x_at | 782 | CACNB1 | NM_000723 | 27.97 | 46.33 | 14.38 | 0.31 | 27.97 | 56.34 | 25.34 | 0.45 |
| 214610_at | 1584 | CYP11B1 | AV702430 | 9.55 | 17.53 | 5.46 | 0.31 | 9.55 | 12.44 | 7.17 | |
| 217651_at | | | BF512531 | 12.51 | 35.00 | 10.90 | 0.31 | 12.51 | 8.77 | 18.57 | |
| 210972_x_at | 28517 | TRA@ | M15565 | 12.56 | 32.98 | 10.29 | 0.31 | 12.56 | 10.13 | 23.94 | |
| | // | /// | | | | | | | | | |
| | | TRDV2 | | | | | | | | | |
| | | /// | | | | | | | | | |
| | | TRAV20 | | | | | | | | | |
| | | /// | | | | | | | | | |
| 206553_at | 4939 | OAS2 | NM_002535 | 97.30 | 226.30 | 70.68 | 0.31 | 97.30 | 201.61 | 83.85 | 0.42 |
| 210773_s_at | 2358 | FPRL1 | U81501 | 17.52 | 50.31 | 15.72 | 0.31 | 17.52 | 26.55 | 16.36 | 0.62 |
| 216866_s_at | 7373 | COL14A1 | M64108 | 2.40 | 12.32 | 3.85 | 0.31 | 2.40 | 2.51 | 1.94 | |
| 209227_at | | | AU158251 | 3.90 | 11.72 | 3.66 | 0.31 | 3.90 | 16.99 | 5.26 | 0.31 |
| 220904_at | 80069 | C6orf208 | NM_025002 | 5.48 | 47.06 | 14.72 | 0.31 | 5.48 | 3.27 | 13.10 | |
| 211249_at | 8111 | GPR68 | U35398 | 61.33 | 99.46 | 31.15 | 0.31 | 61.33 | 33.60 | 5.73 | |
| 205390_s_at | 286 | ANK1 | M28880 | 3.52 | 10.86 | 3.41 | 0.31 | 3.52 | 2.87 | 4.57 | |
| 204582_s_at | 354 | KLK3 | NM_001648 | 4.35 | 33.76 | 10.60 | 0.31 | 4.35 | 22.15 | 5.64 | 0.25 |
| 217462_at | 745 | C11orf9 | AC004770 | 18.53 | 30.50 | 9.57 | 0.31 | 18.53 | 4.34 | 20.41 | |
| 200606_at | 1832 | DSP | NM_004415 | 3.38 | 18.42 | 5.79 | 0.31 | 3.38 | 15.98 | 8.00 | 0.50 |
| 207787_at | 3884 | KRT33B | NM_002279 | 6.33 | 14.14 | 4.45 | 0.31 | 6.33 | 5.25 | 3.89 | |
| 217936_at | 394 | ARHGAP5 | AW044631 | 2.90 | 19.94 | 6.27 | 0.31 | 2.90 | 8.39 | 10.36 | |
| 209573_s_at | 753 | C18orf1 | AF009424 | 25.53 | 39.61 | 12.51 | 0.32 | 25.53 | 11.16 | 16.79 | |
| 206762_at | 3741 | KCNA5 | NM_002234 | 5.05 | 22.65 | 7.16 | 0.32 | 5.05 | 2.47 | 1.79 | |
| 205580_s_at | 3269 | HRH1 | D28481 | 32.54 | 63.41 | 20.05 | 0.32 | 32.54 | 397.39 | 358.02 | |
| 206614_at | 8200 | GDF5 | NM_000557 | 16.19 | 49.52 | 15.67 | 0.32 | 16.19 | 11.98 | 23.70 | |
| 205660_at | 8638 | OASL | NM_003733 | 88.99 | 341.25 | 108.14 | 0.32 | 88.99 | 365.73 | 178.92 | 0.49 |
| 204631_at | 4620 | MYH2 | NM_017534 | 10.24 | 18.93 | 6.00 | 0.32 | 10.24 | 6.33 | 5.12 | |
| 219430_at | 56834 | GPR137 | NM_020155 | 34.07 | 59.77 | 18.96 | 0.32 | 34.07 | 5.67 | 40.13 | |
| 216414_at | | | AL136094 | 2.09 | 10.96 | 3.48 | 0.32 | 2.09 | 3.57 | 6.32 | |
| 204681_s_at | 9771 | RAPGEF5 | NM_012294 | 4.61 | 15.65 | 4.97 | 0.32 | 4.61 | 1.66 | 3.45 | |
| 211736_at | 6668 | SP2 | BC005914 | 7.59 | 18.90 | 6.00 | 0.32 | 7.59 | 19.43 | 17.64 | |
| 219955_at | 54596 | L1TD1 | NM_019079 | 21.94 | 35.06 | 11.16 | 0.32 | 21.94 | 5.10 | 11.34 | |
| 210940_s_at | 2911 | GRM1 | U31216 | 4.20 | 30.54 | 9.72 | 0.32 | 4.20 | 14.81 | 9.69 | 0.65 |
| 207588_at | 8827 | MYT2 | NM_003871 | 2.86 | 13.14 | 4.20 | 0.32 | 2.86 | 6.94 | 15.55 | |
| 209038_s_at | 10938 | EHD1 | AL579035 | 68.55 | 207.66 | 66.35 | 0.32 | 68.55 | 304.89 | 354.40 | |
| 221385_s_at | 2865 /// | FFAR3 | NM_005305 | 6.86 | 17.35 | 5.55 | 0.32 | 6.86 | 1.79 | 21.84 | |
| | | /// | | | | | | | | | |
| | | GPR42 | | | | | | | | | |
| | | /// | | | | | | | | | |
| | | LOC731823 | | | | | | | | | |
| 221359_at | 2668 | GDNF | NM_000514 | 7.77 | 20.60 | 6.60 | 0.32 | 7.77 | 2.79 | 8.03 | |
| 205798_at | 3575 | IL7R | NM_002185 | 25.72 | 372.24 | 119.37 | 0.32 | 25.72 | 10147.44 | 4107.25 | 0.40 |
| 213972_at | 2297 | FOXD1 | AI080288 | 11.48 | 62.74 | 20.13 | 0.32 | 11.48 | 10.73 | 16.16 | |
| 208350_at | 1446 | CSN1S1 | NM_001890 | 9.06 | 18.08 | 5.81 | 0.32 | 9.06 | 9.91 | 9.10 | |
| 203404_at | 9823 | ARMCX2 | NM_014782 | 13.42 | 27.70 | 8.91 | 0.32 | 13.42 | 10.45 | 2.40 | |
| 220704_at | 10320 | IKZF1 | NM_018563 | 45.18 | 143.43 | 46.24 | 0.32 | 45.18 | 177.07 | 159.62 | |
| 203180_at | 220 | ALDH1A3 | NM_000693 | 4.84 | 30.78 | 9.93 | 0.32 | 4.84 | 39.05 | 16.86 | 0.43 |
| 215042_at | 654 | BMP6 | AI123471 | 3.86 | 30.84 | 9.97 | 0.32 | 3.86 | 5.90 | 8.18 | |
| 210056_at | 27289 | RND1 | U69563 | 16.25 | 41.60 | 13.45 | 0.32 | 16.25 | 65.86 | 56.09 | |
| 221384_at | 7350 | UCP1 | NM_021833 | 6.45 | 10.66 | 3.45 | 0.32 | 6.45 | 3.71 | 7.95 | |
| 215721_at | 3500 | IGHG1 | X58397 | 7.93 | 13.41 | 4.35 | 0.32 | 7.93 | 2.45 | 2.82 | |
| 221162_at | 10086 | HHLA1 | NM_005712 | 21.23 | 43.59 | 14.15 | 0.32 | 21.23 | 34.56 | 81.32 | |
| 217502_at | 3433 | IFIT2 | BE888744 | 10.20 | 191.10 | 62.04 | 0.32 | 10.20 | 90.52 | 91.62 | |
| 205921_s_at | 6533 | SLC6A6 | NM_003043 | 8.89 | 39.98 | 12.99 | 0.32 | 8.89 | 32.40 | 80.27 | |
| 205308_at | 51101 | C8orf70 | NM_016010 | 7.04 | 21.89 | 7.13 | 0.33 | 7.04 | 11.29 | 12.61 | |
| 215730_at | 51442 | VGLL1 | BE542323 | 15.71 | 30.24 | 9.86 | 0.33 | 15.71 | 25.82 | 16.11 | 0.62 |
| 206194_at | 3221 | HOXC4 | AW299598 | 26.51 | 73.12 | 23.88 | 0.33 | 26.51 | 19.40 | 34.08 | |
| 220727_at | 54207 | KCNK10 | NM_021161 | 16.31 | 29.32 | 9.58 | 0.33 | 16.31 | 13.18 | 42.57 | |
| 215449_at | 222642 | BZRPL1 | AI052224 | 9.50 | 21.01 | 6.87 | 0.33 | 9.50 | 3.49 | 9.41 | |
| 211891_s_at | 50649 | ARHGEF4 | AB042199 | 13.55 | 33.86 | 11.07 | 0.33 | 13.55 | 3.82 | 9.10 | |
| 212741_at | 4128 | MAOA | AA923354 | 7.77 | 38.91 | 12.74 | 0.33 | 7.77 | 37.80 | 12.63 | 0.33 |
| 219313_at | 54762 | GRAMD1C | NM_017577 | 17.55 | 29.80 | 9.76 | 0.33 | 17.55 | 3.81 | 3.25 | |
| 207571_x_at | 9473 | C1orf38 | NM_004848 | 247.84 | 585.69 | 191.78 | 0.33 | 247.84 | 479.11 | 237.70 | 0.50 |
| 222080_s_at | | | AW188134 | 5.23 | 12.59 | 4.13 | 0.33 | 5.23 | 22.10 | 11.80 | 0.53 |
| 211869_at | | | AF049656 | 4.51 | 11.08 | 3.63 | 0.33 | 4.51 | 4.25 | 6.89 | |
| 219908_at | 27123 | DKK2 | NM_014421 | 297.50 | 552.30 | 181.34 | 0.33 | 297.50 | 118.37 | 164.22 | |
| 206171_at | 140 | ADORA3 | NM_000677 | 18.81 | 31.77 | 10.44 | 0.33 | 18.81 | 17.38 | 11.07 | |
| 202409_at | 3481 /// | IGF2 | X07868 | 9.43 | 36.56 | 12.02 | 0.33 | 9.43 | 100.24 | 40.12 | 0.40 |
| | | /// INS- | | | | | | | | | |
| | | IGF2 | | | | | | | | | |
| 216238_s_at | 2244 | FGB | BG545288 | 3.14 | 17.17 | 5.65 | 0.33 | 3.14 | 7.71 | 5.91 | |
| 208543_at | 26538 | OR10H2 | NM_013939 | 2.93 | 12.83 | 4.23 | 0.33 | 2.93 | 2.72 | 1.59 | |
| 207351_s_at | 9047 | SH2D2A | NM_003975 | 91.41 | 152.14 | 50.15 | 0.33 | 91.41 | 255.43 | 76.03 | 0.30 |
| 205158_at | 6038 | RNASE4 | NM_002937 | 8.41 | 16.86 | 5.56 | 0.33 | 8.41 | 2.15 | 6.69 | |
| 209524_at | 50810 | HDGFRP3 | AK001280 | 12.15 | 27.90 | 9.21 | 0.33 | 12.15 | 19.95 | 4.50 | 0.23 |
| 204747_at | 3437 | IFIT3 | NM_001549 | 85.86 | 453.16 | 149.61 | 0.33 | 85.86 | 428.18 | 165.86 | 0.39 |
| 207471_at | | | AF118086 | 12.05 | 18.79 | 6.21 | 0.33 | 12.05 | 12.80 | 14.03 | |
| 214616_at | 5030 | P2RY4 | NM_003532 | 14.73 | 68.02 | 22.47 | 0.33 | 14.73 | 40.29 | 9.78 | 0.24 |
| 214927_at | 9358 | ITGBL1 | AL359052 | 5.75 | 30.10 | 9.95 | 0.33 | 5.75 | 3.92 | 5.67 | |
| 215654_at | 587 | BCAT2 | AK023909 | 36.62 | 63.94 | 21.18 | 0.33 | 36.62 | 32.84 | 47.95 | |
| 219457_s_at | 79890 | RIN3 | NM_024832 | 145.15 | 326.81 | 108.28 | 0.33 | 145.15 | 154.47 | 69.03 | |
| 205067_at | 3553 | IL1B | NM_000576 | 51.79 | 821.35 | 272.30 | 0.33 | 51.79 | 14950.88 | 13257.54 | |
| AFFX-PheX- | | | AFFX- | 7.27 | 16.84 | 5.59 | 0.33 | 7.27 | 7.55 | 11.48 | |
| 3_at | | | PheX-3 | | | | | | | | |
| 214717_at | 150967 | DKFZp434H1419 | AL137534 | 70.49 | 152.92 | 50.76 | 0.33 | 70.49 | 124.17 | 61.90 | 0.50 |
| 206867_at | 2646 | GCKR | NM_001486 | 15.39 | 35.59 | 11.82 | 0.33 | 15.39 | 26.71 | 94.01 | |
| 213917_at | 7849 | PAX8 | BE465829 | 15.95 | 38.38 | 12.75 | 0.33 | 15.95 | 33.65 | 19.89 | 0.59 |
| 215488_at | 2495 | FTH1 | AF052095 | 8.88 | 25.41 | 8.45 | 0.33 | 8.88 | 8.82 | 8.35 | |
| 204939_s_at | 5350 | PLN | NM_002667 | 2.41 | 12.30 | 4.09 | 0.33 | 2.41 | 1.45 | 2.33 | |
| 207422_at | 8748 | ADAM20 | NM_003814 | 5.74 | 21.40 | 7.13 | 0.33 | 5.74 | 3.32 | 10.99 | |
| 211199_s_at | 23308 | ICOSLG | AF199028 | 4.73 | 115.17 | 38.40 | 0.33 | 4.73 | 176.99 | 171.19 | |
| 215841_at | 2979 | GUCA1B | AL096814 | 38.35 | 93.95 | 31.35 | 0.33 | 38.35 | 54.23 | 58.61 | |
| 214477_at | 4298 | MLLT1 | NM_005934 | 14.37 | 32.52 | 10.87 | 0.33 | 14.37 | 7.65 | 18.08 | |
| 209504_s_at | 58473 | PLEKHB1 | AF081583 | 6.12 | 19.15 | 6.41 | 0.33 | 6.12 | 28.60 | 23.75 | |
| 222213_x_at | | | AU147800 | 14.35 | 38.34 | 12.83 | 0.33 | 14.35 | 29.79 | 11.54 | 0.39 |
| 214834_at | 338427 / | SNRPN | AU118874 | 19.02 | 72.84 | 24.40 | 0.33 | 19.02 | 4.45 | 18.83 | |
| | | /// | | | | | | | | | |
| | | PAR5 | | | | | | | | | |
| | | /// | | | | | | | | | |
| | | SNORD108 | | | | | | | | | |
| | | // | | | | | | | | | |
| 219073_s_at | 114884 | OSBPL10 | NM_017784 | 22.63 | 35.48 | 11.95 | 0.34 | 22.63 | 27.95 | 11.15 | |
| 206932_at | 9023 | CH25H | NM_003956 | 12.68 | 19.37 | 6.52 | 0.34 | 12.68 | 5.11 | 22.82 | |
| 215869_at | 19 | ABCA1 | AK022254 | 12.25 | 31.02 | 10.46 | 0.34 | 12.25 | 15.61 | 12.90 | |
| 205766_at | 8557 | TCAP | NM_003673 | 11.35 | 28.86 | 9.74 | 0.34 | 11.35 | 62.73 | 15.68 | 0.25 |
| 205749_at | 1543 | CYP1A1 | NM_000499 | 4.04 | 19.82 | 6.70 | 0.34 | 4.04 | 10.63 | 8.36 | |
| 220462_at | 80034 | TAIP-2 | NM_024969 | 6.18 | 15.19 | 5.15 | 0.34 | 6.18 | 3.19 | 6.38 | |
| 203649_s_at | 5320 | PLA2G2A | NM_000300 | 16.27 | 24.82 | 8.41 | 0.34 | 16.27 | 11.45 | 7.08 | |
| 210785_s_at | 9473 | C1orf38 | AB035482 | 337.82 | 690.32 | 234.69 | 0.34 | 337.82 | 707.56 | 298.39 | 0.42 |
| 220106_at | 29881 | NPC1L1 | NM_013389 | 5.02 | 30.44 | 10.35 | 0.34 | 5.02 | 3.14 | 5.94 | |
| 206149_at | 63928 | LOC63928 | NM_022097 | 2.22 | 16.22 | 5.52 | 0.34 | 2.22 | 6.62 | 4.92 | |
| 206367_at | 5972 | REN | NM_000537 | 7.44 | 11.60 | 3.95 | 0.34 | 7.44 | 4.31 | 4.56 | |
| 220045_at | 63974 | NEUROD6 | NM_022728 | 8.47 | 23.30 | 7.94 | 0.34 | 8.47 | 3.46 | 14.02 | |
| 213667_at | 10847 | SRCAP | AB002307 | 57.39 | 86.42 | 29.58 | 0.34 | 57.39 | 63.00 | 70.70 | |
| 201006_at | 7001 | PRDX2 | NM_005809 | 45.18 | 94.32 | 32.29 | 0.34 | 45.18 | 60.88 | 55.25 | |
| 214591_at | 56062 | KLHL4 | BF215673 | 4.00 | 20.10 | 6.88 | 0.34 | 4.00 | 7.89 | 16.22 | |
| 215255_at | 22997 | IGSF9B | AB028953 | 22.77 | 49.75 | 17.05 | 0.34 | 22.77 | 21.32 | 23.94 | |
| 216089_at | 645927 / | LOC645927 | BE877397 | 3.51 | 10.76 | 3.69 | 0.34 | 3.51 | 12.02 | 2.80 | 0.23 |
| | | /// | | | | | | | | | |
| | | LOC651111 | | | | | | | | | |
| 209723_at | 5272 | SERPINB9 | BC002538 | 34.25 | 57.27 | 19.65 | 0.34 | 34.25 | 762.07 | 215.01 | 0.28 |
| 203631_s_at | 51704 | GPRC5B | NM_016235 | 5.60 | 12.60 | 4.33 | 0.34 | 5.60 | 20.73 | 13.58 | 0.66 |
| 205234_at | 9122 | SLC16A4 | NM_004696 | 8.29 | 29.56 | 10.17 | 0.34 | 8.29 | 41.22 | 38.14 | |
| 216556_x_at | | | AL135926 | 23.21 | 66.49 | 22.88 | 0.34 | 23.21 | 7.10 | 31.24 | |
| 222187_x_at | 10146 | G3BP1 | X78262 | 131.35 | 305.94 | 105.32 | 0.34 | 131.35 | 82.30 | 116.69 | |
| 201820_at | 3852 | KRT5 | NM_000424 | 5.99 | 16.26 | 5.60 | 0.34 | 5.99 | 3.60 | 9.13 | |
| 219998_at | 29094 | HSPC159 | NM_014181 | 13.89 | 33.82 | 11.65 | 0.34 | 13.89 | 14.65 | 24.13 | |
| 206560_s_at | 8190 | MIA | NM_006533 | 16.79 | 44.64 | 15.38 | 0.34 | 16.79 | 8.50 | 40.53 | |
| 206338_at | 1995 | ELAVL3 | NM_001420 | 5.62 | 13.95 | 4.81 | 0.34 | 5.62 | 5.65 | 16.08 | |
| 211142_x_at | 3111 | HLA- | M38056 | 10.13 | 30.64 | 10.57 | 0.34 | 10.13 | 23.39 | 32.02 | |
| | | DOA | | | | | | | | | |
| 220621_at | 2301 | FOXE3 | NM_012186 | 5.83 | 10.21 | 3.52 | 0.35 | 5.83 | 11.80 | 4.91 | 0.42 |
| 212515_s_at | 1654 | DDX3X | BG492602 | 975.29 | 1688.85 | 582.96 | 0.35 | 975.29 | 948.43 | 1608.43 | |
| 206119_at | 635 | BHMT | NM_001713 | 12.35 | 40.03 | 13.84 | 0.35 | 12.35 | 6.24 | 7.79 | |
| 219840_s_at | 27004 | TCL6 | NM_012468 | 2.19 | 26.04 | 9.02 | 0.35 | 2.19 | 16.14 | 20.37 | |
| 220066_at | 64127 | NOD2 | NM_022162 | 100.06 | 518.34 | 179.49 | 0.35 | 100.06 | 160.53 | 72.19 | 0.45 |
| 216042_at | 8718 | TNFRSF25 | AI275938 | 53.72 | 83.57 | 28.94 | 0.35 | 53.72 | 33.41 | 23.47 | |
| 44673_at | 6614 | SIGLEC1 | N53555 | 110.33 | 240.92 | 83.44 | 0.35 | 110.33 | 133.53 | 58.42 | |
| 220409_at | 157922 | CAMSAP1 | NM_018627 | 13.42 | 44.47 | 15.41 | 0.35 | 13.42 | 33.59 | 33.18 | |
| 206422_at | 2641 | GCG | NM_002054 | 3.30 | 12.49 | 4.33 | 0.35 | 3.30 | 1.14 | 2.57 | |
| 210090_at | 23237 | ARC | AF193421 | 5.62 | 11.23 | 3.90 | 0.35 | 5.62 | 8.31 | 10.06 | |
| 205503_at | 5784 | PTPN14 | NM_005401 | 81.75 | 286.35 | 99.48 | 0.35 | 81.75 | 90.04 | 150.13 | |
| 208937_s_at | 3397 | ID1 | D13889 | 38.28 | 58.63 | 20.38 | 0.35 | 38.28 | 38.87 | 47.81 | |
| 207109_at | 25833 | POU2F3 | NM_014352 | 4.94 | 25.39 | 8.83 | 0.35 | 4.94 | 4.12 | 1.62 | |
| 213516_at | 11214 | AKAP13 | AF126008 | 3.68 | 19.06 | 6.63 | 0.35 | 3.68 | 2.13 | 1.22 | |
| 210770_s_at | 773 | CACNA1A | AF004884 | 90.72 | 165.84 | 57.75 | 0.35 | 90.72 | 65.91 | 43.85 | |
| 221179_at | | | NM_025050 | 5.06 | 21.79 | 7.60 | 0.35 | 5.06 | 8.60 | 3.46 | |
| 211902_x_at | 6955 | TRA@ | L34703 | 15.09 | 65.93 | 23.00 | 0.35 | 15.09 | 31.95 | 8.72 | 0.27 |
| 220779_at | 51702 | PADI3 | NM_016233 | 6.69 | 21.45 | 7.48 | 0.35 | 6.69 | 8.76 | 6.24 | |
| 216575_at | | | AL035604 | 11.84 | 18.69 | 6.52 | 0.35 | 11.84 | 6.15 | 11.71 | |
| 211369_at | | | AF116689 | 8.41 | 15.47 | 5.40 | 0.35 | 8.41 | 5.22 | 25.02 | |
| 217394_at | 6955 | TRA@ | AE000659 | 9.58 | 22.31 | 7.79 | 0.35 | 9.58 | 11.89 | 10.21 | |
| 207517_at | 3918 | LAMC2 | NM_018891 | 7.34 | 13.44 | 4.70 | 0.35 | 7.34 | 4.53 | 2.97 | |
| 216066_at | 19 | ABCA1 | AK024328 | 17.06 | 60.72 | 21.23 | 0.35 | 17.06 | 51.27 | 100.81 | |
| 219659_at | 51761 | ATP8A2 | NM_016529 | 8.08 | 16.98 | 5.94 | 0.35 | 8.08 | 8.76 | 5.69 | |
| 217158_at | 728683 | LOC728683 | X97875 | 4.27 | 13.91 | 4.87 | 0.35 | 4.27 | 10.65 | 2.77 | 0.26 |
| 204751_x_at | 1824 | DSC2 | NM_004949 | 116.12 | 183.32 | 64.28 | 0.35 | 116.12 | 47.95 | 83.23 | |
| 214963_at | 23279 | NUP160 | AK026236 | 8.14 | 31.91 | 11.19 | 0.35 | 8.14 | 21.85 | 4.23 | 0.19 |
| 216219_at | 363 | AQP6 | AL137716 | 20.19 | 58.03 | 20.36 | 0.35 | 20.19 | 53.01 | 32.39 | 0.61 |
| 207854_at | 2996 | GYPE | NM_002102 | 3.22 | 12.33 | 4.33 | 0.35 | 3.22 | 3.89 | 2.19 | |
| 214064_at | 7018 | TF | AI073407 | 10.02 | 33.26 | 11.70 | 0.35 | 10.02 | 9.73 | 10.28 | |
| 215125_s_at | 54575 | UGT1A10 | AV691323 | 8.08 | 20.36 | 7.16 | 0.35 | 8.08 | 6.56 | 13.78 | |
| | // | /// | | | | | | | | | |
| | | UGT1A8 | | | | | | | | | |
| | | /// | | | | | | | | | |
| | | UGT1A7 | | | | | | | | | |
| 219385_at | 56833 | SLAMF8 | NM_020125 | 43.50 | 445.91 | 157.28 | 0.35 | 43.50 | 443.81 | 133.77 | 0.30 |
| 210229_s_at | 1437 | CSF2 | M11734 | 5.89 | 13.16 | 4.64 | 0.35 | 5.89 | 7.30 | 51.72 | |
| 204623_at | 7033 | TFF3 | NM_003226 | 14.34 | 52.14 | 18.42 | 0.35 | 14.34 | 46.35 | 50.03 | |
| 209914_s_at | 9378 | NRXN1 | AW149405 | 10.64 | 25.39 | 9.00 | 0.35 | 10.64 | 4.60 | 30.18 | |
| 218820_at | 56967 | C14orf132 | NM_020215 | 18.31 | 44.99 | 15.94 | 0.35 | 18.31 | 27.67 | 36.85 | |
| 215530_at | 2175 | FANCA | BG484069 | 47.32 | 71.26 | 25.26 | 0.35 | 47.32 | 38.10 | 32.15 | |
| 218600_at | 80774 | LIMD2 | NM_030576 | 121.41 | 404.40 | 143.48 | 0.35 | 121.41 | 130.94 | 95.66 | |
| 208083_s_at | 3694 | ITGB6 | NM_000888 | 5.63 | 10.16 | 3.61 | 0.35 | 5.63 | 0.88 | 1.25 | |
| 214650_x_at | 4340 | MOG | AL050328 | 19.10 | 41.04 | 14.58 | 0.36 | 19.10 | 19.66 | 25.20 | |
| 204642_at | 1901 | EDG1 | NM_001400 | 7.29 | 24.67 | 8.79 | 0.36 | 7.29 | 7.02 | 7.62 | |
| 213831_at | 3117 | HLA- | X00452 | 2.11 | 10.87 | 3.87 | 0.36 | 2.11 | 2.63 | 6.44 | |
| | | DQA1 | | | | | | | | | |
| 214598_at | 9073 | CLDN8 | AL049977 | 2.81 | 12.82 | 4.57 | 0.36 | 2.81 | 9.76 | 8.89 | |
| 213252_at | 9644 | SH3PXD2A | AI739005 | 55.00 | 88.12 | 31.43 | 0.36 | 55.00 | 37.93 | 34.67 | |
| 216017_s_at | 4665 | NAB2 | AJ011081 | 150.35 | 501.93 | 179.05 | 0.36 | 150.35 | 410.79 | 1072.21 | |
| 203541_s_at | 687 | KLF9 | NM_001206 | 8.85 | 14.30 | 5.11 | 0.36 | 8.85 | 18.30 | 17.67 | |
| 220749_at | 79741 | C10orf68 | NM_024688 | 19.62 | 33.19 | 11.86 | 0.36 | 19.62 | 11.38 | 20.86 | |
| AFFX- | 6772 | STAT1 | AFFX- | 333.13 | 792.85 | 283.54 | 0.36 | 333.13 | 356.02 | 297.74 | |
| HUMISGF3A/ | | | HUMISGF3A/ | | | | | | | | |
| M97935_MB_at | | | M97935_MB | | | | | | | | |
| 200795_at | 8404 | SPARCL1 | NM_004684 | 8.32 | 18.73 | 6.70 | 0.36 | 8.32 | 8.98 | 4.29 | |
| 212448_at | 23327 | NEDD4L | AB007899 | 54.98 | 93.15 | 33.38 | 0.36 | 54.98 | 107.56 | 21.85 | 0.20 |
| 216892_at | 3500 | IGHG1 | S65761 | 22.83 | 34.98 | 12.55 | 0.36 | 22.83 | 16.71 | 22.61 | |
| 220194_at | 79730 | NSUN7 | NM_024677 | 4.84 | 12.38 | 4.44 | 0.36 | 4.84 | 3.35 | 4.21 | |
| 213873_at | | | D29810 | 26.79 | 41.92 | 15.04 | 0.36 | 26.79 | 20.44 | 24.28 | |
| 206650_at | 55721 | IQCC | NM_018134 | 22.11 | 48.73 | 17.51 | 0.36 | 22.11 | 41.53 | 20.04 | 0.48 |
| 214325_at | 2813 | GP2 | BF046750 | 3.65 | 10.68 | 3.84 | 0.36 | 3.65 | 7.18 | 3.26 | |
| 213498_at | 90993 | CREB3L1 | BG328407 | 15.99 | 40.69 | 14.65 | 0.36 | 15.99 | 38.50 | 21.47 | 0.56 |
| 219010_at | 55765 | C1orf106 | NM_018265 | 397.55 | 1156.23 | 416.29 | 0.36 | 397.55 | 745.45 | 343.14 | 0.46 |
| 217629_at | 2793 | GNGT2 | AA365670 | 19.33 | 31.12 | 11.21 | 0.36 | 19.33 | 26.85 | 15.35 | |
| 210887_s_at | 2121 | EVC | AF216185 | 10.29 | 23.25 | 8.38 | 0.36 | 10.29 | 17.78 | 32.87 | |
| 210146_x_at | 10288 | LILRB2 | AF004231 | 32.40 | 56.81 | 20.47 | 0.36 | 32.40 | 125.79 | 14.82 | 0.12 |
| | // | /// | | | | | | | | | |
| | | LILRA6 | | | | | | | | | |
| | | /// | | | | | | | | | |
| | | LOC65262 | | | | | | | | | |
| 204707_s_at | 5596 | MAPK4 | BF115223 | 6.65 | 19.98 | 7.21 | 0.36 | 6.65 | 14.25 | 10.83 | |
| 206730_at | 2892 /// | GRIA3 | NM_007325 | 11.00 | 18.63 | 6.74 | 0.36 | 11.00 | 12.47 | 3.01 | |
| | | /// | | | | | | | | | |
| | | FLJ21839 | | | | | | | | | |
| 214856_at | 6711 | SPTBN1 | BF434424 | 4.05 | 20.23 | 7.32 | 0.36 | 4.05 | 8.85 | 8.76 | |
| 1255_g_at | 2978 | GUCA1A | L36861 | 5.92 | 10.68 | 3.87 | 0.36 | 5.92 | 12.38 | 6.69 | 0.54 |
| 210106_at | 5959 | RDH5 | U43559 | 29.55 | 65.32 | 23.68 | 0.36 | 29.55 | 25.65 | 13.68 | |
| 204288_s_at | 8470 | SORBS2 | NM_021069 | 11.55 | 45.28 | 16.43 | 0.36 | 11.55 | 10.67 | 19.57 | |
| 211667_x_at | | | L34698 | 6.36 | 32.98 | 11.97 | 0.36 | 6.36 | 6.08 | 3.46 | |
| 219042_at | 11178 | LZTS1 | NM_021020 | 7.89 | 17.88 | 6.49 | 0.36 | 7.89 | 15.80 | 3.25 | 0.21 |
| 210797_s_at | 8638 | OASL | AF063612 | 72.69 | 379.07 | 137.71 | 0.36 | 72.69 | 326.62 | 199.27 | 0.61 |
| 221303_at | 29930 | PCDHB1 | NM_013340 | 9.73 | 17.04 | 6.21 | 0.36 | 9.73 | 26.11 | 22.12 | |
| 214775_at | 23138 | N4BP3 | AW139448 | 19.41 | 45.72 | 16.67 | 0.36 | 19.41 | 42.11 | 55.89 | |
| 214821_at | 291 | SLC25A4 | AF052119 | 17.95 | 37.73 | 13.76 | 0.36 | 17.95 | 22.55 | 22.74 | |
| 216136_at | | | AF113683 | 1.07 | 17.34 | 6.33 | 0.36 | 1.07 | 8.30 | 4.99 | |
| 214633_at | 6658 | SOX3 | AI824954 | 6.56 | 18.61 | 6.80 | 0.37 | 6.56 | 3.37 | 3.67 | |
| 205116_at | 3908 | LAMA2 | NM_000426 | 25.36 | 41.87 | 15.31 | 0.37 | 25.36 | 34.45 | 50.01 | |
| 216729_at | | | AK025388 | 14.14 | 22.32 | 8.16 | 0.37 | 14.14 | 20.26 | 7.24 | |
| 221462_x_at | 55554 | KLK15 | NM_017509 | 10.37 | 20.99 | 7.68 | 0.37 | 10.37 | 6.76 | 4.62 | |
| 213961_s_at | | | AI077556 | 21.60 | 33.32 | 12.22 | 0.37 | 21.60 | 8.63 | 10.54 | |
| 215266_at | 55567 | DNAH3 | AL096732 | 13.14 | 23.47 | 8.61 | 0.37 | 13.14 | 9.11 | 14.97 | |
| 217326_x_at | 5644 | PRSS1 | AF009787 | 17.26 | 31.28 | 11.49 | 0.37 | 17.26 | 8.14 | 36.57 | |
| 213071_at | 1805 | DPT | AL049798 | 3.99 | 10.14 | 3.72 | 0.37 | 3.99 | 9.40 | 8.16 | |
| 216390_at | 3931 | LCAT | X06537 | 14.96 | 37.53 | 13.80 | 0.37 | 14.96 | 11.49 | 9.37 | |
| 217217_at | 10095 | ARPC1B | X95660 | 6.17 | 15.48 | 5.69 | 0.37 | 6.17 | 11.37 | 2.53 | 0.22 |
| | // | /// | | | | | | | | | |
| | | IGHA1 | | | | | | | | | |
| 219249_s_at | 60681 | FKBP10 | NM_021939 | 3.14 | 10.62 | 3.92 | 0.37 | 3.14 | 5.88 | 5.38 | |
| 206356_s_at | 2774 | GNAL | NM_002071 | 2.16 | 26.24 | 9.69 | 0.37 | 2.16 | 15.47 | 13.67 | |
| 215995_x_at | 348094 | ANKDD1A | AU147598 | 24.03 | 76.27 | 28.16 | 0.37 | 24.03 | 26.84 | 38.03 | |
| 202728_s_at | 4052 | LTBP1 | AI986120 | 8.78 | 18.39 | 6.79 | 0.37 | 8.78 | 13.09 | 6.50 | |
| 204994_at | 4600 | MX2 | NM_002463 | 401.48 | 1048.88 | 387.53 | 0.37 | 401.48 | 1233.73 | 323.72 | 0.26 |
| 211405_x_at | 3451 | IFNA17 | M38289 | 46.09 | 101.45 | 37.49 | 0.37 | 46.09 | 27.81 | 12.90 | |
| 207777_s_at | 11262 | SP140 | NM_007237 | 12.91 | 33.57 | 12.41 | 0.37 | 12.91 | 3.29 | 7.68 | |
| 220644_at | | | NM_014137 | 5.76 | 11.76 | 4.35 | 0.37 | 5.76 | 3.36 | 11.88 | |
| 205294_at | 10458 | BAIAP2 | AB017120 | 118.85 | 178.85 | 66.12 | 0.37 | 118.85 | 331.06 | 321.61 | |
| 206894_at | 337 | APOA4 | NM_000482 | 31.00 | 60.10 | 22.24 | 0.37 | 31.00 | 23.29 | 17.31 | |
| 220588_at | 55653 | BCAS4 | NM_017843 | 13.75 | 29.11 | 10.79 | 0.37 | 13.75 | 34.75 | 29.20 | |
| 211719_x_at | 2335 | FN1 | BC005858 | 3.41 | 30.07 | 11.15 | 0.37 | 3.41 | 71.30 | 59.05 | |
| 208441_at | 3480 | IGF1R | NM_015883 | 16.45 | 35.78 | 13.30 | 0.37 | 16.45 | 4.96 | 21.03 | |
| 215026_x_at | 6337 | SCNN1A | U81961 | 10.47 | 28.85 | 10.75 | 0.37 | 10.47 | 9.92 | 11.44 | |
| 220135_s_at | 11136 | SLC7A9 | NM_014270 | 19.32 | 36.20 | 13.49 | 0.37 | 19.32 | 35.00 | 11.86 | 0.34 |
| 222285_at | 3495 | IGHD | AW134608 | 7.59 | 12.70 | 4.74 | 0.37 | 7.59 | 3.61 | 5.26 | |
| 219691_at | 54809 | SAMD9 | NM_017654 | 181.38 | 431.12 | 160.89 | 0.37 | 181.38 | 224.11 | 63.18 | |
| 220630_s_at | 27159 | CHIA | NM_021797 | 3.94 | 26.37 | 9.85 | 0.37 | 3.94 | 16.09 | 6.27 | 0.39 |
| 214603_at | 266740 / | MAGEA2 | U82671 | 2.18 | 22.06 | 8.24 | 0.37 | 2.18 | 12.12 | 16.08 | |
| | | /// | | | | | | | | | |
| | | MAGEA2B | | | | | | | | | |
| 213294_at | | | AV755522 | 685.49 | 1711.91 | 639.85 | 0.37 | 685.49 | 2174.68 | 567.17 | 0.26 |
| 216127_at | 64714 | PDIA2 | Z84717 | 2.37 | 11.67 | 4.36 | 0.37 | 2.37 | 4.45 | 6.00 | |
| 221911_at | 2115 | ETV1 | BE881590 | 16.14 | 38.28 | 14.32 | 0.37 | 16.14 | 8.57 | 1.62 | |
| 206776_x_at | 56 | ACRV1 | NM_001612 | 30.60 | 47.62 | 17.84 | 0.37 | 30.60 | 22.29 | 25.17 | |
| 209182_s_at | 11067 | C10orf10 | AI302100 | 40.04 | 71.88 | 26.93 | 0.37 | 40.04 | 60.12 | 64.91 | |
| 206863_x_at | | | U76376 | 2.87 | 10.35 | 3.88 | 0.37 | 2.87 | 4.60 | 1.98 | |
| 202669_s_at | 1948 | EFNB2 | U16797 | 11.10 | 23.81 | 8.93 | 0.38 | 11.10 | 31.06 | 59.31 | |
| 217668_at | 388886 | C22orf36 | BF511164 | 5.79 | 14.46 | 5.43 | 0.38 | 5.79 | 15.40 | 11.49 | 0.75 |
| 214294_at | 57235 | KIAA0485 | AI122905 | 13.83 | 35.13 | 13.20 | 0.38 | 13.83 | 13.10 | 18.86 | |
| 205559_s_at | 5125 | PCSK5 | NM_006200 | 6.41 | 27.47 | 10.32 | 0.38 | 6.41 | 62.04 | 151.92 | |
| 219519_s_at | 6614 | SIGLEC1 | NM_023068 | 78.10 | 195.87 | 73.63 | 0.38 | 78.10 | 99.53 | 57.39 | |
| 221631_at | 8911 | CACNA1I | AB032946 | 11.87 | 34.68 | 13.05 | 0.38 | 11.87 | 7.60 | 3.38 | |
| 217207_s_at | 10917 | BTNL3 | AK025267 | 66.36 | 100.17 | 37.73 | 0.38 | 66.36 | 57.86 | 64.04 | |
| 216634_at | 23007 | PLCH1 | AK022610 | 3.85 | 12.91 | 4.88 | 0.38 | 3.85 | 3.29 | 4.48 | |
| 217444_at | | | AL080086 | 7.09 | 26.70 | 10.10 | 0.38 | 7.09 | 8.08 | 20.65 | |
| 204401_at | 3783 | KCNN4 | NM_002250 | 417.08 | 778.30 | 294.43 | 0.38 | 417.08 | 717.37 | 520.42 | 0.73 |
| 207658_s_at | 2290 /// | FOXG1B | NM_004471 | 16.27 | 56.80 | 21.49 | 0.38 | 16.27 | 13.11 | 37.03 | |
| | | /// | | | | | | | | | |
| | | FOXG1A | | | | | | | | | |
| 217684_at | 7298 | TYMS | BG281679 | 22.41 | 46.19 | 17.50 | 0.38 | 22.41 | 9.52 | 11.89 | |
| 217374_x_at | 6978 | TRGV5 | AC006035 | 65.25 | 106.69 | 40.46 | 0.38 | 65.25 | 11.64 | 33.41 | |
| 221386_at | 4995 | OR3A2 | NM_002551 | 11.60 | 25.78 | 9.78 | 0.38 | 11.60 | 16.00 | 2.77 | |
| 214054_at | 9046 | DOK2 | AI828929 | 1119.54 | 1794.24 | 681.22 | 0.38 | 1119.54 | 1676.34 | 597.50 | |
| 221174_at | | | NM_025039 | 5.86 | 10.14 | 3.85 | 0.38 | 5.86 | 3.58 | 7.40 | |
| 206196_s_at | 10900 | RPIP8 | NM_006695 | 15.83 | 37.04 | 14.07 | 0.38 | 15.83 | 50.05 | 7.10 | 0.14 |
| 219412_at | 23682 | RAB38 | NM_022337 | 51.61 | 250.31 | 95.08 | 0.38 | 51.61 | 367.63 | 94.16 | 0.26 |
| 217026_at | 1080 | CFTR | M96936 | 10.26 | 16.74 | 6.36 | 0.38 | 10.26 | 16.06 | 3.71 | 0.23 |
| 217328_at | 5644 | PRSS1 | AF009787 | 6.76 | 10.47 | 3.98 | 0.38 | 6.76 | 5.11 | 26.32 | |
| 207470_at | 54744 | DKFZp566H0824 | NM_017535 | 7.75 | 33.34 | 12.68 | 0.38 | 7.75 | 10.24 | 13.98 | |
| 221346_at | 26476 | OR10J1 | NM_012351 | 11.04 | 82.22 | 31.33 | 0.38 | 11.04 | 39.70 | 15.43 | 0.39 |
| 209447_at | 23345 | SYNE1 | AF043290 | 57.82 | 88.35 | 33.69 | 0.38 | 57.82 | 12.86 | 6.00 | |
| 39402_at | 3553 | IL1B | M15330 | 53.05 | 659.61 | 251.68 | 0.38 | 53.05 | 10198.17 | 10747.99 | |
| 221271_at | 59067 | IL21 | NM_021803 | 4.88 | 25.84 | 9.86 | 0.38 | 4.88 | 8.13 | 6.52 | |
| 33304_at | 3669 | ISG20 | U88964 | 56.23 | 136.14 | 51.99 | 0.38 | 56.23 | 280.62 | 206.24 | 0.73 |
| 209569_x_at | 27065 | D4S234E | BC001745 | 3.37 | 14.40 | 5.50 | 0.38 | 3.37 | 8.65 | 16.76 | |
| 202827_s_at | 4323 | MMP14 | NM_004995 | 295.83 | 742.68 | 283.92 | 0.38 | 295.83 | 138.62 | 266.43 | |
| 207505_at | 5593 | PRKG2 | NM_006259 | 15.99 | 29.45 | 11.26 | 0.38 | 15.99 | 14.12 | 11.02 | |
| 214542_x_at | 8329 | HIST1H2AI | NM_003509 | 42.02 | 74.04 | 28.34 | 0.38 | 42.02 | 12.67 | 26.61 | |
| 220267_at | 192666 | KRT24 | NM_019016 | 9.22 | 17.45 | 6.68 | 0.38 | 9.22 | 10.09 | 17.24 | |
| 222059_at | 63925 | ZNF335 | BE676476 | 5.30 | 31.65 | 12.13 | 0.38 | 5.30 | 45.87 | 6.36 | 0.14 |
| 220400_at | 157680 | VPS13B | NM_017890 | 55.25 | 135.30 | 51.84 | 0.38 | 55.25 | 29.10 | 55.10 | |
| 216546_s_at | 1116 | CHI3L1 | AJ251847 | 56.96 | 88.89 | 34.14 | 0.38 | 56.96 | 232.14 | 88.08 | 0.38 |
| 215018_at | 85459 | KIAA1731 | AB051518 | 43.74 | 129.92 | 49.91 | 0.38 | 43.74 | 83.09 | 54.04 | 0.65 |
| 204400_at | 10278 | EFS | NM_005864 | 11.59 | 17.58 | 6.77 | 0.38 | 11.59 | 10.91 | 5.57 | |
| 206320_s_at | 4093 | SMAD9 | NM_005905 | 4.12 | 17.46 | 6.74 | 0.39 | 4.12 | 23.63 | 13.28 | 0.56 |
| 216131_at | 23150 | FRMD4B | AK000244 | 5.35 | 10.39 | 4.01 | 0.39 | 5.35 | 26.15 | 19.49 | 0.75 |
| 215657_at | 1811 | SLC26A3 | AK025044 | 3.27 | 25.17 | 9.73 | 0.39 | 3.27 | 6.79 | 2.90 | |
| 222257_s_at | 59272 | ACE2 | AK026461 | 4.74 | 25.89 | 10.00 | 0.39 | 4.74 | 3.39 | 6.33 | |
| 209652_s_at | 5228 | PGF | BC001422 | 7.67 | 17.05 | 6.59 | 0.39 | 7.67 | 26.24 | 2.99 | 0.11 |
| 207757_at | 80108 | ZFP2 | NM_030613 | 67.18 | 145.15 | 56.12 | 0.39 | 67.18 | 174.02 | 83.37 | 0.48 |
| 208600_s_at | 2863 | GPR39 | NM_001508 | 5.14 | 17.35 | 6.72 | 0.39 | 5.14 | 7.59 | 9.14 | |
| 206360_s_at | 9021 | SOCS3 | NM_003955 | 24.04 | 85.62 | 33.20 | 0.39 | 24.04 | 77.00 | 135.95 | |
| 215159_s_at | 65220 | NADK | AI239732 | 104.97 | 185.45 | 72.01 | 0.39 | 104.97 | 60.43 | 71.45 | |
| 209291_at | 3400 | ID4 | AW157094 | 4.93 | 19.22 | 7.46 | 0.39 | 4.93 | 9.72 | 2.43 | |
| 214032_at | 7535 | ZAP70 | AI817942 | 3.45 | 14.45 | 5.61 | 0.39 | 3.45 | 20.24 | 20.35 | |
| 210883_x_at | 1949 | EFNB3 | U57001 | 32.26 | 66.56 | 25.87 | 0.39 | 32.26 | 54.90 | 46.17 | |
| 220498_at | 10880 | ACTL7B | NM_006686 | 6.35 | 12.88 | 5.01 | 0.39 | 6.35 | 9.72 | 10.31 | |
| 215617_at | | | AU145711 | 205.43 | 504.63 | 196.27 | 0.39 | 205.43 | 419.40 | 171.84 | 0.41 |
| 205685_at | 942 | CD86 | BG236280 | 30.48 | 81.94 | 31.89 | 0.39 | 30.48 | 115.04 | 45.44 | 0.39 |
| 211374_x_at | | | AF116715 | 51.83 | 89.47 | 34.82 | 0.39 | 51.83 | 49.98 | 27.24 | |
| 204197_s_at | 864 | RUNX3 | NM_004350 | 703.88 | 1070.81 | 416.78 | 0.39 | 703.88 | 1210.69 | 546.84 | 0.45 |
| 214899_at | 163131 | ZNF780B | AC007842 | 8.82 | 17.79 | 6.93 | 0.39 | 8.82 | 11.84 | 16.82 | |
| 206032_at | 1825 | DSC3 | AI797281 | 76.10 | 230.70 | 89.88 | 0.39 | 76.10 | 156.10 | 163.08 | |
| 206985_at | 3293 | HSD17B3 | NM_000197 | 8.11 | 44.81 | 17.48 | 0.39 | 8.11 | 6.01 | 7.71 | |
| 214669_x_at | 3107 | HLA-C | BG485135 | 12.06 | 123.66 | 48.25 | 0.39 | 12.06 | 30.48 | 45.50 | |
| 214084_x_at | 29 | ABR | AW072388 | 209.66 | 634.85 | 247.92 | 0.39 | 209.66 | 192.68 | 69.85 | |
| 214609_at | 401 | PHOX2A | AI469991 | 13.14 | 50.09 | 19.57 | 0.39 | 13.14 | 31.71 | 40.05 | |
| 209371_s_at | 6452 | SH3BP2 | AI393190 | 112.60 | 236.00 | 92.21 | 0.39 | 112.60 | 125.14 | 101.81 | |
| 211325_x_at | 171220 | LOC171220 | U72518 | 170.02 | 257.02 | 100.44 | 0.39 | 170.02 | 153.21 | 185.96 | |
| 216118_at | | | AU148024 | 20.52 | 35.35 | 13.82 | 0.39 | 20.52 | 52.25 | 15.01 | 0.29 |
| 202145_at | 4061 | LY6E | NM_002346 | 309.94 | 600.22 | 235.08 | 0.39 | 309.94 | 284.59 | 162.84 | |
| 211577_s_at | 3479 | IGF1 | M37484 | 16.77 | 62.69 | 24.61 | 0.39 | 16.77 | 18.90 | 30.79 | |
| 213317_at | 53405 | CLIC5 | AL049313 | 6.34 | 33.34 | 13.10 | 0.39 | 6.34 | 119.62 | 151.72 | |
| 207709_at | 5563 | PRKAA2 | NM_006252 | 10.29 | 34.74 | 13.66 | 0.39 | 10.29 | 32.52 | 8.62 | 0.27 |
| 222363_at | | | AW979018 | 12.38 | 124.76 | 49.10 | 0.39 | 12.38 | 36.51 | 35.86 | |
| 213559_s_at | 168544 | ZNF467 | BF223401 | 4.10 | 13.65 | 5.37 | 0.39 | 4.10 | 3.74 | 2.50 | |
| 210441_at | 8556 | CDC14A | AF064102 | 42.69 | 67.52 | 26.60 | 0.39 | 42.69 | 48.45 | 40.23 | |
| 204549_at | 9641 | IKBKE | NM_014002 | 211.41 | 610.47 | 240.71 | 0.39 | 211.41 | 492.09 | 169.75 | 0.34 |
| 217101_at | 22996 | C1orf34 | AB007921 | 6.03 | 21.09 | 8.32 | 0.39 | 6.03 | 4.42 | 3.17 | |
| 206639_x_at | 3346 | HTN1 | NM_002159 | 12.95 | 23.78 | 9.39 | 0.39 | 12.95 | 4.78 | 12.14 | |
| 220101_x_at | | | NM_018628 | 24.49 | 49.27 | 19.46 | 0.40 | 24.49 | 38.90 | 20.71 | 0.53 |
| 218312_s_at | 65982 | ZNF447 | NM_023926 | 3.67 | 29.94 | 11.84 | 0.40 | 3.67 | 5.22 | 6.35 | |
| 215068_s_at | 80028 | FBXL18 | BC004228 | 103.91 | 161.23 | 63.87 | 0.40 | 103.91 | 93.60 | 55.45 | |
| 204211_x_at | 5610 | EIF2AK2 | NM_002759 | 536.17 | 1173.56 | 465.02 | 0.40 | 536.17 | 846.82 | 467.26 | 0.55 |
| 216795_at | | | AK026847 | 5.08 | 37.28 | 14.78 | 0.40 | 5.08 | 16.70 | 3.61 | 0.22 |
| 221990_at | 7849 | PAX8 | AI948472 | 6.46 | 12.60 | 5.00 | 0.40 | 6.46 | 11.75 | 4.10 | 0.35 |
| 206903_at | | | NM_005728 | 13.26 | 20.38 | 8.08 | 0.40 | 13.26 | 21.18 | 11.03 | 0.52 |
| 215717_s_at | 2201 | FBN2 | X62009 | 37.77 | 64.85 | 25.72 | 0.40 | 37.77 | 44.55 | 24.92 | |
| 210751_s_at | 9104 | RGN | D31815 | 12.81 | 20.62 | 8.18 | 0.40 | 12.81 | 3.35 | 5.45 | |
| 211215_x_at | 1734 | DIO2 | AB041843 | 12.09 | 21.26 | 8.44 | 0.40 | 12.09 | 8.02 | 8.57 | |
| 210583_at | 84271 | POLDIP3 | AB055760 | 13.11 | 29.55 | 11.73 | 0.40 | 13.11 | 6.56 | 19.95 | |
| 218744_s_at | 29763 | PACSIN3 | NM_016223 | 7.19 | 16.23 | 6.45 | 0.40 | 7.19 | 5.03 | 12.05 | |
| 217190_x_at | 2099 | ESR1 | S67777 | 7.98 | 32.15 | 12.79 | 0.40 | 7.98 | 6.06 | 13.38 | |
| 219883_at | | | NM_016611 | 36.71 | 67.19 | 26.73 | 0.40 | 36.71 | 46.04 | 20.34 | |
| 220861_at | | | NM_014093 | 11.40 | 22.11 | 8.80 | 0.40 | 11.40 | 16.59 | 29.43 | |
| 216743_at | 112 | ADCY6 | AK024915 | 3.10 | 11.02 | 4.39 | 0.40 | 3.10 | 4.46 | 2.79 | |
| 207072_at | 8807 | IL18RAP | NM_003853 | 45.16 | 229.00 | 91.30 | 0.40 | 45.16 | 445.08 | 92.54 | 0.21 |
| 207561_s_at | 9311 | ACCN3 | NM_020322 | 23.92 | 53.84 | 21.47 | 0.40 | 23.92 | 38.31 | 25.16 | 0.66 |
| 207715_at | 1419 | CRYGB | NM_005210 | 2.99 | 13.40 | 5.35 | 0.40 | 2.99 | 3.79 | 5.19 | |
| 215680_at | 85368 | KIAA1654 | AB051441 | 6.82 | 22.18 | 8.85 | 0.40 | 6.82 | 15.12 | 17.30 | |
| 208134_x_at | 5670 | PSG2 | NM_031246 | 3.32 | 22.18 | 8.85 | 0.40 | 3.32 | 3.18 | 31.50 | |
| 209697_at | 5533 | PPP3CC | BC004864 | 10.69 | 20.97 | 8.38 | 0.40 | 10.69 | 2.62 | 5.81 | |
| 210767_at | 4771 | NF2 | BC003112 | 7.55 | 20.00 | 7.99 | 0.40 | 7.55 | 22.47 | 14.39 | 0.64 |
| 221127_s_at | 10530 | RIG | NM_006394 | 2.03 | 15.06 | 6.02 | 0.40 | 2.03 | 5.55 | 1.17 | |
| 207652_s_at | 1240 | CMKLR1 | NM_004072 | 5.77 | 13.59 | 5.43 | 0.40 | 5.77 | 5.34 | 6.39 | |
| 219947_at | 50856 | CLEC4A | NM_016184 | 1.37 | 29.36 | 11.75 | 0.40 | 1.37 | 17.45 | 13.29 | |
| 211771_s_at | 5452 | POU2F2 | BC006101 | 80.85 | 363.75 | 145.69 | 0.40 | 80.85 | 551.98 | 530.55 | |
| 204248_at | 2767 | GNA11 | NM_002067 | 61.62 | 126.80 | 50.88 | 0.40 | 61.62 | 96.45 | 27.12 | 0.28 |
| 215955_x_at | 23092 | ARHGAP26 | Y10388 | 144.84 | 218.54 | 87.81 | 0.40 | 144.84 | 40.81 | 194.38 | |
| 203217_s_at | 8869 | ST3GAL5 | NM_003896 | 200.17 | 321.04 | 129.03 | 0.40 | 200.17 | 297.64 | 97.14 | |
| 205942_s_at | 6296 | ACSM3 | NM_005622 | 4.28 | 36.98 | 14.86 | 0.40 | 4.28 | 5.39 | 3.77 | |
| 214569_at | 3442 | IFNA5 | NM_002169 | 7.73 | 19.89 | 8.00 | 0.40 | 7.73 | 2.75 | 17.81 | |
| 216374_at | | | AC006986 | 28.50 | 51.65 | 20.78 | 0.40 | 28.50 | 29.47 | 37.57 | |
| 205803_s_at | 7220 | TRPC1 | NM_003304 | 19.99 | 50.43 | 20.31 | 0.40 | 19.99 | 16.57 | 31.48 | |
| 207639_at | 8326 | FZD9 | NM_003508 | 39.73 | 68.93 | 27.76 | 0.40 | 39.73 | 39.09 | 42.81 | |
| 211457_at | 23766 | GABARAPL3 | AF180519 | 4.35 | 25.70 | 10.35 | 0.40 | 4.35 | 16.09 | 3.95 | 0.25 |
| 208712_at | 595 | CCND1 | M73554 | 43.20 | 115.86 | 46.75 | 0.40 | 43.20 | 67.62 | 70.85 | |
| 210304_at | 5158 | PDE6B | BC000249 | 5.05 | 47.16 | 19.03 | 0.40 | 5.05 | 11.49 | 10.98 | |
| 216736_at | 53345 | TM6SF2 | AK024515 | 11.30 | 24.15 | 9.75 | 0.40 | 11.30 | 17.42 | 8.77 | 0.50 |
| 211314_at | 8913 | CACNA1G | AB012043 | 5.24 | 12.16 | 4.91 | 0.40 | 5.24 | 8.03 | 15.36 | |
| AFFX- | 6772 | STAT1 | AFFX- | 445.10 | 1072.24 | 433.37 | 0.40 | 445.10 | 557.35 | 376.96 | |
| HUMISGF3A/ | | | HUMISGF3A/ | | | | | | | | |
| M97935_MA_at | | | M97935_MA | | | | | | | | |
| 217308_at | 26184 | OR1F2 | AJ003145 | 2.89 | 15.87 | 6.42 | 0.40 | 2.89 | 3.11 | 22.98 | |
| 212803_at | 4665 | NAB2 | BF337329 | 254.48 | 796.94 | 322.72 | 0.40 | 254.48 | 944.78 | 1422.16 | |
| 220681_at | 55267 | C22orf26 | NM_018280 | 23.29 | 73.80 | 29.90 | 0.41 | 23.29 | 18.00 | 32.96 | |
| 219958_at | 55321 | C20orf46 | NM_018354 | 27.49 | 45.51 | 18.44 | 0.41 | 27.49 | 39.90 | 45.21 | |
| 221226_s_at | 55515 | ACCN4 | NM_018674 | 6.49 | 14.31 | 5.80 | 0.41 | 6.49 | 6.15 | 6.05 | |
| 212706_at | 10156 | RASA4 | AI738591 | 250.46 | 652.76 | 265.06 | 0.41 | 250.46 | 572.38 | 241.22 | 0.42 |
| 211300_s_at | 7157 | TP53 | K03199 | 14.40 | 32.51 | 13.20 | 0.41 | 14.40 | 13.33 | 19.24 | |
| 221323_at | 80329 | ULBP1 | NM_025218 | 7.96 | 43.18 | 17.53 | 0.41 | 7.96 | 11.10 | 70.11 | |
| 208851_s_at | 7070 | THY1 | AL161958 | 31.49 | 66.80 | 27.13 | 0.41 | 31.49 | 10.19 | 20.89 | |
| 204803_s_at | 6236 | RRAD | NM_004165 | 12.44 | 107.26 | 43.64 | 0.41 | 12.44 | 266.20 | 688.02 | |
| 219091_s_at | 79812 | MMRN2 | NM_024756 | 30.82 | 49.04 | 19.96 | 0.41 | 30.82 | 29.01 | 22.77 | |
| 210800_at | 1678 | TIMM8A | BC005236 | 17.06 | 33.69 | 13.74 | 0.41 | 17.06 | 12.81 | 10.08 | |
| 212921_at | 56950 | SMYD2 | AF070592 | 39.59 | 83.90 | 34.23 | 0.41 | 39.59 | 52.73 | 45.12 | |
| 218410_s_at | 283871 | LOC283871 | NM_024118 | 44.67 | 76.47 | 31.22 | 0.41 | 44.67 | 27.84 | 10.67 | |
| 214398_s_at | 9641 | IKBKE | AW340333 | 192.89 | 641.33 | 261.99 | 0.41 | 192.89 | 244.87 | 155.52 | |
| 206957_at | 189 | AGXT | NM_016236 | 1.69 | 22.37 | 9.14 | 0.41 | 1.69 | 11.70 | 3.88 | 0.33 |
| 216419_at | 9696 | CROCC | AK026910 | 18.87 | 49.78 | 20.34 | 0.41 | 18.87 | 37.46 | 8.61 | 0.23 |
| 203940_s_at | 22846 | VASH1 | NM_014909 | 112.21 | 213.08 | 87.13 | 0.41 | 112.21 | 111.34 | 60.99 | |
| 206133_at | 54739 | BIRC4BP | NM_017523 | 429.25 | 1584.83 | 648.08 | 0.41 | 429.25 | 830.56 | 290.18 | 0.35 |
| 213462_at | 4862 | NPAS2 | AW000928 | 19.40 | 59.25 | 24.27 | 0.41 | 19.40 | 55.39 | 80.46 | |
| 206212_at | 1358 | CPA2 | NM_001869 | 39.95 | 74.70 | 30.60 | 0.41 | 39.95 | 41.82 | 19.46 | |
| 205009_at | 7031 | TFF1 | NM_003225 | 6.32 | 23.62 | 9.68 | 0.41 | 6.32 | 14.42 | 3.85 | 0.27 |
| 219256_s_at | 54436 | SH3TC1 | NM_018986 | 193.32 | 431.34 | 176.95 | 0.41 | 193.32 | 285.80 | 129.56 | |
| AFFX- | | | AFFX- | 2.57 | 11.24 | 4.62 | 0.41 | 2.57 | 5.13 | 3.89 | |
| TrpnX-3_at | | | TrpnX-3 | | | | | | | | |
| 206186_at | 4356 | MPP3 | NM_001932 | 43.70 | 79.92 | 32.88 | 0.41 | 43.70 | 39.10 | 79.49 | |
| 215477_at | | | H49077 | 22.60 | 35.27 | 14.51 | 0.41 | 22.60 | 2.77 | 13.86 | |
| 217601_at | 23511 | NUP188 | AL523184 | 53.17 | 104.72 | 43.10 | 0.41 | 53.17 | 72.84 | 50.59 | |
| 221924_at | 83637 | ZMIZ2 | AW444969 | 232.18 | 805.22 | 331.45 | 0.41 | 232.18 | 821.46 | 529.04 | 0.64 |
| 206317_s_at | 11194 | ABCB8 | NM_007188 | 25.97 | 98.14 | 40.41 | 0.41 | 25.97 | 15.87 | 43.98 | |
| 210064_s_at | 7348 | UPK1B | NM_006952 | 14.42 | 22.55 | 9.29 | 0.41 | 14.42 | 22.60 | 16.50 | 0.73 |
| 214046_at | | | AA017721 | 17.22 | 25.97 | 10.72 | 0.41 | 17.22 | 14.30 | 17.89 | |
| 209834_at | 9469 | CHST3 | AB017915 | 24.75 | 50.34 | 20.79 | 0.41 | 24.75 | 74.27 | 30.91 | 0.42 |
| 205391_x_at | 286 | ANK1 | M28880 | 2.50 | 10.33 | 4.26 | 0.41 | 2.50 | 12.86 | 4.43 | 0.34 |
| 203071_at | 7869 | SEMA3B | NM_004636 | 6.04 | 10.60 | 4.38 | 0.41 | 6.04 | 7.89 | 5.95 | |
| 221106_at | 51310 | SLC22A17 | NM_016609 | 13.05 | 42.50 | 17.57 | 0.41 | 13.05 | 32.31 | 29.55 | |
| 205293_x_at | 10458 | BAIAP2 | AB017120 | 81.77 | 154.22 | 63.80 | 0.41 | 81.77 | 265.59 | 268.76 | |
| 213450_s_at | 23308 | ICOSLG | AI659611 | 141.14 | 445.28 | 184.26 | 0.41 | 141.14 | 855.53 | 713.61 | |
| 216670_at | 26085 | KLK13 | AL050220 | 5.86 | 16.72 | 6.92 | 0.41 | 5.86 | 5.37 | 6.29 | |
| 216919_at | 9537 | TP53I11 | U79302 | 28.48 | 56.21 | 23.28 | 0.41 | 28.48 | 47.67 | 41.85 | |
| 205568_at | 366 | AQP9 | NM_020980 | 3.15 | 12.81 | 5.30 | 0.41 | 3.15 | 57.07 | 96.71 | |
| 215671_at | 5142 | PDE4B | AU144792 | 3.90 | 62.94 | 26.07 | 0.41 | 3.90 | 80.18 | 50.21 | 0.63 |
| 218704_at | 54894 | RNF43 | NM_017763 | 7.69 | 15.77 | 6.53 | 0.41 | 7.69 | 13.71 | 12.34 | |
| 207921_x_at | 7849 | PAX8 | NM_013952 | 14.83 | 34.92 | 14.47 | 0.41 | 14.83 | 17.68 | 19.77 | |
| 208436_s_at | 3665 | IRF7 | NM_004030 | 352.89 | 1349.42 | 559.42 | 0.41 | 352.89 | 1434.35 | 576.39 | 0.40 |
| 204741_at | 636 | BICD1 | NM_001714 | 59.33 | 142.85 | 59.24 | 0.41 | 59.33 | 115.28 | 91.33 | |
| 206378_at | 4250 | SCGB2A2 | NM_002411 | 2.44 | 16.24 | 6.73 | 0.41 | 2.44 | 6.33 | 6.51 | |
| 209589_s_at | 2048 | EPHB2 | AF025304 | 4.95 | 16.88 | 7.00 | 0.41 | 4.95 | 38.60 | 4.17 | 0.11 |
| 206504_at | 1591 | CYP24A1 | NM_000782 | 3.82 | 20.09 | 8.34 | 0.41 | 3.82 | 14.40 | 6.96 | 0.48 |
| 204501_at | 4856 | NOV | NM_002514 | 16.91 | 43.40 | 18.02 | 0.42 | 16.91 | 15.13 | 20.71 | |
| 217220_at | | | AL050153 | 2.47 | 16.81 | 6.98 | 0.42 | 2.47 | 13.14 | 17.75 | |
| 201236_s_at | 7832 | BTG2 | BG339064 | 504.34 | 3730.56 | 1551.96 | 0.42 | 504.34 | 5160.39 | 5204.92 | |
| 203088_at | 10516 | FBLN5 | NM_006329 | 5.51 | 37.36 | 15.55 | 0.42 | 5.51 | 21.14 | 13.55 | 0.64 |
| 205693_at | 7140 | TNNT3 | NM_006757 | 16.96 | 38.24 | 15.92 | 0.42 | 16.96 | 13.02 | 16.63 | |
| 216180_s_at | 8871 | SYNJ2 | AK026758 | 20.07 | 55.74 | 23.22 | 0.42 | 20.07 | 43.87 | 138.70 | |
| 222336_at | 201895 | C4orf34 | AW974915 | 8.31 | 24.32 | 10.14 | 0.42 | 8.31 | 20.02 | 15.58 | |
| 210634_at | 27252 | KLHL20 | BC005253 | 4.10 | 60.74 | 25.34 | 0.42 | 4.10 | 25.74 | 6.05 | 0.24 |
| 220719_at | 80079 | FLJ13769 | NM_025012 | 23.37 | 53.64 | 22.38 | 0.42 | 23.37 | 24.74 | 33.70 | |
| 217049_x_at | 83259 | PCDH11Y | AJ276803 | 29.07 | 49.70 | 20.75 | 0.42 | 29.07 | 57.68 | 36.85 | 0.64 |
| 216184_s_at | 22999 | RIMS1 | AF263310 | 2.28 | 10.50 | 4.38 | 0.42 | 2.28 | 2.00 | 7.78 | |
| 217121_at | 8658 | TNKS | AF082559 | 15.14 | 29.29 | 12.24 | 0.42 | 15.14 | 3.52 | 12.40 | |
| 221357_at | 1132 | CHRM4 | NM_000741 | 23.68 | 39.30 | 16.43 | 0.42 | 23.68 | 17.90 | 19.28 | |
| 214768_x_at | 3107 | HLA-C | BG540628 | 38.74 | 87.89 | 36.75 | 0.42 | 38.74 | 47.79 | 15.63 | |
| 203980_at | 2167 | FABP4 | NM_001442 | 5.97 | 35.60 | 14.89 | 0.42 | 5.97 | 139.54 | 8.74 | 0.06 |
| 207689_at | 347853 | TBX10 | NM_005995 | 16.00 | 30.47 | 12.75 | 0.42 | 16.00 | 19.73 | 5.02 | |
| 204929_s_at | 10791 | VAMP5 | NM_006634 | 56.53 | 88.95 | 37.24 | 0.42 | 56.53 | 33.09 | 35.23 | |
| 220692_at | 246721 | POLR2J2 | NM_014147 | 122.96 | 284.17 | 119.05 | 0.42 | 122.96 | 236.10 | 107.81 | 0.46 |
| 201601_x_at | 8519 | IFITM1 | NM_003641 | 763.85 | 1339.59 | 561.23 | 0.42 | 763.85 | 991.47 | 521.37 | |
| 213949_s_at | 83475 | DOHH | AI370867 | 5.39 | 15.21 | 6.38 | 0.42 | 5.39 | 28.56 | 10.13 | 0.35 |
| 221200_at | | | NM_022155 | 11.57 | 35.50 | 14.90 | 0.42 | 11.57 | 133.61 | 82.10 | 0.61 |
| 206179_s_at | 11076 | TPPP | NM_007030 | 13.85 | 30.12 | 12.66 | 0.42 | 13.85 | 27.82 | 41.06 | |
| 207389_at | 2811 | GP1BA | NM_000173 | 50.73 | 135.31 | 56.94 | 0.42 | 50.73 | 155.89 | 88.98 | 0.57 |
| 215583_at | 9725 | TMEM63A | AU148184 | 76.40 | 127.56 | 53.70 | 0.42 | 76.40 | 56.85 | 20.29 | |
| 214038_at | 6355 | CCL8 | AI984980 | 4.39 | 202.95 | 85.48 | 0.42 | 4.39 | 1064.30 | 160.82 | 0.15 |
| 208397_x_at | 3762 | KCNJ5 | U39195 | 6.90 | 15.97 | 6.73 | 0.42 | 6.90 | 6.32 | 6.81 | |
| 205728_at | | | AL022718 | 7.21 | 20.74 | 8.74 | 0.42 | 7.21 | 17.02 | 13.42 | |
| 221578_at | 83937 | RASSF4 | AF260335 | 237.66 | 705.35 | 298.06 | 0.42 | 237.66 | 605.86 | 441.42 | 0.73 |
| 214223_at | | | AA427737 | 12.07 | 20.87 | 8.83 | 0.42 | 12.07 | 5.10 | 26.09 | |
| 215928_at | 84448 | ABLIM2 | AK022192 | 4.87 | 12.70 | 5.38 | 0.42 | 4.87 | 15.15 | 21.61 | |
| 210237_at | 9048 | ARTN | AF120274 | 8.78 | 18.25 | 7.73 | 0.42 | 8.78 | 21.46 | 10.59 | 0.49 |
| 217302_at | 135948 | OR2F2 | AC004853 | 18.04 | 53.83 | 22.82 | 0.42 | 18.04 | 32.41 | 26.16 | |
| 42361_g_at | 54535 | CCHCR1 | AI588986 | 69.42 | 126.04 | 53.47 | 0.42 | 69.42 | 79.47 | 63.80 | |
| 221119_at | 54848 | FLJ20184 | NM_017700 | 12.86 | 31.29 | 13.28 | 0.42 | 12.86 | 23.69 | 8.38 | 0.35 |
| 210772_at | 2358 | FPRL1 | M88107 | 43.23 | 75.72 | 32.13 | 0.42 | 43.23 | 92.72 | 6.49 | 0.07 |
| 207339_s_at | 4050 | LTB | NM_002341 | 175.05 | 492.42 | 209.20 | 0.42 | 175.05 | 139.08 | 100.65 | |
| 217523_at | 960 | CD44 | AV700298 | 65.00 | 330.57 | 140.49 | 0.42 | 65.00 | 1111.13 | 1674.02 | |
| 215485_s_at | 3383 | ICAM1 | AA284705 | 52.06 | 355.68 | 151.18 | 0.43 | 52.06 | 315.11 | 268.32 | |
| 200785_s_at | 4035 | LRP1 | NM_002332 | 13.80 | 24.68 | 10.51 | 0.43 | 13.80 | 26.69 | 19.12 | 0.72 |
| 220293_at | 79820 | C14orf161 | NM_024764 | 14.18 | 21.38 | 9.10 | 0.43 | 14.18 | 20.25 | 13.64 | |
| 209491_s_at | 272 /// | AMPD3 | AA919119 | 22.46 | 47.78 | 20.36 | 0.43 | 22.46 | 52.85 | 60.46 | |
| | | /// | | | | | | | | | |
| | | PPARA | | | | | | | | | |
| | | /// | | | | | | | | | |
| | | TRMU | | | | | | | | | |
| 216800_at | | | AK027069 | 13.56 | 22.16 | 9.45 | 0.43 | 13.56 | 10.90 | 7.51 | |
| 213688_at | 801 | CALM1 | N25325 | 75.21 | 123.85 | 52.85 | 0.43 | 75.21 | 84.07 | 129.22 | |
| 216955_at | 6872 | TAF1 | X07024 | 19.26 | 57.42 | 24.50 | 0.43 | 19.26 | 16.81 | 12.02 | |
| 206729_at | 943 | TNFRSF8 | NM_001243 | 42.36 | 67.47 | 28.81 | 0.43 | 42.36 | 46.66 | 30.11 | |
| 219867_at | 140578 | CHODL | NM_024944 | 11.63 | 37.14 | 15.86 | 0.43 | 11.63 | 26.83 | 9.91 | 0.37 |
| 221029_s_at | 81029 | WNT5B | NM_030775 | 5.45 | 40.16 | 17.18 | 0.43 | 5.45 | 39.30 | 44.46 | |
| 211302_s_at | 5142 | PDE4B | L20966 | 42.56 | 694.41 | 297.31 | 0.43 | 42.56 | 785.18 | 723.06 | |
| 220280_s_at | 51281 | ANKMY1 | NM_016552 | 20.15 | 30.77 | 13.19 | 0.43 | 20.15 | 7.98 | 17.60 | |
| 216019_x_at | 23187 | PHLDB1 | AK021690 | 7.22 | 34.45 | 14.76 | 0.43 | 7.22 | 41.34 | 42.25 | |
| 206864_s_at | 8739 | HRK | NM_003806 | 16.85 | 42.67 | 18.31 | 0.43 | 16.85 | 7.68 | 17.84 | |
| 216155_at | 89796 | NAV1 | AK024543 | 13.53 | 25.34 | 10.88 | 0.43 | 13.53 | 14.37 | 2.87 | |
| 211399_at | 2263 | FGFR2 | AB030077 | 6.42 | 13.38 | 5.74 | 0.43 | 6.42 | 15.18 | 6.85 | 0.45 |
| 205912_at | 5406 | PNLIP | NM_000936 | 5.34 | 11.56 | 4.96 | 0.43 | 5.34 | 12.41 | 9.04 | 0.73 |
| 212654_at | 122769 / | TPM2 | AL566786 | 37.37 | 59.97 | 25.79 | 0.43 | 37.37 | 27.02 | 44.82 | |
| | | /// | | | | | | | | | |
| | | PPIL5 | | | | | | | | | |
| 222368_at | | | AW972351 | 77.14 | 124.37 | 53.52 | 0.43 | 77.14 | 65.70 | 67.96 | |
| 211031_s_at | 7461 | CYLN2 | BC006259 | 106.20 | 314.64 | 135.40 | 0.43 | 106.20 | 1680.79 | 835.64 | 0.50 |
| 220683_at | 50700 | RDH8 | NM_015725 | 17.71 | 28.18 | 12.13 | 0.43 | 17.71 | 4.07 | 24.89 | |
| 219503_s_at | 55287 | TMEM40 | AI087937 | 18.25 | 42.85 | 18.44 | 0.43 | 18.25 | 19.57 | 20.62 | |
| 220207_at | 541469 | LOC541469 | NM_022125 | 11.46 | 47.81 | 20.58 | 0.43 | 11.46 | 12.60 | 13.52 | |
| 221773_at | 2004 | ELK3 | AW575374 | 539.20 | 858.25 | 369.85 | 0.43 | 539.20 | 866.34 | 237.59 | 0.27 |
| 209969_s_at | 6772 | STAT1 | BC002704 | 182.75 | 975.92 | 420.63 | 0.43 | 182.75 | 1024.44 | 251.62 | 0.25 |
| 204259_at | 4316 | MMP7 | NM_002423 | 4.44 | 14.69 | 6.33 | 0.43 | 4.44 | 8.81 | 33.19 | |
| 209641_s_at | 8714 | ABCC3 | AF009670 | 6.47 | 11.48 | 4.95 | 0.43 | 6.47 | 66.70 | 82.57 | |
| 220733_at | 10861 | SLC26A1 | NM_022042 | 6.30 | 10.59 | 4.57 | 0.43 | 6.30 | 5.69 | 7.52 | |
| 221072_at | 57000 | C9orf31 | NM_020250 | 7.00 | 11.67 | 5.04 | 0.43 | 7.00 | 5.84 | 3.59 | |
| 204961_s_at | 653361 / | NCF1 | NM_000265 | 176.02 | 536.94 | 231.99 | 0.43 | 176.02 | 178.48 | 58.28 | |
| | | /// | | | | | | | | | |
| | | NCF1B | | | | | | | | | |
| | | /// | | | | | | | | | |
| | | NCF1C | | | | | | | | | |
| 221134_at | 51378 | ANGPT4 | NM_015985 | 18.36 | 46.80 | 20.26 | 0.43 | 18.36 | 32.58 | 46.40 | |
| 210348_at | 5414 | 4-Sep | AF176379 | 4.64 | 32.31 | 14.00 | 0.43 | 4.64 | 2.40 | 12.91 | |
| 221122_at | 54979 | HRASLS2 | NM_017878 | 6.52 | 13.21 | 5.72 | 0.43 | 6.52 | 35.01 | 9.47 | 0.27 |
| 213931_at | 3398 /// | ID2 /// | AI819238 | 374.59 | 707.10 | 306.79 | 0.43 | 374.59 | 1599.99 | 626.43 | 0.39 |
| | | ID2B | | | | | | | | | |
| 220204_s_at | 353500 | BMP8A | NM_024732 | 12.62 | 19.16 | 8.32 | 0.43 | 12.62 | 7.06 | 10.70 | |
| 214403_x_at | 25803 | SPDEF | AI307915 | 33.45 | 93.08 | 40.42 | 0.43 | 33.45 | 61.99 | 29.06 | 0.47 |
| 211170_s_at | 10846 | PDE10A | AF127480 | 4.94 | 11.39 | 4.95 | 0.43 | 4.94 | 3.23 | 7.27 | |
| 215782_at | 286526 | LOC286526 | Z95624 | 38.92 | 64.80 | 28.17 | 0.43 | 38.92 | 31.54 | 12.63 | |
| 204560_at | 2289 | FKBP5 | NM_004117 | 345.23 | 708.34 | 308.31 | 0.44 | 345.23 | 501.04 | 544.14 | |
| 220501_at | 10881 | ACTL7A | NM_006687 | 11.38 | 17.22 | 7.50 | 0.44 | 11.38 | 16.65 | 7.05 | |
| 219093_at | 55022 | FLJ20701 | NM_017933 | 24.06 | 45.92 | 19.99 | 0.44 | 24.06 | 312.11 | 118.09 | 0.38 |
| 205712_at | 5789 | PTPRD | NM_002839 | 15.72 | 29.99 | 13.08 | 0.44 | 15.72 | 6.14 | 9.42 | |
| 214025_at | 55794 | DDX28 | AI922937 | 9.71 | 41.80 | 18.24 | 0.44 | 9.71 | 24.81 | 10.46 | 0.42 |
| 218978_s_at | 51312 | SLC25A37 | NM_018586 | 61.04 | 107.49 | 46.92 | 0.44 | 61.04 | 40.60 | 25.61 | |
| 220283_at | 79802 | KIAA1822L | NM_024746 | 6.84 | 24.09 | 10.54 | 0.44 | 6.84 | 38.16 | 35.00 | |
| 218599_at | 9985 | REC8L1 | NM_005132 | 175.87 | 455.17 | 199.13 | 0.44 | 175.87 | 315.80 | 188.42 | 0.60 |
| 211197_s_at | 23308 | ICOSLG | AL355690 | 82.89 | 197.58 | 86.46 | 0.44 | 82.89 | 583.18 | 517.54 | |
| 205850_s_at | 2562 | GABRB3 | NM_000814 | 11.87 | 19.37 | 8.48 | 0.44 | 11.87 | 3.91 | 5.46 | |
| 212657_s_at | 3557 | IL1RN | AW083357 | 720.99 | 1159.37 | 508.51 | 0.44 | 720.99 | 2738.88 | 4547.59 | |
| 221730_at | 1290 | COL5A2 | AL575735 | 7.38 | 14.21 | 6.24 | 0.44 | 7.38 | 234.04 | 114.89 | 0.49 |
| 200644_at | 65108 | MARCKSL1 | NM_023009 | 265.69 | 552.74 | 243.42 | 0.44 | 265.69 | 1538.92 | 952.19 | 0.62 |
| 208012_x_at | 3431 | SP110 | NM_004509 | 699.65 | 1152.86 | 508.05 | 0.44 | 699.65 | 990.50 | 276.84 | |
| 211819_s_at | 10580 | SORBS1 | AF136381 | 36.13 | 64.25 | 28.32 | 0.44 | 36.13 | 37.83 | 42.91 | |
| 216809_at | 1538 | CYLC1 | Z22780 | 23.47 | 37.70 | 16.63 | 0.44 | 23.47 | 34.92 | 41.50 | |
| 216701_at | 553168 | C1orf68 | AF005081 | 10.18 | 24.21 | 10.68 | 0.44 | 10.18 | 9.12 | 12.10 | |
| 214667_s_at | 9537 | TP53I11 | AK026607 | 22.50 | 93.09 | 41.10 | 0.44 | 22.50 | 78.35 | 101.24 | |
| 202206_at | 10123 | ARL4C | AW450363 | 154.68 | 233.51 | 103.15 | 0.44 | 154.68 | 165.44 | 92.25 | |
| 217505_at | 151230 | KLHL23 | BG403790 | 11.10 | 29.89 | 13.22 | 0.44 | 11.10 | 15.93 | 2.20 | |
| 222202_at | 644213 | LOC644213 | AK024355 | 1.30 | 13.55 | 5.99 | 0.44 | 1.30 | 12.69 | 1.22 | 0.10 |
| 209603_at | 2625 | GATA3 | AI796169 | 3.90 | 11.52 | 5.10 | 0.44 | 3.90 | 10.57 | 5.50 | 0.52 |
| 220920_at | 23120 | ATP10B | NM_025153 | 12.31 | 53.50 | 23.70 | 0.44 | 12.31 | 45.10 | 26.64 | 0.59 |
| 213033_s_at | 4781 | NFIB | AI186739 | 3.22 | 12.02 | 5.34 | 0.44 | 3.22 | 2.92 | 2.42 | |
| 210352_at | 10902 | BRD8 | AL136823 | 3.52 | 39.59 | 17.61 | 0.44 | 3.52 | 8.22 | 13.35 | |
| 217074_at | 54498 | SMOX | AK025938 | 4.72 | 26.10 | 11.62 | 0.45 | 4.72 | 21.62 | 33.60 | |
| 215779_s_at | 8339 | HIST1H2BG | BE271470 | 35.37 | 91.29 | 40.64 | 0.45 | 35.37 | 55.16 | 59.76 | |
| 213413_at | 11037 | STON1 | BG434174 | 7.75 | 13.60 | 6.06 | 0.45 | 7.75 | 18.09 | 9.58 | 0.53 |
| 205913_at | 5346 | PLIN | NM_002666 | 4.07 | 12.27 | 5.47 | 0.45 | 4.07 | 2.73 | 3.69 | |
| 211147_s_at | 9127 | P2RXL1 | AF065385 | 6.45 | 14.73 | 6.57 | 0.45 | 6.45 | 7.05 | 15.91 | |
| 215141_at | 317648 | C4orf10 | AA493300 | 14.54 | 43.10 | 19.25 | 0.45 | 14.54 | 9.24 | 9.62 | |
| 205696_s_at | 2674 | GFRA1 | U97144 | 12.08 | 22.04 | 9.85 | 0.45 | 12.08 | 3.91 | 10.50 | |
| 204908_s_at | 602 | BCL3 | NM_005178 | 164.74 | 2982.82 | 1333.44 | 0.45 | 164.74 | 2151.79 | 1227.55 | 0.57 |
| 216292_at | 5797 | PTPRM | AK024455 | 25.55 | 65.71 | 29.42 | 0.45 | 25.55 | 43.71 | 21.62 | 0.49 |
| 208136_s_at | 81854 | MGC3771 | NM_030970 | 15.87 | 40.70 | 18.24 | 0.45 | 15.87 | 18.10 | 26.01 | |
| 215487_x_at | 388358 / | RP13- | AL096727 | 8.75 | 21.94 | 9.83 | 0.45 | 8.75 | 5.45 | 8.55 | |
| | | 401N8.2 | | | | | | | | | |
| | | /// | | | | | | | | | |
| | | LOC728882 | | | | | | | | | |
| 206672_at | 359 | AQP2 | NM_000486 | 20.72 | 98.99 | 44.38 | 0.45 | 20.72 | 62.52 | 50.65 | |
| 210603_at | 84779 | MGC10646 | BC004552 | 35.55 | 55.00 | 24.68 | 0.45 | 35.55 | 26.32 | 39.37 | |
| 205312_at | 6688 | SPI1 | NM_003120 | 892.79 | 2323.94 | 1043.34 | 0.45 | 892.79 | 1948.44 | 1530.45 | |
| 210272_at | 1556 | CYP2B7P1 | M29873 | 26.67 | 50.74 | 22.78 | 0.45 | 26.67 | 16.86 | 11.71 | |
| 213845_at | 2898 | GRIK2 | AL355532 | 85.54 | 145.33 | 65.33 | 0.45 | 85.54 | 104.12 | 89.43 | |
| 205861_at | 6689 | SPIB | NM_003121 | 187.26 | 582.49 | 261.87 | 0.45 | 187.26 | 113.49 | 33.03 | |
| 212423_at | 219654 | C10orf56 | AK024784 | 132.23 | 259.80 | 116.92 | 0.45 | 132.23 | 207.76 | 215.60 | |
| 207462_at | 2742 | GLRA2 | NM_002063 | 13.25 | 21.78 | 9.81 | 0.45 | 13.25 | 15.28 | 37.06 | |
| 205605_at | 3235 | HOXD9 | NM_014213 | 10.80 | 18.86 | 8.50 | 0.45 | 10.80 | 2.85 | 3.80 | |
| 221159_at | | | NM_016414 | 12.92 | 75.39 | 34.01 | 0.45 | 12.92 | 11.37 | 9.72 | |
| 213324_at | 6714 | SRC | AK024281 | 11.16 | 238.31 | 107.56 | 0.45 | 11.16 | 489.69 | 657.07 | |
| 206534_at | 2903 | GRIN2A | NM_000833 | 14.61 | 22.18 | 10.02 | 0.45 | 14.61 | 15.04 | 7.84 | |
| 220365_at | 55821 | ALLC | NM_018436 | 19.60 | 46.86 | 21.16 | 0.45 | 19.60 | 45.24 | 17.49 | 0.39 |
| 220303_at | 79849 | PDZD3 | NM_024791 | 11.35 | 28.37 | 12.82 | 0.45 | 11.35 | 12.57 | 14.38 | |
| 213985_s_at | 91304 | C19orf6 | H45660 | 18.95 | 42.38 | 19.16 | 0.45 | 18.95 | 20.74 | 23.64 | |
| 207407_x_at | 1579 | CYP4A11 | NM_000778 | 10.17 | 16.57 | 7.49 | 0.45 | 10.17 | 4.98 | 23.32 | |
| 207881_at | | | NM_015887 | 14.31 | 23.19 | 10.50 | 0.45 | 14.31 | 19.25 | 3.75 | |
| 214521_at | 54626 | HES2 | NM_019089 | 2.19 | 12.42 | 5.63 | 0.45 | 2.19 | 13.35 | 3.61 | 0.27 |
| 202953_at | 713 | C1QB | NM_000491 | 5.39 | 31.18 | 14.14 | 0.45 | 5.39 | 5.07 | 3.14 | |
| 206893_at | 6299 | SALL1 | NM_002968 | 3.19 | 15.53 | 7.04 | 0.45 | 3.19 | 10.77 | 3.13 | 0.29 |
| 221975_s_at | 755 | C21orf2 | AI539305 | 52.65 | 101.92 | 46.25 | 0.45 | 52.65 | 126.57 | 48.58 | 0.38 |
| 217465_at | 10787 | NCKAP1 | AK001291 | 18.60 | 41.42 | 18.80 | 0.45 | 18.60 | 15.60 | 6.48 | |
| 219983_at | 57110 | HRASLS | NM_020386 | 5.68 | 13.48 | 6.12 | 0.45 | 5.68 | 6.77 | 7.42 | |
| 217867_x_at | 25825 | BACE2 | AF178532 | 13.85 | 24.09 | 10.94 | 0.45 | 13.85 | 10.30 | 8.86 | |
| 210330_at | 6444 | SGCD | U58331 | 2.30 | 11.65 | 5.30 | 0.45 | 2.30 | 25.17 | 1.08 | 0.04 |
| 215331_at | 22989 | MYH15 | BF062942 | 13.07 | 38.31 | 17.41 | 0.45 | 13.07 | 24.30 | 6.75 | 0.28 |
| 214421_x_at | 1559 | CYP2C9 | AV652420 | 16.69 | 42.36 | 19.25 | 0.45 | 16.69 | 24.55 | 17.90 | |
| 221784_at | 58525 | WIZ | AI828531 | 16.16 | 41.37 | 18.80 | 0.45 | 16.16 | 38.04 | 63.61 | |
| 208017_s_at | 4168 | MCF2 | NM_005369 | 3.22 | 15.88 | 7.22 | 0.45 | 3.22 | 12.11 | 7.74 | 0.64 |
| 207217_s_at | 27035 | NOX1 | NM_013955 | 20.84 | 34.03 | 15.48 | 0.45 | 20.84 | 7.81 | 9.77 | |
| 217272_s_at | 5275 | SERPINB13 | AJ001698 | 10.98 | 25.76 | 11.73 | 0.46 | 10.98 | 28.97 | 10.75 | 0.37 |
| 208344_x_at | 3447 | IFNA13 | NM_006900 | 12.26 | 36.18 | 16.47 | 0.46 | 12.26 | 20.59 | 6.14 | 0.30 |
| 217253_at | | | L37198 | 83.15 | 179.63 | 81.80 | 0.46 | 83.15 | 214.50 | 79.03 | 0.37 |
| 221465_at | 8590 | OR6A2 | NM_003696 | 4.47 | 10.29 | 4.69 | 0.46 | 4.47 | 5.20 | 7.49 | |
| 221415_s_at | 81025 | GJA10 | NM_030772 | 16.11 | 64.23 | 29.28 | 0.46 | 16.11 | 37.27 | 36.01 | |
| 218952_at | 27344 | PCSK1N | NM_013271 | 21.06 | 65.45 | 29.86 | 0.46 | 21.06 | 13.22 | 36.47 | |
| 217180_at | 96610 | LOC96610 | X79782 | 7.02 | 11.81 | 5.40 | 0.46 | 7.02 | 18.38 | 5.46 | 0.30 |
| 221184_at | | | NM_025089 | 10.05 | 50.83 | 23.29 | 0.46 | 10.05 | 19.95 | 39.95 | |
| 210839_s_at | 5168 | ENPP2 | D45421 | 30.55 | 89.57 | 41.05 | 0.46 | 30.55 | 13.53 | 30.26 | |
| 215142_at | 25763 | CXorf27 | AA815276 | 4.69 | 19.24 | 8.82 | 0.46 | 4.69 | 13.11 | 5.56 | 0.42 |
| 219775_s_at | 594855 | CPLX3 | NM_024695 | 11.29 | 19.61 | 8.99 | 0.46 | 11.29 | 10.98 | 6.92 | |
| 206150_at | 939 | CD27 | NM_001242 | 9.95 | 39.60 | 18.17 | 0.46 | 9.95 | 11.18 | 20.17 | |
| 206876_at | 6492 | SIM1 | AL121948 | 75.31 | 127.30 | 58.44 | 0.46 | 75.31 | 278.72 | 254.63 | |
| 218293_x_at | 10762 | NUP50 | AF267865 | 32.63 | 77.95 | 35.80 | 0.46 | 32.63 | 27.97 | 16.46 | |
| 214349_at | | | AV764378 | 191.98 | 336.49 | 154.64 | 0.46 | 191.98 | 120.06 | 171.10 | |
| 204595_s_at | 6781 | STC1 | AI300520 | 15.57 | 35.50 | 16.32 | 0.46 | 15.57 | 35.63 | 44.51 | |
| 205119_s_at | 2357 | FPR1 | NM_002029 | 38.89 | 94.38 | 43.40 | 0.46 | 38.89 | 81.06 | 12.66 | 0.16 |
| 202454_s_at | 2065 | ERBB3 | NM_001982 | 13.28 | 24.46 | 11.25 | 0.46 | 13.28 | 28.89 | 6.04 | 0.21 |
| 49306_at | 83937 | RASSF4 | AI890191 | 360.79 | 1084.81 | 499.14 | 0.46 | 360.79 | 888.19 | 700.77 | |
| 203708_at | 5142 | PDE4B | NM_002600 | 58.60 | 1193.32 | 549.36 | 0.46 | 58.60 | 1737.58 | 1227.97 | 0.71 |
| 222033_s_at | 2321 | FLT1 | AA058828 | 8.62 | 13.24 | 6.10 | 0.46 | 8.62 | 118.67 | 76.26 | 0.64 |
| 217227_x_at | 3576 | IL8 | X93006 | 27.81 | 51.38 | 23.68 | 0.46 | 27.81 | 12.34 | 5.71 | |
| 211118_x_at | 2100 | ESR2 | AF051428 | 10.30 | 23.55 | 10.87 | 0.46 | 10.30 | 17.60 | 12.40 | 0.70 |
| 215579_at | 60489 | APOBEC3G | AK022802 | 11.96 | 18.83 | 8.69 | 0.46 | 11.96 | 8.95 | 5.89 | |
| 220929_at | 26290 | GALNT8 | NM_017417 | 39.63 | 71.97 | 33.23 | 0.46 | 39.63 | 19.37 | 43.00 | |
| 206803_at | 5173 | PDYN | NM_024411 | 8.32 | 17.96 | 8.30 | 0.46 | 8.32 | 5.30 | 8.56 | |
| 206222_at | 8794 | TNFRSF10C | NM_003841 | 14.71 | 41.28 | 19.10 | 0.46 | 14.71 | 6.75 | 7.71 | |
| 203280_at | 9667 | SAFB2 | NM_014649 | 125.52 | 214.52 | 99.28 | 0.46 | 125.52 | 208.35 | 142.55 | 0.68 |
| 222326_at | 5142 | PDE4B | AW973834 | 18.87 | 176.42 | 81.76 | 0.46 | 18.87 | 279.14 | 119.85 | 0.43 |
| 205121_at | 6443 | SGCB | NM_000232 | 23.70 | 38.32 | 17.76 | 0.46 | 23.70 | 12.57 | 16.31 | |
| 209762_x_at | 3431 | SP110 | AA969194 | 468.74 | 833.76 | 386.51 | 0.46 | 468.74 | 765.75 | 249.85 | 0.33 |
| 207452_s_at | 53942 | CNTN5 | NM_014361 | 36.19 | 57.53 | 26.68 | 0.46 | 36.19 | 7.88 | 32.43 | |
| 214022_s_at | 8519 | IFITM1 | AA749101 | 874.90 | 1539.56 | 714.39 | 0.46 | 874.90 | 1337.05 | 635.82 | 0.48 |
| 206755_at | 1555 | CYP2B6 | NM_000767 | 4.36 | 19.11 | 8.87 | 0.46 | 4.36 | 4.21 | 14.45 | |
| 213661_at | 25891 | DKFZP586H2123 | AI671186 | 11.02 | 33.16 | 15.41 | 0.46 | 11.02 | 3.37 | 6.58 | |
| 215249_at | 6165 | RPL35A | AK021571 | 12.10 | 28.30 | 13.15 | 0.46 | 12.10 | 18.29 | 8.60 | 0.47 |
| 211219_s_at | 9355 | LHX2 | U11701 | 225.70 | 630.33 | 293.00 | 0.46 | 225.70 | 460.75 | 278.41 | 0.60 |
| 220533_at | | | NM_024853 | 10.11 | 28.88 | 13.44 | 0.47 | 10.11 | 32.85 | 40.91 | |
| 217199_s_at | 6773 | STAT2 | S81491 | 28.74 | 51.90 | 24.17 | 0.47 | 28.74 | 13.82 | 59.58 | |
| 221585_at | 27092 | CACNG4 | BC004504 | 5.44 | 11.27 | 5.25 | 0.47 | 5.44 | 3.30 | 9.45 | |
| 201387_s_at | 7345 | UCHL1 | NM_004181 | 5.97 | 16.79 | 7.85 | 0.47 | 5.97 | 7.00 | 17.94 | |
| 207449_s_at | 23275 | POFUT2 | NM_015227 | 52.68 | 88.49 | 41.37 | 0.47 | 52.68 | 40.11 | 51.73 | |
| 211140_s_at | 835 | CASP2 | AF314174 | 14.11 | 25.56 | 11.97 | 0.47 | 14.11 | 9.99 | 7.62 | |
| 210368_at | 8641 /// | PCDHGB4 | AB002325 | 73.20 | 135.23 | 63.35 | 0.47 | 73.20 | 50.61 | 35.91 | |
| | | /// | | | | | | | | | |
| | | PCDHGA8 | | | | | | | | | |
| 205775_at | 26240 | FAM50B | NM_012135 | 6.53 | 20.45 | 9.58 | 0.47 | 6.53 | 14.80 | 17.01 | |
| 216788_at | | | AK025564 | 13.55 | 40.07 | 18.78 | 0.47 | 13.55 | 6.93 | 14.29 | |
| 216144_at | 57485 | ATXN7L1 | AL137378 | 47.45 | 137.80 | 64.62 | 0.47 | 47.45 | 36.09 | 65.50 | |
| 222221_x_at | 10938 | EHD1 | AY007161 | 231.46 | 681.47 | 319.64 | 0.47 | 231.46 | 1957.26 | 1792.81 | |
| 215510_at | 2116 | ETV2 | AV693985 | 8.22 | 21.71 | 10.19 | 0.47 | 8.22 | 4.96 | 12.44 | |
| 208218_s_at | 91 | ACVR1B | NM_020328 | 13.13 | 43.94 | 20.62 | 0.47 | 13.13 | 15.63 | 21.04 | |
| 217228_s_at | 51666 | ASB4 | AC003079 | 7.17 | 23.45 | 11.01 | 0.47 | 7.17 | 8.87 | 25.70 | |
| 216056_at | 960 | CD44 | AW851559 | 11.06 | 40.70 | 19.11 | 0.47 | 11.06 | 171.10 | 238.29 | |
| 202800_at | 6507 | SLC1A3 | NM_004172 | 152.05 | 448.31 | 210.55 | 0.47 | 152.05 | 824.17 | 299.32 | 0.36 |
| 214443_at | 5817 | PVR | NM_006505 | 53.62 | 81.61 | 38.33 | 0.47 | 53.62 | 62.54 | 76.33 | |
| 216172_at | | | AK025114 | 36.25 | 123.25 | 57.94 | 0.47 | 36.25 | 61.07 | 22.09 | 0.36 |
| 210755_at | 3082 | HGF | U46010 | 16.65 | 34.61 | 16.28 | 0.47 | 16.65 | 8.40 | 4.12 | |
| 202908_at | 7466 | WFS1 | NM_006005 | 34.00 | 59.20 | 27.85 | 0.47 | 34.00 | 39.81 | 28.46 | |
| 214767_s_at | 126393 | HSPB6 | AL551046 | 24.47 | 44.62 | 20.99 | 0.47 | 24.47 | 27.92 | 15.75 | |
| 219386_s_at | 56833 | SLAMF8 | NM_020125 | 64.32 | 442.58 | 208.66 | 0.47 | 64.32 | 584.05 | 158.27 | 0.27 |
| 209037_s_at | 10938 | EHD1 | AW182860 | 139.26 | 404.70 | 190.85 | 0.47 | 139.26 | 1191.93 | 1352.43 | |
| 206671_at | 6295 | SAG | NM_000541 | 14.51 | 26.75 | 12.62 | 0.47 | 14.51 | 11.82 | 32.83 | |
| 215602_at | 221472 | FGD2 | AK024456 | 49.51 | 118.97 | 56.19 | 0.47 | 49.51 | 98.39 | 53.13 | 0.54 |
| 216514_at | | | AF203728 | 21.53 | 46.21 | 21.85 | 0.47 | 21.53 | 30.44 | 19.56 | |
| 202856_s_at | 9123 | SLC16A3 | NM_004207 | 1269.72 | 2800.24 | 1324.35 | 0.47 | 1269.72 | 7277.23 | 5376.38 | 0.74 |
| 220603_s_at | 55784 | MCTP2 | NM_018349 | 18.73 | 33.13 | 15.67 | 0.47 | 18.73 | 32.04 | 22.74 | 0.71 |
| 214637_at | 5008 | OSM | BG437034 | 4.16 | 10.86 | 5.14 | 0.47 | 4.16 | 5.28 | 5.03 | |
| 214425_at | 259 | AMBP | AV645756 | 32.90 | 51.66 | 24.45 | 0.47 | 32.90 | 16.61 | 23.54 | |
| 205606_at | 4040 | LRP6 | NM_002336 | 37.16 | 60.59 | 28.70 | 0.47 | 37.16 | 45.44 | 28.19 | |
| 219564_at | 3773 | KCNJ16 | AF153815 | 2.83 | 13.33 | 6.32 | 0.47 | 2.83 | 17.45 | 3.98 | 0.23 |
| 206478_at | 9834 | KIAA0125 | NM_014792 | 13.88 | 23.21 | 11.02 | 0.47 | 13.88 | 5.67 | 20.65 | |
| 215180_at | | | AL109703 | 16.61 | 25.31 | 12.02 | 0.47 | 16.61 | 9.66 | 11.54 | |
| 214996_at | | | AL079294 | 31.67 | 88.05 | 41.85 | 0.48 | 31.67 | 68.26 | 86.33 | |
| 220648_at | 105 | ADARB2 | NM_018702 | 7.46 | 19.12 | 9.09 | 0.48 | 7.46 | 11.36 | 8.22 | 0.72 |
| 210765_at | 1434 | CSEIL | AF053640 | 51.53 | 107.34 | 51.06 | 0.48 | 51.53 | 109.81 | 75.70 | 0.69 |
| 208100_x_at | 10500 | SEMA6C | NM_030913 | 6.34 | 11.80 | 5.61 | 0.48 | 6.34 | 6.15 | 6.80 | |
| 216415_at | 55567 | DNAH3 | AK026793 | 9.20 | 17.60 | 8.38 | 0.48 | 9.20 | 3.82 | 4.66 | |
| 214563_at | 5098 | PCDHGC3 | AF152524 | 29.47 | 49.54 | 23.62 | 0.48 | 29.47 | 14.72 | 47.38 | |
| 205524_s_at | 1404 | HAPLN1 | NM_001884 | 5.65 | 17.18 | 8.20 | 0.48 | 5.65 | 14.55 | 5.42 | 0.37 |
| 214605_x_at | 2825 | GPR1 | AL046992 | 32.48 | 80.34 | 38.37 | 0.48 | 32.48 | 79.88 | 48.37 | 0.61 |
| 220008_at | 79834 | KIAA2002 | NM_024776 | 7.69 | 36.58 | 17.47 | 0.48 | 7.69 | 46.42 | 28.53 | 0.61 |
| 218687_s_at | 56667 | MUC13 | AW451240 | 7.96 | 35.02 | 16.75 | 0.48 | 7.96 | 31.24 | 13.47 | 0.43 |
| 202855_s_at | 9123 | SLC16A3 | AL513917 | 673.54 | 1409.55 | 674.24 | 0.48 | 673.54 | 2626.14 | 2682.65 | |
| 217104_at | 400410 | LOC400410 | AL109714 | 109.68 | 166.79 | 79.83 | 0.48 | 109.68 | 190.54 | 158.92 | |
| 202485_s_at | 8932 | MBD2 | NM_003927 | 12.05 | 44.48 | 21.29 | 0.48 | 12.05 | 10.55 | 29.58 | |
| 201649_at | 9246 | UBE2L6 | NM_004223 | 567.27 | 926.03 | 443.91 | 0.48 | 567.27 | 477.43 | 372.63 | |
| 207327_at | 2070 | EYA4 | NM_004100 | 12.06 | 18.39 | 8.81 | 0.48 | 12.06 | 8.07 | 7.44 | |
| 219209_at | 64135 | IFIH1 | NM_022168 | 197.26 | 930.22 | 446.15 | 0.48 | 197.26 | 876.21 | 393.94 | 0.45 |
| 207826_s_at | 3399 | ID3 | NM_002167 | 3.33 | 11.06 | 5.31 | 0.48 | 3.33 | 9.21 | 41.75 | |
| 222252_x_at | 56893 | UBQLN4 | AK023354 | 104.84 | 283.27 | 135.97 | 0.48 | 104.84 | 142.02 | 111.97 | |
| AFFX- | | | AFFX- | 12.24 | 79.78 | 38.32 | 0.48 | 12.24 | 47.35 | 40.10 | |
| M27830_3_at | | | M27830_3 | | | | | | | | |
| 219102_at | 57333 | RCN3 | NM_020650 | 12.95 | 58.47 | 28.09 | 0.48 | 12.95 | 4.54 | 11.35 | |
| 215682_at | 440792 | LOC440792 | AB051440 | 10.12 | 93.67 | 45.00 | 0.48 | 10.12 | 21.62 | 16.61 | |
| 215590_x_at | | | AK025619 | 6.56 | 31.30 | 15.05 | 0.48 | 6.56 | 29.21 | 10.33 | 0.35 |
| 205088_at | 10046 | CXorf6 | NM_005491 | 6.61 | 10.84 | 5.22 | 0.48 | 6.61 | 486.96 | 572.08 | |
| 204907_s_at | 602 | BCL3 | AI829875 | 83.78 | 611.50 | 294.34 | 0.48 | 83.78 | 270.71 | 261.81 | |
| 206582_s_at | 9289 | GPR56 | NM_005682 | 3.92 | 16.45 | 7.92 | 0.48 | 3.92 | 8.58 | 7.70 | |
| 216911_s_at | 23119 | HIC2 | AL162003 | 9.93 | 16.44 | 7.92 | 0.48 | 9.93 | 4.52 | 2.50 | |
| 211053_at | 3755 | KCNG1 | BC006367 | 2.55 | 10.37 | 5.00 | 0.48 | 2.55 | 10.62 | 24.18 | |
| 211343_s_at | 1305 | COL13A1 | M33653 | 4.64 | 14.49 | 6.98 | 0.48 | 4.64 | 6.67 | 4.10 | |
| 215844_at | 30000 | TNPO2 | AK022217 | 12.77 | 22.44 | 10.82 | 0.48 | 12.77 | 31.15 | 6.82 | 0.22 |
| 206187_at | 5739 | PTGIR | NM_000960 | 93.00 | 330.43 | 159.70 | 0.48 | 93.00 | 273.60 | 159.75 | 0.58 |
| 53991_at | 27147 | DENND2A | AA127623 | 12.23 | 40.51 | 19.59 | 0.48 | 12.23 | 39.59 | 22.72 | 0.57 |
| 221661_at | 10864 | SLC22A7 | AF210455 | 5.72 | 19.06 | 9.24 | 0.48 | 5.72 | 12.38 | 7.52 | 0.61 |
| 205470_s_at | 11012 | KLK11 | NM_006853 | 16.49 | 61.83 | 29.99 | 0.49 | 16.49 | 32.70 | 22.39 | 0.68 |
| 217585_at | 51131 | PHF11 | BE502910 | 5.90 | 15.70 | 7.62 | 0.49 | 5.90 | 9.03 | 2.60 | |
| 205139_s_at | 10090 | UST | NM_005715 | 71.09 | 288.76 | 140.13 | 0.49 | 71.09 | 208.76 | 355.04 | |
| 207740_s_at | 23636 | NUP62 | NM_012346 | 646.35 | 1052.53 | 511.34 | 0.49 | 646.35 | 552.88 | 618.76 | |
| 205833_s_at | 25859 | PART1 | AI770098 | 4.56 | 11.18 | 5.44 | 0.49 | 4.56 | 15.27 | 8.63 | 0.56 |
| 201641_at | 684 | BST2 | NM_004335 | 955.76 | 1772.88 | 862.77 | 0.49 | 955.76 | 797.12 | 534.68 | |
| 213731_s_at | 6929 | TCF3 | AI871234 | 9.50 | 29.72 | 14.47 | 0.49 | 9.50 | 28.25 | 21.50 | |
| 222292_at | 958 | CD40 | AW298127 | 60.18 | 340.59 | 165.89 | 0.49 | 60.18 | 178.71 | 214.24 | |
| 209419_at | 8563 | THOC5 | AB023200 | 23.64 | 48.24 | 23.50 | 0.49 | 23.64 | 21.43 | 21.17 | |
| 64942_at | 387509 | GPR153 | AI937160 | 144.39 | 224.86 | 109.61 | 0.49 | 144.39 | 246.18 | 158.83 | 0.65 |
| 205632_s_at | 8395 | PIP5K1B | NM_003558 | 141.80 | 413.77 | 201.74 | 0.49 | 141.80 | 80.53 | 201.85 | |
| 209176_at | | | AI332962 | 20.90 | 60.56 | 29.55 | 0.49 | 20.90 | 62.90 | 32.92 | 0.52 |
| 217610_at | 246721 | POLR2J2 | AL047879 | 314.89 | 551.57 | 269.11 | 0.49 | 314.89 | 208.18 | 236.75 | |
| 220819_at | 79981 | FRMD1 | NM_024919 | 15.27 | 23.37 | 11.41 | 0.49 | 15.27 | 8.10 | 6.30 | |
| 207094_at | 3577 | IL8RA | NM_000634 | 11.49 | 17.56 | 8.58 | 0.49 | 11.49 | 4.01 | 15.00 | |
| 221671_x_at | 28299 | IGKC | M63438 | 27.09 | 348.99 | 170.52 | 0.49 | 27.09 | 113.73 | 22.17 | 0.19 |
| | // | /// | | | | | | | | | |
| | | IGKV1- | | | | | | | | | |
| | | 5 /// | | | | | | | | | |
| | | IGKV2- | | | | | | | | | |
| | | 24 | | | | | | | | | |
| 217133_x_at | 1555 | CYP2B6 | X06399 | 18.10 | 48.25 | 23.59 | 0.49 | 18.10 | 19.72 | 20.60 | |
| 206570_s_at | 440533 / | PSG1 | NM_002785 | 11.88 | 19.06 | 9.32 | 0.49 | 11.88 | 9.01 | 7.73 | |
| | | /// | | | | | | | | | |
| | | PSG4 | | | | | | | | | |
| | | /// | | | | | | | | | |
| | | PSG5 | | | | | | | | | |
| | | /// PSG | | | | | | | | | |
| 217657_at | 55973 | BCAP29 | AL583687 | 56.02 | 93.16 | 45.56 | 0.49 | 56.02 | 157.68 | 81.35 | 0.52 |
| 206901_at | 79173 | C19orf57 | NM_024323 | 11.67 | 21.06 | 10.30 | 0.49 | 11.67 | 12.18 | 8.56 | |
| 207324_s_at | 1823 | DSC1 | NM_004948 | 6.65 | 10.95 | 5.36 | 0.49 | 6.65 | 3.38 | 8.05 | |
| 214135_at | 51208 | CLDN18 | BE551219 | 23.44 | 38.38 | 18.78 | 0.49 | 23.44 | 39.81 | 16.77 | 0.42 |
| 210294_at | 6892 | TAPBP | AF067286 | 80.57 | 303.94 | 148.78 | 0.49 | 80.57 | 150.09 | 149.13 | |
| 216535_at | 57863 | IGSF4B | AL050219 | 8.07 | 22.31 | 10.93 | 0.49 | 8.07 | 14.44 | 6.88 | 0.48 |
| 217003_s_at | 255926 | TMDCII | AJ132823 | 7.30 | 44.10 | 21.62 | 0.49 | 7.30 | 21.59 | 19.46 | |
| 215575_at | 9659 | PDE4DIP | AU157078 | 2.77 | 18.79 | 9.22 | 0.49 | 2.77 | 15.56 | 5.20 | 0.33 |
| 210690_at | 8302 | KLRC4 | U96845 | 3.22 | 12.34 | 6.05 | 0.49 | 3.22 | 3.38 | 2.30 | |
| 221171_at | 728621 | RP4- | NM_025030 | 45.14 | 94.75 | 46.50 | 0.49 | 45.14 | 28.58 | 53.10 | |
| | | 692D31 | | | | | | | | | |
| 221723_s_at | 57835 | SLC4A5 | AF243499 | 19.17 | 38.68 | 18.99 | 0.49 | 19.17 | 31.53 | 31.28 | |
| 202307_s_at | 6890 | TAP1 | NM_000593 | 549.39 | 2237.13 | 1098.51 | 0.49 | 549.39 | 965.28 | 591.09 | 0.61 |
| 220271_x_at | 64800 | FLJ23588 | NM_022785 | 4.22 | 11.13 | 5.47 | 0.49 | 4.22 | 6.84 | 6.38 | |
| 208580_x_at | 8362 /// | HIST1H4K | NM_021968 | 26.88 | 73.75 | 36.27 | 0.49 | 26.88 | 23.42 | 12.78 | |
| | | /// | | | | | | | | | |
| | | HIST1H4J | | | | | | | | | |
| 213051_at | 56829 | ZC3HAV1 | AI133727 | 1144.26 | 2471.16 | 1215.72 | 0.49 | 1144.26 | 2873.27 | 1269.58 | 0.44 |
| 204677_at | 1003 | CDH5 | NM_001795 | 2.77 | 24.82 | 12.23 | 0.49 | 2.77 | 14.30 | 13.89 | |
| 208060_at | 5081 | PAX7 | NM_002584 | 19.66 | 30.54 | 15.05 | 0.49 | 19.66 | 12.61 | 28.66 | |
| 220213_at | 128553 | TSHZ2 | NM_018692 | 3.84 | 11.41 | 5.62 | 0.49 | 3.84 | 6.28 | 3.43 | |
| 206675_s_at | 6498 | SKIL | NM_005414 | 7.55 | 68.20 | 33.65 | 0.49 | 7.55 | 88.44 | 197.32 | |
| 206503_x_at | 5371 | PML | NM_002675 | 102.22 | 171.85 | 84.92 | 0.49 | 102.22 | 161.85 | 67.69 | 0.42 |
| 217664_at | 23030 | JMJD2B | AA780524 | 5.04 | 20.00 | 9.88 | 0.49 | 5.04 | 7.97 | 28.25 | |
| 210226_at | 3164 | NR4A1 | D85245 | 23.01 | 56.41 | 27.91 | 0.49 | 23.01 | 55.58 | 21.31 | 0.38 |
| 212977_at | 57007 | CXCR7 | AI817041 | 31.39 | 87.50 | 43.31 | 0.49 | 31.39 | 88.25 | 56.13 | 0.64 |
| 216002_at | 2342 | FNTB | AU147200 | 26.61 | 46.15 | 22.88 | 0.50 | 26.61 | 14.33 | 20.52 | |
| 211731_x_at | 10214 | SSX3 | BC005904 | 23.89 | 40.63 | 20.15 | 0.50 | 23.89 | 45.09 | 26.86 | 0.60 |
| 219914_at | 9427 | ECEL1 | NM_004826 | 5.82 | 35.17 | 17.45 | 0.50 | 5.82 | 19.43 | 11.41 | 0.59 |
| 40560_at | 6909 | TBX2 | U28049 | 9.36 | 23.42 | 11.65 | 0.50 | 9.36 | 65.63 | 55.42 | |
| 201269_s_at | 23386 | NUDCD3 | AB028991 | 23.91 | 50.99 | 25.38 | 0.50 | 23.91 | 20.30 | 23.97 | |
| 209266_s_at | 64116 | SLC39A8 | AW134794 | 21.35 | 35.36 | 17.61 | 0.50 | 21.35 | 10.27 | 24.45 | |
| 217515_s_at | 779 | CACNA1S | AI391509 | 7.48 | 11.24 | 5.60 | 0.50 | 7.48 | 17.00 | 16.99 | |
| 221377_s_at | 11317 | RBPSUHL | NM_014276 | 7.14 | 13.83 | 6.89 | 0.50 | 7.14 | 3.75 | 11.79 | |
| 218223_s_at | 51177 | PLEKHO1 | NM_016274 | 393.57 | 1389.26 | 692.14 | 0.50 | 393.57 | 1535.10 | 1010.63 | 0.66 |
| 210683_at | 4902 | NRTN | AL161995 | 15.78 | 48.19 | 24.02 | 0.50 | 15.78 | 32.18 | 33.72 | |
| 220656_at | | | NM_018527 | 1.01 | 24.02 | 11.98 | 0.50 | 1.01 | 2.47 | 6.09 | |
| 218764_at | 5583 | PRKCH | NM_024064 | 144.61 | 308.76 | 154.17 | 0.50 | 144.61 | 184.83 | 270.13 | |
| 216248_s_at | 4929 | NR4A2 | S77154 | 9.04 | 90.84 | 45.36 | 0.50 | 9.04 | 81.81 | 98.06 | |
| 221913_at | 23410 | SIRT3 | AI492888 | 48.04 | 91.84 | 45.86 | 0.50 | 48.04 | 55.01 | 43.95 | |
| 216848_at | 85374 | KIAA1660 | AB051447 | 6.70 | 10.32 | 5.16 | 0.50 | 6.70 | 4.77 | 3.20 | |
| 206955_at | 364 | AQP7 | NM_001170 | 21.77 | 57.17 | 28.56 | 0.50 | 21.77 | 22.61 | 9.49 | |
| 211866_x_at | 3077 | HFE | AF079409 | 14.49 | 32.13 | 16.07 | 0.50 | 14.49 | 13.20 | 11.87 | |
| 209897_s_at | 9353 | SLIT2 | AF055585 | 14.59 | 45.64 | 22.83 | 0.50 | 14.59 | 66.77 | 52.92 | |
| 205926_at | 9466 | IL27RA | NM_004843 | 164.31 | 324.13 | 162.14 | 0.50 | 164.31 | 876.65 | 395.74 | 0.45 |
| 216416_at | 50807 | DDEF1 | AK027254 | 18.77 | 51.92 | 25.98 | 0.50 | 18.77 | 10.32 | 37.77 | |
| 209305_s_at | 4616 | GADD45B | AF078077 | 200.36 | 512.89 | 256.94 | 0.50 | 200.36 | 352.50 | 879.98 | |
| 212937_s_at | 1291 | COL6A1 | M20776 | 69.99 | 126.27 | 63.26 | 0.50 | 69.99 | 148.85 | 131.90 | |
| 206720_at | 4249 | MGAT5 | NM_002410 | 4.55 | 11.00 | 5.52 | 0.50 | 4.55 | 3.84 | 8.44 | |
| 219694_at | 54491 | FAM105A | NM_019018 | 494.90 | 775.91 | 389.17 | 0.50 | 494.90 | 307.64 | 58.14 | |
| 211660_at | 5452 | POU2F2 | M36653 | 72.75 | 128.97 | 64.72 | 0.50 | 72.75 | 98.45 | 141.99 | |
| 211661_x_at | 5724 | PTAFR | M80436 | 78.00 | 199.45 | 100.13 | 0.50 | 78.00 | 173.60 | 141.87 | |
| 215700_x_at | 9362 | CPNE6 | AL050397 | 4.79 | 13.01 | 6.53 | 0.50 | 4.79 | 6.10 | 5.02 | |
| 222240_s_at | 51477 | ISYNA1 | AL137749 | 18.57 | 36.01 | 18.08 | 0.50 | 18.57 | 24.49 | 38.29 | |
| 213458_at | 317662 | KIAA0974 | AB023191 | 0.67 | 20.68 | 10.39 | 0.50 | 0.67 | 7.58 | 1.14 | |
| 215916_at | 1145 | CHRNE | AL157418 | 8.48 | 28.07 | 14.10 | 0.50 | 8.48 | 21.36 | 14.47 | 0.68 |
| 216173_at | | | AK025360 | 46.10 | 136.69 | 68.68 | 0.50 | 46.10 | 62.41 | 94.51 | |
| 222141_at | 84861 | KLHL22 | AK024369 | 44.89 | 75.72 | 38.05 | 0.50 | 44.89 | 31.34 | 31.06 | |
| 205127_at | 5742 | PTGS1 | NM_000962 | 17.10 | 61.50 | 30.91 | 0.50 | 17.10 | 56.42 | 64.33 | |
| 202446_s_at | 5359 | PLSCR1 | AI825926 | 1441.49 | 2826.59 | 1420.85 | 0.50 | 1441.49 | 2777.81 | 1350.95 | 0.49 |
| 208301_at | | | NM_025033 | 41.19 | 70.16 | 35.29 | 0.50 | 41.19 | 80.98 | 101.32 | |
| 206341_at | 3559 | IL2RA | NM_000417 | 10.70 | 44.28 | 22.28 | 0.50 | 10.70 | 18.41 | 9.94 | 0.54 |
| 214613_at | 2827 | GPR3 | AW024085 | 5.41 | 36.38 | 18.32 | 0.50 | 5.41 | 19.67 | 38.12 | |
| 210890_x_at | 3802 | KIR2DL1 | U24078 | 4.51 | 12.15 | 6.12 | 0.50 | 4.51 | 2.79 | 3.99 | |
| 208569_at | 8335 | HIST1H2AB | NM_003513 | 46.33 | 91.91 | 46.28 | 0.50 | 46.33 | 62.17 | 40.51 | |
| 216349_at | 341651 | LOC341651 | AL136527 | 57.66 | 88.59 | 44.61 | 0.50 | 57.66 | 61.40 | 56.83 | |
| 209811_at | 835 | CASP2 | BC002427 | 81.74 | 126.69 | 63.81 | 0.50 | 81.74 | 68.43 | 29.30 | |
| 201422_at | 10437 | IFI30 | NM_006332 | 224.38 | 359.58 | 181.11 | 0.50 | 224.38 | 321.71 | 226.05 | |
| 216502_at | 81875 | ISG20L2 | AL096734 | 7.82 | 12.41 | 6.26 | 0.50 | 7.82 | 8.05 | 5.95 | |
| 215866_at | 1937 | EEF1G | AK024938 | 71.63 | 171.60 | 86.52 | 0.50 | 71.63 | 129.49 | 128.65 | |
| 210017_at | 10892 | MALT1 | AF070528 | 71.24 | 164.15 | 82.79 | 0.50 | 71.24 | 267.72 | 123.84 | 0.46 |
| 205377_s_at | 43 | ACHE | AI190022 | 5.55 | 50.30 | 25.37 | 0.50 | 5.55 | 26.45 | 8.07 | 0.31 |
| 1438_at | 2049 | EPHB3 | X75208 | 54.66 | 82.35 | 41.57 | 0.50 | 54.66 | 27.75 | 24.53 | |
| 60815_at | 84820 | MGC13098 | AA601208 | 170.03 | 284.91 | 143.82 | 0.50 | 170.03 | 129.40 | 111.24 | |
| 202638_s_at | 3383 | ICAM1 | NM_000201 | 41.96 | 1353.52 | 683.82 | 0.51 | 41.96 | 1508.23 | 1074.85 | 0.71 |
| 207538_at | 3565 | IL4 | NM_000589 | 7.37 | 24.80 | 12.53 | 0.51 | 7.37 | 14.37 | 14.07 | |
| 214354_x_at | 6439 | SFTPB | T91506 | 11.95 | 60.19 | 30.41 | 0.51 | 11.95 | 18.06 | 31.76 | |
| 214557_at | 10744 | PTTG2 | NM_006607 | 8.47 | 43.99 | 22.23 | 0.51 | 8.47 | 38.59 | 10.14 | 0.26 |
| 204927_at | 8045 | RASSF7 | NM_003475 | 223.03 | 352.66 | 178.34 | 0.51 | 223.03 | 170.41 | 130.03 | |
| 211712_s_at | 8416 | ANXA9 | BC005830 | 4.10 | 14.32 | 7.25 | 0.51 | 4.10 | 36.45 | 19.06 | 0.52 |
| 204879_at | 10630 | PDPN | NM_006474 | 43.25 | 127.99 | 64.79 | 0.51 | 43.25 | 24.06 | 50.10 | |
| 49049_at | 196403 | DTX3 | N92708 | 10.96 | 24.73 | 12.52 | 0.51 | 10.96 | 5.77 | 4.24 | |
| 206223_at | 22853 | LMTK2 | NM_014916 | 33.05 | 75.91 | 38.45 | 0.51 | 33.05 | 61.76 | 52.48 | |
| 207334_s_at | 7048 | TGFBR2 | NM_003242 | 36.11 | 66.79 | 33.83 | 0.51 | 36.11 | 13.04 | 40.21 | |
| 207447_s_at | 25834 | MGAT4C | NM_013244 | 2.15 | 12.94 | 6.56 | 0.51 | 2.15 | 23.63 | 15.80 | 0.67 |
| 216370_s_at | 8277 | TKTL1 | Z49258 | 5.13 | 10.24 | 5.19 | 0.51 | 5.13 | 6.95 | 4.77 | |
| 206125_s_at | 11202 | KLK8 | NM_007196 | 6.98 | 23.67 | 12.01 | 0.51 | 6.98 | 16.02 | 11.44 | 0.71 |
| 219367_s_at | 8828 | NRP2 | NM_018534 | 12.04 | 38.01 | 19.30 | 0.51 | 12.04 | 375.70 | 261.71 | 0.70 |
| 214185_at | 10657 | KHDRBS1 | AW592227 | 34.55 | 68.05 | 34.56 | 0.51 | 34.55 | 82.11 | 67.59 | |
| 207050_at | 781 | CACNA2D1 | NM_000722 | 6.33 | 14.14 | 7.18 | 0.51 | 6.33 | 8.90 | 9.45 | |
| 210618_at | 5909 | RAP1GAP | AB007943 | 21.45 | 38.37 | 19.53 | 0.51 | 21.45 | 9.35 | 10.69 | |
| 206970_at | 6900 | CNTN2 | NM_005076 | 6.76 | 16.08 | 8.20 | 0.51 | 6.76 | 13.94 | 6.67 | 0.48 |
| 206193_s_at | 1041 | CDSN | NM_001264 | 8.09 | 19.62 | 10.00 | 0.51 | 8.09 | 3.99 | 12.05 | |
| 207779_at | | | NM_016344 | 28.62 | 95.09 | 48.52 | 0.51 | 28.62 | 15.36 | 37.54 | |
| 217142_at | 442215 | LOC442215 | AL035687 | 55.76 | 158.82 | 81.04 | 0.51 | 55.76 | 72.88 | 145.15 | |
| 220037_s_at | 10894 | XLKD1 | NM_016164 | 6.56 | 12.69 | 6.48 | 0.51 | 6.56 | 4.51 | 11.84 | |
| 216815_at | | | AL136306 | 4.70 | 26.52 | 13.54 | 0.51 | 4.70 | 2.68 | 5.74 | |
| 210799_at | 3351 | HTR1B | M81590 | 16.73 | 88.77 | 45.33 | 0.51 | 16.73 | 20.33 | 12.85 | |
| 219579_at | 5866 | RAB31L1 | NM_013401 | 104.83 | 163.56 | 83.53 | 0.51 | 104.83 | 47.48 | 56.29 | |
| 218543_s_at | 64761 | PARP12 | NM_022750 | 409.83 | 726.14 | 370.96 | 0.51 | 409.83 | 616.31 | 193.02 | 0.31 |
| 219257_s_at | 8877 | SPHK1 | NM_021972 | 82.73 | 145.13 | 74.17 | 0.51 | 82.73 | 358.67 | 603.80 | |
| 222219_s_at | 79816 | TLE6 | AC007766 | 7.19 | 25.99 | 13.29 | 0.51 | 7.19 | 4.94 | 21.03 | |
| 220210_at | 57053 | CHRNA10 | NM_020402 | 28.16 | 61.66 | 31.56 | 0.51 | 28.16 | 40.00 | 58.84 | |
| 209012_at | 7204 | TRIO | AV718192 | 47.49 | 80.66 | 41.33 | 0.51 | 47.49 | 29.63 | 36.45 | |
| 214489_at | 2488 | FSHB | NM_000510 | 6.22 | 12.60 | 6.47 | 0.51 | 6.22 | 22.70 | 31.42 | |
| 218015_s_at | 56897 | WRNIP1 | NM_020135 | 6.63 | 34.34 | 17.64 | 0.51 | 6.63 | 9.00 | 9.76 | |
| 205549_at | 5121 | PCP4 | NM_006198 | 9.85 | 25.52 | 13.11 | 0.51 | 9.85 | 4.40 | 4.32 | |
| 216062_at | 960 | CD44 | AW851559 | 10.10 | 41.48 | 21.34 | 0.51 | 10.10 | 34.89 | 36.40 | |
| 215922_at | 85021 | REPS1 | AL049259 | 39.46 | 66.97 | 34.47 | 0.51 | 39.46 | 30.48 | 23.61 | |
| 219562_at | 25837 | RAB26 | NM_014353 | 13.65 | 20.86 | 10.74 | 0.51 | 13.65 | 22.29 | 25.58 | |
| 206033_s_at | 1825 | DSC3 | NM_001941 | 105.27 | 256.50 | 132.22 | 0.52 | 105.27 | 117.25 | 162.66 | |
| 217476_at | 7067 /// | THRA | M24900 | 7.74 | 12.50 | 6.44 | 0.52 | 7.74 | 6.69 | 14.37 | |
| | | /// | | | | | | | | | |
| | | NR1D1 | | | | | | | | | |
| 216123_x_at | 54879 | ST7L | AK024158 | 42.11 | 68.45 | 35.30 | 0.52 | 42.11 | 35.28 | 18.38 | |
| 214689_at | 60676 | PAPPA2 | BF435151 | 23.05 | 50.15 | 25.88 | 0.52 | 23.05 | 25.00 | 9.44 | |
| 214619_at | 1394 | CRHR1 | X72304 | 11.85 | 37.23 | 19.21 | 0.52 | 11.85 | 40.54 | 41.73 | |
| 220168_at | 55259 | CASC1 | NM_018272 | 3.79 | 11.99 | 6.20 | 0.52 | 3.79 | 8.01 | 13.79 | |
| 207354_at | 6360 | CCL16 | NM_004590 | 4.37 | 15.99 | 8.26 | 0.52 | 4.37 | 5.71 | 12.10 | |
| 216465_at | | | AL110206 | 5.22 | 17.20 | 8.91 | 0.52 | 5.22 | 5.84 | 11.73 | |
| 215939_at | | | AU148005 | 7.97 | 22.86 | 11.85 | 0.52 | 7.97 | 2.85 | 12.83 | |
| 207296_at | 79175 | ZNF343 | NM_024325 | 6.98 | 10.70 | 5.54 | 0.52 | 6.98 | 5.91 | 7.30 | |
| 211203_s_at | 1272 | CNTN1 | U07820 | 16.70 | 34.79 | 18.04 | 0.52 | 16.70 | 28.76 | 18.01 | 0.63 |
| 211873_s_at | 56107 | PCDHGA9 | AF152516 | 4.75 | 18.31 | 9.50 | 0.52 | 4.75 | 40.37 | 10.33 | 0.26 |
| 210355_at | 5744 | PTHLH | J03580 | 4.74 | 14.99 | 7.78 | 0.52 | 4.74 | 17.18 | 6.60 | 0.38 |
| 216773_at | 150684 | COMMD1 | AK025191 | 12.54 | 44.24 | 22.97 | 0.52 | 12.54 | 23.57 | 25.61 | |
| 208485_x_at | 8837 | CFLAR | NM_003879 | 397.61 | 1185.98 | 615.89 | 0.52 | 397.61 | 1197.52 | 1550.82 | |
| 201564_s_at | 6624 | FSCN1 | NM_003088 | 1183.38 | 3054.04 | 1586.08 | 0.52 | 1183.38 | 1959.00 | 1655.62 | |
| 207113_s_at | 7124 | TNF | NM_000594 | 524.28 | 3499.34 | 1817.81 | 0.52 | 524.28 | 1597.18 | 2742.44 | |
| 210133_at | 6356 | CCL11 | D49372 | 9.12 | 23.90 | 12.42 | 0.52 | 9.12 | 27.60 | 22.50 | |
| 213329_at | 23380 | SRGAP2 | AA742261 | 323.54 | 543.95 | 282.78 | 0.52 | 323.54 | 260.56 | 197.79 | |
| 212939_at | 1291 | COL6A1 | M20776 | 3.32 | 12.09 | 6.31 | 0.52 | 3.32 | 9.49 | 17.66 | |
| 208476_s_at | 55691 | FRMD4A | NM_018027 | 37.99 | 69.80 | 36.42 | 0.52 | 37.99 | 9.43 | 6.09 | |
| 211786_at | 3604 | TNFRSF9 | BC006196 | 4.45 | 65.67 | 34.29 | 0.52 | 4.45 | 191.03 | 111.10 | 0.58 |
| 220337_at | 58157 | NGB | NM_021257 | 7.98 | 17.97 | 9.39 | 0.52 | 7.98 | 8.13 | 23.16 | |
| 214289_at | 5689 | PSMB1 | W86293 | 18.85 | 37.45 | 19.57 | 0.52 | 18.85 | 49.41 | 67.72 | |
| 217290_at | | | AL030995 | 6.61 | 12.03 | 6.29 | 0.52 | 6.61 | 7.72 | 4.60 | |
| 209138_x_at | 3535 | IGL@ | M87790 | 41.80 | 71.68 | 37.53 | 0.52 | 41.80 | 59.67 | 28.54 | |
| 201631_s_at | 8870 | IER3 | NM_003897 | 24.79 | 230.83 | 120.90 | 0.52 | 24.79 | 847.54 | 1453.11 | |
| 216365_x_at | 3535 /// | IGL@ | AF047245 | 2.92 | 10.45 | 5.48 | 0.52 | 2.92 | 6.15 | 10.17 | |
| | | /// | | | | | | | | | |
| | | CPVL | | | | | | | | | |
| 219352_at | 55008 | HERC6 | NM_017912 | 174.89 | 298.58 | 156.53 | 0.52 | 174.89 | 356.45 | 121.02 | 0.34 |
| 211817_s_at | 3762 | KCNJ5 | L47208 | 8.40 | 36.77 | 19.28 | 0.52 | 8.40 | 36.80 | 3.87 | 0.11 |
| 216785_at | 2281 | FKBP1B | AK026273 | 5.02 | 15.17 | 7.96 | 0.52 | 5.02 | 2.52 | 16.17 | |
| 209699_x_at | 1646 | AKR1C2 | U05598 | 8.15 | 41.79 | 21.92 | 0.52 | 8.15 | 76.63 | 203.90 | |
| 208047_s_at | 4664 | NAB1 | NM_005966 | 90.73 | 196.23 | 103.05 | 0.53 | 90.73 | 105.29 | 366.13 | |
| 207169_x_at | 780 | DDR1 | NM_001954 | 3.04 | 11.53 | 6.06 | 0.53 | 3.04 | 15.09 | 13.53 | |
| 47553_at | 25861 | DFNB31 | AA813332 | 7.91 | 69.84 | 36.70 | 0.53 | 7.91 | 61.97 | 68.52 | |
| 216544_at | 159162 | RBMY2FP | AC007320 | 7.25 | 18.87 | 9.92 | 0.53 | 7.25 | 12.31 | 8.38 | 0.68 |
| 221376_at | 8822 | FGF17 | NM_003867 | 19.98 | 48.53 | 25.52 | 0.53 | 19.98 | 52.78 | 27.56 | 0.52 |
| 216774_at | | | AK025325 | 32.59 | 50.41 | 26.51 | 0.53 | 32.59 | 34.02 | 29.30 | |
| 216157_at | | | AK024231 | 4.73 | 33.34 | 17.54 | 0.53 | 4.73 | 9.95 | 10.40 | |
| 213299_at | 51341 | ZBTB7 | AW027070 | 70.22 | 108.67 | 57.16 | 0.53 | 70.22 | 40.69 | 32.44 | |
| 219117_s_at | 51303 | FKBP11 | NM_016594 | 225.15 | 376.30 | 197.96 | 0.53 | 225.15 | 248.89 | 180.36 | |
| 219432_at | 2121 | EVC | NM_014556 | 10.39 | 22.76 | 11.97 | 0.53 | 10.39 | 4.02 | 7.30 | |
| 209039_x_at | 10938 | EHD1 | AF001434 | 331.25 | 1308.87 | 689.60 | 0.53 | 331.25 | 3816.89 | 2980.02 | |
| 206459_s_at | 7482 | WNT2B | NM_024494 | 9.02 | 34.17 | 18.02 | 0.53 | 9.02 | 6.66 | 17.72 | |
| 201473_at | 3726 | JUNB | NM_002229 | 220.42 | 845.97 | 446.37 | 0.53 | 220.42 | 502.08 | 765.28 | |
| 216187_x_at | 3831 | KNS2 | AF222691 | 1842.32 | 4024.28 | 2124.02 | 0.53 | 1842.32 | 2025.86 | 2088.13 | |
| 202995_s_at | 2192 | FBLN1 | NM_006486 | 45.62 | 83.27 | 43.98 | 0.53 | 45.62 | 19.19 | 51.05 | |
| 208906_at | 221092 / | BSCL2 | BC004911 | 234.80 | 397.20 | 209.88 | 0.53 | 234.80 | 321.30 | 214.35 | |
| | | /// | | | | | | | | | |
| | | HNRPUL2 | | | | | | | | | |
| 35147_at | 23263 | MCF2L | AB002360 | 6.16 | 20.79 | 10.99 | 0.53 | 6.16 | 13.67 | 7.39 | 0.54 |
| 211924_s_at | 5329 | PLAUR | AY029180 | 381.08 | 1022.12 | 540.21 | 0.53 | 381.08 | 1224.36 | 1708.63 | |
| 210222_s_at | 6252 | RTN1 | BC000314 | 18.29 | 38.69 | 20.47 | 0.53 | 18.29 | 39.32 | 25.70 | 0.65 |
| 220958_at | 54986 | ULK4 | NM_017886 | 9.09 | 15.24 | 8.07 | 0.53 | 9.09 | 4.03 | 9.08 | |
| 220167_s_at | 24150 | TP53TG3 | NM_015369 | 23.33 | 48.72 | 25.83 | 0.53 | 23.33 | 29.66 | 36.24 | |
| | // | /// | | | | | | | | | |
| | | LOC729264 | | | | | | | | | |
| | | /// | | | | | | | | | |
| | | LOC7 | | | | | | | | | |
| 219863_at | 51191 | HERC5 | NM_016323 | 101.11 | 168.99 | 89.63 | 0.53 | 101.11 | 136.43 | 81.59 | |
| 202254_at | 26037 | SIPA1L1 | AB007900 | 11.87 | 34.70 | 18.42 | 0.53 | 11.87 | 57.47 | 23.61 | 0.41 |
| 219993_at | 64321 | SOX17 | NM_022454 | 8.76 | 35.15 | 18.67 | 0.53 | 8.76 | 20.92 | 30.74 | |
| 207547_s_at | 11170 | FAM107A | NM_007177 | 11.27 | 27.99 | 14.87 | 0.53 | 11.27 | 45.91 | 34.03 | 0.74 |
| 202914_s_at | 9826 | ARHGEF11 | NM_014784 | 118.53 | 206.46 | 109.77 | 0.53 | 118.53 | 159.19 | 93.28 | |
| 219992_at | 6866 | TAC3 | NM_013251 | 72.37 | 128.12 | 68.17 | 0.53 | 72.37 | 150.39 | 101.60 | 0.68 |
| 210845_s_at | 5329 | PLAUR | U08839 | 649.50 | 1163.57 | 619.52 | 0.53 | 649.50 | 1604.51 | 1884.17 | |
| 219196_at | 29106 | SCG3 | NM_013243 | 3.94 | 15.34 | 8.17 | 0.53 | 3.94 | 18.72 | 2.40 | 0.13 |
| 219410_at | 55076 | TMEM45A | NM_018004 | 442.15 | 965.75 | 514.69 | 0.53 | 442.15 | 1335.48 | 1180.69 | |
| 217557_s_at | | | AV710357 | 38.92 | 76.27 | 40.65 | 0.53 | 38.92 | 137.55 | 37.00 | 0.27 |
| 216167_at | 10446 | LRRN5 | AK024867 | 2.86 | 13.71 | 7.31 | 0.53 | 2.86 | 8.82 | 2.15 | |
| 205288_at | 8556 | CDC14A | NM_003672 | 29.31 | 82.96 | 44.31 | 0.53 | 29.31 | 58.73 | 56.67 | |
| 213678_at | 441151 | C6orf137 | F09448 | 28.04 | 42.44 | 22.67 | 0.53 | 28.04 | 26.23 | 28.17 | |
| 218686_s_at | 64285 | RHBDF1 | NM_022450 | 170.51 | 261.98 | 139.95 | 0.53 | 170.51 | 203.45 | 155.07 | |
| 209119_x_at | 7026 | NR2F2 | AV703465 | 3.53 | 25.01 | 13.36 | 0.53 | 3.53 | 59.63 | 34.63 | 0.58 |
| 117_at | 3310 | HSPA6 | X51757 | 48.01 | 100.16 | 53.54 | 0.53 | 48.01 | 54.27 | 67.74 | |
| 208223_s_at | 91 | ACVR1B | NM_020327 | 28.94 | 56.75 | 30.34 | 0.53 | 28.94 | 35.26 | 17.15 | |
| 221651_x_at | 28299 | IGKC | BC005332 | 56.33 | 325.62 | 174.17 | 0.53 | 56.33 | 131.91 | 31.89 | 0.24 |
| | // | /// | | | | | | | | | |
| | | IGKV1- | | | | | | | | | |
| | | 5 /// | | | | | | | | | |
| | | IGKV2- | | | | | | | | | |
| | | 24 | | | | | | | | | |
| 208013_s_at | 56 | ACRV1 | NM_020115 | 37.63 | 59.59 | 31.88 | 0.53 | 37.63 | 27.80 | 14.74 | |
| 214891_at | 23014 | FBXO21 | U79257 | 8.02 | 14.28 | 7.65 | 0.54 | 8.02 | 7.88 | 4.75 | |
| 214417_s_at | 26998 | FETUB | N39010 | 21.05 | 48.29 | 25.86 | 0.54 | 21.05 | 24.61 | 42.55 | |
| 205080_at | 5915 | RARB | NM_000965 | 31.70 | 70.57 | 37.84 | 0.54 | 31.70 | 15.93 | 26.32 | |
| 221011_s_at | 81606 | LBH | NM_030915 | 75.88 | 125.33 | 67.22 | 0.54 | 75.88 | 80.47 | 125.37 | |
| 221902_at | 387509 | GPR153 | AL567940 | 51.24 | 78.75 | 42.24 | 0.54 | 51.24 | 111.54 | 64.70 | 0.58 |
| 206841_at | 5149 | PDE6H | NM_006205 | 7.26 | 25.62 | 13.75 | 0.54 | 7.26 | 31.56 | 21.70 | 0.69 |
| 217600_at | 222663 | SCUBE3 | BF511678 | 32.40 | 55.52 | 29.86 | 0.54 | 32.40 | 29.49 | 21.40 | |
| 203904_x_at | 3732 | CD82 | NM_002231 | 190.04 | 419.57 | 225.84 | 0.54 | 190.04 | 1212.74 | 673.84 | 0.56 |
| 213172_at | 23508 | TTC9 | AW235608 | 2.78 | 10.65 | 5.74 | 0.54 | 2.78 | 5.88 | 3.78 | |
| 210933_s_at | 6624 | FSCN1 | BC004908 | 1177.05 | 2869.97 | 1548.32 | 0.54 | 1177.05 | 1368.41 | 1629.56 | |
| 217650_x_at | 6483 /// | ST3GAL2 | AI088162 | 164.40 | 550.85 | 297.22 | 0.54 | 164.40 | 671.65 | 399.21 | 0.59 |
| | | /// | | | | | | | | | |
| | | LOC729518 | | | | | | | | | |
| 216315_x_at | 387522 / | UBE2V1 | AL121873 | 49.77 | 89.94 | 48.58 | 0.54 | 49.77 | 83.11 | 60.79 | 0.73 |
| | | /// | | | | | | | | | |
| | | Kua- | | | | | | | | | |
| | | UEV /// | | | | | | | | | |
| | | LOC7300 | | | | | | | | | |
| 210705_s_at | 85363 | TRIM5 | AF220028 | 80.81 | 147.13 | 79.55 | 0.54 | 80.81 | 71.29 | 32.48 | |
| 213607_x_at | 65220 | NADK | BE551347 | 274.21 | 424.56 | 229.72 | 0.54 | 274.21 | 137.52 | 193.88 | |
| 218675_at | 51310 | SLC22A17 | NM_020372 | 10.21 | 17.21 | 9.33 | 0.54 | 10.21 | 39.31 | 3.90 | 0.10 |
| 211965_at | 677 | ZFP36L1 | BE620915 | 56.38 | 95.87 | 51.99 | 0.54 | 56.38 | 65.97 | 62.81 | |
| 215391_at | 4130 | MAP1A | AA633627 | 7.41 | 15.36 | 8.33 | 0.54 | 7.41 | 19.80 | 10.42 | 0.53 |
| 205976_at | 22868 | FASTKD2 | NM_014929 | 48.86 | 80.50 | 43.68 | 0.54 | 48.86 | 73.66 | 54.61 | 0.74 |
| 206561_s_at | 57016 | AKR1B10 | NM_020299 | 11.56 | 28.31 | 15.37 | 0.54 | 11.56 | 22.33 | 24.19 | |
| 209124_at | 4615 | MYD88 | U70451 | 1576.46 | 2487.91 | 1351.39 | 0.54 | 1576.46 | 1687.04 | 587.50 | |
| 206382_s_at | 627 | BDNF | NM_001709 | 7.01 | 15.96 | 8.67 | 0.54 | 7.01 | 11.11 | 12.87 | |
| 203849_s_at | 547 | KIF1A | NM_004321 | 14.79 | 24.79 | 13.47 | 0.54 | 14.79 | 4.51 | 36.08 | |
| AFFX-BioB- | | | AFFX- | 447.16 | 784.04 | 426.26 | 0.54 | 447.16 | 424.02 | 283.64 | |
| 3_at | | | BioB-3 | | | | | | | | |
| 217433_at | 6867 | TACC1 | AB029026 | 6.98 | 19.88 | 10.81 | 0.54 | 6.98 | 21.66 | 10.39 | 0.48 |
| 212611_at | 23220 | DTX4 | AV728526 | 186.62 | 699.08 | 380.35 | 0.54 | 186.62 | 440.08 | 335.05 | |
| 210873_x_at | 200315 | APOBEC3A | U03891 | 4.78 | 44.43 | 24.18 | 0.54 | 4.78 | 165.32 | 25.92 | 0.16 |
| 215904_at | 4301 | MLLT4 | AL049698 | 1.15 | 26.08 | 14.20 | 0.54 | 1.15 | 7.82 | 19.30 | |
| 202662_s_at | 3709 | ITPR2 | NM_002223 | 94.29 | 154.21 | 84.03 | 0.54 | 94.29 | 93.41 | 125.45 | |
| 205935_at | 2294 | FOXF1 | NM_001451 | 27.17 | 45.28 | 24.67 | 0.54 | 27.17 | 77.08 | 64.65 | |
| 217277_at | | | AL008721 | 14.95 | 43.89 | 23.94 | 0.55 | 14.95 | 28.64 | 29.82 | |
| 211531_x_at | 5542 /// | PRB1 | K03205 | 73.74 | 118.31 | 64.57 | 0.55 | 73.74 | 67.31 | 43.24 | |
| | | /// | | | | | | | | | |
| | | PRB2 | | | | | | | | | |
| 220710_at | 80035 | C15orf28 | NM_024970 | 87.22 | 207.09 | 113.09 | 0.55 | 87.22 | 91.82 | 117.64 | |
| 205569_at | 27074 | LAMP3 | NM_014398 | 2.47 | 124.33 | 67.95 | 0.55 | 2.47 | 5996.80 | 1210.41 | 0.20 |
| 206255_at | 640 | BLK | NM_001715 | 2.15 | 10.69 | 5.84 | 0.55 | 2.15 | 3.75 | 2.77 | |
| 215811_at | 6622 | SNCA | AF238870 | 6.72 | 30.01 | 16.41 | 0.55 | 6.72 | 3.29 | 6.46 | |
| 207670_at | 3891 | KRT85 | NM_002283 | 20.89 | 47.79 | 26.18 | 0.55 | 20.89 | 28.78 | 22.04 | |
| 215284_at | 51429 | SNX9 | AF070575 | 23.02 | 53.74 | 29.46 | 0.55 | 23.02 | 17.55 | 22.15 | |
| 210578_at | 10107 | TRIM10 | AF220122 | 24.21 | 50.62 | 27.75 | 0.55 | 24.21 | 26.88 | 20.04 | |
| 207526_s_at | 9173 | IL1RL1 | NM_003856 | 10.85 | 18.10 | 9.93 | 0.55 | 10.85 | 6.03 | 3.98 | |
| 221905_at | 1540 | CYLD | BF516433 | 82.52 | 405.95 | 222.84 | 0.55 | 82.52 | 216.75 | 159.34 | 0.74 |
| 216499_at | | | AL137590 | 21.45 | 105.17 | 57.74 | 0.55 | 21.45 | 47.06 | 69.81 | |
| 215550_at | 9901 | SRGAP3 | AL137457 | 23.64 | 84.50 | 46.42 | 0.55 | 23.64 | 17.94 | 40.98 | |
| 210433_at | 23509 | POFUT1 | BC000582 | 13.87 | 54.44 | 29.91 | 0.55 | 13.87 | 23.05 | 42.46 | |
| 37512_at | 8630 | HSD17B6 | U89281 | 14.46 | 29.98 | 16.48 | 0.55 | 14.46 | 29.73 | 20.67 | 0.70 |
| 217235_x_at | | | D84140 | 36.97 | 89.71 | 49.32 | 0.55 | 36.97 | 49.06 | 25.02 | |
| 210743_s_at | 8556 | CDC14A | AF064103 | 65.42 | 112.10 | 61.62 | 0.55 | 65.42 | 63.56 | 26.99 | |
| 205744_at | 8448 | DOC2A | NM_003586 | 49.94 | 77.70 | 42.74 | 0.55 | 49.94 | 55.52 | 55.74 | |
| AFFX- | | | AFFX- | 100.84 | 176.11 | 96.90 | 0.55 | 100.84 | 51.70 | 212.23 | |
| HUMRGE/M10098_5_at | | | HUMRGE/ | | | | | | | | |
| | | | M10098_5 | | | | | | | | |
| 207928_s_at | 8001 | GLRA3 | NM_006529 | 2.26 | 13.80 | 7.59 | 0.55 | 2.26 | 2.02 | 2.85 | |
| 205425_at | 3092 | HIP1 | NM_005338 | 203.31 | 395.38 | 217.94 | 0.55 | 203.31 | 214.11 | 196.43 | |
| 204980_at | 9575 | CLOCK | NM_004898 | 271.80 | 435.45 | 240.07 | 0.55 | 271.80 | 464.62 | 220.13 | 0.47 |
| 213362_at | 5789 | PTPRD | N73931 | 17.26 | 31.03 | 17.12 | 0.55 | 17.26 | 8.46 | 21.60 | |
| 219480_at | 6615 | SNAI1 | NM_005985 | 55.89 | 160.37 | 88.48 | 0.55 | 55.89 | 159.78 | 157.05 | |
| 216265_x_at | 4625 | MYH7 | AI292276 | 1.73 | 13.20 | 7.28 | 0.55 | 1.73 | 3.32 | 10.19 | |
| 220073_s_at | 55200 | PLEKHG6 | NM_018173 | 26.25 | 53.79 | 29.70 | 0.55 | 26.25 | 7.48 | 27.49 | |
| 206699_x_at | 4861 | NPAS1 | NM_002517 | 8.54 | 18.47 | 10.20 | 0.55 | 8.54 | 21.49 | 5.57 | 0.26 |
| 221898_at | 10630 | PDPN | AU154455 | 66.68 | 153.19 | 84.64 | 0.55 | 66.68 | 19.34 | 43.79 | |
| 202222_s_at | 1674 | DES | NM_001927 | 29.18 | 62.95 | 34.80 | 0.55 | 29.18 | 20.71 | 37.45 | |
| 216372_at | | | AF103295 | 5.55 | 34.13 | 18.87 | 0.55 | 5.55 | 8.29 | 15.86 | |
| 219853_at | 79147 | FKRP | BC002612 | 12.33 | 67.30 | 37.22 | 0.55 | 12.33 | 47.93 | 55.53 | |
| 51226_at | | | N53536 | 26.09 | 42.12 | 23.29 | 0.55 | 26.09 | 13.40 | 11.21 | |
| 210197_at | 3705 | ITPK1 | BC003622 | 11.15 | 24.41 | 13.50 | 0.55 | 11.15 | 8.20 | 8.34 | |
| 220752_at | 51145 | LOC51145 | NM_016158 | 11.12 | 36.72 | 20.31 | 0.55 | 11.12 | 21.35 | 6.78 | 0.32 |
| 207461_at | | | NM_001272 | 15.57 | 27.78 | 15.37 | 0.55 | 15.57 | 16.05 | 3.46 | |
| 216984_x_at | 3535 | IGL@ | D84143 | 4.91 | 13.58 | 7.51 | 0.55 | 4.91 | 7.88 | 15.03 | |
| 214904_at | 7592 | ZNF41 | AI927984 | 28.59 | 53.02 | 29.34 | 0.55 | 28.59 | 35.44 | 25.29 | |
| 204363_at | 2152 | F3 | NM_001993 | 224.43 | 764.37 | 423.36 | 0.55 | 224.43 | 371.32 | 748.25 | |
| 204958_at | 1263 | PLK3 | NM_004073 | 55.80 | 180.65 | 100.24 | 0.55 | 55.80 | 298.27 | 1262.77 | |
| 211315_s_at | 8913 | CACNA1G | AB012043 | 4.82 | 23.79 | 13.20 | 0.55 | 4.82 | 11.24 | 12.00 | |
| 205888_s_at | 23040 | JAKMIP2 | AI962693 | 5.51 | 17.36 | 9.64 | 0.56 | 5.51 | 4.54 | 2.70 | |
| | // | /// | | | | | | | | | |
| | | MYT1L | | | | | | | | | |
| 214516_at | 8366 | HIST1H4B | NM_003544 | 4.22 | 23.10 | 12.83 | 0.56 | 4.22 | 23.87 | 10.37 | 0.43 |
| 215221_at | 27086 | FOXP1 | AK025064 | 226.27 | 401.58 | 223.02 | 0.56 | 226.27 | 180.79 | 183.70 | |
| 205262_at | 3757 | KCNH2 | NM_000238 | 7.98 | 139.77 | 77.73 | 0.56 | 7.98 | 25.24 | 5.72 | 0.23 |
| 217024_x_at | 140885 | SIRPA | AC004832 | 69.21 | 194.86 | 108.36 | 0.56 | 69.21 | 85.81 | 170.74 | |
| 215387_x_at | 10082 | GPC6 | AK021505 | 68.22 | 142.87 | 79.46 | 0.56 | 68.22 | 89.10 | 26.94 | |
| 220711_at | | | NM_024978 | 77.24 | 144.22 | 80.23 | 0.56 | 77.24 | 87.96 | 91.39 | |
| 210230_at | 728965 | LOC728965 | BC003629 | 653.12 | 1704.32 | 948.25 | 0.56 | 653.12 | 1578.41 | 1354.99 | |
| 203394_s_at | 3280 | HES1 | BE973687 | 11.64 | 26.95 | 15.01 | 0.56 | 11.64 | 41.23 | 28.61 | 0.69 |
| 202545_at | 5580 | PRKCD | NM_006254 | 1738.33 | 3145.25 | 1752.71 | 0.56 | 1738.33 | 2390.31 | 2411.49 | |
| 206973_at | 8499 | PPF1A2 | NM_003625 | 15.84 | 124.67 | 69.49 | 0.56 | 15.84 | 76.28 | 98.64 | |
| 217179_x_at | 96610 | LOC96610 | X79782 | 9.33 | 25.97 | 14.47 | 0.56 | 9.33 | 40.96 | 32.12 | |
| 218810_at | 80149 | ZC3H12A | NM_025079 | 120.05 | 822.86 | 458.68 | 0.56 | 120.05 | 1177.82 | 2182.59 | |
| 212091_s_at | 1291 | COL6A1 | AI141603 | 135.96 | 364.34 | 203.09 | 0.56 | 135.96 | 674.75 | 506.24 | |
| 207987_s_at | 2796 | GNRH1 | NM_000825 | 9.00 | 36.96 | 20.60 | 0.56 | 9.00 | 25.18 | 49.24 | |
| 214128_at | 747 | C11orf11 | AB014559 | 72.53 | 114.27 | 63.79 | 0.56 | 72.53 | 129.18 | 104.34 | |
| 222114_x_at | 54853 | WDR55 | BE409994 | 31.53 | 48.60 | 27.15 | 0.56 | 31.53 | 29.37 | 17.55 | |
| 206966_s_at | 11278 | KLF12 | NM_016285 | 56.94 | 103.37 | 57.80 | 0.56 | 56.94 | 39.40 | 41.11 | |
| 217270_s_at | 9149 | DYRK1B | AC005393 | 28.10 | 42.38 | 23.70 | 0.56 | 28.10 | 31.73 | 20.31 | |
| 206780_at | 2572 | GAD2 | NM_000818 | 26.45 | 50.02 | 27.97 | 0.56 | 26.45 | 32.32 | 23.56 | |
| 219227_at | 79616 | CCNJL | NM_024565 | 3.73 | 29.44 | 16.48 | 0.56 | 3.73 | 79.41 | 75.44 | |
| 205346_at | 6483 | ST3GAL2 | NM_006927 | 62.50 | 220.88 | 123.65 | 0.56 | 62.50 | 161.43 | 126.83 | |
| 216813_at | | | AL512728 | 3.95 | 22.37 | 12.53 | 0.56 | 3.95 | 7.16 | 28.67 | |
| 206100_at | 1368 | CPM | NM_001874 | 1155.85 | 3121.43 | 1748.94 | 0.56 | 1155.85 | 5890.11 | 2966.29 | 0.50 |
| 220374_at | 54813 | BTBD5 | NM_017658 | 13.47 | 55.62 | 31.17 | 0.56 | 13.47 | 39.98 | 28.82 | 0.72 |
| 207025_at | 57165 | GJA12 | NM_020435 | 16.01 | 37.12 | 20.81 | 0.56 | 16.01 | 25.71 | 35.71 | |
| 216812_at | | | AF308291 | 2.62 | 13.46 | 7.55 | 0.56 | 2.62 | 5.61 | 8.83 | |
| 215149_at | | | AF052109 | 14.42 | 29.68 | 16.64 | 0.56 | 14.42 | 14.76 | 14.35 | |
| 204621_s_at | 4929 | NR4A2 | AI935096 | 33.55 | 73.04 | 40.95 | 0.56 | 33.55 | 127.15 | 80.23 | 0.63 |
| 216147_at | 55752 | 11-Sep | AL353942 | 23.66 | 232.22 | 130.20 | 0.56 | 23.66 | 112.72 | 192.96 | |
| 38037_at | 1839 | HBEGF | M60278 | 53.04 | 83.15 | 46.62 | 0.56 | 53.04 | 148.66 | 126.52 | |
| 207093_s_at | 4974 | OMG | NM_002544 | 43.54 | 80.98 | 45.41 | 0.56 | 43.54 | 55.02 | 36.52 | |
| 206191_at | 956 | ENTPD3 | NM_001248 | 9.27 | 17.66 | 9.91 | 0.56 | 9.27 | 20.77 | 9.67 | 0.47 |
| 218801_at | 55757 | UGCGL2 | NM_020121 | 110.85 | 169.53 | 95.15 | 0.56 | 110.85 | 126.67 | 139.82 | |
| 219752_at | 8437 | RASAL1 | NM_004658 | 31.29 | 66.68 | 37.43 | 0.56 | 31.29 | 13.95 | 20.90 | |
| 220137_at | 54621 | FLJ20674 | NM_019086 | 76.53 | 137.23 | 77.05 | 0.56 | 76.53 | 74.70 | 67.30 | |
| 207630_s_at | 1390 | CREM | NM_001881 | 114.54 | 194.88 | 109.43 | 0.56 | 114.54 | 211.42 | 224.82 | |
| 207007_at | 9970 | NR1I3 | NM_005122 | 7.81 | 57.54 | 32.33 | 0.56 | 7.81 | 23.95 | 18.69 | |
| 217212_s_at | 3581 /// | IL9R | Z84723 | 35.78 | 81.25 | 45.65 | 0.56 | 35.78 | 57.74 | 51.02 | |
| | | /// | | | | | | | | | |
| | | LOC729486 | | | | | | | | | |
| 210726_at | 1576 | CYP3A4 | J04449 | 12.41 | 41.65 | 23.41 | 0.56 | 12.41 | 8.08 | 19.72 | |
| 219400_at | 8506 | CNTNAP1 | NM_003632 | 20.59 | 41.58 | 23.37 | 0.56 | 20.59 | 47.40 | 23.08 | 0.49 |
| 221653_x_at | 23780 | APOL2 | BC004395 | 69.69 | 106.76 | 60.03 | 0.56 | 69.69 | 49.07 | 20.89 | |
| 206648_at | 51276 | ZNF571 | NM_016536 | 12.62 | 31.85 | 17.92 | 0.56 | 12.62 | 28.30 | 27.06 | |
| 217141_at | 55727 | BTBD7 | AL049394 | 31.90 | 83.12 | 46.80 | 0.56 | 31.90 | 27.25 | 47.58 | |
| 206209_s_at | 762 | CA4 | NM_000717 | 20.90 | 43.68 | 24.61 | 0.56 | 20.90 | 3.71 | 17.64 | |
| 221681_s_at | 1834 | DSPP | AF094508 | 17.38 | 57.96 | 32.67 | 0.56 | 17.38 | 25.33 | 27.25 | |
| AFFX- | 6772 | STAT1 | AFFX- | 1011.38 | 1734.07 | 978.40 | 0.56 | 1011.38 | 1278.43 | 866.67 | |
| HUMISGF3A/ | | | HUMISGF3A/ | | | | | | | | |
| M97935_3_at | | | M97935_3 | | | | | | | | |
| 217541_x_at | 125893 / | LOC125893 | BG290532 | 82.50 | 138.83 | 78.39 | 0.56 | 82.50 | 70.12 | 41.45 | |
| | | /// | | | | | | | | | |
| | | LOC731901 | | | | | | | | | |
| 210601_at | 1004 | CDH6 | BC000019 | 6.61 | 16.90 | 9.54 | 0.56 | 6.61 | 13.62 | 7.58 | 0.56 |
| AFFX-r2-Ec- | | | AFFX-r2- | 677.00 | 1126.52 | 636.20 | 0.56 | 677.00 | 628.74 | 410.49 | |
| bioB-M_at | | | Ec-bioB-M | | | | | | | | |
| 205723_at | 1271 | CNTFR | NM_001842 | 37.14 | 124.98 | 70.61 | 0.56 | 37.14 | 75.70 | 97.83 | |
| 37566_at | 23349 | KIAA1045 | AB028968 | 7.74 | 31.79 | 17.97 | 0.57 | 7.74 | 40.64 | 17.20 | 0.42 |
| 217778_at | 27173 | SLC39A1 | NM_014437 | 323.99 | 551.36 | 311.62 | 0.57 | 323.99 | 304.88 | 225.76 | |
| 214445_at | 22936 | ELL2 | NM_012081 | 11.49 | 25.86 | 14.62 | 0.57 | 11.49 | 6.72 | 44.76 | |
| 205303_at | 3764 | KCNJ8 | BF514158 | 3.75 | 14.59 | 8.26 | 0.57 | 3.75 | 5.67 | 9.91 | |
| 215513_at | 57061 | HYMA1 | AF241534 | 88.17 | 181.76 | 102.86 | 0.57 | 88.17 | 181.58 | 199.14 | |
| AFFX-BioB- | | | AFFX- | 733.24 | 1184.42 | 670.42 | 0.57 | 733.24 | 612.06 | 377.41 | |
| M_at | | | BioB-M | | | | | | | | |
| 216565_x_at | 391020 | LOC391020 | AL121994 | 301.64 | 510.32 | 288.88 | 0.57 | 301.64 | 192.14 | 225.44 | |
| 214230_at | 998 | CDC42 | R37664 | 5.92 | 22.80 | 12.92 | 0.57 | 5.92 | 5.70 | 21.06 | |
| 205265_s_at | 10290 | SPEG | NM_005876 | 15.45 | 30.76 | 17.42 | 0.57 | 15.45 | 14.02 | 26.29 | |
| 215421_at | | | AI821657 | 27.25 | 45.53 | 25.79 | 0.57 | 27.25 | 47.99 | 21.39 | 0.45 |
| 216810_at | 85287 | KRTAP4-7 | AJ406939 | 4.49 | 15.30 | 8.67 | 0.57 | 4.49 | 19.39 | 19.83 | |
| 211549_s_at | 3248 | HPGD | U63296 | 6.87 | 10.78 | 6.11 | 0.57 | 6.87 | 3.08 | 21.19 | |
| 221978_at | 3134 | HLA-F | BE138825 | 43.93 | 80.39 | 45.71 | 0.57 | 43.93 | 60.69 | 61.09 | |
| 216322_at | 965 | CD58 | D28586 | 29.83 | 46.82 | 26.63 | 0.57 | 29.83 | 74.55 | 33.15 | 0.44 |
| 215661_at | 23139 | MAST2 | AK025352 | 12.29 | 40.70 | 23.15 | 0.57 | 12.29 | 15.76 | 24.58 | |
| 206286_s_at | 6997 /// | TDGF1 | NM_003212 | 12.89 | 20.90 | 11.90 | 0.57 | 12.89 | 20.82 | 5.05 | 0.24 |
| | | /// | | | | | | | | | |
| | | TDGF3 | | | | | | | | | |
| 211436_at | | | AF130053 | 4.74 | 11.97 | 6.81 | 0.57 | 4.74 | 9.80 | 1.09 | |
| 221329_at | 23538 | OR52A1 | NM_012375 | 17.32 | 33.04 | 18.81 | 0.57 | 17.32 | 5.16 | 14.84 | |
| 210605_s_at | 4240 | MFGE8 | BC003610 | 49.85 | 90.54 | 51.56 | 0.57 | 49.85 | 33.15 | 20.67 | |
| 207868_at | 1135 | CHRNA2 | NM_000742 | 58.88 | 89.58 | 51.01 | 0.57 | 58.88 | 64.98 | 52.14 | |
| 220232_at | 79966 | SCD5 | NM_024906 | 116.44 | 257.09 | 146.48 | 0.57 | 116.44 | 112.90 | 121.68 | |
| 211232_x_at | 2740 | GLP1R | L23503 | 5.80 | 13.69 | 7.80 | 0.57 | 5.80 | 6.15 | 9.21 | |
| 210841_s_at | 8828 | NRP2 | AF280546 | 47.60 | 97.39 | 55.50 | 0.57 | 47.60 | 98.87 | 135.69 | |
| 206682_at | 10462 | CLEC10A | NM_006344 | 46.54 | 84.46 | 48.17 | 0.57 | 46.54 | 28.26 | 18.35 | |
| AFFX-BioB- | | | AFFX- | 471.26 | 804.59 | 459.65 | 0.57 | 471.26 | 471.94 | 322.91 | |
| 5_at | | | BioB-5 | | | | | | | | |
| 214984_at | 23049 | SMG1 | AC003007 | 13.67 | 40.98 | 23.43 | 0.57 | 13.67 | 28.19 | 11.93 | 0.42 |
| | // | /// | | | | | | | | | |
| | | DKFZp547E087 | | | | | | | | | |
| | | /// | | | | | | | | | |
| | | LOC4 | | | | | | | | | |
| 214137_at | 5795 | PTPRJ | AI806482 | 12.88 | 33.65 | 19.24 | 0.57 | 12.88 | 26.53 | 25.52 | |
| 204438_at | 414308 / | MRC1 | NM_002438 | 7.79 | 12.12 | 6.94 | 0.57 | 7.79 | 9.37 | 8.37 | |
| | | /// | | | | | | | | | |
| | | MRC1L1 | | | | | | | | | |
| 218425_at | 54476 | TRIAD3 | BC000787 | 363.64 | 594.58 | 340.41 | 0.57 | 363.64 | 374.30 | 369.69 | |
| 205092_x_at | 22890 | ZBTB1 | NM_014950 | 18.37 | 37.65 | 21.56 | 0.57 | 18.37 | 42.10 | 59.26 | |
| 207575_at | 342096 / | GOLGA | NM_018652 | 15.87 | 29.29 | 16.77 | 0.57 | 15.87 | 5.57 | 18.62 | |
| | | /// | | | | | | | | | |
| | | GOLGA6 | | | | | | | | | |
| | | /// | | | | | | | | | |
| | | LOC653641 | | | | | | | | | |
| 216191_s_at | 64919 | TRA@ | X72501 | 0.96 | 17.62 | 10.10 | 0.57 | 0.96 | 7.13 | 5.41 | |
| | // | /// | | | | | | | | | |
| | | TRD@ | | | | | | | | | |
| | | /// | | | | | | | | | |
| | | BCL11B | | | | | | | | | |
| 205153_s_at | 958 | CD40 | NM_001250 | 264.54 | 1239.11 | 710.48 | 0.57 | 264.54 | 1797.70 | 1931.70 | |
| 210804_x_at | 6546 | SLC8A1 | AF128524 | 42.68 | 67.98 | 38.99 | 0.57 | 42.68 | 16.40 | 14.98 | |
| 209525_at | 50810 | HDGFRP3 | BG285017 | 3.78 | 10.56 | 6.06 | 0.57 | 3.78 | 1.75 | 3.52 | |
| 207194_s_at | 3386 | ICAM4 | NM_001544 | 20.19 | 47.25 | 27.12 | 0.57 | 20.19 | 29.54 | 17.25 | |
| 206766_at | 8515 | ITGA10 | AF112345 | 53.44 | 109.42 | 62.86 | 0.57 | 53.44 | 52.56 | 55.10 | |
| AFFX-BioC- | | | AFFX- | 1489.47 | 2318.83 | 1332.44 | 0.57 | 1489.47 | 1341.35 | 937.98 | |
| 5_at | | | BioC-5 | | | | | | | | |
| 204287_at | 9145 | SYNGR1 | NM_004711 | 46.52 | 71.02 | 40.83 | 0.57 | 46.52 | 36.47 | 76.27 | |
| 203595_s_at | 24138 | IFIT5 | N47725 | 27.91 | 198.45 | 114.11 | 0.58 | 27.91 | 239.63 | 98.57 | 0.41 |
| 206508_at | 970 | CD70 | NM_001252 | 877.18 | 3190.96 | 1835.10 | 0.58 | 877.18 | 1948.79 | 1672.51 | |
| 201750_s_at | 1889 | ECE1 | NM_001397 | 20.76 | 76.46 | 44.00 | 0.58 | 20.76 | 182.20 | 191.67 | |
| 215498_s_at | 5606 | MAP2K3 | AA780381 | 179.29 | 518.27 | 298.30 | 0.58 | 179.29 | 985.59 | 1021.49 | |
| 203242_s_at | 10611 | PDLIM5 | BG054550 | 260.99 | 593.87 | 342.05 | 0.58 | 260.99 | 265.58 | 464.58 | |
| 217201_at | 9462 | RASAL2 | AB007970 | 22.70 | 64.09 | 36.92 | 0.58 | 22.70 | 54.47 | 30.33 | 0.56 |
| 220771_at | 51152 | LOC51152 | NM_016181 | 38.29 | 73.33 | 42.26 | 0.58 | 38.29 | 42.00 | 42.67 | |
| 220272_at | 54796 | BNC2 | NM_017637 | 24.44 | 38.36 | 22.11 | 0.58 | 24.44 | 12.21 | 5.86 | |
| 220905_at | | | NM_025007 | 16.72 | 62.26 | 35.90 | 0.58 | 16.72 | 51.93 | 24.26 | 0.47 |
| 207312_at | 5260 | PHKG1 | NM_006213 | 32.44 | 59.02 | 34.07 | 0.58 | 32.44 | 9.42 | 50.25 | |
| 211407_at | 4713 | NDUFB7 | M33374 | 25.58 | 53.84 | 31.10 | 0.58 | 25.58 | 34.20 | 20.41 | |
| 206539_s_at | 66002 | CYP4F12 | NM_023944 | 11.43 | 79.74 | 46.06 | 0.58 | 11.43 | 19.47 | 17.14 | |
| 210173_at | 5795 | PTPRJ | D37781 | 79.43 | 119.78 | 69.22 | 0.58 | 79.43 | 40.66 | 106.77 | |
| 204654_s_at | 7020 | TFAP2A | NM_003220 | 9.12 | 114.94 | 66.47 | 0.58 | 9.12 | 217.09 | 225.23 | |
| 216072_at | 3275 | PRMT2 | AL050065 | 11.83 | 34.17 | 19.76 | 0.58 | 11.83 | 36.46 | 3.50 | 0.10 |
| 214707_x_at | 7840 | ALMS1 | AB002326 | 100.13 | 414.70 | 239.87 | 0.58 | 100.13 | 127.94 | 81.47 | |
| 215627_at | 440925 | LOC440925 | AK023515 | 19.11 | 53.40 | 30.93 | 0.58 | 19.11 | 48.09 | 14.09 | 0.29 |
| 208596_s_at | 54575 | UGT1A10 | NM_019093 | 7.00 | 15.77 | 9.13 | 0.58 | 7.00 | 13.37 | 16.34 | |
| | // | /// | | | | | | | | | |
| | | UGT1A8 | | | | | | | | | |
| | | /// | | | | | | | | | |
| | | UGT1A7 | | | | | | | | | |
| 207375_s_at | 3601 | IL15RA | NM_002189 | 71.76 | 121.46 | 70.36 | 0.58 | 71.76 | 245.72 | 303.93 | |
| 202637_s_at | 3383 | ICAM1 | AI608725 | 37.89 | 1474.78 | 854.57 | 0.58 | 37.89 | 1749.04 | 1165.92 | 0.67 |
| 207718_x_at | 1548 /// | CYP2A6 | NM_000764 | 26.66 | 46.31 | 26.84 | 0.58 | 26.66 | 51.38 | 24.89 | 0.48 |
| | | /// | | | | | | | | | |
| | | CYP2A7 | | | | | | | | | |
| | | /// | | | | | | | | | |
| | | CYP2A7P1 | | | | | | | | | |
| 209785_s_at | 8605 | PLA2G4C | AF065214 | 128.84 | 631.78 | 366.27 | 0.58 | 128.84 | 654.05 | 528.35 | |
| 206999_at | 3595 | IL12RB2 | NM_001559 | 1114.24 | 1860.35 | 1078.75 | 0.58 | 1114.24 | 1418.15 | 1004.03 | |
| 209886_s_at | 4091 | SMAD6 | AF035528 | 20.93 | 52.85 | 30.66 | 0.58 | 20.93 | 13.94 | 17.92 | |
| 219869_s_at | 64116 | SLC39A8 | AW139759 | 736.35 | 1327.90 | 770.56 | 0.58 | 736.35 | 1684.93 | 1731.26 | |
| 212503_s_at | 22982 | DIP2C | N22859 | 10.57 | 27.11 | 15.73 | 0.58 | 10.57 | 12.39 | 20.14 | |
| 211371_at | 5607 | MAP2K5 | U71088 | 3.85 | 10.74 | 6.24 | 0.58 | 3.85 | 6.96 | 12.59 | |
| 214424_s_at | | | AV650852 | 4.23 | 11.25 | 6.53 | 0.58 | 4.23 | 7.40 | 4.06 | |
| 205241_at | 9997 | SCO2 | NM_005138 | 454.58 | 968.47 | 562.22 | 0.58 | 454.58 | 1567.77 | 688.65 | 0.44 |
| 209493_at | 23037 | PDZD2 | AF338650 | 4.64 | 22.46 | 13.04 | 0.58 | 4.64 | 17.01 | 5.43 | 0.32 |
| 207733_x_at | 5678 | PSG9 | NM_002784 | 14.26 | 42.81 | 24.89 | 0.58 | 14.26 | 13.42 | 4.78 | |
| 215555_at | 57035 | C1orf63 | AU158442 | 42.37 | 171.04 | 99.46 | 0.58 | 42.37 | 43.08 | 61.25 | |
| 207667_s_at | 5606 | MAP2K3 | NM_002756 | 220.08 | 487.84 | 283.74 | 0.58 | 220.08 | 531.34 | 1158.48 | |
| 203729_at | 2014 | EMP3 | NM_001425 | 2819.63 | 5234.38 | 3044.73 | 0.58 | 2819.63 | 5665.66 | 5895.52 | |
| 221866_at | 7942 | TFEB | AL035588 | 238.25 | 397.89 | 231.47 | 0.58 | 238.25 | 341.25 | 296.07 | |
| 211862_x_at | 8837 | CFLAR | AF015451 | 409.00 | 1233.50 | 717.66 | 0.58 | 409.00 | 1333.04 | 1749.55 | |
| 222040_at | 3178 /// | HNRPA1 | AI144007 | 165.78 | 276.66 | 161.00 | 0.58 | 165.78 | 416.16 | 350.46 | |
| | | /// | | | | | | | | | |
| | | LOC728844 | | | | | | | | | |
| | | /// | | | | | | | | | |
| | | LOC73 | | | | | | | | | |
| 205225_at | 2099 | ESR1 | NM_000125 | 2.14 | 17.41 | 10.13 | 0.58 | 2.14 | 3.98 | 8.51 | |
| 219794_at | 55275 | VPS53 | NM_018289 | 49.29 | 87.53 | 50.94 | 0.58 | 49.29 | 31.43 | 61.86 | |
| 217200_x_at | 1534 | CYB561 | U06715 | 167.13 | 257.90 | 150.23 | 0.58 | 167.13 | 224.54 | 146.87 | |
| 205801_s_at | 25780 | RASGRP3 | NM_015376 | 3.46 | 13.15 | 7.66 | 0.58 | 3.46 | 3.17 | 1.50 | |
| 217208_s_at | 1739 /// | DLG1 | AL121981 | 220.18 | 355.90 | 207.43 | 0.58 | 220.18 | 148.54 | 153.67 | |
| | | /// | | | | | | | | | |
| | | TNFRSF11B | | | | | | | | | |
| 217704_x_at | 440423 | SUZ12P | AI820796 | 289.95 | 583.77 | 340.31 | 0.58 | 289.95 | 258.87 | 278.56 | |
| 208965_s_at | 3428 | IFI16 | BG256677 | 341.07 | 521.43 | 303.99 | 0.58 | 341.07 | 319.55 | 157.68 | |
| 206538_at | 22808 | MRAS | NM_012219 | 56.09 | 95.39 | 55.62 | 0.58 | 56.09 | 133.55 | 143.15 | |
| 211012_s_at | 161527 / | PML /// | BC000080 | 130.51 | 223.62 | 130.43 | 0.58 | 130.51 | 277.30 | 61.72 | 0.22 |
| | | LOC161527 | | | | | | | | | |
| 203882_at | 10379 | ISGF3G | NM_006084 | 553.66 | 1503.24 | 877.98 | 0.58 | 553.66 | 1354.94 | 1005.75 | 0.74 |
| 207998_s_at | 776 | CACNA1D | NM_000720 | 6.39 | 13.31 | 7.78 | 0.58 | 6.39 | 8.64 | 18.29 | |
| 206339_at | 9607 | CARTPT | NM_004291 | 28.40 | 49.14 | 28.75 | 0.58 | 28.40 | 40.01 | 24.85 | |
| 205174_s_at | 25797 | QPCT | NM_012413 | 60.94 | 236.11 | 138.15 | 0.59 | 60.94 | 309.07 | 161.82 | 0.52 |
| 204902_s_at | 23192 | ATG4B | NM_013325 | 96.08 | 152.52 | 89.25 | 0.59 | 96.08 | 200.99 | 111.85 | 0.56 |
| | // | /// | | | | | | | | | |
| | | LOC727737 | | | | | | | | | |
| 210282_at | 7750 | ZMYM2 | AL136621 | 49.96 | 95.03 | 55.68 | 0.59 | 49.96 | 115.43 | 64.80 | 0.56 |
| 220484_at | 55283 | MCOLN3 | NM_018298 | 374.54 | 824.00 | 482.88 | 0.59 | 374.54 | 615.67 | 1008.13 | |
| 211802_x_at | 8913 | CACNA1G | AF227750 | 17.00 | 34.12 | 20.00 | 0.59 | 17.00 | 17.98 | 18.23 | |
| 209016_s_at | 3855 | KRT7 | BC002700 | 8.76 | 51.50 | 30.19 | 0.59 | 8.76 | 43.08 | 29.90 | 0.69 |
| 221755_at | 254102 | EHBP1L1 | BG334196 | 381.85 | 698.02 | 409.19 | 0.59 | 381.85 | 124.69 | 219.63 | |
| 206995_x_at | 8578 | SCARF1 | NM_003693 | 29.69 | 49.43 | 28.99 | 0.59 | 29.69 | 80.71 | 81.92 | |
| 204682_at | 4053 | LTBP2 | NM_000428 | 41.55 | 69.71 | 40.90 | 0.59 | 41.55 | 25.89 | 46.87 | |
| 221440_s_at | 10741 | RBBP9 | NM_006606 | 8.25 | 18.93 | 11.12 | 0.59 | 8.25 | 19.01 | 18.60 | |
| 214058_at | 4610 | MYCL1 | M19720 | 299.15 | 821.66 | 482.94 | 0.59 | 299.15 | 298.31 | 216.24 | |
| 211552_s_at | 8659 | ALDH4A1 | U24267 | 25.77 | 40.80 | 23.99 | 0.59 | 25.77 | 14.35 | 24.14 | |
| 217054_at | | | AF007194 | 20.63 | 52.61 | 30.93 | 0.59 | 20.63 | 15.92 | 18.64 | |
| 206850_at | 10633 | RRP22 | NM_006477 | 55.76 | 100.63 | 59.20 | 0.59 | 55.76 | 205.20 | 295.67 | |
| 215425_at | 10950 | BTG3 | AL049332 | 20.75 | 44.40 | 26.13 | 0.59 | 20.75 | 30.33 | 21.44 | |
| 216432_at | 9153 | SLC28A2 | AK025121 | 5.99 | 12.07 | 7.11 | 0.59 | 5.99 | 12.71 | 2.85 | 0.22 |
| 215496_at | 23034 | SAMD4A | AL117523 | 11.78 | 32.17 | 18.94 | 0.59 | 11.78 | 30.63 | 40.68 | |
| 210594_x_at | 9019 | MPZL1 | AF239756 | 53.60 | 110.93 | 65.33 | 0.59 | 53.60 | 35.30 | 91.06 | |
| 219411_at | 79767 | ELMO3 | NM_024712 | 63.48 | 103.14 | 60.74 | 0.59 | 63.48 | 117.35 | 113.52 | |
| 203441_s_at | 1000 | CDH2 | NM_001792 | 56.46 | 87.33 | 51.56 | 0.59 | 56.46 | 53.16 | 80.65 | |
| 211653_x_at | 1646 | AKR1C2 | M33376 | 25.79 | 59.50 | 35.14 | 0.59 | 25.79 | 62.69 | 198.51 | |
| 211571_s_at | 1462 | CSPG2 | D32039 | 91.45 | 197.62 | 116.73 | 0.59 | 91.45 | 143.81 | 123.83 | |
| 214590_s_at | 7321 | UBE2D1 | AL545760 | 25.37 | 41.26 | 24.37 | 0.59 | 25.37 | 36.59 | 44.37 | |
| 206830_at | 57282 | SLC4A10 | NM_022058 | 8.17 | 18.16 | 10.72 | 0.59 | 8.17 | 10.63 | 2.69 | |
| 216745_x_at | | | AK024606 | 114.85 | 201.71 | 119.18 | 0.59 | 114.85 | 130.79 | 82.49 | |
| 211668_s_at | 5328 | PLAU | K03226 | 177.67 | 396.21 | 234.14 | 0.59 | 177.67 | 579.34 | 2489.29 | |
| AFFX-r2-Ec- | | | AFFX-r2- | 1801.29 | 2723.74 | 1610.18 | 0.59 | 1801.29 | 1638.71 | 976.91 | |
| bioC-5_at | | | Ec-bioC-5 | | | | | | | | |
| 210364_at | 6327 | SCN2B | U87555 | 5.28 | 38.85 | 22.97 | 0.59 | 5.28 | 18.91 | 12.20 | 0.64 |
| 208504_x_at | 56125 | PCDHB11 | NM_018931 | 10.82 | 21.09 | 12.47 | 0.59 | 10.82 | 7.45 | 5.04 | |
| 214068_at | 146227 | BEAN | AF070610 | 20.28 | 31.31 | 18.53 | 0.59 | 20.28 | 4.38 | 23.37 | |
| 203141_s_at | 8546 | AP3B1 | NM_003664 | 161.30 | 242.06 | 143.30 | 0.59 | 161.30 | 100.15 | 153.71 | |
| 206132_at | 4163 | MCC | NM_002387 | 33.27 | 67.28 | 39.85 | 0.59 | 33.27 | 51.38 | 70.01 | |
| 221920_s_at | 51312 | SLC25A37 | BE677761 | 209.09 | 337.45 | 200.01 | 0.59 | 209.09 | 78.66 | 217.19 | |
| 215868_x_at | 6660 | SOX5 | AK026238 | 38.29 | 63.01 | 37.35 | 0.59 | 38.29 | 143.81 | 82.81 | 0.58 |
| 216964_at | | | AL162082 | 26.50 | 49.35 | 29.25 | 0.59 | 26.50 | 29.39 | 32.70 | |
| 215385_at | 79068 | FTO | AK022473 | 114.44 | 203.90 | 120.88 | 0.59 | 114.44 | 93.30 | 83.09 | |
| 218631_at | 60370 | AVPI1 | NM_021732 | 59.68 | 108.10 | 64.08 | 0.59 | 59.68 | 252.57 | 159.91 | 0.63 |
| 214724_at | 85458 | DIXDC1 | AF070621 | 101.05 | 314.69 | 186.64 | 0.59 | 101.05 | 991.37 | 525.56 | 0.53 |
| 217259_at | | | AL117549 | 24.30 | 40.75 | 24.18 | 0.59 | 24.30 | 12.24 | 20.98 | |
| 208167_s_at | 4325 | MMP16 | NM_022564 | 6.60 | 22.80 | 13.54 | 0.59 | 6.60 | 5.44 | 7.73 | |
| 201308_s_at | 55752 | 11-Sep | NM_018243 | 137.44 | 234.40 | 139.25 | 0.59 | 137.44 | 130.93 | 250.19 | |
| 204901_at | 8945 | BTRC | AA824369 | 6.95 | 34.26 | 20.36 | 0.59 | 6.95 | 16.62 | 30.25 | |
| 211208_s_at | 8573 | CASK | AB039327 | 40.01 | 96.46 | 57.33 | 0.59 | 40.01 | 24.58 | 64.81 | |
| 213693_s_at | 4582 | MUC1 | AI610869 | 326.98 | 699.61 | 416.00 | 0.59 | 326.98 | 446.59 | 298.14 | |
| 200760_s_at | 10550 | ARL6IP5 | N92494 | 2140.43 | 3933.07 | 2338.70 | 0.59 | 2140.43 | 2566.86 | 973.29 | |
| 216154_at | 2262 | GPC5 | AF339787 | 7.03 | 33.40 | 19.88 | 0.60 | 7.03 | 2.65 | 5.02 | |
| 206423_at | 10218 | ANGPTL7 | NM_021146 | 6.35 | 16.98 | 10.11 | 0.60 | 6.35 | 3.05 | 10.19 | |
| 221103_s_at | 55779 | WDR52 | NM_018338 | 70.57 | 116.04 | 69.10 | 0.60 | 70.57 | 47.66 | 67.76 | |
| 216354_at | | | AL031669 | 56.16 | 96.11 | 57.29 | 0.60 | 56.16 | 95.15 | 87.22 | |
| 214873_at | 91355 | LRP5L | AL137651 | 16.41 | 42.77 | 25.51 | 0.60 | 16.41 | 39.00 | 13.15 | 0.34 |
| 207211_at | 9099 | USP2 | AF079564 | 6.52 | 10.00 | 5.97 | 0.60 | 6.52 | 7.24 | 9.44 | |
| 202971_s_at | 8445 | DYRK2 | Y09216 | 387.05 | 585.88 | 349.80 | 0.60 | 387.05 | 623.69 | 238.02 | 0.38 |
| 217933_s_at | 51056 | LAP3 | NM_015907 | 2126.63 | 4839.78 | 2891.17 | 0.60 | 2126.63 | 2787.93 | 2332.74 | |
| 213295_at | 1540 | CYLD | AA555096 | 181.04 | 590.06 | 352.62 | 0.60 | 181.04 | 438.26 | 382.32 | |
| 202897_at | 140885 | SIRPA | AB023430 | 702.89 | 1479.62 | 884.64 | 0.60 | 702.89 | 1787.89 | 1917.70 | |
| 214578_s_at | 6093 | ROCK1 | AV683882 | 298.04 | 514.05 | 307.49 | 0.60 | 298.04 | 313.88 | 391.02 | |
| 220874_at | | | NM_018575 | 25.33 | 44.84 | 26.83 | 0.60 | 25.33 | 37.80 | 16.04 | |
| 203666_at | 6387 | CXCL12 | NM_000609 | 34.89 | 82.37 | 49.36 | 0.60 | 34.89 | 115.21 | 169.19 | |
| 217542_at | 1368 | CPM | BE930512 | 30.13 | 51.25 | 30.72 | 0.60 | 30.13 | 98.01 | 70.73 | 0.72 |
| 216789_at | | | AK026439 | 5.94 | 14.12 | 8.47 | 0.60 | 5.94 | 23.56 | 2.65 | 0.11 |
| 217282_at | 10905 | MAN1A2 | AK001970 | 34.88 | 53.83 | 32.31 | 0.60 | 34.88 | 50.06 | 36.78 | |
| 216165_at | 84253 | GARNL3 | AK025650 | 6.93 | 12.48 | 7.49 | 0.60 | 6.93 | 3.79 | 4.12 | |
| 208242_at | 30062 | RAX | NM_013435 | 6.10 | 15.60 | 9.37 | 0.60 | 6.10 | 10.96 | 6.24 | 0.57 |
| 216751_at | 284040 | CDRT4 | AK024879 | 39.91 | 69.31 | 41.61 | 0.60 | 39.91 | 38.04 | 27.35 | |
| 212364_at | 4430 | MYO1B | BF432550 | 29.60 | 47.70 | 28.68 | 0.60 | 29.60 | 238.94 | 298.92 | |
| 210539_at | 23093 | TTLL5 | AK024259 | 35.15 | 90.38 | 54.34 | 0.60 | 35.15 | 63.24 | 77.27 | |
| 213488_at | 25992 | SNED1 | N73970 | 11.35 | 35.03 | 21.07 | 0.60 | 11.35 | 28.68 | 19.39 | 0.68 |
| 201150_s_at | 7078 | TIMP3 | NM_000362 | 2147.20 | 3422.85 | 2058.64 | 0.60 | 2147.20 | 1655.18 | 3952.57 | |
| 209160_at | 8644 | AKR1C3 | AB018580 | 5.27 | 16.10 | 9.68 | 0.60 | 5.27 | 43.53 | 16.81 | 0.39 |
| 220532_s_at | 28959 | TMEM176B | NM_014020 | 438.50 | 740.35 | 445.31 | 0.60 | 438.50 | 389.25 | 397.64 | |
| AFFX- | | | AFFX- | 135.66 | 286.88 | 172.71 | 0.60 | 135.66 | 117.60 | 229.38 | |
| HUMRGE/M10098_3_at | | | HUMRGE/ | | | | | | | | |
| | | | M10098_3 | | | | | | | | |
| 205637_s_at | 6457 | SH3GL3 | NM_003027 | 31.91 | 63.71 | 38.36 | 0.60 | 31.91 | 56.14 | 25.27 | 0.45 |
| 214909_s_at | 23564 | DDAH2 | AK026191 | 225.56 | 347.32 | 209.48 | 0.60 | 225.56 | 225.55 | 176.27 | |
| 203300_x_at | 8905 | AP1S2 | NM_003916 | 5225.34 | 9029.88 | 5446.98 | 0.60 | 5225.34 | 4677.36 | 5667.04 | |
| 209392_at | 5168 | ENPP2 | L35594 | 21.35 | 77.30 | 46.63 | 0.60 | 21.35 | 21.59 | 46.53 | |
| 219788_at | 29992 | PILRA | NM_013439 | 21.06 | 48.36 | 29.18 | 0.60 | 21.06 | 43.82 | 56.30 | |
| 218986_s_at | 55601 | FLJ20035 | NM_017631 | 256.69 | 451.79 | 272.81 | 0.60 | 256.69 | 549.85 | 467.75 | |
| 213954_at | 26049 | KIAA0888 | AB020695 | 18.81 | 66.78 | 40.35 | 0.60 | 18.81 | 28.75 | 79.01 | |
| 208078_s_at | 150094 | SNF1LK | NM_030751 | 125.45 | 211.31 | 127.72 | 0.60 | 125.45 | 497.13 | 983.86 | |
| 205333_s_at | 9986 | RCE1 | NM_005133 | 182.26 | 299.99 | 181.34 | 0.60 | 182.26 | 204.63 | 176.89 | |
| 221272_s_at | 81563 | C1orf21 | NM_030806 | 18.45 | 83.54 | 50.53 | 0.60 | 18.45 | 100.12 | 192.92 | |
| 212813_at | 83700 | JAM3 | AA149644 | 32.85 | 60.98 | 36.90 | 0.61 | 32.85 | 39.08 | 18.84 | |
| 213148_at | 257407 | LOC257407 | AW156899 | 13.99 | 33.39 | 20.20 | 0.61 | 13.99 | 43.98 | 41.82 | |
| 205616_at | | | AW134812 | 9.33 | 28.11 | 17.02 | 0.61 | 9.33 | 8.20 | 16.11 | |
| 210499_s_at | 10084 | PQBP1 | AB041834 | 83.01 | 156.38 | 94.75 | 0.61 | 83.01 | 93.77 | 120.50 | |
| 221496_s_at | 10766 | TOB2 | AL008582 | 270.75 | 447.07 | 270.96 | 0.61 | 270.75 | 307.28 | 257.74 | |
| 217359_s_at | 4684 | NCAM1 | M22094 | 6.80 | 29.72 | 18.02 | 0.61 | 6.80 | 25.25 | 18.10 | 0.72 |
| 203637_s_at | 4281 | MID1 | NM_000381 | 24.68 | 41.22 | 25.00 | 0.61 | 24.68 | 50.22 | 23.04 | 0.46 |
| 200887_s_at | 6772 | STAT1 | NM_007315 | 1429.05 | 3114.99 | 1890.80 | 0.61 | 1429.05 | 3209.97 | 1374.12 | 0.43 |
| 219780_at | 51333 | ZNF771 | NM_016643 | 8.07 | 26.89 | 16.34 | 0.61 | 8.07 | 15.30 | 20.25 | |
| AFFX- | | | AFFX- | 3048.13 | 4817.97 | 2928.98 | 0.61 | 3048.13 | 2782.67 | 1846.70 | |
| BioDn-5_at | | | BioDn-5 | | | | | | | | |
| 213075_at | 169611 | OLFML2A | AL050002 | 21.79 | 46.54 | 28.30 | 0.61 | 21.79 | 35.70 | 15.66 | 0.44 |
| 216822_x_at | | | AL359763 | 45.36 | 80.49 | 48.94 | 0.61 | 45.36 | 52.86 | 53.90 | |
| 160020_at | 4323 | MMP14 | Z48481 | 547.97 | 925.24 | 562.64 | 0.61 | 547.97 | 316.72 | 464.30 | |
| 207793_s_at | 2035 | EPB41 | NM_004437 | 18.13 | 44.48 | 27.06 | 0.61 | 18.13 | 24.54 | 26.01 | |
| 207195_at | 27255 | CNTN6 | NM_014461 | 3.92 | 12.13 | 7.38 | 0.61 | 3.92 | 3.84 | 5.70 | |
| 206450_at | 1621 | DBH | NM_000787 | 5.26 | 10.33 | 6.29 | 0.61 | 5.26 | 4.42 | 13.50 | |
| 216837_at | 2044 | EPHA5 | L36644 | 11.84 | 21.23 | 12.93 | 0.61 | 11.84 | 24.23 | 15.12 | 0.62 |
| 204049_s_at | 9749 | PHACTR2 | NM_014721 | 661.40 | 1322.14 | 805.65 | 0.61 | 661.40 | 1798.06 | 2022.93 | |
| 220906_at | | | NM_025016 | 8.92 | 40.75 | 24.85 | 0.61 | 8.92 | 21.42 | 7.53 | 0.35 |
| 208112_x_at | 10938 | EHD1 | NM_006795 | 292.95 | 836.69 | 510.35 | 0.61 | 292.95 | 2309.52 | 1481.01 | 0.64 |
| 206911_at | 7706 | TRIM25 | NM_005082 | 198.49 | 318.97 | 194.69 | 0.61 | 198.49 | 123.88 | 319.98 | |
| 205212_s_at | 9744 | CENTB1 | NM_014716 | 22.41 | 37.36 | 22.82 | 0.61 | 22.41 | 5.03 | 32.16 | |
| 214284_s_at | 8817 | FGF18 | AA022949 | 21.93 | 34.85 | 21.29 | 0.61 | 21.93 | 27.34 | 23.52 | |
| 217537_x_at | | | M78162 | 11.51 | 33.67 | 20.57 | 0.61 | 11.51 | 20.58 | 9.53 | 0.46 |
| 214028_x_at | 81550 | TDRD3 | AU156998 | 38.03 | 80.42 | 49.14 | 0.61 | 38.03 | 52.37 | 52.62 | |
| 215797_at | 348035 | MGC40069 | AE000659 | 15.84 | 35.77 | 21.85 | 0.61 | 15.84 | 28.28 | 27.43 | |
| 215852_x_at | 140710 | C20orf117 | AK022023 | 32.52 | 60.04 | 36.69 | 0.61 | 32.52 | 39.85 | 30.73 | |
| 58780_s_at | 55701 | FLJ10357 | R42449 | 502.95 | 777.06 | 475.08 | 0.61 | 502.95 | 1286.29 | 474.40 | 0.37 |
| 221392_at | 50832 | TAS2R4 | NM_016944 | 17.85 | 32.03 | 19.59 | 0.61 | 17.85 | 27.80 | 13.99 | 0.50 |
| 220090_at | 49860 | CRNN | NM_016190 | 9.55 | 14.94 | 9.14 | 0.61 | 9.55 | 7.34 | 11.96 | |
| 204096_s_at | 8178 | ELL | AL136771 | 61.79 | 97.89 | 59.90 | 0.61 | 61.79 | 67.93 | 78.75 | |
| 219622_at | 55647 | RAB20 | NM_017817 | 280.66 | 533.00 | 326.50 | 0.61 | 280.66 | 353.58 | 426.69 | |
| 206965_at | 11278 | KLF12 | NM_016285 | 7.50 | 35.23 | 21.59 | 0.61 | 7.50 | 15.40 | 9.19 | 0.60 |
| 220364_at | 54508 | FLJ11235 | NM_019033 | 11.10 | 24.76 | 15.17 | 0.61 | 11.10 | 6.53 | 13.74 | |
| 206487_at | 23353 | UNC84A | NM_025154 | 8.09 | 20.07 | 12.30 | 0.61 | 8.09 | 3.65 | 13.03 | |
| 220524_at | 54566 | EPB41L4B | NM_024823 | 3.99 | 10.81 | 6.62 | 0.61 | 3.99 | 8.00 | 5.31 | |
| 202273_at | 5159 | PDGFRB | NM_002609 | 27.54 | 49.04 | 30.07 | 0.61 | 27.54 | 15.71 | 7.50 | |
| 210424_s_at | 23015 | GOLGA8A | AF164622 | 165.95 | 281.58 | 172.67 | 0.61 | 165.95 | 159.59 | 199.51 | |
| | // | /// | | | | | | | | | |
| | | GOLGA8B | | | | | | | | | |
| 211402_x_at | 2649 | NR6A1 | AF004291 | 6.85 | 34.80 | 21.35 | 0.61 | 6.85 | 53.15 | 41.38 | |
| 222054_at | 664727 | LOC664727 | BF511556 | 61.57 | 110.25 | 67.66 | 0.61 | 61.57 | 96.20 | 125.55 | |
| 215437_x_at | 11176 | BAZ2A | BE513659 | 37.23 | 79.45 | 48.76 | 0.61 | 37.23 | 42.10 | 44.56 | |
| 217691_x_at | 9123 | SLC16A3 | AA853175 | 259.50 | 496.83 | 305.07 | 0.61 | 259.50 | 453.55 | 460.67 | |
| 221887_s_at | 25861 | DFNB31 | BE045998 | 8.92 | 49.81 | 30.60 | 0.61 | 8.92 | 39.37 | 57.36 | |
| 211799_x_at | 3107 | HLA-C | U62824 | 1250.72 | 2051.02 | 1260.54 | 0.61 | 1250.72 | 1070.82 | 1619.81 | |
| 213924_at | 65258 | MPPE1 | BF476502 | 6.82 | 14.22 | 8.74 | 0.61 | 6.82 | 11.07 | 24.41 | |
| 204891_s_at | 3932 | LCK | NM_005356 | 5.63 | 10.98 | 6.75 | 0.62 | 5.63 | 6.94 | 4.22 | |
| 202895_s_at | 140885 | SIRPA | D86043 | 110.67 | 206.40 | 127.01 | 0.62 | 110.67 | 105.38 | 191.50 | |
| 211681_s_at | 10611 | PDLIM5 | AF116705 | 153.09 | 237.78 | 146.52 | 0.62 | 153.09 | 128.69 | 205.71 | |
| 204770_at | 6891 | TAP2 | NM_000544 | 74.12 | 118.40 | 72.99 | 0.62 | 74.12 | 61.99 | 47.33 | |
| 211139_s_at | 4664 | NAB1 | AF045452 | 264.84 | 633.15 | 390.50 | 0.62 | 264.84 | 431.83 | 772.97 | |
| 215874_at | 6482 | ST3GAL1 | AK026820 | 45.38 | 108.71 | 67.05 | 0.62 | 45.38 | 57.62 | 47.46 | |
| 210109_at | 27099 | NAG8 | AF191492 | 97.09 | 229.39 | 141.67 | 0.62 | 97.09 | 167.40 | 199.58 | |
| 209935_at | 27032 | ATP2C1 | AF225981 | 97.38 | 166.37 | 102.77 | 0.62 | 97.38 | 496.95 | 571.50 | |
| 207497_s_at | 2206 | MS4A2 | D10583 | 4.70 | 38.40 | 23.72 | 0.62 | 4.70 | 33.23 | 9.25 | 0.28 |
| 211259_s_at | 655 | BMP7 | BC004248 | 9.88 | 27.92 | 17.25 | 0.62 | 9.88 | 21.05 | 27.41 | |
| 206763_at | 8468 | FKBP6 | NM_003602 | 16.91 | 26.85 | 16.59 | 0.62 | 16.91 | 31.50 | 18.80 | 0.60 |
| 201148_s_at | 7078 | TIMP3 | AW338933 | 699.60 | 1285.56 | 794.53 | 0.62 | 699.60 | 758.77 | 2063.65 | |
| 219353_at | 374354 | NHLRC2 | NM_017687 | 96.42 | 168.98 | 104.44 | 0.62 | 96.42 | 172.95 | 74.99 | 0.43 |
| AFFX-r2-Ec- | | | AFFX-r2- | 674.50 | 1038.38 | 641.82 | 0.62 | 674.50 | 635.36 | 415.27 | |
| bioB-3_at | | | Ec-bioB-3 | | | | | | | | |
| 210564_x_at | 8837 | CFLAR | AF009619 | 220.24 | 702.70 | 434.38 | 0.62 | 220.24 | 729.67 | 1093.87 | |
| 205878_at | 5463 | POU6F1 | NM_002702 | 4.24 | 44.00 | 27.21 | 0.62 | 4.24 | 44.54 | 14.65 | 0.33 |
| 210359_at | 9788 | MTSS1 | AF116674 | 21.07 | 46.89 | 29.00 | 0.62 | 21.07 | 52.63 | 102.29 | |
| 220180_at | 80323 | CCDC68 | NM_025214 | 6.00 | 35.74 | 22.10 | 0.62 | 6.00 | 90.64 | 116.81 | |
| 214971_s_at | 6480 | ST6GAL1 | AV695711 | 37.97 | 81.03 | 50.12 | 0.62 | 37.97 | 22.60 | 86.03 | |
| 203233_at | 3566 | IL4R | NM_000418 | 279.19 | 505.68 | 312.95 | 0.62 | 279.19 | 842.42 | 452.06 | 0.54 |
| 205473_at | 525 | ATP6V1B1 | NM_001692 | 29.82 | 46.35 | 28.69 | 0.62 | 29.82 | 41.51 | 11.88 | |
| 210461_s_at | 3983 | ABLIM1 | BC002448 | 9.24 | 30.29 | 18.75 | 0.62 | 9.24 | 18.80 | 22.94 | |
| 215121_x_at | 28786 | IGL@ | AA680302 | 50.69 | 83.03 | 51.46 | 0.62 | 50.69 | 61.94 | 46.91 | |
| | // | /// | | | | | | | | | |
| | | IGLV4- | | | | | | | | | |
| | | 3 /// | | | | | | | | | |
| | | IGLV3- | | | | | | | | | |
| | | 25 | | | | | | | | | |
| 212201_at | 23141 | KIAA0692 | AW274877 | 463.04 | 714.47 | 443.48 | 0.62 | 463.04 | 913.05 | 1097.30 | |
| 216793_x_at | | | AK026856 | 12.86 | 21.40 | 13.28 | 0.62 | 12.86 | 8.43 | 23.38 | |
| 210925_at | 4261 | CIITA | U18288 | 18.82 | 59.46 | 36.91 | 0.62 | 18.82 | 42.50 | 30.85 | 0.73 |
| 220229_s_at | 23431 | AP4E1 | AB030653 | 24.81 | 54.42 | 33.79 | 0.62 | 24.81 | 23.25 | 38.14 | |
| 206118_at | 6775 | STAT4 | NM_003151 | 84.34 | 383.05 | 238.05 | 0.62 | 84.34 | 5306.00 | 3824.38 | 0.72 |
| 221028_s_at | 81577 | GFOD2 | NM_030819 | 79.24 | 122.03 | 75.85 | 0.62 | 79.24 | 100.55 | 74.12 | |
| 202865_at | 54788 | DNAJB12 | AI695173 | 47.05 | 73.85 | 45.91 | 0.62 | 47.05 | 70.06 | 72.33 | |
| 205947_s_at | 7434 | VIPR2 | NM_003382 | 6.25 | 28.43 | 17.68 | 0.62 | 6.25 | 17.85 | 23.55 | |
| 207976_at | 23276 | KLHL18 | NM_025010 | 11.95 | 62.85 | 39.10 | 0.62 | 11.95 | 36.86 | 14.11 | 0.38 |
| 202734_at | 9322 | TRIP10 | NM_004240 | 188.84 | 413.86 | 257.63 | 0.62 | 188.84 | 1185.35 | 1386.27 | |
| 209084_s_at | 9364 | RAB28 | BE504689 | 204.05 | 475.92 | 296.32 | 0.62 | 204.05 | 172.88 | 340.37 | |
| 207379_at | 10085 | EDIL3 | NM_005711 | 25.90 | 47.37 | 29.50 | 0.62 | 25.90 | 11.98 | 20.84 | |
| 208520_at | 26532 | OR10H3 | NM_013938 | 13.74 | 31.43 | 19.57 | 0.62 | 13.74 | 9.25 | 23.60 | |
| 219672_at | 51327 | ERAF | NM_016633 | 13.47 | 21.30 | 13.27 | 0.62 | 13.47 | 47.87 | 24.96 | 0.52 |
| 222142_at | 1540 | CYLD | AK024212 | 8.90 | 28.42 | 17.71 | 0.62 | 8.90 | 30.39 | 15.98 | 0.53 |
| AFFX-r2-Ec- | | | AFFX-r2- | 6369.45 | 9725.48 | 6063.32 | 0.62 | 6369.45 | 5914.31 | 4171.27 | |
| bioD-5_at | | | Ec-bioD-5 | | | | | | | | |
| 203299_s_at | 653653 / | AP1S2 | AF251295 | 1151.83 | 1852.40 | 1155.30 | 0.62 | 1151.83 | 1843.22 | 1410.88 | |
| | | /// | | | | | | | | | |
| | | LOC653653 | | | | | | | | | |
| | | /// | | | | | | | | | |
| | | LOC654 | | | | | | | | | |
| 213135_at | 7074 | TIAM1 | U90902 | 6.65 | 10.46 | 6.53 | 0.62 | 6.65 | 25.05 | 5.00 | 0.20 |
| 215029_at | | | AL117451 | 136.94 | 264.93 | 165.28 | 0.62 | 136.94 | 137.77 | 195.80 | |
| 216368_s_at | 1285 | COL4A3 | U02520 | 18.57 | 39.49 | 24.65 | 0.62 | 18.57 | 21.42 | 21.89 | |
| 215006_at | 2146 | EZH2 | AK023816 | 161.19 | 356.14 | 222.37 | 0.62 | 161.19 | 294.09 | 224.74 | |
| 204858_s_at | 1890 | ECGF1 | NM_001953 | 164.15 | 328.49 | 205.11 | 0.62 | 164.15 | 220.27 | 145.54 | |
| 35150_at | 958 | CD40 | X60592 | 367.39 | 1633.92 | 1020.81 | 0.62 | 367.39 | 2347.53 | 2383.94 | |
| 217556_at | | | W26966 | 15.92 | 35.28 | 22.05 | 0.62 | 15.92 | 12.87 | 7.59 | |
| 219011_at | 57664 | PLEKHA4 | NM_020904 | 5.33 | 12.80 | 8.00 | 0.63 | 5.33 | 10.41 | 3.98 | 0.38 |
| 206608_s_at | 57096 | RPGRIP1 | NM_020366 | 19.76 | 58.99 | 36.88 | 0.63 | 19.76 | 34.04 | 60.21 | |
| 211619_s_at | 250 /// | ALPP | M13077 | 11.13 | 21.72 | 13.58 | 0.63 | 11.13 | 17.16 | 26.41 | |
| | | /// | | | | | | | | | |
| | | ALPPL2 | | | | | | | | | |
| 221397_at | 50839 | TAS2R10 | NM_023921 | 15.51 | 33.66 | 21.06 | 0.63 | 15.51 | 20.38 | 3.36 | |
| 204881_s_at | 7357 | UGCG | NM_003358 | 577.92 | 1184.67 | 741.69 | 0.63 | 577.92 | 2246.79 | 1180.95 | 0.53 |
| 205023_at | 5888 | RAD51 | D14134 | 77.01 | 115.82 | 72.58 | 0.63 | 77.01 | 84.63 | 52.56 | |
| 208365_s_at | 2868 | GRK4 | NM_005307 | 8.06 | 21.42 | 13.43 | 0.63 | 8.06 | 29.13 | 31.82 | |
| 220389_at | 60494 | CCDC81 | NM_021827 | 7.68 | 42.33 | 26.54 | 0.63 | 7.68 | 26.54 | 28.73 | |
| 201789_at | 51635 | DHRS7 | BC000637 | 1.18 | 19.32 | 12.12 | 0.63 | 1.18 | 12.37 | 9.44 | |
| 208919_s_at | 65220 | NADK | BC001709 | 900.45 | 1413.99 | 886.93 | 0.63 | 900.45 | 924.31 | 516.42 | |
| 212583_at | 9716 | AQR | AB011132 | 101.92 | 153.15 | 96.07 | 0.63 | 101.92 | 124.34 | 99.96 | |
| 201203_s_at | 6238 | RRBP1 | NM_004587 | 68.61 | 127.48 | 80.08 | 0.63 | 68.61 | 44.36 | 76.03 | |
| 221466_at | 5030 | P2RY4 | NM_002565 | 33.22 | 52.02 | 32.69 | 0.63 | 33.22 | 47.60 | 21.74 | |
| AFFX-r2-Ec- | | | AFFX-r2- | 7130.20 | 11037.18 | 6938.94 | 0.63 | 7130.20 | 7113.06 | 4857.27 | |
| bioD-3_at | | | Ec-bioD-3 | | | | | | | | |
| 213825_at | 10215 | OLIG2 | AA757419 | 177.42 | 397.25 | 250.20 | 0.63 | 177.42 | 647.08 | 471.46 | 0.73 |
| 213247_at | 79987 | SVEP1 | AA716107 | 19.38 | 35.31 | 22.27 | 0.63 | 19.38 | 27.66 | 19.37 | |
| 219046_s_at | 63876 | PKNOX2 | NM_022062 | 12.30 | 25.09 | 15.83 | 0.63 | 12.30 | 29.64 | 12.87 | 0.43 |
| 205847_at | 64063 | PRSS22 | NM_022119 | 77.26 | 124.77 | 78.79 | 0.63 | 77.26 | 84.60 | 19.03 | |
| 206828_at | 7294 | TXK | NM_003328 | 58.25 | 93.71 | 59.24 | 0.63 | 58.25 | 88.35 | 70.68 | |
| 211222_s_at | 9001 | HAP1 | AF040723 | 19.73 | 37.72 | 23.85 | 0.63 | 19.73 | 28.98 | 8.75 | |
| 201124_at | 3693 | ITGB5 | AL048423 | 28.88 | 45.90 | 29.02 | 0.63 | 28.88 | 19.91 | 11.77 | |
| 221447_s_at | 83468 | GLT8D2 | NM_031302 | 6.94 | 10.87 | 6.87 | 0.63 | 6.94 | 20.95 | 30.40 | |
| 212404_s_at | 89910 | UBE3B | AL096740 | 127.44 | 252.10 | 159.49 | 0.63 | 127.44 | 82.31 | 117.26 | |
| 210748_at | | | AF116696 | 19.49 | 39.78 | 25.17 | 0.63 | 19.49 | 50.46 | 7.42 | 0.15 |
| 210735_s_at | 771 | CA12 | BC000278 | 37.66 | 57.90 | 36.65 | 0.63 | 37.66 | 25.17 | 40.37 | |
| 204685_s_at | 491 | ATP2B2 | R52647 | 17.49 | 33.62 | 21.29 | 0.63 | 17.49 | 33.78 | 15.47 | 0.46 |
| 201251_at | 5315 | PKM2 | NM_002654 | 4116.97 | 6968.84 | 4411.92 | 0.63 | 4116.97 | 8731.66 | 7064.40 | |
| 204844_at | 2028 | ENPEP | L12468 | 7.27 | 19.46 | 12.34 | 0.63 | 7.27 | 7.10 | 6.45 | |
| 219442_at | 79014 | C16orf67 | NM_024048 | 130.89 | 342.36 | 217.13 | 0.63 | 130.89 | 216.35 | 88.77 | 0.41 |
| 208544_at | 151 | ADRA2B | NM_000682 | 10.78 | 28.32 | 17.97 | 0.63 | 10.78 | 22.18 | 29.97 | |
| 210754_s_at | 4067 | LYN | M79321 | 1603.33 | 2731.93 | 1735.29 | 0.64 | 1603.33 | 4974.01 | 7894.67 | |
| 217209_at | | | X16454 | 33.99 | 52.11 | 33.13 | 0.64 | 33.99 | 30.09 | 37.82 | |
| 220867_s_at | 25769 | SLC24A2 | NM_020344 | 12.47 | 29.90 | 19.03 | 0.64 | 12.47 | 20.15 | 22.04 | |
| 204101_at | 4534 | MTM1 | NM_000252 | 29.95 | 84.37 | 53.71 | 0.64 | 29.95 | 52.62 | 63.75 | |
| 207516_at | 1143 | CHRNB4 | NM_000750 | 7.30 | 32.53 | 20.71 | 0.64 | 7.30 | 19.23 | 23.79 | |
| 221086_s_at | 55079 | FEZF2 | NM_018008 | 2.39 | 11.54 | 7.35 | 0.64 | 2.39 | 6.01 | 6.06 | |
| 212294_at | 55970 | GNG12 | BG111761 | 49.62 | 100.69 | 64.17 | 0.64 | 49.62 | 199.96 | 85.64 | 0.43 |
| 212707_s_at | 10156 | RASA4 | AI738591 | 167.72 | 381.62 | 243.36 | 0.64 | 167.72 | 354.83 | 261.21 | 0.74 |
| | // | /// | | | | | | | | | |
| | | FLJ21767 | | | | | | | | | |
| 221412_at | 57191 | VN1R1 | NM_020633 | 44.07 | 84.16 | 53.67 | 0.64 | 44.07 | 23.25 | 25.14 | |
| 210669_at | 7020 | TFAP2A | M61156 | 15.56 | 27.33 | 17.43 | 0.64 | 15.56 | 37.30 | 28.93 | |
| 201015_s_at | 3728 | JUP | NM_021991 | 70.44 | 168.05 | 107.21 | 0.64 | 70.44 | 57.99 | 43.14 | |
| 208438_s_at | 2268 | FGR | NM_005248 | 345.06 | 645.11 | 411.87 | 0.64 | 345.06 | 1087.42 | 708.01 | 0.65 |
| 215377_at | 1488 | CTBP2 | AK024129 | 25.33 | 58.74 | 37.50 | 0.64 | 25.33 | 30.86 | 35.84 | |
| 215825_at | | | AF070579 | 39.51 | 80.54 | 51.45 | 0.64 | 39.51 | 51.44 | 49.85 | |
| 220732_at | 80243 | DEPDC2 | NM_025170 | 15.45 | 29.50 | 18.85 | 0.64 | 15.45 | 13.48 | 23.18 | |
| 212365_at | 4430 | MYO1B | BF215996 | 76.00 | 116.31 | 74.35 | 0.64 | 76.00 | 428.19 | 521.05 | |
| 213143_at | 257407 | LOC257407 | BE856707 | 10.40 | 42.99 | 27.49 | 0.64 | 10.40 | 17.10 | 49.55 | |
| 207329_at | 4317 | MMP8 | NM_002424 | 7.50 | 14.80 | 9.46 | 0.64 | 7.50 | 22.29 | 22.05 | |
| 34187_at | 5939 | RBMS2 | D28483 | 26.05 | 66.50 | 42.54 | 0.64 | 26.05 | 66.53 | 78.57 | |
| 203902_at | 9843 | HEPH | AU148222 | 6.49 | 16.97 | 10.86 | 0.64 | 6.49 | 4.22 | 5.90 | |
| 206004_at | 7053 | TGM3 | NM_003245 | 20.39 | 53.13 | 34.04 | 0.64 | 20.39 | 43.49 | 36.15 | |
| 218292_s_at | 51422 | PRKAG2 | AF087875 | 193.43 | 294.73 | 188.90 | 0.64 | 193.43 | 189.82 | 330.38 | |
| 216112_at | 5586 | PKN2 | AU157200 | 121.57 | 194.69 | 124.82 | 0.64 | 121.57 | 121.46 | 87.33 | |
| 203961_at | 10529 | NEBL | AL157398 | 46.52 | 87.50 | 56.10 | 0.64 | 46.52 | 84.83 | 44.14 | 0.52 |
| 202430_s_at | 5359 | PLSCR1 | NM_021105 | 273.08 | 513.90 | 329.56 | 0.64 | 273.08 | 845.17 | 326.33 | 0.39 |
| 216004_s_at | 5316 | PKNOX1 | AP001748 | 31.67 | 59.63 | 38.26 | 0.64 | 31.67 | 24.52 | 48.07 | |
| 212617_at | 23060 | ZNF609 | AB002293 | 33.73 | 83.85 | 53.80 | 0.64 | 33.73 | 74.90 | 57.01 | |
| 206283_s_at | 6886 | TAL1 | NM_003189 | 21.49 | 34.84 | 22.36 | 0.64 | 21.49 | 30.31 | 32.34 | |
| 220407_s_at | 7042 | TGFB2 | NM_003238 | 5.47 | 37.06 | 23.80 | 0.64 | 5.47 | 9.87 | 7.58 | |
| 209156_s_at | 1292 | COL6A2 | AY029208 | 217.33 | 433.74 | 278.89 | 0.64 | 217.33 | 1081.05 | 796.10 | 0.74 |
| 205518_s_at | 8418 | CMAH | NM_003570 | 22.59 | 38.51 | 24.78 | 0.64 | 22.59 | 7.27 | 19.58 | |
| 205870_at | 624 | BDKRB2 | NM_000623 | 19.92 | 40.48 | 26.06 | 0.64 | 19.92 | 86.27 | 116.82 | |
| 203399_x_at | 5671 | PSG3 | NM_021016 | 7.60 | 17.11 | 11.02 | 0.64 | 7.60 | 2.11 | 7.82 | |
| 219647_at | 64091 | POPDC2 | NM_022135 | 22.61 | 52.03 | 33.52 | 0.64 | 22.61 | 47.62 | 69.30 | |
| 215805_at | | | BF574664 | 9.76 | 29.21 | 18.83 | 0.64 | 9.76 | 4.86 | 10.20 | |
| 215713_at | 9839 | ZFHX1B | AK026778 | 4.56 | 38.66 | 24.93 | 0.64 | 4.56 | 26.10 | 14.02 | 0.54 |
| 212660_at | 23338 | PHF15 | AI735639 | 576.96 | 1009.94 | 651.54 | 0.65 | 576.96 | 2432.97 | 977.41 | 0.40 |
| 220977_x_at | 57669 | EPB41L5 | NM_020909 | 36.16 | 67.69 | 43.67 | 0.65 | 36.16 | 43.84 | 27.97 | |
| 215462_at | 1263 | PLK3 | AI978990 | 72.42 | 120.29 | 77.62 | 0.65 | 72.42 | 126.22 | 142.78 | |
| 221901_at | 85352 | KIAA1644 | BF516072 | 89.67 | 184.24 | 119.00 | 0.65 | 89.67 | 139.05 | 142.26 | |
| 216531_at | 404281 | YY2 | U73479 | 56.91 | 95.22 | 61.53 | 0.65 | 56.91 | 83.46 | 42.28 | |
| 209304_x_at | 4616 | GADD45B | AF087853 | 272.11 | 534.74 | 345.60 | 0.65 | 272.11 | 703.61 | 1002.36 | |
| 217322_x_at | | | AL024509 | 80.30 | 180.68 | 116.85 | 0.65 | 80.30 | 85.42 | 125.58 | |
| 210029_at | 3620 | INDO | M34455 | 19.57 | 45.50 | 29.43 | 0.65 | 19.57 | 149.19 | 74.08 | 0.50 |
| 207820_at | 124 | ADH1A | NM_000667 | 4.69 | 19.47 | 12.60 | 0.65 | 4.69 | 20.26 | 10.12 | 0.50 |
| 215761_at | 23312 | DMXL2 | AK000156 | 57.79 | 174.51 | 112.94 | 0.65 | 57.79 | 111.22 | 108.10 | |
| 32088_at | 8548 | BLZF1 | U79751 | 78.47 | 129.18 | 83.62 | 0.65 | 78.47 | 169.25 | 98.06 | 0.58 |
| 214272_at | 1540 | CYLD | AI362018 | 9.15 | 81.94 | 53.05 | 0.65 | 9.15 | 21.09 | 53.43 | |
| 221695_s_at | 10746 | MAP3K2 | AF239798 | 204.53 | 349.71 | 226.49 | 0.65 | 204.53 | 205.21 | 279.05 | |
| 205896_at | 6583 | SLC22A4 | NM_003059 | 102.96 | 162.72 | 105.48 | 0.65 | 102.96 | 147.16 | 219.11 | |
| 217237_at | 23090 | ZNF423 | Y10615 | 7.58 | 15.55 | 10.08 | 0.65 | 7.58 | 6.09 | 5.46 | |
| 215755_at | 51149 | LOC51149 | AK022006 | 28.89 | 47.14 | 30.58 | 0.65 | 28.89 | 34.06 | 36.84 | |
| 210193_at | 4336 | MOBP | D28114 | 13.75 | 29.91 | 19.41 | 0.65 | 13.75 | 27.28 | 19.52 | 0.72 |
| 220586_at | 80205 | CHD9 | NM_025134 | 34.86 | 90.77 | 58.91 | 0.65 | 34.86 | 45.65 | 37.24 | |
| 215511_at | 6942 | TCF20 | U19345 | 40.77 | 68.61 | 44.55 | 0.65 | 40.77 | 61.34 | 40.04 | 0.65 |
| 206910_x_at | 3080 | CFHR2 | NM_005666 | 21.31 | 51.41 | 33.39 | 0.65 | 21.31 | 55.07 | 38.04 | 0.69 |
| 216474_x_at | 7177 | TPSAB1 | AF206667 | 46.42 | 103.87 | 67.49 | 0.65 | 46.42 | 47.42 | 85.79 | |
| 220826_at | 55264 | C21orf77 | NM_018277 | 23.39 | 40.19 | 26.12 | 0.65 | 23.39 | 30.92 | 28.53 | |
| 212655_at | 23174 | ZCCHC14 | AB011151 | 541.29 | 811.99 | 527.88 | 0.65 | 541.29 | 720.52 | 826.97 | |
| 207851_s_at | 3643 | INSR | NM_000208 | 5.86 | 18.96 | 12.33 | 0.65 | 5.86 | 24.04 | 11.25 | 0.47 |
| 219593_at | 51296 | SLC15A3 | NM_016582 | 128.93 | 261.56 | 170.14 | 0.65 | 128.93 | 216.13 | 125.41 | 0.58 |
| 205727_at | 7011 | TEP1 | NM_007110 | 87.10 | 154.59 | 100.57 | 0.65 | 87.10 | 32.07 | 39.01 | |
| 206036_s_at | 5966 | REL | NM_002908 | 288.39 | 572.98 | 373.04 | 0.65 | 288.39 | 3020.79 | 3918.11 | |
| 216888_at | 11155 | LDB3 | AJ133768 | 4.21 | 15.48 | 10.08 | 0.65 | 4.21 | 29.68 | 19.46 | 0.66 |
| 216373_at | 202018 | FLJ90013 | AF189251 | 31.94 | 49.50 | 32.24 | 0.65 | 31.94 | 41.00 | 28.04 | |
| 214972_at | 10724 | MGEA5 | AU144791 | 50.75 | 92.11 | 60.03 | 0.65 | 50.75 | 41.31 | 51.48 | |
| 222284_at | 23094 | SIPA1L3 | AI734111 | 27.78 | 95.64 | 62.35 | 0.65 | 27.78 | 45.75 | 38.47 | |
| 214357_at | 92346 | C1orf105 | AL035295 | 9.21 | 39.89 | 26.05 | 0.65 | 9.21 | 43.65 | 41.23 | |
| 222281_s_at | | | AW517716 | 6.67 | 39.48 | 25.78 | 0.65 | 6.67 | 45.09 | 4.33 | 0.10 |
| 207009_at | 8929 | PHOX2B | NM_003924 | 11.20 | 34.54 | 22.56 | 0.65 | 11.20 | 30.64 | 3.63 | 0.12 |
| 207752_x_at | 5542 /// | PRB1 | NM_005039 | 13.83 | 28.05 | 18.32 | 0.65 | 13.83 | 28.25 | 10.97 | 0.39 |
| | | /// | | | | | | | | | |
| | | PRB2 | | | | | | | | | |
| 214066_x_at | 4882 | NPR2 | AA565715 | 24.25 | 36.88 | 24.10 | 0.65 | 24.25 | 30.60 | 22.81 | |
| 203320_at | 10019 | SH2B3 | NM_005475 | 708.53 | 2437.96 | 1593.10 | 0.65 | 708.53 | 3672.37 | 2311.01 | 0.63 |
| 217702_at | 9466 | IL27RA | AW295066 | 5.75 | 10.47 | 6.85 | 0.65 | 5.75 | 5.86 | 11.24 | |
| 215637_at | 95681 | TSGA14 | AU155621 | 12.04 | 25.20 | 16.48 | 0.65 | 12.04 | 5.22 | 9.44 | |
| 207062_at | 3375 | IAPP | NM_000415 | 7.22 | 28.80 | 18.84 | 0.65 | 7.22 | 23.04 | 13.54 | 0.59 |
| 214844_s_at | 55816 | DOK5 | AL050069 | 12.45 | 28.42 | 18.59 | 0.65 | 12.45 | 35.85 | 15.47 | 0.43 |
| 217019_at | | | AL137162 | 64.66 | 129.60 | 84.81 | 0.65 | 64.66 | 49.61 | 77.68 | |
| AFFX-r2-Ec- | | | AFFX-r2- | 764.61 | 1149.20 | 752.18 | 0.65 | 764.61 | 752.17 | 499.17 | |
| bioB-5_at | | | Ec-bioB-5 | | | | | | | | |
| 220931_at | 79024 | MGC5590 | NM_024058 | 15.69 | 39.04 | 25.56 | 0.65 | 15.69 | 19.40 | 22.77 | |
| 204727_at | 11169 | WDHD1 | AW772140 | 40.69 | 71.36 | 46.75 | 0.66 | 40.69 | 57.06 | 52.92 | |
| 216679_at | | | AL137624 | 9.42 | 18.12 | 11.88 | 0.66 | 9.42 | 23.07 | 19.74 | |
| 220909_at | 80128 | TRIM46 | NM_025058 | 35.35 | 54.50 | 35.76 | 0.66 | 35.35 | 3.32 | 3.51 | |
| 206169_x_at | 23264 | ZC3H7B | NM_025013 | 115.23 | 248.26 | 163.09 | 0.66 | 115.23 | 148.70 | 109.75 | |
| 212124_at | 57178 | ZMIZ1 | AF070622 | 681.19 | 1160.43 | 762.78 | 0.66 | 681.19 | 2326.64 | 1756.20 | |
| 209636_at | 4791 | NFKB2 | BC002844 | 34.99 | 322.88 | 212.26 | 0.66 | 34.99 | 617.38 | 671.08 | |
| 202626_s_at | 4067 | LYN | AI356412 | 1438.88 | 2329.78 | 1531.95 | 0.66 | 1438.88 | 5562.31 | 7279.54 | |
| 201529_s_at | 6117 | RPA1 | NM_002945 | 565.07 | 906.18 | 595.97 | 0.66 | 565.07 | 370.61 | 443.33 | |
| 207056_s_at | 9498 | SLC4A8 | NM_004858 | 11.63 | 30.36 | 19.98 | 0.66 | 11.63 | 19.99 | 14.94 | 0.75 |
| 218815_s_at | 55092 | TMEM51 | NM_018022 | 263.16 | 447.68 | 294.72 | 0.66 | 263.16 | 363.55 | 428.74 | |
| 220310_at | 79861 | TUBAL3 | NM_024803 | 7.45 | 28.67 | 18.88 | 0.66 | 7.45 | 11.66 | 2.19 | 0.19 |
| AFFX-r2-P1- | | | AFFX-r2- | 17179.16 | 26443.23 | 17413.63 | 0.66 | 17179.16 | 15993.52 | 12055.68 | |
| cre-5_at | | | P1-cre-5 | | | | | | | | |
| 216437_at | 80314 | EPC1 | AK024949 | 6.46 | 31.89 | 21.01 | 0.66 | 6.46 | 23.12 | 20.84 | |
| 202504_at | 23650 | TRIM29 | NM_012101 | 15.56 | 35.32 | 23.26 | 0.66 | 15.56 | 13.39 | 20.47 | |
| 217509_x_at | 2901 | GRIK5 | BG151527 | 46.33 | 121.35 | 79.94 | 0.66 | 46.33 | 67.38 | 76.62 | |
| 203927_at | 4794 | NFKB1E | NM_004556 | 382.24 | 2087.41 | 1375.43 | 0.66 | 382.24 | 2383.78 | 1855.26 | |
| AFFX-CreX- | | | AFFX- | 14271.66 | 21688.15 | 14293.15 | 0.66 | 14271.66 | 13842.55 | 9433.75 | |
| 5_at | | | CreX-5 | | | | | | | | |
| 220269_at | 79740 | FLJ23049 | NM_024687 | 2.30 | 20.39 | 13.45 | 0.66 | 2.30 | 78.87 | 63.00 | |
| 221875_x_at | 3134 | HLA-F | AW514210 | 1488.70 | 2288.67 | 1509.87 | 0.66 | 1488.70 | 1369.52 | 1841.85 | |
| 218835_at | 6436 /// | SFTPA2 | NM_006926 | 24.85 | 41.58 | 27.43 | 0.66 | 24.85 | 11.62 | 35.79 | |
| | | /// | | | | | | | | | |
| | | LOC729238 | | | | | | | | | |
| 205465_x_at | 9957 | HS3ST1 | BF000296 | 6.96 | 11.78 | 7.78 | 0.66 | 6.96 | 7.72 | 7.20 | |
| 215531_s_at | 2558 /// | GABRA5 | BF966183 | 9.23 | 19.41 | 12.81 | 0.66 | 9.23 | 7.65 | 7.03 | |
| | | /// | | | | | | | | | |
| | | LOC727729 | | | | | | | | | |
| 201884_at | 1048 | CEACAM5 | NM_004363 | 9.75 | 39.13 | 25.85 | 0.66 | 9.75 | 32.73 | 25.97 | |
| 201108_s_at | 7057 | THBS1 | NM_003246 | 13.53 | 78.51 | 51.89 | 0.66 | 13.53 | 286.55 | 163.79 | 0.57 |
| 204790_at | 4092 | SMAD7 | NM_005904 | 163.77 | 490.09 | 323.93 | 0.66 | 163.77 | 450.51 | 673.61 | |
| 215536_at | 3120 | HLA- | X87344 | 12.47 | 40.20 | 26.60 | 0.66 | 12.47 | 25.79 | 44.07 | |
| | | DQB2 | | | | | | | | | |
| 212200_at | 23141 | KIAA0692 | AW274877 | 305.95 | 515.18 | 340.98 | 0.66 | 305.95 | 770.21 | 724.43 | |
| 200777_s_at | 151579 / | BZW1 | NM_014670 | 2959.96 | 4701.01 | 3113.34 | 0.66 | 2959.96 | 5836.96 | 5351.54 | |
| | | /// | | | | | | | | | |
| | | LOC151579 | | | | | | | | | |
| 216638_s_at | 5618 /// | PRLR | S78505 | 17.20 | 28.84 | 19.10 | 0.66 | 17.20 | 23.11 | 6.20 | |
| | | /// | | | | | | | | | |
| | | CLDN1 | | | | | | | | | |
| 201502_s_at | 4792 | NFKBIA | NM_020529 | 1349.53 | 15293.14 | 10129.70 | 0.66 | 1349.53 | 10601.20 | 11863.66 | |
| 215529_x_at | 23181 | DIP2A | AI590053 | 298.88 | 563.23 | 373.18 | 0.66 | 298.88 | 449.03 | 401.69 | |
| 210623_at | 51035 | LOC51035 | BC001372 | 41.58 | 77.67 | 51.47 | 0.66 | 41.58 | 53.36 | 64.66 | |
| 217662_x_at | 55973 | BCAP29 | AI393960 | 59.25 | 148.94 | 98.72 | 0.66 | 59.25 | 178.48 | 83.81 | 0.47 |
| 215156_at | 80349 | WDR61 | AL109709 | 69.90 | 119.82 | 79.49 | 0.66 | 69.90 | 68.35 | 110.86 | |
| 212658_at | 10184 | LHFPL2 | N66633 | 124.37 | 207.60 | 137.75 | 0.66 | 124.37 | 142.95 | 178.91 | |
| 221181_at | 642452 / | LOC642452 | NM_025062 | 8.40 | 12.78 | 8.48 | 0.66 | 8.40 | 5.53 | 6.84 | |
| | | /// | | | | | | | | | |
| | | LOC651791 | | | | | | | | | |
| 205802_at | 7220 | TRPC1 | NM_003304 | 5.78 | 15.55 | 10.32 | 0.66 | 5.78 | 11.44 | 32.69 | |
| 211122_s_at | 6373 | CXCL11 | AF002985 | 3.85 | 345.79 | 229.58 | 0.66 | 3.85 | 238.41 | 69.88 | 0.29 |
| 205964_at | 79088 | ZNF426 | NM_024106 | 111.74 | 170.09 | 112.94 | 0.66 | 111.74 | 120.12 | 151.55 | |
| 216512_s_at | 1638 | DCT | AL139318 | 14.23 | 40.62 | 26.98 | 0.66 | 14.23 | 17.37 | 20.91 | |
| 210151_s_at | 8444 | DYRK3 | AF186773 | 8.17 | 28.18 | 18.73 | 0.66 | 8.17 | 62.20 | 29.79 | 0.48 |
| 206322_at | 8224 | SYN3 | NM_003490 | 24.27 | 38.25 | 25.44 | 0.66 | 24.27 | 7.80 | 8.83 | |
| 217628_at | 53405 | CLIC5 | BF032808 | 8.09 | 22.63 | 15.05 | 0.66 | 8.09 | 35.52 | 35.14 | |
| 216250_s_at | 9404 | LPXN | X77598 | 383.36 | 687.48 | 457.32 | 0.67 | 383.36 | 3109.00 | 837.35 | 0.27 |
| 206306_at | 6263 | RYR3 | NM_001036 | 7.70 | 16.97 | 11.29 | 0.67 | 7.70 | 11.05 | 9.63 | |
| 211883_x_at | 634 | CEACAM1 | M76742 | 59.09 | 94.50 | 62.87 | 0.67 | 59.09 | 60.15 | 78.24 | |
| AFFX- | | | AFFX- | 404.65 | 737.51 | 490.80 | 0.67 | 404.65 | 658.49 | 737.56 | |
| M27830_5_at | | | M27830_5 | | | | | | | | |
| 201786_s_at | 103 | ADAR | NM_001111 | 4417.18 | 7250.24 | 4827.70 | 0.67 | 4417.18 | 6909.82 | 3641.86 | 0.53 |
| 220599_s_at | 79092 | CARD14 | NM_024110 | 28.08 | 49.68 | 33.08 | 0.67 | 28.08 | 39.54 | 11.72 | |
| 208221_s_at | 6585 | SLIT1 | NM_003061 | 18.40 | 32.96 | 21.97 | 0.67 | 18.40 | 4.63 | 18.65 | |
| 219615_s_at | 8645 | KCNK5 | NM_003740 | 135.71 | 205.14 | 136.73 | 0.67 | 135.71 | 450.17 | 409.57 | |
| 204793_at | 9737 | GPRASP1 | NM_014710 | 49.09 | 74.57 | 49.72 | 0.67 | 49.09 | 69.12 | 44.06 | |
| 210001_s_at | 8651 | SOCS1 | AB005043 | 46.09 | 79.23 | 52.85 | 0.67 | 46.09 | 66.05 | 49.38 | |
| 210403_s_at | 3758 | KCNJ1 | U03884 | 3.22 | 17.46 | 11.65 | 0.67 | 3.22 | 13.88 | 8.36 | 0.60 |
| 205221_at | 3081 | HGD | NM_000187 | 16.25 | 81.03 | 54.08 | 0.67 | 16.25 | 288.86 | 209.61 | 0.73 |
| 205954_at | 6258 | RXRG | NM_006917 | 6.40 | 26.99 | 18.02 | 0.67 | 6.40 | 4.34 | 4.95 | |
| 221153_s_at | | | NM_020355 | 15.01 | 22.98 | 15.35 | 0.67 | 15.01 | 3.04 | 16.28 | |
| 216277_at | 699 | BUB1 | AK023540 | 35.36 | 54.42 | 36.35 | 0.67 | 35.36 | 40.78 | 27.75 | |
| 220691_at | 259230 | TMEM23 | NM_014114 | 57.55 | 88.51 | 59.13 | 0.67 | 57.55 | 58.22 | 55.49 | |
| 208288_at | 8647 | ABCB11 | NM_003742 | 6.70 | 15.07 | 10.08 | 0.67 | 6.70 | 0.99 | 1.87 | |
| 211459_at | | | AF234262 | 18.55 | 47.38 | 31.68 | 0.67 | 18.55 | 28.50 | 22.51 | |
| 212736_at | 89927 | C16orf45 | BE299456 | 42.03 | 69.82 | 46.68 | 0.67 | 42.03 | 64.33 | 39.84 | 0.62 |
| 207115_x_at | 54799 | MBTD1 | NM_017643 | 115.01 | 215.85 | 144.37 | 0.67 | 115.01 | 104.26 | 121.70 | |
| 213870_at | 1302 | COL11A2 | AL031228 | 26.53 | 41.65 | 27.87 | 0.67 | 26.53 | 43.95 | 49.04 | |
| 53720_at | 55337 | FLJ11286 | AI862559 | 274.92 | 414.86 | 278.08 | 0.67 | 274.92 | 374.88 | 143.18 | |
| 212463_at | 966 | CD59 | BE379006 | 31.03 | 110.17 | 73.88 | 0.67 | 31.03 | 127.77 | 111.22 | |
| 211887_x_at | 4481 | MSR1 | AF037351 | 3.67 | 16.64 | 11.16 | 0.67 | 3.67 | 9.83 | 3.29 | |
| 221071_at | | | NM_021651 | 58.63 | 107.90 | 72.42 | 0.67 | 58.63 | 88.54 | 68.52 | |
| 211964_at | 1284 | COL4A2 | X05610 | 127.61 | 295.89 | 198.62 | 0.67 | 127.61 | 722.90 | 294.05 | 0.41 |
| 217077_s_at | 9568 | GABBR2 | AF095723 | 16.77 | 54.73 | 36.74 | 0.67 | 16.77 | 6.92 | 23.36 | |
| 204105_s_at | 4897 | NRCAM | NM_005010 | 5.87 | 86.82 | 58.30 | 0.67 | 5.87 | 509.88 | 780.69 | |
| 222341_x_at | 64770 | CCDC14 | AW973235 | 32.80 | 67.63 | 45.42 | 0.67 | 32.80 | 26.35 | 22.53 | |
| 215504_x_at | 55608 | ANKRD10 | AF131777 | 253.88 | 462.49 | 310.69 | 0.67 | 253.88 | 417.14 | 261.90 | 0.63 |
| 222207_x_at | 441258 | LOC441258 | AK024602 | 366.92 | 831.22 | 558.40 | 0.67 | 366.92 | 413.34 | 477.86 | |
| 209508_x_at | 8837 | CFLAR | AF005774 | 308.24 | 873.96 | 587.65 | 0.67 | 308.24 | 1200.33 | 1459.02 | |
| 207973_x_at | 56 | ACRV1 | NM_020110 | 32.88 | 66.09 | 44.47 | 0.67 | 32.88 | 45.71 | 35.70 | |
| 209655_s_at | 83604 | TMEM47 | AL136550 | 23.21 | 43.76 | 29.44 | 0.67 | 23.21 | 32.91 | 80.18 | |
| 209994_s_at | 5243 /// | ABCB1 | AF016535 | 145.68 | 251.85 | 169.48 | 0.67 | 145.68 | 132.14 | 178.21 | |
| | | /// | | | | | | | | | |
| | | ABCB4 | | | | | | | | | |
| 213389_at | 9640 | ZNF592 | BF508616 | 147.72 | 234.99 | 158.13 | 0.67 | 147.72 | 216.83 | 162.18 | |
| 206812_at | 155 | ADRB3 | NM_000025 | 15.09 | 34.26 | 23.08 | 0.67 | 15.09 | 4.60 | 15.91 | |
| 206673_at | 11245 | GPR176 | NM_007223 | 9.50 | 49.00 | 33.03 | 0.67 | 9.50 | 17.19 | 10.05 | 0.58 |
| 205676_at | 1594 | CYP27B1 | NM_000785 | 76.64 | 128.14 | 86.37 | 0.67 | 76.64 | 406.20 | 296.39 | 0.73 |
| 202597_at | 3664 | IRF6 | AU144284 | 30.92 | 66.00 | 44.51 | 0.67 | 30.92 | 47.75 | 16.65 | 0.35 |
| 219334_s_at | 64859 | OBFC2A | AU157541 | 11.78 | 40.74 | 27.48 | 0.67 | 11.78 | 425.71 | 373.06 | |
| 220458_at | 55104 | FLJ10246 | NM_018038 | 90.06 | 143.96 | 97.27 | 0.68 | 90.06 | 62.89 | 93.47 | |
| 201005_at | 928 | CD9 | NM_001769 | 235.01 | 367.10 | 248.04 | 0.68 | 235.01 | 208.11 | 245.69 | |
| 219730_at | 54797 | MED18 | NM_017638 | 54.32 | 120.86 | 81.69 | 0.68 | 54.32 | 47.50 | 51.91 | |
| 220943_s_at | 55471 | PRO1853 | NM_018607 | 55.91 | 89.90 | 60.78 | 0.68 | 55.91 | 80.23 | 56.71 | |
| 202724_s_at | 2308 | FOXO1A | NM_002015 | 56.81 | 96.81 | 65.46 | 0.68 | 56.81 | 222.43 | 81.28 | 0.37 |
| 204715_at | 24145 | PANX1 | NM_015368 | 357.33 | 551.48 | 373.20 | 0.68 | 357.33 | 661.21 | 936.84 | |
| 214851_at | 3172 | HNF4A | X87870 | 14.90 | 32.65 | 22.10 | 0.68 | 14.90 | 14.21 | 37.81 | |
| 219847_at | 79885 | HDAC11 | NM_024827 | 45.72 | 106.67 | 72.23 | 0.68 | 45.72 | 54.92 | 27.70 | |
| 208811_s_at | 10049 | DNAJB6 | AF080569 | 707.33 | 1076.06 | 728.62 | 0.68 | 707.33 | 582.11 | 1436.08 | |
| 220841_s_at | 54806 | AHI1 | NM_017651 | 62.67 | 138.62 | 93.87 | 0.68 | 62.67 | 93.44 | 82.63 | |
| 214964_at | 84629 | KIAA1856 | AA554430 | 275.73 | 463.31 | 313.77 | 0.68 | 275.73 | 179.13 | 143.34 | |
| 217382_at | 390363 / | LOC390363 | U85978 | 12.97 | 49.25 | 33.36 | 0.68 | 12.97 | 36.11 | 6.55 | 0.18 |
| | | /// | | | | | | | | | |
| | | LOC732322 | | | | | | | | | |
| 214392_at | 56269 | IRGC | AA431984 | 16.75 | 35.66 | 24.17 | 0.68 | 16.75 | 3.69 | 6.71 | |
| 214888_at | 824 | CAPN2 | AK023851 | 108.56 | 176.72 | 119.82 | 0.68 | 108.56 | 178.68 | 171.90 | |
| 211766_s_at | 5408 | PNLIPRP2 | BC005989 | 5.67 | 17.43 | 11.82 | 0.68 | 5.67 | 9.67 | 7.10 | |
| 214091_s_at | 2878 | GPX3 | AW149846 | 19.74 | 55.27 | 37.49 | 0.68 | 19.74 | 40.85 | 52.40 | |
| 206744_s_at | 9205 | ZMYM5 | NM_014242 | 31.48 | 50.75 | 34.43 | 0.68 | 31.48 | 46.77 | 39.03 | |
| 216053_x_at | 731501 | LOC731501 | AI143919 | 7.46 | 36.01 | 24.44 | 0.68 | 7.46 | 15.55 | 17.15 | |
| 39582_at | 1540 | CYLD | AL050166 | 99.81 | 306.63 | 208.15 | 0.68 | 99.81 | 254.18 | 235.07 | |
| 203395_s_at | 3280 | HES1 | NM_005524 | 18.30 | 52.79 | 35.84 | 0.68 | 18.30 | 60.77 | 81.60 | |
| 200880_at | 3301 | DNAJA1 | AL534104 | 2771.33 | 4157.01 | 2823.89 | 0.68 | 2771.33 | 3513.12 | 4162.73 | |
| 211484_s_at | 1826 | DSCAM | AF023450 | 11.59 | 19.76 | 13.43 | 0.68 | 11.59 | 22.61 | 14.12 | 0.62 |
| 217497_at | 1890 | ECGF1 | AW613387 | 141.27 | 597.75 | 406.19 | 0.68 | 141.27 | 528.61 | 247.50 | 0.47 |
| 217436_x_at | 730399 / | LOC730399 | M80469 | 1133.05 | 1809.86 | 1230.34 | 0.68 | 1133.05 | 1080.95 | 1477.65 | |
| | | /// | | | | | | | | | |
| | | LOC731974 | | | | | | | | | |
| 210286_s_at | 9497 | SLC4A7 | AF053755 | 30.70 | 58.08 | 39.48 | 0.68 | 30.70 | 28.82 | 47.62 | |
| 218345_at | 55365 | TMEM176A | NM_018487 | 52.95 | 119.25 | 81.15 | 0.68 | 52.95 | 44.91 | 53.65 | |
| 221556_at | 8555 | CDC14B | BF792631 | 227.90 | 355.10 | 241.72 | 0.68 | 227.90 | 141.47 | 178.16 | |
| 205933_at | 26040 | SETBP1 | NM_015559 | 29.98 | 47.57 | 32.39 | 0.68 | 29.98 | 76.14 | 61.58 | |
| 212817_at | 25822 | DNAJB5 | AK023253 | 42.19 | 92.38 | 62.92 | 0.68 | 42.19 | 171.62 | 254.15 | |
| 215135_at | 23549 | DNPEP | AI690583 | 22.67 | 47.55 | 32.39 | 0.68 | 22.67 | 55.90 | 41.32 | 0.74 |
| 207882_at | 55566 | HSAJ2425 | NM_017532 | 15.72 | 34.81 | 23.71 | 0.68 | 15.72 | 31.86 | 15.80 | 0.50 |
| 207031_at | 579 | BAPX1 | NM_001189 | 279.73 | 425.74 | 290.04 | 0.68 | 279.73 | 234.35 | 119.73 | |
| 216746_at | | | AK024606 | 13.21 | 33.88 | 23.08 | 0.68 | 13.21 | 17.89 | 11.68 | |
| 214818_at | 284001 | CCDC57 | AF007146 | 11.76 | 66.59 | 45.39 | 0.68 | 11.76 | 139.35 | 76.55 | 0.55 |
| 213748_at | 9866 | TRIM66 | AW271713 | 81.36 | 139.78 | 95.35 | 0.68 | 81.36 | 67.68 | 71.22 | |
| 205003_at | 9732 | DOCK4 | NM_014705 | 22.06 | 72.34 | 49.35 | 0.68 | 22.06 | 175.01 | 94.98 | 0.54 |
| 205925_s_at | 5865 | RAB3B | NM_002867 | 5.37 | 29.30 | 19.99 | 0.68 | 5.37 | 12.94 | 21.66 | |
| 220792_at | 11107 | PRDM5 | NM_018699 | 34.77 | 59.18 | 40.40 | 0.68 | 34.77 | 29.53 | 29.85 | |
| 210077_s_at | 6430 | SFRS5 | U30884 | 93.50 | 169.73 | 116.02 | 0.68 | 93.50 | 80.57 | 178.43 | |
| 217639_at | | | BF674749 | 12.20 | 27.20 | 18.59 | 0.68 | 12.20 | 31.32 | 16.76 | 0.54 |
| 222074_at | 4088 /// | SMAD3 | AW614435 | 30.64 | 55.55 | 37.98 | 0.68 | 30.64 | 42.99 | 36.35 | |
| | | /// | | | | | | | | | |
| | | UROD | | | | | | | | | |
| 215777_at | | | AW405975 | 7.08 | 18.26 | 12.48 | 0.68 | 7.08 | 21.42 | 19.79 | |
| 215849_x_at | 118491 | TTC18 | AK022235 | 56.56 | 113.07 | 77.37 | 0.68 | 56.56 | 55.85 | 71.80 | |
| 207555_s_at | 6915 | TBXA2R | U27325 | 27.10 | 70.10 | 47.99 | 0.68 | 27.10 | 15.67 | 30.22 | |
| 202132_at | 25937 | WWTR1 | AA081084 | 25.28 | 68.69 | 47.02 | 0.68 | 25.28 | 37.60 | 15.29 | |
| 221212_x_at | 55193 | PB1 | NM_018313 | 7.87 | 20.15 | 13.80 | 0.68 | 7.87 | 6.13 | 23.64 | |
| 220580_at | 80114 | BICC1 | NM_025044 | 14.04 | 38.40 | 26.31 | 0.69 | 14.04 | 34.35 | 19.74 | 0.57 |
| 201169_s_at | 8553 | BHLHB2 | BG326045 | 51.47 | 195.62 | 134.05 | 0.69 | 51.47 | 113.56 | 529.80 | |
| 208849_at | | | AF118091 | 18.84 | 35.27 | 24.19 | 0.69 | 18.84 | 25.63 | 25.23 | |
| 220221_at | 55187 | VPS13D | NM_018156 | 26.24 | 91.20 | 62.57 | 0.69 | 26.24 | 40.97 | 52.36 | |
| 213620_s_at | 3384 | ICAM2 | AA126728 | 156.89 | 254.36 | 174.67 | 0.69 | 156.89 | 105.03 | 106.01 | |
| 204760_s_at | 7067 /// | THRA | NM_021724 | 25.60 | 57.52 | 39.51 | 0.69 | 25.60 | 5.62 | 20.69 | |
| | | /// | | | | | | | | | |
| | | NR1D1 | | | | | | | | | |
| 220546_at | 79951 | FLJ11783 | NM_024891 | 29.44 | 50.10 | 34.42 | 0.69 | 29.44 | 23.99 | 33.02 | |
| 216765_at | 5607 | MAP2K5 | AK025177 | 42.07 | 85.02 | 58.42 | 0.69 | 42.07 | 28.79 | 7.33 | |
| 215066_at | 5792 | PTPRF | AU158443 | 51.32 | 86.06 | 59.18 | 0.69 | 51.32 | 77.06 | 38.82 | 0.50 |
| 214405_at | 10659 | CUGBP2 | Z39557 | 32.89 | 50.92 | 35.04 | 0.69 | 32.89 | 40.18 | 3.17 | |
| 219275_at | 9141 | PDCD5 | NM_004708 | 385.85 | 629.08 | 433.98 | 0.69 | 385.85 | 298.47 | 396.25 | |
| 39548_at | 4862 | NPAS2 | U77970 | 13.34 | 35.64 | 24.59 | 0.69 | 13.34 | 45.56 | 26.10 | 0.57 |
| 211135_x_at | 10288 | LILRB2 | AF009644 | 91.64 | 151.04 | 104.25 | 0.69 | 91.64 | 164.72 | 36.60 | 0.22 |
| | // | /// | | | | | | | | | |
| | | LILRB3 | | | | | | | | | |
| 216347_s_at | 23368 | PPP1R13B | AK023188 | 65.18 | 124.91 | 86.23 | 0.69 | 65.18 | 119.13 | 129.16 | |
| 202181_at | 9766 | KIAA0247 | NM_014734 | 735.40 | 1507.19 | 1041.22 | 0.69 | 735.40 | 1731.91 | 2576.81 | |
| 207930_at | 3933 | LCN1 | NM_002297 | 14.44 | 24.72 | 17.09 | 0.69 | 14.44 | 9.34 | 17.46 | |
| 209458_x_at | 3039 /// | HBA1 | AF105974 | 11.10 | 17.92 | 12.40 | 0.69 | 11.10 | 17.81 | 15.91 | |
| | | /// | | | | | | | | | |
| | | HBA2 | | | | | | | | | |
| 204975_at | 2013 | EMP2 | NM_001424 | 232.08 | 355.93 | 246.35 | 0.69 | 232.08 | 517.43 | 296.99 | 0.57 |
| 206107_at | 8786 | RGS11 | NM_003834 | 4.01 | 11.10 | 7.68 | 0.69 | 4.01 | 1.75 | 4.99 | |
| 214212_x_at | 10979 | PLEKHC1 | AI928241 | 19.85 | 52.50 | 36.36 | 0.69 | 19.85 | 30.61 | 45.65 | |
| 203548_s_at | 4023 | LPL | BF672975 | 461.02 | 988.96 | 685.13 | 0.69 | 461.02 | 4167.52 | 3450.99 | |
| 215898_at | 23093 | TTLL5 | AK021879 | 56.91 | 120.39 | 83.42 | 0.69 | 56.91 | 91.67 | 88.85 | |
| 204679_at | 3775 | KCNK1 | NM_002245 | 8.73 | 27.27 | 18.90 | 0.69 | 8.73 | 26.87 | 15.82 | 0.59 |
| 209967_s_at | 1390 | CREM | D14826 | 30.25 | 79.64 | 55.21 | 0.69 | 30.25 | 253.82 | 111.36 | 0.44 |
| 202153_s_at | 23636 | NUP62 | NM_016553 | 1873.97 | 2816.31 | 1952.46 | 0.69 | 1873.97 | 3418.31 | 1641.28 | 0.48 |
| 207758_at | 9647 | PPM1F | NM_025056 | 26.35 | 89.57 | 62.25 | 0.69 | 26.35 | 44.72 | 34.09 | |
| 219288_at | 57415 | C3orf14 | NM_020685 | 699.06 | 1097.64 | 763.02 | 0.70 | 699.06 | 1133.14 | 864.49 | |
| 217344_at | 2229 | FDPSL5 | AL022163 | 17.20 | 79.57 | 55.32 | 0.70 | 17.20 | 47.86 | 78.75 | |
| 211734_s_at | 2205 | FCER1A | BC00591 | 17.35 | 28.27 | 19.66 | 0.70 | 17.35 | 22.27 | 18.70 | |
| 217578_at | 7514 | XPO1 | AW576871 | 17.42 | 31.53 | 21.93 | 0.70 | 17.42 | 27.50 | 13.39 | 0.49 |
| 204116_at | 3561 | IL2RG | NM_000206 | 92.60 | 246.00 | 171.22 | 0.70 | 92.60 | 376.96 | 259.29 | 0.69 |
| 210556_at | 4775 | NFATC3 | U85430 | 77.19 | 148.23 | 103.21 | 0.70 | 77.19 | 82.31 | 90.46 | |
| 210408_s_at | 9362 | CPNE6 | AB009288 | 4.49 | 12.99 | 9.05 | 0.70 | 4.49 | 4.85 | 3.57 | |
| 214618_at | 8837 | CFLAR | AF015452 | 9.87 | 31.97 | 22.28 | 0.70 | 9.87 | 5.81 | 31.64 | |
| 214343_s_at | 222255 / | ATXN7L1 | AI017382 | 52.37 | 91.80 | 63.98 | 0.70 | 52.37 | 49.84 | 38.69 | |
| | | /// | | | | | | | | | |
| | | ATXN7L4 | | | | | | | | | |
| 205237_at | 2219 | FCN1 | NM_002003 | 3.99 | 29.59 | 20.63 | 0.70 | 3.99 | 10.70 | 14.67 | |
| 205414_s_at | 9912 | KIAA0672 | NM_014859 | 48.82 | 78.12 | 54.50 | 0.70 | 48.82 | 47.12 | 63.57 | |
| 210271_at | 4761 | NEUROD2 | AB021742 | 23.19 | 37.91 | 26.45 | 0.70 | 23.19 | 29.18 | 22.76 | |
| 202875_s_at | 5089 | PBX2 | BE397715 | 294.09 | 465.56 | 324.89 | 0.70 | 294.09 | 357.83 | 439.61 | |
| 220113_x_at | 84172 | POLR1B | NM_019014 | 256.95 | 415.65 | 290.08 | 0.70 | 256.95 | 310.41 | 253.47 | |
| 208519_x_at | 2797 | GNRH2 | NM_001501 | 24.16 | 62.16 | 43.40 | 0.70 | 24.16 | 8.30 | 22.42 | |
| 215996_at | | | AI446234 | 20.24 | 60.88 | 42.53 | 0.70 | 20.24 | 9.09 | 21.65 | |
| 204575_s_at | 4327 /// | MMP19 | NM_002429 | 40.59 | 153.74 | 107.44 | 0.70 | 40.59 | 115.04 | 142.93 | |
| | | /// | | | | | | | | | |
| | | LOC732415 | | | | | | | | | |
| 205810_s_at | 8976 | WASL | NM_003941 | 9.21 | 15.01 | 10.49 | 0.70 | 9.21 | 9.28 | 10.75 | |
| 213339_at | 57212 | KIAA0495 | AB007964 | 4.85 | 14.33 | 10.02 | 0.70 | 4.85 | 3.39 | 15.79 | |
| 222367_at | 123720 / | WHDC1 | AI921841 | 27.52 | 49.30 | 34.49 | 0.70 | 27.52 | 47.56 | 37.50 | |
| | | /// | | | | | | | | | |
| | | WHDC1L1 | | | | | | | | | |
| | | /// | | | | | | | | | |
| | | WHDC1L2 | | | | | | | | | |
| 202612_s_at | 9282 | CRSP2 | NM_004229 | 10.57 | 34.96 | 24.46 | 0.70 | 10.57 | 13.94 | 6.92 | |
| 208385_at | 10002 | NR2E3 | NM_016346 | 12.25 | 27.65 | 19.35 | 0.70 | 12.25 | 10.73 | 31.10 | |
| 211524_at | 4791 | NFKB2 | U09609 | 7.25 | 33.99 | 23.79 | 0.70 | 7.25 | 22.40 | 18.46 | |
| 202573_at | 1455 | CSNK1G2 | AL530441 | 739.18 | 1157.56 | 811.77 | 0.70 | 739.18 | 783.40 | 701.67 | |
| 219815_at | 79690 | GAL3ST4 | NM_024637 | 7.79 | 34.14 | 23.95 | 0.70 | 7.79 | 28.10 | 11.07 | 0.39 |
| 214245_at | 6208 | RPS14 | AI734124 | 38.44 | 62.38 | 43.82 | 0.70 | 38.44 | 50.88 | 69.66 | |
| 206888_s_at | 398 | ARHGDIG | NM_001176 | 43.64 | 79.24 | 55.66 | 0.70 | 43.64 | 10.95 | 46.39 | |
| 43934_at | 56834 | GPR137 | AA479495 | 103.17 | 176.44 | 123.95 | 0.70 | 103.17 | 95.46 | 82.15 | |
| 209325_s_at | 6004 | RGS16 | U94829 | 54.35 | 705.07 | 495.85 | 0.70 | 54.35 | 202.77 | 525.09 | |
| 200799_at | 3303 | HSPA1A | NM_005345 | 1731.57 | 3603.51 | 2534.45 | 0.70 | 1731.57 | 1558.32 | 3436.70 | |
| 215926_x_at | 6621 | SNAPC4 | AK023513 | 173.53 | 390.00 | 274.39 | 0.70 | 173.53 | 415.34 | 401.38 | |
| 215491_at | 4610 | MYCL1 | AI273812 | 32.29 | 82.32 | 57.93 | 0.70 | 32.29 | 46.20 | 41.30 | |
| 207979_s_at | 926 | CD8B | NM_004931 | 6.30 | 13.00 | 9.15 | 0.70 | 6.30 | 3.51 | 10.97 | |
| 203060_s_at | 9060 | PAPSS2 | NM_004670 | 173.75 | 274.97 | 193.64 | 0.70 | 173.75 | 799.55 | 281.77 | 0.35 |
| 212466_at | 200734 | SPRED2 | AW138902 | 88.45 | 137.26 | 96.72 | 0.70 | 88.45 | 141.53 | 148.36 | |
| 204687_at | 25849 | DKFZP564O0823 | NM_015393 | 67.65 | 104.66 | 73.75 | 0.70 | 67.65 | 253.99 | 331.38 | |
| 216756_at | | | AK024995 | 14.65 | 47.22 | 33.29 | 0.70 | 14.65 | 36.12 | 33.85 | |
| 207749_s_at | 5523 | PPP2R3A | NM_002718 | 108.09 | 177.74 | 125.34 | 0.71 | 108.09 | 265.00 | 162.78 | 0.61 |
| 209700_x_at | 9659 | PDE4DIP | AB042555 | 16.02 | 27.28 | 19.26 | 0.71 | 16.02 | 46.00 | 81.08 | |
| 222082_at | 51341 | ZBTB7A | AI568395 | 36.44 | 57.42 | 40.55 | 0.71 | 36.44 | 23.16 | 58.37 | |
| 202828_s_at | 4323 | MMP14 | NM_004995 | 177.23 | 397.93 | 281.14 | 0.71 | 177.23 | 123.47 | 160.18 | |
| 208526_at | 26211 | OR2F1 | NM_012369 | 3.84 | 31.67 | 22.39 | 0.71 | 3.84 | 17.23 | 11.98 | 0.70 |
| 222272_x_at | 85477 | SCIN | BG283584 | 23.03 | 62.73 | 44.46 | 0.71 | 23.03 | 42.05 | 27.60 | 0.66 |
| 214102_at | 116984 | CENTD1 | AK023737 | 105.60 | 175.15 | 124.14 | 0.71 | 105.60 | 187.22 | 152.88 | |
| 220658_s_at | 56938 | ARNTL2 | NM_020183 | 169.38 | 289.53 | 205.21 | 0.71 | 169.38 | 221.27 | 200.56 | |
| 208222_at | 91 | ACVR1B | NM_020327 | 26.84 | 51.57 | 36.58 | 0.71 | 26.84 | 36.30 | 10.73 | |
| 208729_x_at | 3106 | HLA-B | D83043 | 5128.16 | 8066.14 | 5723.93 | 0.71 | 5128.16 | 5663.19 | 7587.90 | |
| 210162_s_at | 4772 | NFATC1 | U08015 | 225.35 | 448.14 | 318.04 | 0.71 | 225.35 | 460.85 | 474.70 | |
| 213060_s_at | 1117 | CHI3L2 | U58515 | 27.54 | 47.50 | 33.71 | 0.71 | 27.54 | 36.76 | 47.84 | |
| 206818_s_at | 54805 | CNNM2 | NM_017649 | 42.84 | 77.56 | 55.06 | 0.71 | 42.84 | 69.79 | 58.98 | |
| 205707_at | 23765 | IL17RA | NM_014339 | 1085.55 | 1641.52 | 1165.83 | 0.71 | 1085.55 | 811.14 | 500.43 | |
| 209082_s_at | 80781 | COL18A1 | AF018081 | 54.12 | 104.13 | 73.97 | 0.71 | 54.12 | 72.40 | 48.81 | |
| 213503_x_at | 302 | ANXA2 | BE908217 | 2409.95 | 5924.72 | 4209.75 | 0.71 | 2409.95 | 9550.19 | 6945.62 | 0.73 |
| 206512_at | 7310 | U2AF1L1 | NM_005083 | 42.28 | 88.95 | 63.22 | 0.71 | 42.28 | 82.91 | 156.40 | |
| 211544_s_at | 2692 | GHRHR | AB058895 | 19.35 | 52.71 | 37.48 | 0.71 | 19.35 | 18.42 | 26.14 | |
| 216738_at | 3269 | HRH1 | AK024553 | 18.91 | 60.19 | 42.83 | 0.71 | 18.91 | 41.72 | 18.14 | 0.43 |
| 206023_at | 10874 | NMU | NM_006681 | 5.57 | 25.45 | 18.11 | 0.71 | 5.57 | 8.84 | 8.67 | |
| 207328_at | 246 | ALOX15 | NM_001140 | 9.50 | 14.52 | 10.33 | 0.71 | 9.50 | 8.12 | 6.93 | |
| 216662_at | 4648 | MYO7B | AK000145 | 9.69 | 48.75 | 34.70 | 0.71 | 9.69 | 19.62 | 23.19 | |
| 220027_s_at | 54922 | RASIP1 | NM_017805 | 22.59 | 38.03 | 27.10 | 0.71 | 22.59 | 10.95 | 23.04 | |
| 220762_a_at | 54584 | GNB1L | NM_022446 | 71.47 | 151.97 | 108.46 | 0.71 | 71.47 | 160.83 | 81.86 | 0.51 |
| 205479_s_at | 5328 | PLAU | NM_002658 | 328.74 | 609.31 | 435.17 | 0.71 | 328.74 | 1710.50 | 5039.60 | |
| 207644_at | 8928 | FOXH1 | NM_003923 | 20.29 | 32.44 | 23.19 | 0.71 | 20.29 | 32.93 | 7.91 | 0.24 |
| 222038_s_at | 51096 | UTP18 | AA993099 | 21.39 | 41.09 | 29.38 | 0.71 | 21.39 | 31.54 | 36.47 | |
| 211317_s_at | 8837 | CFLAR | AF041461 | 422.39 | 932.17 | 666.58 | 0.72 | 422.39 | 1157.89 | 1851.36 | |
| 210538_s_at | 330 | BIRC3 | U37546 | 30.89 | 602.11 | 430.60 | 0.72 | 30.89 | 1431.13 | 1068.78 | 0.75 |
| 217161_x_at | 176 | AGC1 | X17406 | 15.56 | 63.17 | 45.22 | 0.72 | 15.56 | 36.67 | 47.88 | |
| 210412_at | 2904 | GRIN2B | U11287 | 25.81 | 46.16 | 33.06 | 0.72 | 25.81 | 5.87 | 5.68 | |
| 214486_x_at | 8837 | CFLAR | AF041459 | 218.81 | 575.70 | 412.73 | 0.72 | 218.81 | 900.37 | 851.11 | |
| 216683_at | 6902 | TBCA | AL353949 | 44.55 | 115.59 | 82.92 | 0.72 | 44.55 | 79.97 | 117.89 | |
| 211691_x_at | 4946 | OAZ1 | AF293339 | 47.28 | 116.92 | 83.87 | 0.72 | 47.28 | 52.36 | 60.19 | |
| 202015_x_at | | | NM_006838 | 16.29 | 34.09 | 24.46 | 0.72 | 16.29 | 30.26 | 19.42 | 0.64 |
| 216182_at | 8871 | SYNJ2 | AK026758 | 3.81 | 16.23 | 11.65 | 0.72 | 3.81 | 4.79 | 4.34 | |
| 221205_at | | | NM_018041 | 122.51 | 299.38 | 215.02 | 0.72 | 122.51 | 306.62 | 68.72 | 0.22 |
| 222137_at | 54862 | CC2D1A | AK023399 | 22.45 | 38.39 | 27.59 | 0.72 | 22.45 | 20.82 | 32.54 | |
| 209999_x_at | 8651 | SOCS1 | AB005043 | 5.83 | 27.74 | 19.94 | 0.72 | 5.83 | 5.18 | 9.28 | |
| 204091_at | 5147 | PDE6D | NM_002601 | 451.23 | 703.39 | 505.73 | 0.72 | 451.23 | 443.35 | 448.02 | |
| 213665_at | 6659 | SOX4 | AI989477 | 12.12 | 23.66 | 17.01 | 0.72 | 12.12 | 57.44 | 30.21 | 0.53 |
| 210997_at | 3082 | HGF | M77227 | 4.14 | 13.85 | 9.96 | 0.72 | 4.14 | 14.31 | 12.80 | |
| 212080_at | 4297 | MLL | AV714029 | 283.66 | 448.78 | 322.84 | 0.72 | 283.66 | 381.89 | 321.51 | |
| 211875_x_at | 56106 | PCDHGA10 | AF152503 | 8.55 | 16.65 | 11.98 | 0.72 | 8.55 | 4.14 | 2.59 | |
| 214286_at | 2779 | GNAT1 | X63749 | 12.71 | 20.17 | 14.52 | 0.72 | 12.71 | 13.90 | 15.13 | |
| 221085_at | 9966 | TNFSF15 | NM_005118 | 8.80 | 20.63 | 14.85 | 0.72 | 8.80 | 24.56 | 10.05 | 0.41 |
| 209267_s_at | 64116 | SLC39A8 | AB040120 | 1635.93 | 2844.81 | 2048.19 | 0.72 | 1635.93 | 4808.74 | 3856.87 | |
| 215648_at | 23386 | NUDCD3 | AU144324 | 25.16 | 77.11 | 55.57 | 0.72 | 25.16 | 30.41 | 30.58 | |
| 217525_at | 283298 | OLFML1 | AW305097 | 5.75 | 19.19 | 13.83 | 0.72 | 5.75 | 2.08 | 8.02 | |
| 209210_s_at | 10979 | PLEKHC1 | Z24725 | 56.67 | 118.90 | 85.72 | 0.72 | 56.67 | 100.19 | 167.64 | |
| 211195_s_at | 8626 | TP73L | AF116771 | 6.25 | 19.12 | 13.79 | 0.72 | 6.25 | 22.64 | 8.11 | 0.36 |
| 215499_at | 5606 | MAP2K3 | AA780381 | 182.54 | 460.47 | 332.07 | 0.72 | 182.54 | 617.86 | 825.58 | |
| 216290_x_at | 1804 | DPP6 | AK000864 | 15.93 | 26.70 | 19.27 | 0.72 | 15.93 | 22.96 | 27.65 | |
| 206862_at | 9534 | ZNF254 | NM_004876 | 46.65 | 70.24 | 50.71 | 0.72 | 46.65 | 56.73 | 30.82 | |
| 213428_s_at | 1291 | COL6A1 | AA292373 | 438.55 | 938.69 | 677.63 | 0.72 | 438.55 | 3655.70 | 1864.75 | 0.51 |
| 215746_at | 8602 | C4orf9 | L34409 | 8.12 | 25.55 | 18.44 | 0.72 | 8.12 | 13.67 | 8.54 | 0.62 |
| 208926_at | 4758 | NEU1 | U84246 | 1604.71 | 2448.22 | 1767.75 | 0.72 | 1604.71 | 1619.31 | 3343.65 | |
| 207030_s_at | 1466 | CSRP2 | NM_001321 | 48.31 | 111.01 | 80.19 | 0.72 | 48.31 | 149.19 | 159.50 | |
| 201590_x_at | 302 | ANXA2 | NM_004039 | 2409.73 | 5747.46 | 4152.64 | 0.72 | 2409.73 | 9510.93 | 6896.18 | 0.73 |
| 207535_s_at | 4791 | NFKB2 | NM_002502 | 139.41 | 902.97 | 652.74 | 0.72 | 139.41 | 1117.81 | 1464.21 | |
| 204261_s_at | 5664 | PSEN2 | AA716657 | 50.50 | 85.16 | 61.64 | 0.72 | 50.50 | 12.15 | 36.70 | |
| 209098_s_at | 182 | JAG1 | U73936 | 35.69 | 127.71 | 92.43 | 0.72 | 35.69 | 33.00 | 89.02 | |
| 211171_s_at | 10846 | PDE10A | AB026816 | 13.95 | 35.66 | 25.81 | 0.72 | 13.95 | 18.90 | 23.73 | |
| 207232_s_at | 9666 | DZIP3 | NM_014648 | 38.22 | 105.69 | 76.53 | 0.72 | 38.22 | 18.12 | 27.74 | |
| 217377_x_at | 4916 | NTRK3 | AF041811 | 39.30 | 66.21 | 47.95 | 0.72 | 39.30 | 50.71 | 43.68 | |
| 221996_s_at | 1212 | CLTB | AW170546 | 19.37 | 42.67 | 30.90 | 0.72 | 19.37 | 40.90 | 32.15 | |
| 207825_s_at | 2692 | GHRHR | NM_000823 | 6.05 | 10.50 | 7.60 | 0.72 | 6.05 | 3.71 | 7.87 | |
| 200986_at | 710 | SERP1NG1 | NM_000062 | 15.14 | 44.75 | 32.42 | 0.72 | 15.14 | 25.12 | 24.94 | |
| 220338_at | 55103 | RALGPS2 | NM_018037 | 26.80 | 53.88 | 39.03 | 0.72 | 26.80 | 33.05 | 32.84 | |
| 211126_s_at | 1466 | CSRP2 | U46006 | 52.36 | 91.81 | 66.53 | 0.72 | 52.36 | 85.38 | 72.83 | |
| 219474_at | 79669 | C3orf52 | NM_024616 | 8.92 | 44.75 | 32.43 | 0.72 | 8.92 | 51.14 | 57.45 | |
| 215611_at | 6938 | TCF12 | AU146580 | 222.71 | 340.01 | 246.53 | 0.73 | 222.71 | 304.64 | 180.51 | |
| 209683_at | 81553 | FAM49A | AA243659 | 44.97 | 92.36 | 67.02 | 0.73 | 44.97 | 40.93 | 92.65 | |
| 210427_x_at | 302 | ANXA2 | BC001388 | 2547.05 | 6066.78 | 4402.80 | 0.73 | 2547.05 | 9806.56 | 7091.67 | 0.72 |
| 216832_at | 862 | RUNX1T1 | AF018283 | 3.98 | 32.05 | 23.26 | 0.73 | 3.98 | 8.57 | 37.80 | |
| 205192_at | 9020 | MAP3K14 | NM_003954 | 46.53 | 97.49 | 70.76 | 0.73 | 46.53 | 219.31 | 183.82 | |
| 208240_s_at | 2246 | FGF1 | NM_013394 | 17.00 | 29.36 | 21.32 | 0.73 | 17.00 | 11.33 | 31.57 | |
| 207662_at | 6899 | TBX1 | NM_005992 | 39.67 | 60.62 | 44.14 | 0.73 | 39.67 | 44.68 | 21.03 | |
| 206458_s_at | 7482 | WNT2B | NM_024494 | 5.76 | 11.09 | 8.07 | 0.73 | 5.76 | 11.49 | 10.21 | |
| 217192_s_at | 639 | PRDM1 | AL022067 | 126.87 | 342.39 | 249.38 | 0.73 | 126.87 | 779.61 | 928.47 | |
| 214617_at | 5551 | PRF1 | AI445650 | 29.24 | 71.34 | 51.97 | 0.73 | 29.24 | 73.46 | 36.46 | 0.50 |
| 202643_s_at | 7128 | TNFAIP3 | AI738896 | 312.99 | 3082.51 | 2245.80 | 0.73 | 312.99 | 3593.12 | 3504.58 | |
| 205938_at | 22843 | PPM1E | NM_014906 | 33.99 | 65.46 | 47.69 | 0.73 | 33.99 | 13.33 | 56.25 | |
| 202339_at | 8189 | SYMPK | NM_004819 | 133.93 | 242.65 | 176.90 | 0.73 | 133.93 | 111.83 | 129.42 | |
| 215577_at | 7324 | UBE2E1 | AU146791 | 180.70 | 374.26 | 272.86 | 0.73 | 180.70 | 235.88 | 267.04 | |
| 213248_at | 221362 / | LOC221362 | AL577024 | 123.28 | 284.56 | 207.53 | 0.73 | 123.28 | 135.07 | 79.46 | |
| | | /// | | | | | | | | | |
| | | LOC730101 | | | | | | | | | |
| 210831_s_at | 5733 | PTGER3 | L27489 | 12.76 | 30.14 | 21.98 | 0.73 | 12.76 | 17.53 | 27.07 | |
| 201505_at | 3912 | LAMB1 | NM_002291 | 23.77 | 38.44 | 28.08 | 0.73 | 23.77 | 208.19 | 279.87 | |
| 204888_s_at | 9148 | NEURL | AF029729 | 36.39 | 54.96 | 40.14 | 0.73 | 36.39 | 37.50 | 6.15 | |
| 211175_at | 11250 | GPR45 | U92642 | 7.68 | 15.92 | 11.63 | 0.73 | 7.68 | 6.61 | 2.47 | |
| 210717_at | | | AF116659 | 59.74 | 117.80 | 86.09 | 0.73 | 59.74 | 61.98 | 72.92 | |
| 213285_at | 161291 | TMEM30B | AV691491 | 1.17 | 25.10 | 18.35 | 0.73 | 1.17 | 21.13 | 7.88 | 0.37 |
| 219125_s_at | 55974 | RAG1AP1 | NM_018845 | 168.92 | 278.15 | 203.41 | 0.73 | 168.92 | 156.94 | 175.81 | |
| 220514_at | | | NM_018568 | 7.93 | 16.45 | 12.03 | 0.73 | 7.93 | 6.32 | 6.59 | |
| 216166_at | 399 | RHOH | AK024909 | 11.46 | 23.08 | 16.90 | 0.73 | 11.46 | 14.07 | 39.95 | |
| 216961_s_at | 219464 / | RPAIN | M69039 | 13.06 | 23.82 | 17.44 | 0.73 | 13.06 | 10.66 | 20.69 | |
| | | /// | | | | | | | | | |
| | | OR5T2 | | | | | | | | | |
| 214449_s_at | 23433 | RHOQ | NM_012249 | 206.49 | 343.30 | 251.43 | 0.73 | 206.49 | 238.15 | 330.53 | |
| 210597_x_at | 5542 /// | PRB1 | K03206 | 37.29 | 112.61 | 82.49 | 0.73 | 37.29 | 56.94 | 35.33 | 0.62 |
| | | /// | | | | | | | | | |
| | | PRB2 | | | | | | | | | |
| 208597_at | 1270 | CNTF | NM_000614 | 4.15 | 21.29 | 15.59 | 0.73 | 4.15 | 7.40 | 12.45 | |
| 215754_at | 950 | SCARB2 | AU148040 | 82.00 | 146.92 | 107.65 | 0.73 | 82.00 | 99.96 | 55.50 | |
| 212143_s_at | 3486 | IGFBP3 | BF340228 | 74.80 | 3263.19 | 2391.41 | 0.73 | 74.80 | 5049.49 | 4818.18 | |
| 214878_at | 256112 / | ZNF37A | AU118165 | 11.28 | 37.39 | 27.41 | 0.73 | 11.28 | 15.59 | 16.74 | |
| | | /// | | | | | | | | | |
| | | ZNF37B | | | | | | | | | |
| 218690_at | 8572 | PDLIM4 | NM_003687 | 15.56 | 37.51 | 27.50 | 0.73 | 15.56 | 6.25 | 15.55 | |
| 204735_at | 5141 | PDE4A | NM_006202 | 176.91 | 293.85 | 215.50 | 0.73 | 176.91 | 397.51 | 260.30 | 0.65 |
| 203962_s_at | 10529 | NEBL | NM_006393 | 55.10 | 122.55 | 89.88 | 0.73 | 55.10 | 85.79 | 58.86 | 0.69 |
| 222351_at | 5519 | PPP2R1B | AW009884 | 37.76 | 81.19 | 59.55 | 0.73 | 37.76 | 39.04 | 62.35 | |
| 209604_s_at | 2625 | GATA3 | AI796169 | 17.30 | 37.85 | 27.79 | 0.73 | 17.30 | 5.89 | 34.18 | |
| 217239_x_at | 54504 | CPVL | AF044592 | 28.25 | 45.19 | 33.19 | 0.73 | 28.25 | 17.13 | 32.54 | |
| 206140_at | 9355 | LHX2 | NM_004789 | 616.12 | 1806.76 | 1330.17 | 0.74 | 616.12 | 3969.65 | 1537.51 | 0.39 |
| 215540_at | 6955 | TRA@ | AW950865 | 9.67 | 18.26 | 13.44 | 0.74 | 9.67 | 29.82 | 12.99 | 0.44 |
| 202509_s_at | 7127 | TNFAIP2 | AI862445 | 75.77 | 308.86 | 227.45 | 0.74 | 75.77 | 298.03 | 231.05 | |
| 206380_s_at | 5199 | CFP | NM_002621 | 568.57 | 884.27 | 651.42 | 0.74 | 568.57 | 723.19 | 625.69 | |
| 211562_s_at | 25802 | LMOD1 | BC001755 | 4.98 | 12.19 | 8.98 | 0.74 | 4.98 | 2.67 | 15.51 | |
| 210701_at | 10428 | CFDP1 | D85939 | 114.69 | 264.71 | 195.15 | 0.74 | 114.69 | 136.68 | 192.28 | |
| 203125_x_at | 4891 | SLC11A2 | AF046997 | 145.10 | 241.83 | 178.30 | 0.74 | 145.10 | 293.44 | 452.97 | |
| 215139_at | 9639 | ARHGEF10 | AL137508 | 11.84 | 43.15 | 31.83 | 0.74 | 11.84 | 14.58 | 18.67 | |
| 209032_s_at | 23705 | IGSF4 | AF132811 | 45.27 | 69.45 | 51.30 | 0.74 | 45.27 | 70.64 | 71.10 | |
| 215676_at | 2972 | BRF1 | N91109 | 7.90 | 12.00 | 8.87 | 0.74 | 7.90 | 4.22 | 7.20 | |
| 204048_s_at | 9749 | PHACTR2 | AA551142 | 1150.04 | 1904.21 | 1408.09 | 0.74 | 1150.04 | 2925.25 | 2638.90 | |
| 214980_at | 7337 | UBE3A | AF037219 | 78.69 | 124.24 | 91.94 | 0.74 | 78.69 | 75.95 | 103.58 | |
| 207730_x_at | 84717 | HDGF2 | NM_017932 | 554.00 | 1070.47 | 792.80 | 0.74 | 554.00 | 634.45 | 604.72 | |
| 210911_at | 84099 | ID2B | M96843 | 6.20 | 21.77 | 16.13 | 0.74 | 6.20 | 45.87 | 35.87 | |
| 213935_at | 51099 | ABHD5 | AF007132 | 49.01 | 76.15 | 56.43 | 0.74 | 49.01 | 81.02 | 80.62 | |
| 215287_at | | | AA975427 | 127.18 | 199.13 | 147.59 | 0.74 | 127.18 | 164.54 | 134.28 | |
| 213191_at | 148022 | TICAM1 | AF070530 | 85.01 | 268.56 | 199.09 | 0.74 | 85.01 | 247.05 | 332.20 | |
| 201958_s_at | 4660 | PPP1R12B | NM_002481 | 9.53 | 27.57 | 20.46 | 0.74 | 9.53 | 3.26 | 4.25 | |
| 203915_at | 4283 | CXCL9 | NM_002416 | 4.66 | 104.97 | 77.88 | 0.74 | 4.66 | 45.12 | 45.89 | |
| 215562_at | 229964 | C1orf30 | AK00022 | 6.06 | 14.14 | 10.50 | 0.74 | 6.06 | 8.70 | 9.18 | |
| 200706_s_at | 9516 | LITAF | NM_004862 | 925.98 | 1700.53 | 1262.37 | 0.74 | 925.98 | 2016.91 | 3255.18 | |
| 202625_at | 4067 | LYN | AI356412 | 784.60 | 1348.47 | 1001.54 | 0.74 | 784.60 | 3249.61 | 4428.80 | |
| 215887_at | 11179 | ZNF277P | AK027128 | 73.49 | 116.69 | 86.72 | 0.74 | 73.49 | 113.96 | 75.56 | 0.66 |
| 219235_s_at | 65979 | PHACTR4 | NM_023923 | 423.25 | 687.48 | 511.22 | 0.74 | 423.25 | 582.92 | 293.31 | |
| 221281_at | 6714 | SRC | NM_005417 | 6.79 | 17.05 | 12.68 | 0.74 | 6.79 | 12.50 | 8.70 | 0.70 |
| 204174_at | 241 | ALOX5AP | NM_001629 | 2262.85 | 3704.92 | 2758.46 | 0.74 | 2262.85 | 1721.14 | 1584.03 | |
| 206938_at | 6716 | SRD5A2 | NM_000348 | 26.96 | 44.81 | 33.37 | 0.74 | 26.96 | 33.12 | 32.28 | |
| 210654_at | 8793 | TNFRSF10D | AF021233 | 3.46 | 11.05 | 8.23 | 0.74 | 3.46 | 12.56 | 11.86 | |
| 207694_at | 5456 | POU3F4 | NM_000307 | 8.90 | 29.11 | 21.69 | 0.75 | 8.90 | 36.13 | 21.15 | 0.59 |
| 206573_at | 3786 | KCNQ3 | NM_004519 | 60.10 | 101.32 | 75.52 | 0.75 | 60.10 | 197.20 | 185.01 | |
| 210540_s_at | 8702 | B4GALT4 | BC004523 | 70.20 | 107.49 | 80.16 | 0.75 | 70.20 | 169.52 | 215.59 | |
| 215556_at | 57035 | C1orf63 | AU158442 | 20.90 | 51.01 | 38.06 | 0.75 | 20.90 | 25.14 | 33.10 | |
| 222214_at | | | AK024988 | 314.69 | 540.49 | 403.52 | 0.75 | 314.69 | 261.90 | 333.53 | |
| 210808_s_at | 27035 | NOX1 | AF166327 | 5.40 | 35.62 | 26.64 | 0.75 | 5.40 | 33.74 | 32.98 | |
| 205603_s_at | 1730 | DIAPH2 | NM_007309 | 464.65 | 897.67 | 671.33 | 0.75 | 464.65 | 680.31 | 801.99 | |
| 204864_s_at | 3572 | IL6ST | BE856546 | 71.14 | 127.89 | 95.75 | 0.75 | 71.14 | 66.71 | 69.86 | |
| 214104_at | 23432 | GPR161 | AI703188 | 54.59 | 99.83 | 74.77 | 0.75 | 54.59 | 92.54 | 55.67 | 0.60 |
| 207795_s_at | 3824 | KLRD1 | AB009597 | 3.96 | 11.53 | 8.64 | 0.75 | 3.96 | 6.67 | 29.06 | |
| 206268_at | 10637 | LEFTY1 | NM_020997 | 7.01 | 39.24 | 29.40 | 0.75 | 7.01 | 36.79 | 23.25 | 0.63 |
| 205242_at | 10563 | CXCL13 | NM_006419 | 2.93 | 6.96 | 2.00 | | 2.93 | 1873.21 | 32.28 | 0.02 |
| 207533_at | 6346 | CCL1 | NM_002981 | 3.46 | 6.54 | 3.17 | | 3.46 | 45.96 | 1.14 | 0.02 |
| 210815_s_at | 10203 | CALCRL | U17473 | 21.24 | 6.31 | 7.18 | | 21.24 | 116.75 | 4.13 | 0.04 |
| 206526_at | 26150 | RIBC2 | NM_015653 | 36.64 | 28.66 | 7.00 | | 36.64 | 109.41 | 4.89 | 0.04 |
| 211533_at | 5156 | PDGFRA | M22734 | 1.41 | 5.14 | 8.82 | | 1.41 | 24.09 | 1.22 | 0.05 |
| 213856_at | 961 | CD47 | BG230614 | 1.53 | 8.03 | 5.38 | | 1.53 | 36.17 | 1.86 | 0.05 |
| 220649_at | | | NM_024856 | 9.33 | 13.12 | 13.43 | | 9.33 | 19.80 | 1.02 | 0.05 |
| 211064_at | 284443 | ZNF493 | BC006408 | 4.81 | 1.20 | 24.79 | | 4.81 | 20.29 | 1.10 | 0.05 |
| 215814_at | 667 | DST | AL049215 | 4.59 | 4.72 | 11.78 | | 4.59 | 21.74 | 1.28 | 0.06 |
| 207901_at | 3593 | IL12B | NM_002187 | 4.24 | 13.40 | 10.51 | | 4.24 | 1057.89 | 63.74 | 0.06 |
| 206298_at | 58504 | ARHGAP22 | NM_021226 | 2.00 | 3.11 | 1.70 | | 2.00 | 79.90 | 4.91 | 0.06 |
| 208547_at | 3018 | HIST1H2BB | NM_021062 | 9.30 | 8.17 | 7.89 | | 9.30 | 24.98 | 1.55 | 0.06 |
| 219935_at | 11096 | ADAMTS5 | NM_007038 | 11.43 | 4.56 | 12.57 | | 11.43 | 30.43 | 2.05 | 0.07 |
| 205112_at | 51196 | PLCE1 | NM_016341 | 3.30 | 6.34 | 8.80 | | 3.30 | 15.36 | 1.14 | 0.07 |
| 215420_at | 3549 | IHH | BE869172 | 9.72 | 7.00 | 9.08 | | 9.72 | 33.48 | 2.50 | 0.07 |
| 208046_at | 8359 | HIST1H4A | NM_003538 | 13.52 | 10.07 | 16.64 | | 13.52 | 28.02 | 2.12 | 0.08 |
| 221288_at | 2845 | GPR22 | NM_005295 | 26.31 | 31.34 | 28.77 | | 26.31 | 55.91 | 4.35 | 0.08 |
| 208488_s_at | 1378 | CR1 | NM_000651 | 15.88 | 28.28 | 26.11 | | 15.88 | 44.78 | 3.56 | 0.08 |
| 221175_at | 80111 | C3orf36 | NM_025041 | 8.41 | 6.91 | 13.14 | | 8.41 | 30.48 | 2.44 | 0.08 |
| 215957_at | 7321 | UBE2D1 | AV731367 | 4.33 | 8.76 | 12.54 | | 4.33 | 16.08 | 1.30 | 0.08 |
| 217020_at | 5915 | RARB | X04014 | 22.75 | 11.10 | 9.59 | | 22.75 | 39.30 | 3.24 | 0.08 |
| 219230_at | 55273 | TMEM100 | NM_018286 | 7.17 | 10.72 | 9.09 | | 7.17 | 24.12 | 2.01 | 0.08 |
| 221182_at | 80133 | C1orf129 | NM_025063 | 10.43 | 4.49 | 14.03 | | 10.43 | 15.85 | 1.33 | 0.08 |
| 207016_s_at | 8854 | ALDH1A2 | AB015228 | 2.05 | 4.47 | 4.39 | | 2.05 | 66.58 | 5.58 | 0.08 |
| 210852_s_at | 10157 | AASS | AF229180 | 8.75 | 22.62 | 22.24 | | 8.75 | 26.45 | 2.22 | 0.08 |
| 214370_at | 6279 | S100A8 | AW238654 | 97.05 | 52.09 | 34.27 | | 97.05 | 356.34 | 30.25 | 0.08 |
| 220218_at | 55064 | C9orf68 | NM_017985 | 6.12 | 2.62 | 1.64 | | 6.12 | 12.22 | 1.05 | 0.09 |
| 211238_at | 8756 | ADAM7 | AF215824 | 8.93 | 10.69 | 23.62 | | 8.93 | 30.85 | 2.65 | 0.09 |
| 207696_at | 10690 | FUT9 | NM_006581 | 1.18 | 3.07 | 1.62 | | 1.18 | 10.55 | 0.92 | 0.09 |
| 215465_at | 26154 | ABCA12 | AL080207 | 2.34 | 7.60 | 14.22 | | 2.34 | 11.92 | 1.04 | 0.09 |
| 216223_at | 1370 | CPN2 | J05158 | 7.15 | 2.87 | 8.63 | | 7.15 | 24.25 | 2.14 | 0.09 |
| 216405_at | 3956 | LGALS1 | M14087 | 9.45 | 5.65 | 8.22 | | 9.45 | 46.18 | 4.09 | 0.09 |
| 207861_at | 6367 | CCL22 | NM_002990 | 161.27 | 125.24 | 74.15 | | 161.27 | 7023.34 | 623.19 | 0.09 |
| 207615_s_at | 750 | C16orf3 | NM_001214 | 2.65 | 5.63 | 11.28 | | 2.65 | 21.92 | 1.98 | 0.09 |
| 215621_s_at | 3495 | IGHD | BG340670 | 3.63 | 5.24 | 7.05 | | 3.63 | 46.24 | 4.21 | 0.09 |
| 203393_at | 3280 | HES1 | BE973687 | 10.70 | 1.20 | 10.45 | | 10.70 | 36.00 | 3.30 | 0.09 |
| 206645_s_at | 190 | NR0B1 | NM_000475 | 12.07 | 3.16 | 1.94 | | 12.07 | 38.55 | 3.53 | 0.09 |
| 207926_at | 2814 | GP5 | NM_004488 | 12.79 | 6.12 | 7.19 | | 12.79 | 31.92 | 2.95 | 0.09 |
| 210895_s_at | 942 | CD86 | L25259 | 38.25 | 14.63 | 32.29 | | 38.25 | 96.28 | 8.90 | 0.09 |
| 206165_s_at | 9635 | CLCA2 | NM_006536 | 5.74 | 4.51 | 8.60 | | 5.74 | 11.98 | 1.13 | 0.09 |
| 216764_at | 150684 | COMMD1 | AK025191 | 11.06 | 11.57 | 3.97 | | 11.06 | 19.86 | 1.89 | 0.10 |
| 217524_x_at | | | AA018923 | 5.50 | 7.34 | 1.44 | | 5.50 | 17.40 | 1.66 | 0.10 |
| 220830_at | 50939 | IMPG2 | NM_016247 | 2.81 | 6.70 | 8.75 | | 2.81 | 25.59 | 2.44 | 0.10 |
| 207465_at | | | NM_014134 | 2.26 | 6.18 | 12.11 | | 2.26 | 20.22 | 1.94 | 0.10 |
| 205267_at | 5450 | POU2AF1 | NM_006235 | 12.46 | 26.93 | 24.31 | | 12.46 | 23.23 | 2.24 | 0.10 |
| 214073_at | 220 | ALDH1A3 | BG475299 | 3.88 | 2.97 | 7.21 | | 3.88 | 13.96 | 1.38 | 0.10 |
| 210078_s_at | 7881 | KCNAB1 | L39833 | 16.85 | 50.04 | 43.10 | | 16.85 | 39.05 | 3.96 | 0.10 |
| 219857_at | 79949 | C10orf81 | NM_024889 | 2.31 | 4.02 | 2.39 | | 2.31 | 14.23 | 1.45 | 0.10 |
| 220653_at | 23619 | ZIM2 | NM_015363 | 41.48 | 27.61 | 32.47 | | 41.48 | 69.88 | 7.13 | 0.10 |
| 205960_at | 5166 | PDK4 | NM_002612 | 2.59 | 2.75 | 6.29 | | 2.59 | 44.19 | 4.57 | 0.10 |
| 207599_at | 9313 | MMP20 | NM_004771 | 4.86 | 2.35 | 2.08 | | 4.86 | 15.32 | 1.60 | 0.10 |
| 215619_at | 473 | RERE | AU148274 | 4.45 | 4.40 | 2.05 | | 4.45 | 18.91 | 2.01 | 0.11 |
| 222353_at | 8994 | LIMD1 | AV720842 | 4.06 | 10.20 | 12.37 | | 4.06 | 23.29 | 2.50 | 0.11 |
| 215853_at | 10806 | SDCCAG8 | AK021640 | 33.80 | 40.89 | 34.08 | | 33.80 | 55.94 | 6.12 | 0.11 |
| 209101_at | 1490 | CTGF | M92934 | 23.07 | 5.24 | 26.98 | | 23.07 | 368.37 | 40.43 | 0.11 |
| 214467_at | 8477 | GPR65 | NM_003608 | 63.08 | 41.38 | 38.21 | | 63.08 | 140.86 | 15.55 | 0.11 |
| 208458_at | 6339 | SCNN1D | NM_002978 | 7.51 | 4.50 | 9.77 | | 7.51 | 29.51 | 3.26 | 0.11 |
| 212952_at | 811 | CALR | AA910371 | 1744.78 | 841.49 | 1191.85 | | 1744.748 | 4006.99 | 446.49 | 0.11 |
| 210834_s_at | 5733 | PTGER3 | AL031429 | 4.55 | 11.37 | 11.05 | | 4.55 | 19.20 | 2.16 | 0.11 |
| 208394_x_at | 11082 | ESM1 | NM_007036 | 2.24 | 2.12 | 6.74 | | 2.24 | 17.43 | 1.96 | 0.11 |
| 212488_at | 1289 | COL5A1 | N30339 | 3.89 | 9.49 | 3.11 | | 3.89 | 34.08 | 3.84 | 0.11 |
| 202310_s_at | 1277 | COL1A1 | K01228 | 5.86 | 13.13 | 19.21 | | 5.86 | 80.30 | 9.13 | 0.11 |
| 217401_at | | | AL035555 | 5.60 | 8.79 | 3.42 | | 5.60 | 11.74 | 1.35 | 0.11 |
| 203636_at | 4281 | MID1 | BE967532 | 10.55 | 14.34 | 18.76 | | 10.55 | 65.51 | 7.54 | 0.12 |
| 220877_at | | | NM_018580 | 7.74 | 9.89 | 2.24 | | 7.74 | 17.99 | 2.10 | 0.12 |
| 208329_at | 59351 | PBOV1 | NM_021635 | 4.46 | 5.91 | 3.64 | | 4.46 | 27.69 | 3.25 | 0.12 |
| 214000_s_at | 6001 | RGS10 | AI744627 | 46.17 | 25.13 | 39.60 | | 46.17 | 101.85 | 12.00 | 0.12 |
| 220882_at | | | NM_018612 | 3.41 | 4.60 | 2.97 | | 3.41 | 111.91 | 13.24 | 0.12 |
| 213755_s_at | 137872 | ADHFE1 | BF431501 | 13.09 | 17.73 | 26.10 | | 13.09 | 40.50 | 4.90 | 0.12 |
| 213964_x_at | 79946 | C10orf95 | AI307586 | 2.10 | 1.66 | 6.98 | | 2.10 | 21.40 | 2.60 | 0.12 |
| 214889_at | 25854 | DKFZP564J102 | AL080065 | 14.32 | 3.25 | 3.82 | | 14.32 | 27.91 | 3.41 | 0.12 |
| 206069_s_at | 33 | ACADL | NM_001608 | 2.39 | 2.05 | 3.28 | | 2.39 | 10.40 | 1.30 | 0.12 |
| 210394_x_at | 548313 / | SSX4 | BC005325 | 1.87 | 8.11 | 6.97 | | 1.87 | 17.25 | 2.16 | 0.13 |
| | | /// | | | | | | | | | |
| | | SSX4B | | | | | | | | | |
| 216982_x_at | 51412 | ACTL6B | U50277 | 5.34 | 4.44 | 7.45 | | 5.34 | 13.24 | 1.70 | 0.13 |
| 220759_at | 64184 | FAM12B | NM_022360 | 2.44 | 6.28 | 18.52 | | 2.44 | 12.09 | 1.56 | 0.13 |
| 205834_s_at | 25859 | PART1 | AI770098 | 11.84 | 16.34 | 23.40 | | 11.84 | 27.34 | 3.55 | 0.13 |
| 217306_at | 645749 | LOC645749 | AL031119 | 5.18 | 15.57 | 12.73 | | 5.18 | 23.60 | 3.13 | 0.13 |
| 213871_s_at | 10591 | C6orf108 | AA523444 | 46.45 | 25.19 | 44.27 | | 46.45 | 83.50 | 11.44 | 0.14 |
| 210931_at | 6049 | RNF6 | AF293342 | 3.24 | 4.89 | 1.85 | | 3.24 | 12.59 | 1.73 | 0.14 |
| 216030_s_at | 6407 | SEMG2 | AL049767 | 3.60 | 3.09 | 13.98 | | 3.60 | 30.38 | 4.18 | 0.14 |
| 210339_s_at | 3817 | KLK2 | BC005196 | 8.22 | 45.01 | 40.89 | | 8.22 | 38.68 | 5.36 | 0.14 |
| 214026_s_at | 200734 | SPRED2 | AI860246 | 2.26 | 4.87 | 1.27 | | 2.26 | 23.31 | 3.24 | 0.14 |
| 206692_at | 3766 | KCNJ10 | NM_002241 | 4.22 | 4.80 | 3.59 | | 4.22 | 17.37 | 2.42 | 0.14 |
| 205764_at | | | NM_002932 | 4.35 | 5.28 | 3.09 | | 4.35 | 21.96 | 3.08 | 0.14 |
| 205419_at | 1880 | EBI2 | NM_004951 | 255.75 | 261.91 | 87.33 | | 255.75 | 852.74 | 120.26 | 0.14 |
| 206331_at | 10203 | CALCRL | NM_005795 | 19.42 | 23.36 | 6.18 | | 19.42 | 143.22 | 20.23 | 0.14 |
| 205343_at | 6819 | SULT1C1 | NM_001056 | 5.74 | 2.18 | 15.63 | | 5.74 | 24.45 | 3.46 | 0.14 |
| 201860_s_at | 5327 | PLAT | NM_000930 | 7.32 | 21.56 | 24.05 | | 7.32 | 100.69 | 14.28 | 0.14 |
| 207473_at | 4295 | MLN | NM_002418 | 11.58 | 18.81 | 42.05 | | 11.58 | 24.61 | 3.53 | 0.14 |
| 210631_at | 4763 | NF1 | D42072 | 7.09 | 7.92 | 14.61 | | 7.09 | 20.15 | 2.90 | 0.14 |
| 207811_at | 3859 | KRT12 | NM_000223 | 1.73 | 3.85 | 1.57 | | 1.73 | 19.20 | 2.77 | 0.14 |
| 207321_s_at | 23457 | ABCB9 | NM_019625 | 5.23 | 7.04 | 3.46 | | 5.23 | 16.01 | 2.31 | 0.14 |
| 216235_s_at | 1909 | EDNRA | S81545 | 20.10 | 18.68 | 28.08 | | 20.10 | 43.48 | 6.33 | 0.15 |
| 207288_at | 80161 | CXYorf2 | NM_025091 | 9.38 | 6.34 | 5.04 | | 9.38 | 19.90 | 2.90 | 0.15 |
| 216435_at | 5607 | MAP2K5 | AK025177 | 1.30 | 6.64 | 0.39 | | 1.30 | 18.65 | 2.73 | 0.15 |
| 205374_at | 6588 | SLN | NM_003063 | 18.84 | 29.69 | 23.74 | | 18.84 | 28.40 | 4.17 | 0.15 |
| 207864_at | 6332 | SCN7A | NM_002976 | 4.00 | 2.12 | 2.40 | | 4.00 | 17.62 | 2.60 | 0.15 |
| 222350_at | 607 | BCL9 | AW969913 | 14.66 | 7.42 | 10.98 | | 14.66 | 30.87 | 4.56 | 0.15 |
| 212667_at | 6678 | SPARC | AL575922 | 7.43 | 9.22 | 7.46 | | 7.43 | 18.63 | 2.75 | 0.15 |
| 217126_at | | | K00627 | 5.23 | 5.75 | 12.19 | | 5.23 | 17.64 | 2.61 | 0.15 |
| 221180_at | 80122 | YSK4 | NM_025052 | 14.60 | 25.74 | 39.06 | | 14.60 | 57.17 | 8.47 | 0.15 |
| 201416_at | 6659 | SOX4 | NM_003107 | 148.60 | 99.80 | 137.83 | | 148.60 | 392.36 | 58.16 | 0.15 |
| 214074_s_at | 2017 | CTTN | BG475299 | 8.91 | 4.37 | 20.30 | | 8.91 | 32.78 | 4.90 | 0.15 |
| 208162_s_at | 55099 | FLJ10232 | NM_018033 | 2.39 | 5.20 | 4.16 | | 2.39 | 14.20 | 2.15 | 0.15 |
| 205597_at | 80736 | SLC44A4 | NM_025257 | 6.91 | 2.92 | 22.26 | | 6.91 | 19.19 | 2.92 | 0.15 |
| 205399_at | 9201 | DCAMKL1 | NM_004734 | 1.83 | 1.76 | 1.65 | | 1.83 | 13.20 | 2.01 | 0.15 |
| 218002_s_at | 9547 | CXCL14 | AF144103 | 3.00 | 2.86 | 4.84 | | 3.00 | 589.79 | 90.26 | 0.15 |
| 213806_at | 5813 | PURA | BE222739 | 6.66 | 7.83 | 7.23 | | 6.66 | 47.39 | 7.27 | 0.15 |
| 220428_at | 50489 | CD207 | NM_015717 | 11.74 | 8.08 | 18.05 | | 11.74 | 19.08 | 2.93 | 0.15 |
| 205620_at | 2159 | F10 | NM_000504 | 7.12 | 15.80 | 15.94 | | 7.12 | 14.59 | 2.24 | 0.15 |
| 205529_s_at | 862 | RUNX1T1 | NM_004349 | 13.10 | 8.22 | 6.87 | | 13.10 | 250.58 | 38.59 | 0.15 |
| 220401_at | 79860 | FLJ21369 | NM_024802 | 8.28 | 5.68 | 4.34 | | 8.28 | 17.77 | 2.75 | 0.15 |
| 207710_at | 26239 | LCE2B | NM_014357 | 1.60 | 6.93 | 5.57 | | 1.60 | 14.66 | 2.30 | 0.16 |
| 208790_s_at | 284119 | PTRF | AF312393 | 17.21 | 10.92 | 28.04 | | 17.21 | 36.43 | 5.73 | 0.16 |
| 219195_at | 10891 | PPARGC1A | NM_013261 | 3.79 | 4.92 | 4.29 | | 3.79 | 16.01 | 2.53 | 0.16 |
| 215678_at | 440792 | LOC440792 | AB051440 | 21.38 | 11.43 | 6.70 | | 21.38 | 34.02 | 5.40 | 0.16 |
| 206048_at | 58495 | OVOL2 | NM_021220 | 7.06 | 8.74 | 18.75 | | 7.06 | 14.90 | 2.36 | 0.16 |
| 220551_at | 57084 | SLC17A6 | NM_020346 | 8.16 | 3.85 | 19.98 | | 8.16 | 22.01 | 3.50 | 0.16 |
| 206065_s_at | 1807 | DPYS | NM_001385 | 9.17 | 3.69 | 4.49 | | 9.17 | 82.41 | 13.20 | 0.16 |
| 206909_at | | | AF068863 | 9.84 | 12.72 | 9.25 | | 9.84 | 21.17 | 3.40 | 0.16 |
| 206144_at | 9223 | MAGI1 | NM_004742 | 1.68 | 3.25 | 3.11 | | 1.68 | 11.64 | 1.88 | 0.16 |
| 217169_at | 3493 | IGHA1 | M31949 | 13.76 | 9.29 | 13.34 | | 13.76 | 25.97 | 4.20 | 0.16 |
| 210993_s_at | 4086 | SMAD1 | U54826 | 14.71 | 22.00 | 2.50 | | 14.71 | 26.78 | 4.35 | 0.16 |
| 207178_s_at | 2444 | FRK | NM_002031 | 0.85 | 6.65 | 1.60 | | 0.85 | 28.65 | 4.68 | 0.16 |
| 208131_s_at | 5740 | PTGIS | NM_000961 | 11.40 | 4.49 | 29.49 | | 11.40 | 40.16 | 6.58 | 0.16 |
| 211274_at | 6899 | TBX1 | AF012130 | 5.88 | 11.06 | 26.86 | | 5.88 | 17.48 | 2.88 | 0.16 |
| 217426_at | | | L11372 | 7.25 | 6.86 | 23.14 | | 7.25 | 36.10 | 5.95 | 0.16 |
| 207097_s_at | 10246 | SLC17A2 | NM_005835 | 12.15 | 14.23 | 8.44 | | 12.15 | 26.05 | 4.30 | 0.17 |
| 216462_at | | | X79200 | 2.04 | 5.32 | 9.55 | | 2.04 | 10.20 | 1.70 | 0.17 |
| 208183_at | 6870 | TACR3 | NM_001059 | 11.48 | 25.88 | 25.95 | | 11.48 | 20.45 | 3.43 | 0.17 |
| 205908_s_at | 4958 | OMD | NM_005014 | 1.91 | 4.37 | 4.10 | | 1.91 | 18.65 | 3.14 | 0.17 |
| 37892_at | 1301 | COL11A1 | J04177 | 3.39 | 4.84 | 1.90 | | 3.39 | 10.84 | 1.83 | 0.17 |
| 217689_at | 5770 | PTPN1 | BG109555 | 6.99 | 3.74 | 2.80 | | 6.99 | 17.35 | 2.93 | 0.17 |
| 216185_at | 10690 | FUT9 | BC001879 | 5.09 | 4.00 | 4.70 | | 5.09 | 11.52 | 1.96 | 0.17 |
| 208471_at | 3250 | HPR | NM_020995 | 5.06 | 23.85 | 17.93 | | 5.06 | 22.71 | 3.91 | 0.17 |
| 206528_at | 7225 | TRPC6 | NM_004621 | 6.90 | 4.37 | 15.12 | | 6.90 | 14.83 | 2.57 | 0.17 |
| 205815_at | 5068 | REG3A | NM_002580 | 8.92 | 17.49 | 14.83 | | 8.92 | 19.74 | 3.42 | 0.17 |
| 221846_s_at | 57513 | CASK1N2 | AI970096 | 26.83 | 26.35 | 12.92 | | 26.83 | 112.50 | 19.50 | 0.17 |
| 219614_s_at | 54716 | SLC6A20 | NM_020208 | 3.56 | 3.79 | 4.21 | | 3.56 | 16.12 | 2.79 | 0.17 |
| 205863_at | 6283 | S100A12 | NM_005621 | 66.09 | 59.11 | 38.25 | | 66.09 | 515.83 | 89.58 | 0.17 |
| 213397_x_at | 6038 | RNASE4 | AI761728 | 2.07 | 2.88 | 5.52 | | 2.07 | 12.87 | 2.24 | 0.17 |
| 213747_at | | | AA047234 | 315.44 | 147.79 | 570.49 | | 315.44 | 1067.62 | 186.27 | 0.17 |
| 210836_x_at | 5144 | PDE4D | AF012073 | 29.15 | 39.13 | 28.28 | | 29.15 | 60.45 | 10.63 | 0.18 |
| 213757_at | | | AA393940 | 2393.48 | 1651.77 | 2565.92 | | 2393.48 | 8391.34 | 1487.69 | 0.18 |
| 201744_s_at | 4060 | LUM | NM_002345 | 0.37 | 1.17 | 0.63 | | 0.37 | 25.70 | 4.56 | 0.18 |
| 220231_at | 10842 | C7orf16 | NM_006658 | 13.68 | 49.24 | 42.07 | | 13.68 | 193.09 | 34.45 | 0.18 |
| 208105_at | 2696 | GIPR | NM_000164 | 14.84 | 8.24 | 7.34 | | 14.84 | 31.72 | 5.67 | 0.18 |
| 211959_at | 3488 | IGFBP5 | AW007532 | 5.17 | 18.35 | 16.85 | | 5.17 | 218.01 | 39.11 | 0.18 |
| 214176_s_at | 57326 | PBXIP1 | AI348545 | 196.90 | 92.43 | 162.05 | | 196.90 | 459.56 | 83.74 | 0.18 |
| 202835_at | 10907 | TXNL4A | BC001046 | 38.95 | 27.16 | 56.43 | | 38.95 | 124.33 | 22.75 | 0.18 |
| 200865_at | | | AI001896 | 4.64 | 2.75 | 3.44 | | 4.64 | 15.74 | 2.89 | 0.18 |
| 205163_at | 29895 | MYLPF | NM_013292 | 23.78 | 21.01 | 8.06 | | 23.78 | 57.41 | 10.58 | 0.18 |
| 210640_s_at | 2852 | GPR30 | U63917 | 11.00 | 8.27 | 18.88 | | 11.00 | 20.53 | 3.80 | 0.19 |
| 208098_at | 442192 / | OR5V1 | NM_030876 | 1.24 | 3.03 | 5.65 | | 1.24 | 13.79 | 2.55 | 0.19 |
| | | /// | | | | | | | | | |
| | | OR12D3 | | | | | | | | | |
| | | /// | | | | | | | | | |
| | | LOC442192 | | | | | | | | | |
| 207631_at | 10230 | NBR2 | NM_005821 | 22.40 | 32.64 | 58.94 | | 22.40 | 34.25 | 6.36 | 0.19 |
| 214935_at | 23636 | NUP62 | BE794962 | 8.23 | 12.22 | 9.93 | | 8.23 | 38.87 | 7.22 | 0.19 |
| | // | /// | | | | | | | | | |
| | | IL4I1 | | | | | | | | | |
| 220111_s_at | 57101 | TMEM16B | NM_020373 | 6.22 | 5.82 | 8.71 | | 6.22 | 11.75 | 2.19 | 0.19 |
| 206328_at | 1013 | CDH15 | NM_004933 | 3.35 | 2.94 | 3.93 | | 3.35 | 11.87 | 2.22 | 0.19 |
| 206407_s_at | 6357 | CCL13 | NM_005408 | 32.00 | 21.50 | 33.31 | | 32.00 | 64.22 | 12.12 | 0.19 |
| 216649_at | 6239 | RREB1 | AF072826 | 3.17 | 5.68 | 5.50 | | 3.17 | 11.98 | 2.26 | 0.19 |
| 214217_at | 2915 | GRM5 | D60132 | 3.19 | 2.08 | 1.47 | | 3.19 | 17.57 | 3.34 | 0.19 |
| 210373_at | 10641 | TUSC4 | AF040708 | 11.47 | 12.99 | 14.43 | | 11.47 | 23.87 | 4.55 | 0.19 |
| 220433_at | 79057 | PRRG3 | NM_024082 | 8.64 | 6.28 | 12.15 | | 8.64 | 19.81 | 3.78 | 0.19 |
| 205101_at | 4261 | CIITA | NM_000246 | 9.09 | 6.20 | 7.09 | | 9.09 | 34.78 | 6.66 | 0.19 |
| 216987_at | 3662 | IRF4 | D78261 | 2.16 | 1.84 | 0.62 | | 2.16 | 19.18 | 3.69 | 0.19 |
| 214644_at | 8330 | HIST1H2AK | BF061074 | 8.10 | 6.77 | 4.53 | | 8.10 | 19.06 | 3.69 | 0.19 |
| 209547_s_at | 57794 | SF4 | BC001043 | 25.31 | 20.05 | 16.50 | | 25.31 | 66.37 | 12.99 | 0.20 |
| 207489_at | 80052 | FLJ12331 | NM_024986 | 12.21 | 13.81 | 27.22 | | 12.21 | 29.52 | 5.79 | 0.20 |
| 206705_at | 7287 | TULP1 | NM_003322 | 11.15 | 12.61 | 14.49 | | 11.15 | 39.08 | 7.67 | 0.20 |
| 219728_at | 9499 | MYOT | NM_006790 | 3.09 | 3.62 | 13.34 | | 3.09 | 22.62 | 4.45 | 0.20 |
| 215183_at | | | AF090886 | 10.72 | 7.42 | 20.86 | | 10.72 | 39.25 | 7.74 | 0.20 |
| 221395_at | 50838 | TAS2R13 | NM_023920 | 3.30 | 4.05 | 3.70 | | 3.30 | 13.83 | 2.73 | 0.20 |
| 214776_x_at | 9942 | XYLB | AA777793 | 5.12 | 14.72 | 24.05 | | 5.12 | 28.33 | 5.61 | 0.20 |
| 220265_at | 57720 | GPR107 | NM_020960 | 20.75 | 31.14 | 34.05 | | 20.75 | 32.82 | 6.52 | 0.20 |
| 218517_at | 79960 | PHF17 | NM_024900 | 671.89 | 541.57 | 620.61 | | 671.89 | 3234.03 | 645.07 | 0.20 |
| 213991_s_at | 9957 | HS3ST1 | BF940710 | 16.80 | 9.92 | 5.45 | | 16.80 | 31.00 | 6.19 | 0.20 |
| 208339_at | 353515 / | XKRY | NM_004677 | 1.77 | 6.40 | 9.09 | | 1.77 | 15.95 | 3.19 | 0.20 |
| | | /// | | | | | | | | | |
| | | XKRY2 | | | | | | | | | |
| 219768_at | 79679 | VTCN1 | NM_024626 | 7.04 | 26.08 | 19.70 | | 7.04 | 26.59 | 5.33 | 0.20 |
| 218974_at | 55084 | FLJ10159 | NM_018013 | 222.38 | 310.14 | 140.60 | | 222.38 | 485.33 | 97.37 | 0.20 |
| 206618_at | 8809 | IL18R1 | NM_003855 | 6.10 | 156.73 | 122.43 | | 6.10 | 214.33 | 43.08 | 0.20 |
| 217345_at | | | U91640 | 2.59 | 8.63 | 19.63 | | 2.59 | 16.96 | 3.41 | 0.20 |
| 210049_at | 462 | SERPINC1 | D29832 | 8.65 | 11.33 | 4.76 | | 8.65 | 19.46 | 3.92 | 0.20 |
| 207024_at | 1144 | CHRND | NM_000751 | 21.20 | 3.50 | 11.38 | | 21.20 | 35.01 | 7.10 | 0.20 |
| 215768_at | 6660 | SOX5 | AL049337 | 261.96 | 376.89 | 230.64 | | 261.96 | 1435.56 | 292.59 | 0.20 |
| 215181_at | 64405 | CDH22 | AF035300 | 4.05 | 9.69 | 4.56 | | 4.05 | 24.42 | 4.98 | 0.20 |
| 220332_at | 10686 | CLDN16 | NM_006580 | 6.96 | 10.59 | 9.02 | | 6.96 | 18.69 | 3.82 | 0.20 |
| 220330_s_at | 64092 | SAMSN1 | NM_022136 | 299.66 | 283.79 | 140.95 | | 299.66 | 4794.99 | 979.81 | 0.20 |
| 215618_at | 6251 | RSU1 | AK024109 | 13.95 | 15.40 | 32.54 | | 13.95 | 34.33 | 7.05 | 0.21 |
| 220418_at | 51098 | IFT52 | NM_018961 | 1.80 | 6.21 | 2.53 | | 1.80 | 20.92 | 4.30 | 0.21 |
| | // | /// | | | | | | | | | |
| | | UBASH3A | | | | | | | | | |
| 209985_s_at | 429 | ASCL1 | BC001638 | 11.20 | 13.94 | 6.81 | | 11.20 | 26.33 | 5.45 | 0.21 |
| 220984_s_at | 81796 | SLCO5A1 | NM_030958 | 5.65 | 4.67 | 14.49 | | 5.65 | 32.62 | 6.77 | 0.21 |
| 201286_at | 6382 | SDC1 | Z48199 | 6.04 | 6.77 | 1.92 | | 6.04 | 12.55 | 2.61 | 0.21 |
| 207748_at | | | NM_014122 | 15.39 | 16.95 | 43.66 | | 15.39 | 56.06 | 11.66 | 0.21 |
| 208413_at | 5915 | RARB | NM_015854 | 2.15 | 4.01 | 3.06 | | 2.15 | 10.33 | 2.15 | 0.21 |
| 207703_at | 22829 | NLGN4Y | NM_014893 | 79.40 | 98.88 | 108.46 | | 79.40 | 564.37 | 118.03 | 0.21 |
| 211083_s_at | 9175 | MAP3K13 | Z25428 | 1.33 | 2.63 | 6.52 | | 1.33 | 12.08 | 2.54 | 0.21 |
| 214375_at | 440091 / | PPFIBP1 | AI962377 | 69.98 | 62.54 | 97.33 | | 69.98 | 178.23 | 37.50 | 0.21 |
| | | /// | | | | | | | | | |
| | | LOC440091 | | | | | | | | | |
| | | /// | | | | | | | | | |
| | | LOC7 | | | | | | | | | |
| 219064_at | 80760 | ITIH5 | NM_030569 | 5.73 | 3.79 | 9.11 | | 5.73 | 18.69 | 3.94 | 0.21 |
| 217082_at | | | U82306 | 4.93 | 2.01 | 8.21 | | 4.93 | 15.90 | 3.37 | 0.21 |
| 207688_s_at | 3626 | INHBC | NM_005538 | 327.66 | 191.98 | 326.23 | | 327.66 | 619.35 | 131.36 | 0.21 |
| 213774_s_at | | | AW614578 | 4.03 | 6.26 | 6.70 | | 4.03 | 14.53 | 3.10 | 0.21 |
| 206344_at | 5444 | PON1 | U53784 | 10.98 | 11.36 | 7.03 | | 10.98 | 16.90 | 3.61 | 0.21 |
| 220717_at | 80070 | ADAMTS20 | NM_025030 | 7.96 | 8.10 | 13.19 | | 7.96 | 23.35 | 5.00 | 0.21 |
| 207744_at | | | NM_014124 | 1.74 | 0.95 | 5.13 | | 1.74 | 11.11 | 2.39 | 0.21 |
| 215514_at | | | AL080072 | 5.34 | 9.83 | 11.46 | | 5.34 | 16.28 | 3.51 | 0.22 |
| 216558_x_at | | | AF044595 | 8.61 | 4.05 | 14.62 | | 8.61 | 26.20 | 5.69 | 0.22 |
| 208512_s_at | 4301 | MLLT4 | NM_005936 | 5.48 | 6.08 | 12.11 | | 5.48 | 16.17 | 3.52 | 0.22 |
| 220959_s_at | 29989 | OBP2B | NM_014581 | 12.73 | 9.84 | 7.55 | | 12.73 | 30.86 | 6.75 | 0.22 |
| | // | /// | | | | | | | | | |
| | | OBP2A | | | | | | | | | |
| 216704_at | 6902 | TBCA | AL353949 | 4.35 | 10.14 | 13.34 | | 4.35 | 21.21 | 4.65 | 0.22 |
| 204388_s_at | 4128 | MAOA | NM_000240 | 1.23 | 3.23 | 7.34 | | 1.23 | 13.35 | 2.95 | 0.22 |
| 219529_at | 9022 /// | CLIC3 | NM_004669 | 3.99 | 9.37 | 0.95 | | 3.99 | 20.79 | 4.60 | 0.22 |
| | | /// | | | | | | | | | |
| | | RABEP1 | | | | | | | | | |
| 220313_at | 54112 | GPR88 | NM_022049 | 140.60 | 189.27 | 109.25 | | 140.60 | 688.55 | 152.32 | 0.22 |
| 217561_at | 796 | CALCA | BF447272 | 10.92 | 8.14 | 4.82 | | 10.92 | 17.97 | 3.98 | 0.22 |
| 215307_at | 57711 | ZNF529 | AL109722 | 16.02 | 10.53 | 26.57 | | 16.02 | 25.91 | 5.74 | 0.22 |
| 220392_at | 64641 | EBF2 | NM_022659 | 3.92 | 3.87 | 3.17 | | 3.92 | 10.08 | 2.24 | 0.22 |
| 210664_s_at | 7035 | TFP1 | AF021834 | 6.05 | 10.38 | 31.32 | | 6.05 | 34.95 | 7.80 | 0.22 |
| 203432_at | 7112 | TMPO | AW272611 | 775.83 | 593.57 | 714.25 | | 775.83 | 1280.81 | 287.30 | 0.22 |
| 208075_s_at | 6354 | CCL7 | NM_006273 | 3.14 | 12.34 | 9.73 | | 3.14 | 26.25 | 5.90 | 0.22 |
| 220616_at | | | NM_006448 | 19.78 | 8.07 | 48.79 | | 19.78 | 57.14 | 12.87 | 0.23 |
| 203295_s_at | 477 | ATP1A2 | NM_000702 | 24.20 | 7.78 | 9.26 | | 24.20 | 37.13 | 8.38 | 0.23 |
| 208416_s_at | 6710 | SPTB | NM_000347 | 21.16 | 16.14 | 46.95 | | 21.16 | 55.42 | 12.57 | 0.23 |
| 213979_s_at | 1487 | CTBP1 | BF984434 | 766.52 | 549.77 | 790.85 | | 766.52 | 1794.22 | 407.70 | 0.23 |
| 201340_s_at | 8507 | ENC1 | AF010314 | 71.63 | 61.36 | 7.00 | | 71.63 | 120.85 | 27.48 | 0.23 |
| 221765_at | | | AI378044 | 167.05 | 244.29 | 252.38 | | 167.05 | 1035.54 | 237.06 | 0.23 |
| 215036_at | | | AI952772 | 10.40 | 7.19 | 15.22 | | 10.40 | 33.59 | 7.71 | 0.23 |
| 216995_x_at | 23609 | MKRN2 | X06409 | 2.68 | 7.51 | 5.44 | | 2.68 | 17.02 | 3.92 | 0.23 |
| 220825_s_at | 55243 | KIRREL | NM_018240 | 4.23 | 3.87 | 6.69 | | 4.23 | 10.05 | 2.32 | 0.23 |
| 207731_at | | | NM_020118 | 5.86 | 7.35 | 3.54 | | 5.86 | 11.27 | 2.61 | 0.23 |
| 220502_s_at | 6561 | SLC13A1 | AF260824 | 3.52 | 4.09 | 12.12 | | 3.52 | 17.15 | 3.97 | 0.23 |
| 214101_s_at | 728896 | LOC728896 | BG153399 | 137.38 | 136.88 | 180.71 | | 137.38 | 533.68 | 123.56 | 0.23 |
| 210382_at | 6344 | SCTR | U13989 | 8.09 | 6.06 | 6.03 | | 8.09 | 36.49 | 8.46 | 0.23 |
| 207256_at | 4153 | MBL2 | NM_000242 | 2.50 | 1.33 | 4.14 | | 2.50 | 10.44 | 2.44 | 0.23 |
| 221529_s_at | 83483 | PLVAP | AF326591 | 11.30 | 7.18 | 25.72 | | 11.30 | 28.83 | 6.76 | 0.23 |
| 209690_s_at | 55715 | DOK4 | BC003541 | 6.83 | 10.54 | 8.79 | | 6.83 | 27.09 | 6.41 | 0.24 |
| 210861_s_at | 8838 | WISP3 | AF143679 | 2.09 | 1.21 | 7.79 | | 2.09 | 13.25 | 3.17 | 0.24 |
| 220657_at | 55175 | KLHL11 | NM_018143 | 7.81 | 6.55 | 6.63 | | 7.81 | 23.72 | 5.69 | 0.24 |
| 209290_s_at | 4781 | NFIB | BC001283 | 5.22 | 5.72 | 13.05 | | 5.22 | 26.40 | 6.33 | 0.24 |
| 210356_x_at | 931 | MS4A1 | BC002807 | 7.05 | 9.75 | 23.10 | | 7.05 | 11.77 | 2.83 | 0.24 |
| 214587_at | 1295 | COL8A1 | BE877796 | 10.90 | 16.26 | 6.51 | | 10.90 | 19.94 | 4.80 | 0.24 |
| 213565_s_at | 4091 | SMAD6 | A1193899 | 10.35 | 12.84 | 13.06 | | 10.35 | 28.61 | 6.91 | 0.24 |
| 216456_at | 5101 | PCDH9 | AL162044 | 7.61 | 10.97 | 6.21 | | 7.61 | 21.07 | 5.09 | 0.24 |
| 220334_at | 26575 | RGS17 | NM_012419 | 4.67 | 18.44 | 27.58 | | 4.67 | 45.54 | 11.02 | 0.24 |
| 216124_at | 54879 | ST7L | AK024158 | 13.20 | 8.80 | 12.57 | | 13.20 | 22.08 | 5.34 | 0.24 |
| 215927_at | 10564 | ARFGEF2 | AV657604 | 6.00 | 8.79 | 12.23 | | 6.00 | 23.67 | 5.73 | 0.24 |
| 214142_at | 123887 | ZG16 | AI732905 | 6.01 | 6.78 | 8.19 | | 6.01 | 22.17 | 5.37 | 0.24 |
| 206080_at | 9651 | PLCH2 | NM_014638 | 5.81 | 5.60 | 4.44 | | 5.81 | 17.57 | 4.27 | 0.24 |
| 205326_at | 10268 | RAMP3 | NM_005856 | 30.47 | 13.41 | 35.39 | | 30.47 | 46.09 | 11.22 | 0.24 |
| 222062_at | 9466 | IL27RA | AI983115 | 193.02 | 338.97 | 259.12 | | 193.02 | 2386.38 | 583.92 | 0.24 |
| 209888_s_at | 4632 | MYL1 | M20643 | 3.85 | 5.66 | 4.67 | | 3.85 | 10.77 | 2.64 | 0.24 |
| 220595_at | 29951 | PDZRN4 | NM_013377 | 7.48 | 10.67 | 3.92 | | 7.48 | 13.90 | 3.41 | 0.25 |
| 205572_at | 285 | ANGPT2 | NM_001147 | 8.61 | 7.15 | 15.35 | | 8.61 | 28.81 | 7.10 | 0.25 |
| 215432_at | 116285 | ACSM1 | AC003034 | 6.62 | 5.97 | 13.02 | | 6.62 | 12.53 | 3.09 | 0.25 |
| 210080_x_at | 10136 | ELA3A | D00306 | 14.51 | 8.79 | 4.88 | | 14.51 | 25.96 | 6.44 | 0.25 |
| 203398_s_at | 2591 | GALNT3 | NM_004482 | 7.66 | 38.18 | 29.55 | | 7.66 | 30.22 | 7.50 | 0.25 |
| 206439_at | 1833 | EPYC | NM_004950 | 6.75 | 3.28 | 1.90 | | 6.75 | 15.50 | 3.85 | 0.25 |
| 206598_at | 3630 | INS | NM_000207 | 3.02 | 2.70 | 2.00 | | 3.02 | 18.42 | 4.57 | 0.25 |
| 220676_at | 11095 | ADAMTS8 | NM_007037 | 5.35 | 4.47 | 16.73 | | 5.35 | 25.79 | 6.41 | 0.25 |
| 210665_at | 7035 | TFPI | AF021834 | 5.80 | 8.57 | 5.04 | | 5.80 | 22.17 | 5.51 | 0.25 |
| 213655_at | 5048 | PAFAH1B1 | AA502643 | 2539.35 | 1808.95 | 2842.37 | | 2539.35 | 7026.66 | 1754.25 | 0.25 |
| 218875_s_at | 26271 | FBXO5 | NM_012177 | 594.34 | 574.83 | 520.55 | | 594.34 | 1893.84 | 473.23 | 0.25 |
| 207145_at | 2660 | GDF8 | NM_005259 | 2.84 | 2.28 | 4.17 | | 2.84 | 10.33 | 2.58 | 0.25 |
| 218574_s_at | 29995 | LMCD1 | NM_014583 | 23.55 | 4.03 | 17.24 | | 23.55 | 46.87 | 11.73 | 0.25 |
| 207038_at | 9120 | SLC16A6 | NM_004694 | 6.97 | 34.06 | 28.86 | | 6.97 | 23.55 | 5.89 | 0.25 |
| 206721_at | 57821 | C1orf114 | NM_021179 | 10.31 | 2.87 | 6.29 | | 10.31 | 22.21 | 5.57 | 0.25 |
| 207669_at | 3889 | KRT83 | NM_002282 | 5.60 | 6.60 | 6.45 | | 5.60 | 18.63 | 4.67 | 0.25 |
| 202969_at | | | AI216690 | 284.42 | 326.45 | 231.60 | | 284.42 | 616.25 | 155.30 | 0.25 |
| 221872_at | 5918 | RARRES1 | AI669229 | 0.95 | 7.19 | 22.24 | | 0.95 | 13.73 | 3.46 | 0.25 |
| 204198_s_at | 864 | RUNX3 | AA541630 | 804.37 | 1177.81 | 431.84 | | 804.37 | 1672.19 | 424.29 | 0.25 |
| 211161_s_at | 1281 | COL3A1 | AF130082 | 3.16 | 3.75 | 5.35 | | 3.16 | 20.76 | 5.27 | 0.25 |
| 221698_s_at | 64581 | CLEC7A | AF313468 | 42.75 | 59.27 | 51.05 | | 42.75 | 173.71 | 44.19 | 0.25 |
| 215360_at | | | AK022063 | 1.37 | 1.37 | 4.89 | | 1.37 | 13.55 | 3.45 | 0.25 |
| 211348_s_at | 8555 | CDC14B | AF064105 | 21.81 | 5.56 | 12.85 | | 21.81 | 33.15 | 8.45 | 0.25 |
| 221884_at | 2122 | EVI1 | BE466525 | 8.67 | 5.58 | 14.16 | | 8.67 | 15.88 | 4.05 | 0.25 |
| 200761_s_at | 10550 | ARL6IP5 | NM_006407 | 2021.41 | 2829.28 | 2410.96 | | 2021.41 | 3837.72 | 980.80 | 0.26 |
| 205102_at | 7113 | TMPRSS2 | NM_005656 | 10.49 | 5.08 | 19.42 | | 10.49 | 21.90 | 5.60 | 0.26 |
| 214418_at | 196993 | LOC196993 | AI656822 | 5.48 | 6.03 | 2.89 | | 5.48 | 10.05 | 2.57 | 0.26 |
| 214862_x_at | | | AL080082 | 2.29 | 6.41 | 4.25 | | 2.29 | 13.63 | 3.50 | 0.26 |
| 217194_at | 9462 | RASAL2 | AB007970 | 2.86 | 9.03 | 13.06 | | 2.86 | 10.85 | 2.79 | 0.26 |
| 218413_s_at | 51193 | ZNF639 | NM_016331 | 9.14 | 5.19 | 20.07 | | 9.14 | 39.77 | 10.24 | 0.26 |
| 206027_at | 6274 | S100A3 | NM_002960 | 28.61 | 28.50 | 13.55 | | 28.61 | 81.06 | 20.98 | 0.26 |
| 205674_x_at | 486 | FXYD2 | NM_001680 | 22.37 | 29.36 | 27.36 | | 22.37 | 85.81 | 22.41 | 0.26 |
| 205809_s_at | 8976 | WASL | BE504979 | 111.25 | 90.27 | 153.75 | | 111.25 | 240.88 | 62.99 | 0.26 |
| 213868_s_at | 51635 | DHRS7 | AW243128 | 2.80 | 7.04 | 6.93 | | 2.80 | 11.65 | 3.05 | 0.26 |
| 206167_s_at | 395 | ARHGAP6 | NM_001174 | 3.65 | 19.02 | 20.45 | | 3.65 | 64.74 | 16.96 | 0.26 |
| 210025_s_at | 29775 | CARD10 | AW205153 | 16.25 | 22.02 | 35.74 | | 16.25 | 60.78 | 16.00 | 0.26 |
| 205629_s_at | 1392 | CRH | BC002599 | 11.88 | 16.57 | 4.74 | | 11.88 | 25.96 | 6.84 | 0.26 |
| 209854_s_at | 3817 | KLK2 | AF188747 | 5.18 | 3.03 | 2.57 | | 5.18 | 14.86 | 3.92 | 0.26 |
| 221997_s_at | 122704 | MRPL52 | AI560951 | 110.57 | 75.97 | 120.11 | | 110.57 | 183.34 | 48.45 | 0.26 |
| 206824_at | 51716 | CES4 | NM_016280 | 4.98 | 5.15 | 2.55 | | 4.98 | 17.68 | 4.67 | 0.26 |
| 207925_at | 1473 | CST5 | NM_001900 | 8.59 | 3.74 | 31.35 | | 8.59 | 15.42 | 4.08 | 0.26 |
| 216600_x_at | | | AK026411 | 50.51 | 67.20 | 54.99 | | 50.51 | 76.01 | 20.11 | 0.26 |
| 209335_at | 1634 | DCN | BC005322 | 9.45 | 16.25 | 20.72 | | 9.45 | 30.15 | 7.98 | 0.26 |
| 207395_at | 696 | BTN1A1 | NM_001732 | 4.95 | 5.50 | 8.07 | | 4.95 | 15.30 | 4.06 | 0.27 |
| 217481_x_at | 388336 | FLJ45455 | AL110201 | 15.87 | 20.39 | 16.45 | | 15.87 | 31.98 | 8.49 | 0.27 |
| 214468_at | 4624 | MYH6 | D00943 | 3.84 | 6.60 | 8.61 | | 3.84 | 24.96 | 6.63 | 0.27 |
| 206616_s_at | 53616 | ADAM22 | AF155382 | 5.67 | 3.75 | 3.53 | | 5.67 | 11.44 | 3.04 | 0.27 |
| 206683_at | 7718 | ZNF165 | NM_003447 | 16.95 | 23.59 | 18.69 | | 16.95 | 46.90 | 12.48 | 0.27 |
| 220862_s_at | 23326 | USP22 | NM_014093 | 13.23 | 8.50 | 4.20 | | 13.23 | 28.35 | 7.57 | 0.27 |
| 220872_at | 55415 | PRO2964 | NM_018547 | 44.15 | 37.40 | 33.91 | | 44.15 | 124.12 | 33.27 | 0.27 |
| 1320_at | 11099 | PTPN21 | X79510 | 4.10 | 14.79 | 17.18 | | 4.10 | 20.28 | 5.44 | 0.27 |
| 207929_at | 2925 | GRPR | NM_005314 | 12.63 | 23.86 | 37.49 | | 12.63 | 35.34 | 9.48 | 0.27 |
| 220114_s_at | 55576 | STAB2 | NM_017564 | 30.48 | 20.22 | 30.38 | | 30.48 | 50.95 | 13.67 | 0.27 |
| 202489_s_at | 5349 | FXYD3 | BC005238 | 7.01 | 4.63 | 5.16 | | 7.01 | 49.52 | 13.29 | 0.27 |
| 210750_s_at | 9229 | DLGAP1 | AB000277 | 13.66 | 6.37 | 36.86 | | 13.66 | 52.95 | 14.24 | 0.27 |
| 212533_at | 7465 | WEE1 | X62048 | 740.96 | 666.06 | 664.70 | | 740.96 | 1614.33 | 435.31 | 0.27 |
| 206021_at | 54581 | SCAND2 | NM_022050 | 9.69 | 11.16 | 8.56 | | 9.69 | 19.97 | 5.39 | 0.27 |
| 208247_at | 711 | C3orf51 | NM_001213 | 4.74 | 1.98 | 4.78 | | 4.74 | 14.60 | 3.98 | 0.27 |
| 206392_s_at | 5918 | RARRES1 | NM_002888 | 8.04 | 1.67 | 6.79 | | 8.04 | 28.04 | 7.67 | 0.27 |
| 214388_at | 10443 | PFAAP5 | AI732885 | 56.87 | 77.68 | 82.68 | | 56.87 | 134.22 | 36.79 | 0.27 |
| 215145_s_at | 26047 | CNTNAP2 | AC005378 | 7.58 | 4.27 | 4.30 | | 7.58 | 11.89 | 3.26 | 0.27 |
| 202600_s_at | 8204 | NRIP1 | AI824012 | 695.26 | 743.82 | 853.54 | | 695.26 | 3249.25 | 892.90 | 0.27 |
| 203424_s_at | 3488 | IGFBP5 | AW157548 | 3.79 | 22.29 | 21.70 | | 3.79 | 72.37 | 19.93 | 0.28 |
| 207799_x_at | | | NM_015437 | 24.85 | 18.13 | 6.90 | | 24.85 | 49.19 | 13.56 | 0.28 |
| 201242_s_at | 481 | ATP1B1 | BC000006 | 751.38 | 1068.01 | 600.04 | | 751.38 | 11642.50 | 3222.21 | 0.28 |
| 216217_at | 23228 | PLCL2 | AK023546 | 9.08 | 14.39 | 39.09 | | 9.08 | 20.86 | 5.77 | 0.28 |
| 207315_at | 10666 | CD226 | NM_006566 | 8.96 | 7.90 | 8.80 | | 8.96 | 16.30 | 4.53 | 0.28 |
| 216240_at | 5820 | PVT1 | M34428 | 5.56 | 9.88 | 18.54 | | 5.56 | 34.97 | 9.71 | 0.28 |
| 206035_at | 5966 | REL | NM_002908 | 24.22 | 12.38 | 20.40 | | 24.22 | 331.30 | 92.07 | 0.28 |
| 221795_at | 4915 | NTRK2 | AA707199 | 4.24 | 3.95 | 3.10 | | 4.24 | 14.43 | 4.01 | 0.28 |
| 207415_at | 22925 | PLA2R1 | NM_007366 | 5.81 | 3.64 | 2.72 | | 5.81 | 13.21 | 3.69 | 0.28 |
| 216864_at | | | U05589 | 8.67 | 4.20 | 2.79 | | 8.67 | 13.95 | 3.89 | 0.28 |
| 211567_at | | | J04168 | 2.33 | 5.41 | 4.93 | | 2.33 | 11.84 | 3.32 | 0.28 |
| 216190_x_at | 3688 | ITGB1 | AA215854 | 6.44 | 4.31 | 25.88 | | 6.44 | 15.82 | 4.43 | 0.28 |
| 211131_s_at | 1896 | EDA | AF061193 | 4.77 | 26.98 | 25.43 | | 4.77 | 20.71 | 5.81 | 0.28 |
| 208303_s_at | 64109 | CRLF2 | NM_022148 | 16.51 | 7.18 | 8.63 | | 16.51 | 1356.51 | 381.58 | 0.28 |
| 208232_x_at | 3084 | NRG1 | L12260 | 12.44 | 43.25 | 33.69 | | 12.44 | 37.46 | 10.54 | 0.28 |
| 216642_at | 6397 | SEC14L1 | AL110190 | 22.54 | 25.08 | 13.40 | | 22.54 | 37.62 | 10.60 | 0.28 |
| 212827_at | 3507 | IGHM | X17115 | 15.53 | 21.19 | 13.40 | | 15.53 | 65.44 | 18.48 | 0.28 |
| 204591_at | 10752 | CHL1 | NM_006614 | 4.95 | 8.02 | 7.14 | | 4.95 | 20.37 | 5.75 | 0.28 |
| 208004_at | 58503 | PROL1 | NM_021225 | 14.22 | 36.53 | 29.12 | | 14.22 | 40.19 | 11.40 | 0.28 |
| 207887_s_at | 799 | CALCR | AB022177 | 5.05 | 14.08 | 11.84 | | 5.05 | 26.49 | 7.56 | 0.29 |
| 204814_at | 8618 | CADPS | NM_003716 | 8.11 | 5.96 | 23.10 | | 8.11 | 29.57 | 8.46 | 0.29 |
| 37996_s_at | 1760 | DMPK | L08835 | 72.89 | 100.17 | 52.84 | | 72.89 | 110.11 | 31.49 | 0.29 |
| 218350_s_at | 51053 | GMNN | NM_015895 | 870.50 | 736.93 | 698.81 | | 870.50 | 1727.31 | 494.58 | 0.29 |
| 219804_at | 79933 | SYNPO2L | NM_024875 | 2.59 | 1.83 | 1.02 | | 2.59 | 13.90 | 3.99 | 0.29 |
| 221273_s_at | 727800 | LOC727800 | NM_031297 | 21.10 | 21.42 | 15.67 | | 21.10 | 36.39 | 10.46 | 0.29 |
| 209816_at | 5727 | PTCH1 | AL044175 | 10.00 | 5.16 | 10.88 | | 10.00 | 24.92 | 7.17 | 0.29 |
| 216803_at | 10611 | PDLIM5 | AK027217 | 5.28 | 14.04 | 16.42 | | 5.28 | 11.18 | 3.22 | 0.29 |
| 205828_at | 4314 | MMP3 | NM_002422 | 13.63 | 13.06 | 21.41 | | 13.63 | 23.26 | 6.71 | 0.29 |
| 220249_at | 23553 | HYAL4 | NM_012269 | 7.20 | 7.28 | 2.48 | | 7.20 | 28.92 | 8.37 | 0.29 |
| 211201_at | 2492 | FSHR | M95489 | 6.10 | 3.63 | 2.28 | | 6.10 | 19.21 | 5.56 | 0.29 |
| 219739_at | 54546 | RNF186 | NM_019062 | 7.82 | 5.51 | 8.38 | | 7.82 | 18.93 | 5.49 | 0.29 |
| 211403_x_at | 26609 | VCY | AF167079 | 15.03 | 10.65 | 26.10 | | 15.03 | 30.62 | 8.91 | 0.29 |
| | // | /// | | | | | | | | | |
| | | VCX | | | | | | | | | |
| | | /// | | | | | | | | | |
| | | VCX2 | | | | | | | | | |
| | | /// | | | | | | | | | |
| | | VCX3A | | | | | | | | | |
| 209924_at | 6362 | CCL18 | AB000221 | 5.01 | 8.22 | 3.71 | | 5.01 | 35.20 | 10.24 | 0.29 |
| 205514_at | 55786 | ZNF415 | NM_018355 | 1.70 | 1.56 | 1.47 | | 1.70 | 15.58 | 4.54 | 0.29 |
| 219585_at | 79140 | CCDC28B | NM_024296 | 7.89 | 8.44 | 7.73 | | 7.89 | 17.29 | 5.04 | 0.29 |
| 206109_at | 2523 | FUT1 | NM_000148 | 19.28 | 38.51 | 53.68 | | 19.28 | 62.90 | 18.33 | 0.29 |
| 204184_s_at | 157 | ADRBK2 | NM_005160 | 135.61 | 147.65 | 260.75 | | 135.61 | 215.58 | 62.91 | 0.29 |
| 209740_s_at | 8228 | PNPLA4 | U03886 | 11.84 | 14.37 | 40.29 | | 11.84 | 31.67 | 9.26 | 0.29 |
| 213765_at | 8076 | MFAP5 | AW665892 | 5.45 | 6.98 | 3.18 | | 5.45 | 21.59 | 6.31 | 0.29 |
| 207331_at | 1063 | CENPF | NM_016343 | 39.68 | 61.25 | 58.36 | | 39.68 | 60.75 | 17.78 | 0.29 |
| 216108_at | | | AU147017 | 12.31 | 2.04 | 9.10 | | 12.31 | 19.45 | 5.70 | 0.29 |
| 216784_at | | | AK025422 | 17.39 | 4.52 | 29.39 | | 17.39 | 38.10 | 11.18 | 0.29 |
| 215748_at | 283212 | FLJ33790 | AL050370 | 6.42 | 9.30 | 23.55 | | 6.42 | 36.45 | 10.70 | 0.29 |
| 222337_at | 114883 | OSBPL9 | AW968210 | 25.07 | 35.61 | 27.75 | | 25.07 | 50.29 | 14.80 | 0.29 |
| 211148_s_at | 285 | ANGPT2 | AF187858 | 4.10 | 3.82 | 12.64 | | 4.10 | 12.25 | 3.62 | 0.30 |
| 216778_s_at | 1538 | CYLC1 | Z22780 | 3.82 | 15.76 | 13.38 | | 3.82 | 14.75 | 4.37 | 0.30 |
| 212923_s_at | 221749 | C6orf145 | AK024828 | 61.65 | 189.49 | 144.47 | | 61.65 | 1582.04 | 469.53 | 0.30 |
| 205805_s_at | 4919 | ROR1 | NM_005012 | 12.55 | 13.97 | 9.88 | | 12.55 | 19.30 | 5.75 | 0.30 |
| 213906_at | 4603 | MYBL1 | AW592266 | 363.55 | 455.99 | 206.56 | | 363.55 | 611.58 | 182.64 | 0.30 |
| 214077_x_at | 4213 | MEIS3P1 | H15129 | 189.79 | 245.42 | 198.01 | | 189.79 | 381.83 | 114.12 | 0.30 |
| 207982_at | 3010 | HIST1H1T | NM_005323 | 19.26 | 14.47 | 17.82 | | 19.26 | 54.57 | 16.36 | 0.30 |
| 221204_s_at | 55118 | CRTAC1 | NM_018058 | 17.94 | 20.34 | 31.82 | | 17.94 | 45.97 | 13.89 | 0.30 |
| 206093_x_at | 7148 | TNXB | NM_007116 | 6.80 | 15.62 | 13.63 | | 6.80 | 21.78 | 6.58 | 0.30 |
| 209074_s_at | 11170 | FAM107A | AF089853 | 3.00 | 7.21 | 1.94 | | 3.00 | 17.86 | 5.40 | 0.30 |
| 208389_s_at | 6506 | SLC1A2 | NM_004171 | 10.38 | 7.55 | 35.09 | | 10.38 | 46.41 | 14.04 | 0.30 |
| 214464_at | 8476 | CDC42BPA | NM_003607 | 15.67 | 10.70 | 13.80 | | 15.67 | 23.78 | 7.19 | 0.30 |
| 219602_s_at | 63895 | FAM38B | AW269818 | 58.88 | 86.45 | 36.48 | | 58.88 | 115.47 | 34.99 | 0.30 |
| 213962_s_at | 23141 | KIAA0692 | AI924382 | 16.60 | 9.97 | 34.09 | | 16.60 | 38.22 | 11.58 | 0.30 |
| 217206_at | | | D00267 | 13.50 | 9.39 | 17.16 | | 13.50 | 34.63 | 10.50 | 0.30 |
| 208265_at | 56918 | DKFZp547H025 | NM_020161 | 2.47 | 4.08 | 6.57 | | 2.47 | 16.82 | 5.12 | 0.30 |
| 207500_at | 838 | CASP5 | NM_004347 | 11.33 | 15.19 | 12.14 | | 11.33 | 69.59 | 21.32 | 0.31 |
| 213350_at | 6205 | RPS11 | BF680255 | 522.85 | 650.26 | 632.61 | | 522.85 | 1583.80 | 485.58 | 0.31 |
| 217460_at | 7142 | TNP2 | X63759 | 4.90 | 5.01 | 17.20 | | 4.90 | 20.34 | 6.24 | 0.31 |
| 220907_at | 266977 | GPR110 | NM_025048 | 10.97 | 23.38 | 19.46 | | 10.97 | 19.55 | 6.02 | 0.31 |
| 208382_s_at | 11144 | DMC1 | NM_007068 | 5.84 | 16.49 | 25.88 | | 5.84 | 26.24 | 8.08 | 0.31 |
| 213573_at | | | AA861608 | 513.04 | 429.35 | 294.03 | | 513.04 | 925.56 | 285.39 | 0.31 |
| 213306_at | 8777 | MPDZ | AA917899 | 7.02 | 3.95 | 2.60 | | 7.02 | 30.84 | 9.52 | 0.31 |
| 214182_at | 382 | ARF6 | AA243143 | 651.99 | 537.07 | 751.31 | | 651.99 | 2197.77 | 679.05 | 0.31 |
| 217332_at | 647288 / | LOC647288 | AL133018 | 9.71 | 9.52 | 9.16 | | 9.71 | 30.15 | 9.32 | 0.31 |
| | | /// | | | | | | | | | |
| | | LOC730587 | | | | | | | | | |
| 206946_at | 10021 | HCN4 | NM_005477 | 13.59 | 4.80 | 10.52 | | 13.59 | 24.47 | 7.58 | 0.31 |
| 214944_at | 23035 | PHLPPL | AK001854 | 5.05 | 3.85 | 12.01 | | 5.05 | 34.90 | 10.81 | 0.31 |
| 215417_at | 23233 | EXOC6B | AI803703 | 8.77 | 12.13 | 9.54 | | 8.77 | 16.96 | 5.26 | 0.31 |
| 210498_at | 1213 | CLTC | AF130062 | 5.41 | 4.41 | 7.39 | | 5.41 | 25.68 | 7.97 | 0.31 |
| 203844_at | 7428 | VHL | NM_000551 | 2.39 | 10.46 | 9.02 | | 2.39 | 24.05 | 7.46 | 0.31 |
| 213705_at | | | AW301861 | 127.37 | 89.65 | 121.65 | | 127.37 | 276.88 | 86.04 | 0.31 |
| 213258_at | 7035 | TFPI | BF511231 | 15.91 | 39.97 | 30.15 | | 15.91 | 82.52 | 25.70 | 0.31 |
| 211266_s_at | 2828 | GPR4 | U35399 | 11.30 | 12.25 | 16.11 | | 11.30 | 25.51 | 7.97 | 0.31 |
| 208229_at | 2263 | FGFR2 | NM_022975 | 81.11 | 68.49 | 96.56 | | 81.11 | 141.64 | 44.29 | 0.31 |
| 215298_at | | | U79300 | 6.89 | 9.82 | 17.53 | | 6.89 | 27.16 | 8.51 | 0.31 |
| 214391_x_at | 5731 | PTGER1 | AI762344 | 14.66 | 11.33 | 10.32 | | 14.66 | 28.55 | 8.97 | 0.31 |
| 204871_at | 7978 | MTERF | NM_006980 | 132.49 | 135.92 | 147.33 | | 132.49 | 309.83 | 97.41 | 0.31 |
| 217554_at | | | AV719355 | 61.28 | 69.60 | 87.23 | | 61.28 | 252.86 | 79.69 | 0.32 |
| 210374_x_at | 5733 | PTGER3 | D38300 | 20.97 | 26.75 | 27.04 | | 20.97 | 38.35 | 12.10 | 0.32 |
| 211413_s_at | 23569 | PADI4 | AF229067 | 2.14 | 6.32 | 6.88 | | 2.14 | 10.50 | 3.32 | 0.32 |
| 212372_at | 4628 | MYH10 | AK026977 | 333.25 | 341.82 | 245.40 | | 333.25 | 752.49 | 237.95 | 0.32 |
| 220923_s_at | 29944 | PNMA3 | NM_013364 | 21.89 | 37.09 | 45.05 | | 21.89 | 42.88 | 13.60 | 0.32 |
| 204069_at | 4211 | MEIS1 | NM_002398 | 3.14 | 3.09 | 5.46 | | 3.14 | 21.60 | 6.85 | 0.32 |
| 219900_s_at | 55663 | ZNF446 | NM_017908 | 29.67 | 25.80 | 54.96 | | 29.67 | 47.50 | 15.07 | 0.32 |
| 211848_s_at | 1087 | CEACAM7 | AF006623 | 12.22 | 2.08 | 9.93 | | 12.22 | 20.52 | 6.52 | 0.32 |
| 202191_s_at | 8522 | GAS7 | BE439987 | 259.02 | 363.60 | 322.08 | | 259.02 | 549.39 | 175.24 | 0.32 |
| 203863_at | | | NM_001103 | 7.32 | 5.41 | 4.31 | | 7.32 | 15.23 | 4.88 | 0.32 |
| 221131_at | 51146 | A4GNT | NM_016161 | 23.52 | 13.63 | 44.66 | | 23.52 | 51.14 | 16.42 | 0.32 |
| 209905_at | 3205 | HOXA9 | AI246769 | 1336.86 | 1328.69 | 1205.90 | | 1336.86 | 2314.70 | 744.44 | 0.32 |
| 220316_at | 64067 | NPAS3 | NM_022123 | 6.17 | 4.88 | 23.19 | | 6.17 | 34.56 | 11.11 | 0.32 |
| 214253_s_at | 1838 | DTNB | AI672185 | 7.17 | 12.94 | 15.91 | | 7.17 | 12.10 | 3.89 | 0.32 |
| 206915_at | 4821 | NKX2-2 | NM_002509 | 5.74 | 8.40 | 7.53 | | 5.74 | 15.61 | 5.03 | 0.32 |
| 209640_at | 5371 | PML | M79462 | 103.93 | 152.75 | 52.72 | | 103.93 | 161.12 | 52.02 | 0.32 |
| 214071_at | 65258 | MPPE1 | AI082827 | 4.98 | 4.67 | 21.53 | | 4.98 | 13.49 | 4.37 | 0.32 |
| 217384_x_at | 3576 | IL8 | AJ275374 | 5.03 | 3.46 | 3.73 | | 5.03 | 29.54 | 9.58 | 0.32 |
| 204134_at | 5138 | PDE2A | NM_002599 | 5.69 | 8.74 | 16.12 | | 5.69 | 13.72 | 4.46 | 0.32 |
| 216566_at | | | D84140 | 7.95 | 8.95 | 5.72 | | 7.95 | 17.59 | 5.72 | 0.33 |
| 219493_at | 79801 | SHCBP1 | NM_024745 | 909.34 | 971.91 | 874.17 | | 909.34 | 1517.50 | 494.29 | 0.33 |
| 213609_s_at | 23544 | SEZ6L | AB023144 | 13.11 | 23.86 | 19.65 | | 13.11 | 33.07 | 10.78 | 0.33 |
| 200938_s_at | 473 | RERE | AI920976 | 7.75 | 7.65 | 2.65 | | 7.75 | 25.51 | 8.32 | 0.33 |
| 202517_at | 1400 | CRMP1 | NM_001313 | 15.83 | 22.69 | 26.13 | | 15.83 | 31.26 | 10.20 | 0.33 |
| 216609_at | 7295 | TXN | AF065241 | 300.13 | 264.66 | 432.34 | | 300.13 | 866.37 | 283.09 | 0.33 |
| 221222_s_at | 54964 | C1orf56 | NM_017860 | 161.08 | 189.41 | 223.25 | | 161.08 | 581.43 | 190.02 | 0.33 |
| 209529_at | 8612 | PPAP2C | AF047760 | 6.36 | 5.52 | 5.90 | | 6.36 | 36.40 | 11.90 | 0.33 |
| 217504_at | 23460 | ABCA6 | AA099357 | 15.12 | 15.97 | 15.05 | | 15.12 | 44.34 | 14.49 | 0.33 |
| 207977_s_at | 1805 | DPT | NM_001937 | 4.94 | 3.09 | 12.24 | | 4.94 | 27.25 | 8.92 | 0.33 |
| 213387_at | 54454 | ATAD2B | AB033066 | 432.35 | 479.83 | 550.43 | | 432.35 | 1079.37 | 353.82 | 0.33 |
| 221682_s_at | 56100 | PCDHGB6 | AF135156 | 3.09 | 3.29 | 3.87 | | 3.09 | 15.01 | 4.93 | 0.33 |
| 208003_s_at | 10725 | NFAT5 | NM_006599 | 111.69 | 183.35 | 294.93 | | 111.69 | 1165.92 | 384.13 | 0.33 |
| 217526_at | 84901 | NFATC2IP | AI478300 | 908.12 | 990.46 | 727.01 | | 908.12 | 1459.88 | 481.18 | 0.33 |
| 205839_s_at | 9256 | BZRAP1 | NM_004758 | 116.03 | 105.86 | 46.45 | | 116.03 | 216.00 | 71.22 | 0.33 |
| 213872_at | 81688 | C6orf62 | BE465032 | 4467.07 | 3291.94 | 4231.06 | | 4467.07 | 12032.13 | 3979.21 | 0.33 |
| 207386_at | 9420 | CYP7B1 | NM_004820 | 56.11 | 104.17 | 87.13 | | 56.11 | 695.28 | 230.00 | 0.33 |
| 209071_s_at | 8490 | RGS5 | AF159570 | 140.34 | 118.62 | 82.31 | | 140.34 | 233.03 | 77.16 | 0.33 |
| 215450_at | | | W87901 | 639.72 | 381.40 | 669.98 | | 639.72 | 987.74 | 327.24 | 0.33 |
| 217115_at | | | AL031686 | 9.70 | 9.55 | 3.21 | | 9.70 | 16.53 | 5.48 | 0.33 |
| 216058_s_at | 1557 | CYP2C19 | X65962 | 8.87 | 19.70 | 17.86 | | 8.87 | 34.18 | 11.42 | 0.33 |
| 214057_at | 4170 | MCL1 | H71805 | 195.42 | 199.20 | 194.76 | | 195.42 | 326.34 | 109.24 | 0.33 |
| 214640_at | 54346 | UNC93A | AL021331 | 0.99 | 6.45 | 21.82 | | 0.99 | 17.46 | 5.86 | 0.34 |
| 201996_s_at | 23013 | SPEN | AL524033 | 381.63 | 394.58 | 479.29 | | 381.63 | 942.35 | 316.24 | 0.34 |
| 213344_s_at | 3014 | H2AFX | H51429 | 120.93 | 133.97 | 157.21 | | 120.93 | 223.29 | 74.95 | 0.34 |
| 201645_at | 3371 | TNC | NM_002160 | 6.94 | 31.79 | 29.12 | | 6.94 | 131.77 | 44.27 | 0.34 |
| 205934_at | 5334 | PLCL1 | NM_006262 | 5.30 | 5.74 | 14.52 | | 5.30 | 51.15 | 17.21 | 0.34 |
| 205978_at | 9365 | KL | NM_004795 | 25.59 | 8.20 | 23.07 | | 25.59 | 39.47 | 13.28 | 0.34 |
| 216972_at | 6677 | SPAM1 | L13779 | 2.62 | 9.30 | 15.14 | | 2.62 | 17.41 | 5.88 | 0.34 |
| 208610_s_at | 23524 | SRRM2 | AI655799 | 1427.12 | 1168.86 | 1364.26 | | 1427.12 | 2409.21 | 814.03 | 0.34 |
| 208417_at | 2251 | FGF6 | NM_020996 | 36.94 | 54.61 | 51.29 | | 36.94 | 84.36 | 28.67 | 0.34 |
| 220326_s_at | 55701 | FLJ10357 | NM_018071 | 429.44 | 555.41 | 333.11 | | 429.44 | 736.94 | 250.54 | 0.34 |
| 212524_x_at | 3014 | H2AFX | AA760862 | 23.77 | 11.49 | 21.80 | | 23.77 | 36.95 | 12.56 | 0.34 |
| 219912_s_at | | | NM_005021 | 11.29 | 15.07 | 20.40 | | 11.29 | 17.42 | 5.93 | 0.34 |
| 205507_at | 22899 | ARHGEF15 | NM_014958 | 23.23 | 26.16 | 14.73 | | 23.23 | 36.70 | 12.51 | 0.34 |
| 215951_at | 23102 | TBC1D2B | AL137303 | 11.55 | 14.61 | 23.73 | | 11.55 | 19.07 | 6.50 | 0.34 |
| 208377_s_at | 778 | CACNA1F | NM_005183 | 5.04 | 4.45 | 5.17 | | 5.04 | 10.57 | 3.61 | 0.34 |
| 222183_x_at | 22938 | SNW1 | AL390153 | 82.73 | 103.80 | 73.58 | | 82.73 | 236.25 | 80.60 | 0.34 |
| 215198_s_at | 800 | CALD1 | AU147402 | 18.54 | 4.85 | 9.83 | | 18.54 | 30.40 | 10.38 | 0.34 |
| 204515_at | 3283 | HSD3B1 | NM_000862 | 4.44 | 11.33 | 13.05 | | 4.44 | 17.76 | 6.08 | 0.34 |
| 210357_s_at | 54498 | SMOX | BC000669 | 66.63 | 86.93 | 75.62 | | 66.63 | 524.99 | 179.82 | 0.34 |
| 206897_at | 8712 | PAGE1 | NM_003785 | 8.41 | 40.30 | 32.65 | | 8.41 | 34.41 | 11.79 | 0.34 |
| 216792_at | | | AK026867 | 18.65 | 33.57 | 31.05 | | 18.65 | 49.35 | 16.93 | 0.34 |
| 214329_x_at | 8743 | TNFSF10 | AW474434 | 151.51 | 178.28 | 68.69 | | 151.51 | 433.65 | 148.93 | 0.34 |
| 60794_f_at | 400721 / | LOC400721 | AI400621 | 356.46 | 239.02 | 331.46 | | 356.46 | 623.85 | 214.26 | 0.34 |
| | | /// | | | | | | | | | |
| | | LOC730051 | | | | | | | | | |
| 208465_at | 2912 | GRM2 | NM_000839 | 7.79 | 16.53 | 14.58 | | 7.79 | 14.96 | 5.14 | 0.34 |
| 212732_at | 55384 | MEG3 | AI950273 | 15.23 | 25.15 | 28.80 | | 15.23 | 31.46 | 10.81 | 0.34 |
| 208155_x_at | 2543 /// | GAGE1 | NM_001476 | 9.84 | 8.04 | 30.01 | | 9.84 | 49.98 | 17.22 | 0.34 |
| | | /// | | | | | | | | | |
| | | GAGE4 | | | | | | | | | |
| | | /// | | | | | | | | | |
| | | GAGE5 | | | | | | | | | |
| | | /// | | | | | | | | | |
| 210483_at | 254896 | MGC31957 | BC005043 | 19.25 | 9.94 | 14.77 | | 19.25 | 49.42 | 17.04 | 0.34 |
| 207181_s_at | 840 | CASP7 | NM_001227 | 199.41 | 273.14 | 181.19 | | 199.41 | 439.34 | 151.80 | 0.35 |
| 217195_at | | | L42230 | 17.67 | 25.91 | 25.78 | | 17.67 | 32.57 | 11.26 | 0.35 |
| 215103_at | 1562 | CYP2C18 | AW192911 | 9.33 | 3.30 | 3.14 | | 9.33 | 19.68 | 6.81 | 0.35 |
| 222161_at | 10003 | NAALAD2 | AJ012370 | 1.77 | 1.26 | 1.96 | | 1.77 | 15.60 | 5.41 | 0.35 |
| 215674_at | 85373 | KIAA1659 | AB051446 | 8.80 | 12.26 | 16.16 | | 8.80 | 15.42 | 5.35 | 0.35 |
| 216550_x_at | 23253 | ANKRD12 | X80821 | 196.14 | 195.58 | 256.91 | | 196.14 | 371.92 | 129.14 | 0.35 |
| 221387_at | 64106 | NPFFR1 | NM_022146 | 27.32 | 30.67 | 40.07 | | 27.32 | 60.87 | 21.14 | 0.35 |
| 218963_s_at | 25984 | KRT23 | NM_015515 | 5.73 | 2.59 | 5.20 | | 5.73 | 19.36 | 6.75 | 0.35 |
| 215058_at | 160518 | MGC24039 | AU144041 | 33.68 | 35.36 | 15.58 | | 33.68 | 71.97 | 25.15 | 0.35 |
| 214495_at | 10369 | CACNG2 | NM_006078 | 4.65 | 4.70 | 3.04 | | 4.65 | 14.24 | 4.98 | 0.35 |
| 216285_at | 8220 | DGCR14 | AL137713 | 3.98 | 10.95 | 17.29 | | 3.98 | 12.68 | 4.44 | 0.35 |
| 201893_x_at | 1634 | DCN | AF138300 | 19.80 | 13.96 | 30.66 | | 19.80 | 89.35 | 31.29 | 0.35 |
| 213710_s_at | | | AL523275 | 348.81 | 270.83 | 187.51 | | 348.81 | 637.83 | 223.75 | 0.35 |
| 209587_at | 5307 | PITX1 | U70370 | 10.52 | 3.90 | 5.44 | | 10.52 | 18.31 | 6.43 | 0.35 |
| 216910_at | 7512 | XPNPEP2 | AF195953 | 17.00 | 4.10 | 3.52 | | 17.00 | 27.17 | 9.55 | 0.35 |
| 202023_at | 1942 | EFNA1 | NM_004428 | 4.86 | 3.99 | 14.49 | | 4.86 | 19.13 | 6.72 | 0.35 |
| 214316_x_at | 811 | CALR | AI378706 | 199.68 | 206.15 | 199.34 | | 199.68 | 370.83 | 130.51 | 0.35 |
| 216834_at | 5996 | RGS1 | S59049 | 7.14 | 9.99 | 3.55 | | 7.14 | 93.55 | 32.97 | 0.35 |
| 217303_s_at | 155 | ADRB3 | X70812 | 3.75 | 6.15 | 3.42 | | 3.75 | 10.34 | 3.65 | 0.35 |
| 206986_at | 728985 / | LOC728985 | AB007422 | 16.61 | 15.65 | 18.93 | | 16.61 | 34.67 | 12.25 | 0.35 |
| | | /// | | | | | | | | | |
| | | LOC732125 | | | | | | | | | |
| 208327_at | 1553 | CYP2A13 | NM_000766 | 6.81 | 7.84 | 8.79 | | 6.81 | 14.40 | 5.09 | 0.35 |
| 213002_at | 4082 | MARCKS | AA770596 | 370.19 | 489.58 | 1055.64 | | 370.19 | 2017.85 | 714.48 | 0.35 |
| 215282_at | 25847 | ANAPC13 | BE677493 | 5.91 | 5.95 | 6.73 | | 5.91 | 13.08 | 4.63 | 0.35 |
| 217006_x_at | | | U52428 | 10.10 | 8.02 | 10.17 | | 10.10 | 17.38 | 6.16 | 0.35 |
| 210718_s_at | 51326 | ARL17P1 | AF119889 | 14.94 | 24.37 | 24.62 | | 14.94 | 28.28 | 10.03 | 0.35 |
| 205665_at | 10867 | TSPAN9 | NM_006675 | 10.18 | 13.77 | 28.16 | | 10.18 | 22.39 | 7.95 | 0.36 |
| 220017_x_at | 1559 | CYP2C9 | NM_000771 | 7.86 | 8.50 | 5.59 | | 7.86 | 28.00 | 9.96 | 0.36 |
| 206555_s_at | 55623 | THUMPD1 | NM_017736 | 940.98 | 866.21 | 958.89 | | 940.98 | 1978.37 | 703.71 | 0.36 |
| 201890_at | 6241 | RRM2 | BE966236 | 3071.14 | 3710.39 | 2785.24 | | 3071.14 | 8099.15 | 2881.88 | 0.36 |
| 221968_s_at | 51333 | ZNF771 | AW014373 | 4.06 | 8.94 | 3.05 | | 4.06 | 14.87 | 5.29 | 0.36 |
| 206794_at | 2066 | ERBB4 | NM_005235 | 9.87 | 16.07 | 24.06 | | 9.87 | 22.16 | 7.92 | 0.36 |
| 217258_x_at | 3712 | IVD | AF043583 | 4.47 | 5.58 | 3.34 | | 4.47 | 15.85 | 5.67 | 0.36 |
| 205523_at | 1404 | HAPLN1 | U43328 | 5.27 | 3.68 | 3.97 | | 5.27 | 16.08 | 5.75 | 0.36 |
| 205179_s_at | 101 | ADAM8 | AI814527 | 9.57 | 9.98 | 11.66 | | 9.57 | 25.65 | 9.19 | 0.36 |
| 220422_at | 50613 | UBQLN3 | NM_017481 | 4.26 | 5.90 | 8.50 | | 4.26 | 13.85 | 4.96 | 0.36 |
| 222237_s_at | 7771 | ZNF228 | AC084239 | 10.39 | 5.86 | 1.98 | | 10.39 | 15.89 | 5.70 | 0.36 |
| 213904_at | | | AL390170 | 6.41 | 7.16 | 2.18 | | 6.41 | 29.24 | 10.49 | 0.36 |
| 221334_s_at | 50943 | FOXP3 | NM_014009 | 13.69 | 17.98 | 14.22 | | 13.69 | 42.24 | 15.24 | 0.36 |
| 204511_at | 9855 | FARP2 | NM_014808 | 19.61 | 22.92 | 43.63 | | 19.61 | 40.91 | 14.76 | 0.36 |
| 220563_s_at | 50944 | SHANK1 | NM_016148 | 5.28 | 7.48 | 22.93 | | 5.28 | 17.38 | 6.28 | 0.36 |
| 212924_s_at | 25804 | LSM4 | N37057 | 19.72 | 22.11 | 16.08 | | 19.72 | 40.62 | 14.72 | 0.36 |
| 220318_at | 55040 | EPN3 | NM_017957 | 3.63 | 4.45 | 22.15 | | 3.63 | 10.33 | 3.75 | 0.36 |
| 214676_x_at | 57876 | MUC3B | AF113616 | 5.04 | 9.67 | 20.68 | | 5.04 | 21.70 | 7.88 | 0.36 |
| 201205_at | | | AF006751 | 74.84 | 86.79 | 59.19 | | 74.84 | 114.24 | 41.55 | 0.36 |
| 204863_s_at | 3572 /// | IL6ST | BE856546 | 76.81 | 107.86 | 161.33 | | 76.81 | 126.00 | 45.85 | 0.36 |
| | | /// | | | | | | | | | |
| | | MAGEA4 | | | | | | | | | |
| 202743_at | 8503 | PIK3R3 | BE622627 | 180.26 | 182.78 | 177.64 | | 180.26 | 303.90 | 110.66 | 0.36 |
| 208475_at | 55691 | FRMD4A | NM_018027 | 10.54 | 8.48 | 22.45 | | 10.54 | 40.18 | 14.64 | 0.36 |
| 211010_s_at | 259197 | NCR3 | AF031138 | 5.40 | 3.95 | 3.98 | | 5.40 | 10.75 | 3.92 | 0.37 |
| 205378_s_at | 43 | ACHE | NM_015831 | 13.73 | 3.61 | 7.30 | | 13.73 | 26.60 | 9.73 | 0.37 |
| 204636_at | 1308 | COL17A1 | NM_000494 | 4.91 | 4.80 | 5.16 | | 4.91 | 10.80 | 3.95 | 0.37 |
| 211813_x_at | 1634 | DCN | AF138303 | 4.70 | 9.69 | 2.65 | | 4.70 | 22.83 | 8.38 | 0.37 |
| 203443_at | 256364 | EML3 | AB012922 | 3.94 | 6.30 | 8.37 | | 3.94 | 14.74 | 5.42 | 0.37 |
| 219692_at | 79412 | KREMEN2 | NM_024507 | 11.95 | 19.09 | 29.50 | | 11.95 | 86.90 | 31.96 | 0.37 |
| 213048_s_at | 6418 | SET | W26593 | 9208.00 | 6033.37 | 8658.77 | | 9208.00 | 15146.32 | 5612.76 | 0.37 |
| 209297_at | 6453 | ITSN1 | AF114488 | 86.55 | 18.49 | 30.93 | | 86.55 | 168.69 | 62.52 | 0.37 |
| 217681_at | 647836 / | WNT7B | BE736994 | 7.96 | 4.86 | 19.28 | | 7.96 | 29.00 | 10.77 | 0.37 |
| | | /// | | | | | | | | | |
| | | LOC647836 | | | | | | | | | |
| | | /// | | | | | | | | | |
| | | LOC649 | | | | | | | | | |
| 209869_at | 150 | ADRA2A | AF284095 | 11.63 | 3.30 | 30.99 | | 11.63 | 27.61 | 10.28 | 0.37 |
| 211867_s_at | 56139 | PCDHA10 | AF152475 | 4.45 | 8.98 | 38.48 | | 4.45 | 14.66 | 5.47 | 0.37 |
| 218829_s_at | 55636 | CHD7 | AI475906 | 326.71 | 342.47 | 271.31 | | 326.71 | 836.14 | 313.27 | 0.37 |
| 201372_s_at | 8452 | CUL3 | AU145232 | 26.77 | 44.53 | 44.71 | | 26.77 | 49.16 | 18.50 | 0.38 |
| 220411_x_at | 79883 | PODNL1 | NM_024825 | 49.87 | 66.89 | 76.29 | | 49.87 | 84.49 | 31.84 | 0.38 |
| 217061_s_at | 2115 | ETV1 | AC004857 | 6.46 | 9.05 | 7.30 | | 6.46 | 21.22 | 8.00 | 0.38 |
| 221383_at | 10316 | NMUR1 | NM_006056 | 6.97 | 7.96 | 7.68 | | 6.97 | 13.07 | 4.94 | 0.38 |
| 211726_s_at | 2327 | FMO2 | BC005894 | 25.42 | 16.53 | 27.82 | | 25.42 | 44.26 | 16.87 | 0.38 |
| 213928_s_at | 3267 | HRB | AI742626 | 32.74 | 16.92 | 30.17 | | 32.74 | 71.28 | 27.23 | 0.38 |
| 209613_s_at | 125 | ADH1B | M21692 | 6.29 | 1.79 | 2.03 | | 6.29 | 12.06 | 4.62 | 0.38 |
| 207244_x_at | 1548 | CYP2A6 | NM_000762 | 14.24 | 34.95 | 94.24 | | 14.24 | 56.36 | 21.59 | 0.38 |
| 205814_at | 2913 | GRM3 | NM_000840 | 6.86 | 6.00 | 11.54 | | 6.86 | 19.30 | 7.42 | 0.38 |
| 207015_s_at | 8854 | ALDH1A2 | AB015228 | 4.60 | 3.38 | 6.32 | | 4.60 | 31.35 | 12.06 | 0.38 |
| 208410_x_at | 265 | AMELX | NM_001142 | 8.39 | 2.14 | 7.54 | | 8.39 | 19.05 | 7.33 | 0.38 |
| 209773_s_at | 6241 | RRM2 | BC001886 | 1867.24 | 2216.54 | 1532.67 | | 1867.24 | 3367.85 | 1299.07 | 0.39 |
| 209633_at | 5523 | PPP2R3A | AL389975 | 131.13 | 294.89 | 253.58 | | 131.13 | 764.33 | 294.90 | 0.39 |
| 203535_at | 6280 | S100A9 | NM_002965 | 1263.49 | 1163.30 | 1045.22 | | 1263.49 | 3171.00 | 1225.31 | 0.39 |
| 207610_s_at | 30817 | EMR2 | NM_013447 | 94.47 | 125.29 | 59.34 | | 94.47 | 249.34 | 96.83 | 0.39 |
| 214052_x_at | 23215 | BAT2D1 | AW238632 | 149.09 | 158.40 | 262.06 | | 149.09 | 382.59 | 148.64 | 0.39 |
| 207931_s_at | 5208 | PFKFB2 | NM_006212 | 12.56 | 50.71 | 56.20 | | 12.56 | 58.13 | 22.64 | 0.39 |
| 205576_at | 3053 | SERPIND1 | NM_000185 | 9.86 | 5.57 | 4.10 | | 9.86 | 43.03 | 16.80 | 0.39 |
| 215151_at | 55619 | DOCK10 | AB014594 | 24.13 | 71.37 | 55.78 | | 24.13 | 57.01 | 22.28 | 0.39 |
| 205180_s_at | 101 | ADAM8 | NM_001109 | 16.30 | 61.13 | 50.65 | | 16.30 | 99.62 | 38.95 | 0.39 |
| 220246_at | 57118 | CAMK1D | NM_020397 | 9.06 | 24.85 | 40.97 | | 9.06 | 30.15 | 11.81 | 0.39 |
| 215725_at | 25786 | DGCR11 | L77561 | 15.18 | 71.39 | 78.99 | | 15.18 | 75.80 | 29.69 | 0.39 |
| 213171_s_at | 10893 | MMP24 | AL121753 | 6.12 | 7.71 | 4.79 | | 6.12 | 16.87 | 6.61 | 0.39 |
| 220729_at | | | NM_014092 | 31.59 | 11.78 | 32.52 | | 31.59 | 66.41 | 26.05 | 0.39 |
| 217439_at | | | AL122122 | 2.70 | 13.68 | 22.54 | | 2.70 | 23.61 | 9.30 | 0.39 |
| 220803_at | 57559 | STAMBPL1 | NM_017597 | 3.00 | 3.98 | 5.83 | | 3.00 | 16.53 | 6.51 | 0.39 |
| 221018_s_at | 56165 | TDRD1 | NM_031278 | 2.90 | 6.63 | 11.57 | | 2.90 | 13.84 | 5.46 | 0.39 |
| 203407_at | 5493 | PPL | NM_002705 | 2.92 | 5.10 | 7.59 | | 2.92 | 11.66 | 4.60 | 0.39 |
| 205099_s_at | 1230 | CCR1 | AI421071 | 804.75 | 1012.48 | 541.74 | | 804.75 | 1440.10 | 570.64 | 0.40 |
| 207775_at | 79150 | MGC4859 | NM_024304 | 4.36 | 9.06 | 8.16 | | 4.36 | 10.46 | 4.15 | 0.40 |
| 211428_at | 5265 | SERPINA1 | AF119873 | 4.03 | 3.20 | 5.74 | | 4.03 | 16.60 | 6.58 | 0.40 |
| 203058_s_at | 9060 | PAPSS2 | AW299958 | 79.27 | 90.71 | 68.55 | | 79.27 | 375.47 | 149.15 | 0.40 |
| 60084_at | 1540 | CYLD | AI453099 | 12.53 | 23.80 | 18.53 | | 12.53 | 35.76 | 14.21 | 0.40 |
| 209632_at | 5523 | PPP2R3A | AI760130 | 75.46 | 154.21 | 142.61 | | 75.46 | 400.92 | 159.37 | 0.40 |
| 215552_s_at | 2099 | ESR1 | AI073549 | 4.14 | 2.41 | 15.34 | | 4.14 | 10.02 | 3.98 | 0.40 |
| 205034_at | 9134 | CCNE2 | NM_004702 | 1116.62 | 1437.07 | 698.33 | | 1116.62 | 2004.21 | 797.88 | 0.40 |
| 203687_at | 6376 | CX3CL1 | NM_002996 | 10.84 | 8.12 | 4.80 | | 10.84 | 22.94 | 9.18 | 0.40 |
| 206430_at | 1044 | CDX1 | NM_001804 | 12.44 | 32.40 | 31.28 | | 12.44 | 31.23 | 12.51 | 0.40 |
| 210575_at | 10726 | NUDC | AF241788 | 7.05 | 7.31 | 5.79 | | 7.05 | 20.65 | 8.30 | 0.40 |
| 213808_at | | | BE674466 | 1.09 | 4.72 | 3.15 | | 1.09 | 20.31 | 8.18 | 0.40 |
| 210550_s_at | 5923 | RASGRF1 | L26584 | 94.20 | 111.73 | 56.90 | | 94.20 | 143.98 | 58.08 | 0.40 |
| 210026_s_at | 29775 | CARD10 | AW205153 | 21.18 | 30.96 | 17.66 | | 21.18 | 118.17 | 47.70 | 0.40 |
| 205098_at | 1230 | CCR1 | AI421071 | 1280.61 | 1340.44 | 761.52 | | 1280.61 | 2817.21 | 1137.51 | 0.40 |
| 206297_at | 11330 | CTRC | NM_007272 | 51.41 | 60.91 | 49.80 | | 51.41 | 80.42 | 32.50 | 0.40 |
| 202436_s_at | 1545 | CYP1B1 | AU144855 | 1273.78 | 1404.79 | 570.44 | | 1273.78 | 3487.26 | 1411.86 | 0.40 |
| 207985_at | 26237 | ENO1B | NM_014355 | 10.30 | 9.33 | 3.42 | | 10.30 | 17.10 | 6.93 | 0.41 |
| 212883_at | 10452 | TOMM40 | AI358867 | 4.36 | 4.49 | 12.30 | | 4.36 | 22.58 | 9.15 | 0.41 |
| 212616_at | 80205 | CHD9 | BF668950 | 1390.38 | 1491.23 | 1262.55 | | 1390.38 | 2361.22 | 956.76 | 0.41 |
| 211980_at | 1282 | COL4A1 | AI922605 | 475.72 | 286.61 | 365.89 | | 475.72 | 882.69 | 357.69 | 0.41 |
| 201348_at | 2878 | GPX3 | NM_002084 | 20.47 | 20.58 | 20.86 | | 20.47 | 41.39 | 16.77 | 0.41 |
| 208789_at | 284119 | PTRF | BC004295 | 19.22 | 10.73 | 12.27 | | 19.22 | 129.45 | 52.49 | 0.41 |
| 217922_at | | | BE543064 | 741.45 | 838.83 | 535.61 | | 741.45 | 1578.20 | 640.41 | 0.41 |
| 216439_at | 10188 | TNK2 | AK024904 | 17.26 | 8.49 | 7.98 | | 17.26 | 31.64 | 12.87 | 0.41 |
| 221796_at | 4915 | NTRK2 | AA707199 | 3.64 | 6.73 | 5.77 | | 3.64 | 12.98 | 5.28 | 0.41 |
| 222068_s_at | 123872 | LRRC50 | AW663632 | 4.83 | 25.80 | 27.87 | | 4.83 | 249.46 | 101.58 | 0.41 |
| 221994_at | 10611 | PDLIM5 | AA196325 | 16.49 | 36.08 | 32.58 | | 16.49 | 33.85 | 13.79 | 0.41 |
| 204115_at | 2791 | GNG11 | NM_004126 | 32.07 | 14.31 | 5.25 | | 32.07 | 56.54 | 23.06 | 0.41 |
| 205302_at | 3484 | IGFBP1 | NM_000596 | 5.25 | 6.77 | 20.29 | | 5.25 | 23.84 | 9.73 | 0.41 |
| 216408_at | 81697 | OR2B2 | AJ302584 | 6.46 | 1.47 | 3.61 | | 6.46 | 14.37 | 5.87 | 0.41 |
| 214164_x_at | 771 | CA12 | BF752277 | 42.49 | 49.48 | 12.32 | | 42.49 | 108.50 | 44.31 | 0.41 |
| 208123_at | 9312 | KCNB2 | AF338730 | 5.82 | 8.54 | 13.80 | | 5.82 | 24.41 | 9.97 | 0.41 |
| 204762_s_at | 2775 | GNAO1 | NM_020988 | 19.28 | 28.64 | 13.84 | | 19.28 | 33.05 | 13.52 | 0.41 |
| 214345_at | | | BF058643 | 24.69 | 9.90 | 13.55 | | 24.69 | 38.84 | 15.94 | 0.41 |
| 210343_s_at | 9356 | SLC22A6 | AF124373 | 9.97 | 11.76 | 7.50 | | 9.97 | 15.70 | 6.45 | 0.41 |
| 207519_at | 6532 | SLC6A4 | NM_001045 | 10.05 | 6.24 | 13.68 | | 10.05 | 28.59 | 11.75 | 0.41 |
| 221030_s_at | 83478 | ARHGAP24 | NM_031305 | 17.03 | 21.38 | 19.82 | | 17.03 | 89.13 | 36.67 | 0.41 |
| 216494_at | 645468 / | LOC645468 | AL023775 | 13.30 | 7.00 | 6.72 | | 13.30 | 21.64 | 8.92 | 0.41 |
| | | /// | | | | | | | | | |
| | | LOC651107 | | | | | | | | | |
| 205184_at | 2786 | GNG4 | NM_004485 | 116.95 | 138.87 | 77.98 | | 116.95 | 529.58 | 218.43 | 0.41 |
| 213582_at | 23250 | ATP11A | BF439472 | 62.30 | 89.60 | 72.02 | | 62.30 | 138.39 | 57.12 | 0.41 |
| 201130_s_at | 999 | CDH1 | NM_004360 | 1.20 | 4.97 | 4.07 | | 1.20 | 15.70 | 6.48 | 0.41 |
| 218044_x_at | 5763 | PTMS | M24398 | 18.05 | 21.39 | 33.02 | | 18.05 | 30.50 | 12.59 | 0.41 |
| 205625_s_at | 793 | CALB1 | AW014927 | 0.92 | 2.19 | 3.47 | | 0.92 | 12.19 | 5.03 | 0.41 |
| 202437_s_at | 1545 | CYP1B1 | NM_000104 | 625.40 | 780.26 | 321.65 | | 625.40 | 2128.34 | 880.49 | 0.41 |
| 205765_at | 1577 | CYP3A5 | NM_000777 | 2.51 | 6.23 | 3.78 | | 2.51 | 21.03 | 8.72 | 0.41 |
| 216842_x_at | 159163 / | RBMY1A1 | U38460 | 29.69 | 13.64 | 23.56 | | 29.69 | 53.36 | 22.13 | 0.41 |
| | | /// | | | | | | | | | |
| | | RBMY1F | | | | | | | | | |
| | | /// | | | | | | | | | |
| | | RBMY1B | | | | | | | | | |
| 211002_s_at | 23650 | TRIM29 | AF230389 | 4.84 | 8.94 | 3.99 | | 4.84 | 15.29 | 6.35 | 0.42 |
| 219520_s_at | 55841 | WWC3 | NM_018458 | 207.01 | 252.15 | 208.72 | | 207.01 | 428.87 | 178.48 | 0.42 |
| 207222_at | 8399 | PLA2G10 | NM_003561 | 14.38 | 47.25 | 38.13 | | 14.38 | 26.77 | 11.14 | 0.42 |
| 219636_s_at | 80210 | ARMC9 | NM_025139 | 5.75 | 19.00 | 31.84 | | 5.75 | 49.45 | 20.59 | 0.42 |
| 203917_at | 1525 | CXADR | NM_001338 | 39.30 | 41.28 | 30.52 | | 39.30 | 77.08 | 32.10 | 0.42 |
| 204491_at | 5144 | PDE4D | R40917 | 133.25 | 174.31 | 211.33 | | 133.25 | 452.65 | 188.74 | 0.42 |
| 209648_x_at | 9655 | SOCS5 | AL136896 | 174.11 | 197.15 | 205.62 | | 174.11 | 308.15 | 128.78 | 0.42 |
| 220378_at | 6954 | TCP11 | NM_018679 | 11.33 | 5.72 | 2.44 | | 11.33 | 21.25 | 8.91 | 0.42 |
| 203921_at | 9435 | CHST2 | NM_004267 | 79.60 | 498.72 | 401.55 | | 79.60 | 1003.43 | 420.69 | 0.42 |
| 214660_at | 53918 | PELO | X68742 | 16.48 | 4.64 | 7.45 | | 16.48 | 30.51 | 12.80 | 0.42 |
| 215894_at | 5729 | PTGDR | AI460323 | 7.42 | 5.46 | 10.82 | | 7.42 | 11.56 | 4.85 | 0.42 |
| 205528_s_at | 862 | RUNX1T1 | X79990 | 3.53 | 4.30 | 20.47 | | 3.53 | 91.06 | 38.29 | 0.42 |
| 210610_at | 634 | CEACAM1 | M69176 | 13.88 | 20.35 | 15.83 | | 13.88 | 53.82 | 22.65 | 0.42 |
| 200618_at | 3927 | LASP1 | NM_006148 | 2009.27 | 2011.50 | 1515.01 | | 2009.27 | 3899.85 | 1645.64 | 0.42 |
| 217244_at | | | AL021327 | 2.80 | 2.10 | 1.70 | | 2.80 | 13.67 | 5.77 | 0.42 |
| 201417_at | 6659 | SOX4 | AL136179 | 340.42 | 399.80 | 350.07 | | 340.42 | 899.40 | 380.77 | 0.42 |
| 215321_at | 154661 | RPIB9 | AI825798 | 32.51 | 9.19 | 55.07 | | 32.51 | 64.21 | 27.20 | 0.42 |
| 207454_at | 2899 | GRIK3 | NM_000831 | 3.38 | 4.62 | 8.93 | | 3.38 | 10.83 | 4.59 | 0.42 |
| 206002_at | 10149 | GPR64 | NM_005756 | 10.15 | 30.78 | 31.88 | | 10.15 | 29.61 | 12.55 | 0.42 |
| 206151_x_at | 23436 | ELA3B | NM_007352 | 7.00 | 7.53 | 5.59 | | 7.00 | 12.58 | 5.34 | 0.42 |
| 218856_at | 27242 | TNFRSF21 | NM_016629 | 652.00 | 768.42 | 677.72 | | 652.00 | 2467.23 | 1048.09 | 0.42 |
| 213443_at | 8717 | TRADD | N36774 | 3.48 | 8.92 | 6.89 | | 3.48 | 15.94 | 6.78 | 0.43 |
| 211675_s_at | 29969 | MDFIC | AF054589 | 1419.27 | 1260.63 | 1322.18 | | 1419.27 | 7904.07 | 3364.37 | 0.43 |
| 208484_at | 3024 | HIST1H1A | NM_005325 | 3.38 | 3.93 | 1.77 | | 3.38 | 10.48 | 4.47 | 0.43 |
| 203354_s_at | 23362 | PSD3 | NM_015310 | 112.90 | 188.34 | 253.36 | | 112.90 | 707.21 | 302.01 | 0.43 |
| 220208_at | 11093 | ADAMTS13 | NM_017587 | 33.95 | 41.07 | 57.04 | | 33.95 | 62.92 | 26.91 | 0.43 |
| 203063_at | 9647 | PPM1F | NM_014634 | 383.51 | 489.93 | 381.53 | | 383.51 | 621.16 | 266.36 | 0.43 |
| 204603_at | 9156 | EXO1 | NM_003686 | 625.28 | 614.87 | 484.97 | | 625.28 | 1128.18 | 484.19 | 0.43 |
| 215252_at | 7266 | DNAJC7 | AW814026 | 330.12 | 346.32 | 484.63 | | 330.12 | 627.49 | 269.31 | 0.43 |
| 207311_at | 8447 | DOC2B | NM_003585 | 8.97 | 16.99 | 34.64 | | 8.97 | 35.37 | 15.20 | 0.43 |
| 206137_at | 9699 | RIMS2 | NM_014677 | 23.93 | 45.32 | 86.23 | | 23.93 | 38.87 | 16.71 | 0.43 |
| 215822_x_at | 4661 | MYT1 | M96980 | 8.95 | 16.17 | 17.17 | | 8.95 | 56.78 | 24.44 | 0.43 |
| 214420_s_at | 1559 | CYP2C9 | AV657878 | 7.92 | 5.49 | 10.96 | | 7.92 | 13.54 | 5.83 | 0.43 |
| 205286_at | 7022 | TFAP2C | U85658 | 38.85 | 28.24 | 24.65 | | 38.85 | 113.24 | 48.79 | 0.43 |
| 209540_at | 3479 | IGF1 | M29644 | 11.09 | 6.04 | 7.71 | | 11.09 | 27.62 | 11.91 | 0.43 |
| 219855_at | 55190 | NUDT11 | NM_018159 | 5.61 | 8.07 | 5.64 | | 5.61 | 21.56 | 9.30 | 0.43 |
| 212927_at | 23137 | SMC5 | AW183677 | 221.82 | 271.10 | 221.26 | | 221.82 | 445.92 | 192.87 | 0.43 |
| 215675_at | 7008 | TEF | AB051442 | 4.94 | 5.16 | 7.99 | | 4.94 | 26.37 | 11.43 | 0.43 |
| 206336_at | 6372 | CXCL6 | NM_002993 | 7.54 | 2.50 | 26.49 | | 7.54 | 1643.05 | 714.11 | 0.43 |
| 212013_at | 7837 | PXDN | D86983 | 9.40 | 6.95 | 14.24 | | 9.40 | 14.55 | 6.33 | 0.43 |
| 206765_at | 3759 | KCNJ2 | AF153820 | 127.19 | 124.00 | 196.39 | | 127.19 | 531.69 | 231.32 | 0.44 |
| 203477_at | 1306 | COL15A1 | NM_001855 | 80.00 | 59.46 | 62.52 | | 80.00 | 138.69 | 60.37 | 0.44 |
| 210596_at | | | AF130104 | 454.39 | 374.73 | 527.00 | | 454.39 | 755.48 | 329.13 | 0.44 |
| 205966_at | 6884 | TAF13 | NM_005645 | 20.01 | 12.80 | 22.70 | | 20.01 | 45.51 | 19.84 | 0.44 |
| 210303_at | 10586 | MAB21L2 | AF262032 | 5.40 | 3.92 | 10.70 | | 5.40 | 19.36 | 8.46 | 0.44 |
| 35776_at | 6453 | ITSN1 | AF064243 | 117.00 | 129.34 | 114.72 | | 117.00 | 190.05 | 83.12 | 0.44 |
| 214386_at | 3166 | HMX1 | AI939402 | 4.40 | 10.88 | 17.53 | | 4.40 | 11.89 | 5.21 | 0.44 |
| 213277_at | 677 | ZFP36L1 | AI344045 | 7.92 | 5.59 | 12.63 | | 7.92 | 18.80 | 8.25 | 0.44 |
| 215538_at | 730062 / | LOC730062 | BF057493 | 10.95 | 11.98 | 6.18 | | 10.95 | 26.97 | 11.85 | 0.44 |
| | | /// | | | | | | | | | |
| | | LOC731972 | | | | | | | | | |
| 216891_at | | | U00956 | 4.77 | 5.53 | 11.17 | | 4.77 | 22.36 | 9.83 | 0.44 |
| 215277_at | 5097 | PCDH1 | AA481656 | 9.69 | 3.38 | 6.78 | | 9.69 | 41.92 | 18.44 | 0.44 |
| 207784_at | 414 | ARSD | NM_009589 | 4.60 | 6.05 | 36.57 | | 4.60 | 14.49 | 6.38 | 0.44 |
| 202688_at | 8743 | TNFSF10 | NM_003810 | 412.50 | 502.19 | 210.74 | | 412.50 | 775.60 | 341.63 | 0.44 |
| 210163_at | 6373 | CXCL11 | AF030514 | 11.28 | 89.98 | 73.90 | | 11.28 | 74.88 | 32.99 | 0.44 |
| 204146_at | 10635 | RAD51AP1 | BE966146 | 508.40 | 392.56 | 578.83 | | 508.40 | 890.24 | 392.25 | 0.44 |
| 205187_at | 4090 | SMAD5 | AF010601 | 95.62 | 69.51 | 93.56 | | 95.62 | 171.06 | 75.40 | 0.44 |
| 219896_at | 50632 | DRD1IP | NM_015722 | 4.03 | 6.84 | 12.02 | | 4.03 | 11.75 | 5.18 | 0.44 |
| 215698_at | 5927 | JARID1A | AF007135 | 98.34 | 80.27 | 93.60 | | 98.34 | 165.22 | 72.99 | 0.44 |
| 220362_at | 170679 | PSORS1C1 | NM_014068 | 13.00 | 10.64 | 25.72 | | 13.00 | 19.56 | 8.66 | 0.44 |
| 205505_at | 2650 | GCNT1 | NM_001490 | 232.59 | 229.26 | 217.79 | | 232.59 | 426.47 | 188.95 | 0.44 |
| 206660_at | 3543 | IGLL1 | NM_020070 | 8.10 | 4.03 | 3.89 | | 8.10 | 31.18 | 13.82 | 0.44 |
| 205931_s_at | 9586 | CREB5 | NM_004904 | 16.50 | 5.89 | 5.71 | | 16.50 | 37.16 | 16.47 | 0.44 |
| 202285_s_at | 4070 | TACSTD2 | NM_002353 | 8.02 | 17.65 | 18.13 | | 8.02 | 39.00 | 17.31 | 0.44 |
| 212077_at | 800 | CALD1 | AL583520 | 9.65 | 19.45 | 16.60 | | 9.65 | 19.71 | 8.76 | 0.44 |
| 208701_at | 334 | APLP2 | BC000373 | 210.25 | 207.51 | 224.80 | | 210.25 | 356.38 | 158.69 | 0.45 |
| 218995_s_at | 1906 | EDN1 | J05008 | 5.90 | 10.77 | 10.13 | | 5.90 | 14.55 | 6.49 | 0.45 |
| 218399_s_at | 55038 | CDCA4 | NM_017955 | 909.74 | 1088.69 | 908.84 | | 909.74 | 1836.61 | 819.95 | 0.45 |
| 212477_at | 23527 | CENTB2 | D26069 | 38.01 | 38.65 | 49.77 | | 38.01 | 64.81 | 28.94 | 0.45 |
| 221816_s_at | 158852 / | PHF11 | BF055474 | 760.35 | 1093.60 | 863.09 | | 760.35 | 1141.83 | 510.50 | 0.45 |
| | | /// | | | | | | | | | |
| | | RP13- | | | | | | | | | |
| | | 36C9.1 | | | | | | | | | |
| | | /// | | | | | | | | | |
| | | RP13 | | | | | | | | | |
| 207501_s_at | 2257 | FGF12 | NM_004113 | 1.88 | 7.00 | 6.63 | | 1.88 | 14.42 | 6.45 | 0.45 |
| 213866_at | 201191 | SAMD14 | AL134453 | 21.47 | 68.44 | 60.23 | | 21.47 | 78.33 | 35.11 | 0.45 |
| 221960_s_at | 5862 | RAB2 | AI189609 | 132.04 | 104.98 | 154.40 | | 132.04 | 213.39 | 95.87 | 0.45 |
| 215547_at | 9819 | TSC22D2 | AF201291 | 13.78 | 37.76 | 39.86 | | 13.78 | 33.46 | 15.04 | 0.45 |
| 215610_at | 728263 / | LOC728263 | AK022038 | 52.22 | 68.45 | 28.01 | | 52.22 | 98.87 | 44.62 | 0.45 |
| | | /// | | | | | | | | | |
| | | LOC731183 | | | | | | | | | |
| 208388_at | 10002 | NR2E3 | NM_014249 | 9.26 | 7.12 | 7.75 | | 9.26 | 27.39 | 12.36 | 0.45 |
| 206447_at | 63036 | ELA2A | NM_001971 | 2.69 | 6.57 | 1.64 | | 2.69 | 16.10 | 7.27 | 0.45 |
| 216856_s_at | | | AF264787 | 35.94 | 19.42 | 61.68 | | 35.94 | 107.46 | 48.57 | 0.45 |
| 217163_at | 2099 | ESR1 | X63118 | 5.38 | 2.41 | 12.26 | | 5.38 | 10.05 | 4.54 | 0.45 |
| 203745_at | 3052 | HCCS | AI801013 | 356.54 | 380.83 | 345.60 | | 356.54 | 814.86 | 368.71 | 0.45 |
| 206020_at | 9306 | SOCS6 | NM_016387 | 115.84 | 116.66 | 104.12 | | 115.84 | 202.24 | 91.57 | 0.45 |
| 221912_s_at | 79140 | CCDC28B | AL049795 | 31.72 | 36.80 | 62.87 | | 31.72 | 56.43 | 25.55 | 0.45 |
| 222330_at | 5140 | PDE3B | AW974995 | 8.56 | 64.82 | 63.97 | | 8.56 | 59.50 | 26.95 | 0.45 |
| 214704_at | 22980 | TCF25 | AK024679 | 9.35 | 9.88 | 11.30 | | 9.35 | 14.99 | 6.80 | 0.45 |
| 215204_at | 26054 | SENP6 | AU147295 | 280.16 | 518.45 | 510.30 | | 280.16 | 473.39 | 214.66 | 0.45 |
| 217183_at | 3949 | LDLR | S70123 | 8.35 | 9.65 | 12.06 | | 8.35 | 23.01 | 10.44 | 0.45 |
| 217575_s_at | 6655 | SOS2 | BF692958 | 2.55 | 4.81 | 5.58 | | 2.55 | 15.29 | 6.95 | 0.45 |
| 219806_s_at | 56935 | C11orf75 | NM_020179 | 813.80 | 868.61 | 1189.71 | | 813.80 | 1426.85 | 648.44 | 0.45 |
| 203425_s_at | 3488 | IGFBP5 | M65062 | 27.36 | 27.39 | 47.86 | | 27.36 | 69.42 | 31.57 | 0.45 |
| 213832_at | | | AA530995 | 23.92 | 4.85 | 1.79 | | 23.92 | 43.91 | 20.00 | 0.46 |
| 209121_x_at | 7026 | NR2F2 | M64497 | 64.03 | 69.03 | 48.15 | | 64.03 | 145.52 | 66.30 | 0.46 |
| 213087_s_at | 1936 | EEF1D | BF690020 | 79.37 | 48.97 | 70.10 | | 79.37 | 132.63 | 60.58 | 0.46 |
| 208942_s_at | 7095 | TLOC1 | U93239 | 2339.60 | 2197.18 | 2676.33 | | 2339.60 | 5369.52 | 2454.47 | 0.46 |
| 221559_s_at | 79003 | MIS12 | BC000229 | 287.87 | 302.62 | 325.60 | | 287.87 | 617.00 | 282.21 | 0.46 |
| 210906_x_at | 361 | AQP4 | U34846 | 3.92 | 2.75 | 5.10 | | 3.92 | 10.50 | 4.81 | 0.46 |
| 206919_at | 2005 | ELK4 | NM_021795 | 4.63 | 25.69 | 32.95 | | 4.63 | 38.85 | 17.81 | 0.46 |
| 202687_s_at | 8743 | TNFSF10 | NM_003810 | 216.68 | 253.09 | 115.06 | | 216.68 | 338.68 | 155.25 | 0.46 |
| 213437_at | 22902 | RUFY3 | AA861784 | 17.65 | 24.37 | 38.78 | | 17.65 | 56.41 | 25.93 | 0.46 |
| 213951_s_at | 29893 | PSMC3IP | BE964655 | 249.95 | 319.91 | 280.94 | | 249.95 | 559.59 | 257.29 | 0.46 |
| 222070_at | 373863 | DND1 | AW090043 | 160.67 | 169.26 | 127.16 | | 160.67 | 245.61 | 112.96 | 0.46 |
| 213092_x_at | 23234 | DNAJC9 | AW241779 | 382.02 | 292.84 | 320.19 | | 382.02 | 636.50 | 292.89 | 0.46 |
| 220627_at | 10047 | CST8 | NM_005492 | 12.36 | 12.60 | 5.89 | | 12.36 | 19.00 | 8.75 | 0.46 |
| 219973_at | 79642 | ARSJ | NM_024590 | 1.61 | 7.93 | 18.04 | | 1.61 | 18.20 | 8.39 | 0.46 |
| 202792_s_at | 9701 | SAPS2 | NM_014678 | 146.47 | 217.94 | 90.70 | | 146.47 | 225.64 | 104.08 | 0.46 |
| 220859_at | | | NM_014136 | 5.44 | 9.37 | 2.72 | | 5.44 | 23.20 | 10.71 | 0.46 |
| 203544_s_at | 8027 | STAM | NM_003473 | 218.80 | 200.55 | 256.16 | | 218.80 | 419.30 | 193.54 | 0.46 |
| 206813_at | 1489 | CTF1 | NM_001330 | 5.75 | 7.96 | 9.63 | | 5.75 | 10.60 | 4.90 | 0.46 |
| 219229_at | 28232 | SLCO3A1 | NM_013272 | 344.12 | 546.43 | 561.62 | | 344.12 | 782.05 | 361.57 | 0.46 |
| 209501_at | 1039 | CDR2 | AL582414 | 406.72 | 480.56 | 528.64 | | 406.72 | 797.87 | 369.33 | 0.46 |
| 212620_at | 23060 | ZNF609 | AW165979 | 70.02 | 92.79 | 93.35 | | 70.02 | 142.24 | 65.85 | 0.46 |
| 211441_x_at | 64816 | CYP3A43 | AF280113 | 6.46 | 8.14 | 0.73 | | 6.46 | 13.15 | 6.09 | 0.46 |
| 222304_x_at | 26628 | OR7E47P | AW514038 | 13.29 | 9.56 | 11.52 | | 13.29 | 27.80 | 12.88 | 0.46 |
| 219609_at | 79446 | WDR25 | NM_024515 | 19.82 | 11.57 | 12.34 | | 19.82 | 36.03 | 16.70 | 0.46 |
| 221516_s_at | 54471 | SMCR7L | BC002587 | 1051.50 | 967.40 | 934.79 | | 1051.50 | 1937.54 | 899.75 | 0.46 |
| 202892_at | 8697 | CDC23 | NM_004661 | 764.29 | 768.29 | 640.18 | | 764.29 | 1204.26 | 559.41 | 0.46 |
| 213523_at | 898 | CCNE1 | AI671049 | 493.85 | 602.45 | 399.25 | | 493.85 | 1201.18 | 558.12 | 0.46 |
| 220420_at | 79748 | LMAN1L | NM_021819 | 9.01 | 6.96 | 4.92 | | 9.01 | 17.17 | 7.98 | 0.46 |
| 215871_at | 5322 | PLA2G5 | AL158172 | 2.20 | 4.35 | 9.63 | | 2.20 | 23.94 | 11.14 | 0.47 |
| 218623_at | 51617 | HMP19 | NM_015980 | 15.56 | 3.63 | 3.13 | | 15.56 | 24.20 | 11.27 | 0.47 |
| 214677_x_at | 28786 | IGL@ | X57812 | 13.39 | 17.75 | 8.11 | | 13.39 | 20.51 | 9.55 | 0.47 |
| | // | /// | | | | | | | | | |
| | | IGLV4- | | | | | | | | | |
| | | 3 /// | | | | | | | | | |
| | | IGLV3- | | | | | | | | | |
| | | 25 | | | | | | | | | |
| 211652_s_at | 3929 | LBP | M35533 | 11.66 | 24.39 | 20.69 | | 11.66 | 53.50 | 24.95 | 0.47 |
| 206545_at | 940 | CD28 | NM_006139 | 1.37 | 11.63 | 9.81 | | 1.37 | 17.41 | 8.12 | 0.47 |
| 204068_at | 6788 | STK3 | NM_006281 | 609.39 | 661.92 | 1145.95 | | 609.39 | 1394.06 | 650.54 | 0.47 |
| 207719_x_at | 9859 | CEP170 | NM_014812 | 971.09 | 936.59 | 1042.16 | | 971.09 | 1480.30 | 691.59 | 0.47 |
| 220013_at | 79852 | ABHD9 | NM_024794 | 5.32 | 8.11 | 22.55 | | 5.32 | 17.75 | 8.29 | 0.47 |
| 206578_at | 1482 | NKX2-5 | NM_004387 | 10.81 | 7.42 | 9.58 | | 10.81 | 23.51 | 10.99 | 0.47 |
| 212759_s_at | 6934 | TCF7L2 | AI703074 | 15.90 | 11.57 | 15.01 | | 15.90 | 42.71 | 20.02 | 0.47 |
| 204753_s_at | 3131 | HLF | AI810712 | 5.84 | 2.18 | 14.68 | | 5.84 | 10.81 | 5.08 | 0.47 |
| 213913_s_at | 23329 | KIAA0984 | AW134976 | 23.11 | 17.80 | 19.53 | | 23.11 | 38.51 | 18.10 | 0.47 |
| 216846_at | 3535 | IGL@ | AF234254 | 6.09 | 2.99 | 8.14 | | 6.09 | 10.78 | 5.08 | 0.47 |
| 213219_at | 108 | ADCY2 | AB028983 | 6.52 | 11.00 | 9.57 | | 6.52 | 13.02 | 6.13 | 0.47 |
| 201330_at | 5917 | RARS | NM_002887 | 1949.10 | 1463.27 | 1700.46 | | 1949.10 | 3004.78 | 1415.94 | 0.47 |
| 203753_at | 6925 | TCF4 | NM_003199 | 145.94 | 114.22 | 158.65 | | 145.94 | 258.80 | 121.99 | 0.47 |
| 207840_at | 11126 | CD160 | NM_007053 | 19.05 | 12.04 | 10.09 | | 19.05 | 38.49 | 18.15 | 0.47 |
| 206838_at | 9095 | TBX19 | NM_005149 | 86.99 | 123.34 | 89.60 | | 86.99 | 137.86 | 65.08 | 0.47 |
| 206933_s_at | 9563 | H6PD | NM_004285 | 4.94 | 7.38 | 8.02 | | 4.94 | 13.62 | 6.45 | 0.47 |
| 208615_s_at | 8073 | PTP4A2 | AF208850 | 1057.30 | 1306.62 | 1139.73 | | 1057.30 | 2562.19 | 1217.48 | 0.48 |
| 203414_at | 23531 | MMD | NM_012329 | 604.70 | 836.25 | 733.27 | | 604.70 | 1332.71 | 633.42 | 0.48 |
| 216286_at | 9331 | B4GALT6 | AV760769 | 28.57 | 34.12 | 44.78 | | 28.57 | 42.95 | 20.43 | 0.48 |
| 214257_s_at | 9554 | SEC22B | AA890010 | 2185.00 | 1534.51 | 2935.81 | | 2185.00 | 4901.57 | 2331.41 | 0.48 |
| 205923_at | 5649 | RELN | NM_005045 | 3.60 | 16.30 | 38.60 | | 3.60 | 21.45 | 10.25 | 0.48 |
| 217168_s_at | 9709 | HERPUD1 | AF217990 | 1669.26 | 2386.03 | 3476.03 | | 1669.26 | 6156.64 | 2948.91 | 0.48 |
| 218249_at | 64429 | ZDHHC6 | NM_022494 | 580.61 | 726.38 | 550.70 | | 580.61 | 1058.00 | 506.88 | 0.48 |
| 205159_at | 1439 | CSF2RB | AV756141 | 673.88 | 846.20 | 718.47 | | 673.88 | 1162.18 | 558.04 | 0.48 |
| 211984_at | 801 | CALM1 | AI653730 | 1080.10 | 1123.86 | 811.09 | | 1080.10 | 2044.09 | 981.64 | 0.48 |
| 209250_at | 8560 | DEGS1 | BC000961 | 2245.39 | 3471.41 | 3212.51 | | 2245.39 | 9207.58 | 4424.00 | 0.48 |
| 202256_at | 10421 | CD2BP2 | BF793888 | 178.58 | 267.73 | 123.49 | | 178.58 | 312.71 | 150.39 | 0.48 |
| 200685_at | 9295 | SFRS11 | AU146237 | 236.45 | 158.40 | 186.72 | | 236.45 | 371.71 | 178.88 | 0.48 |
| 37547_at | 27241 | PTHB1 | U85995 | 3.19 | 5.31 | 5.62 | | 3.19 | 10.37 | 4.99 | 0.48 |
| 210057_at | 23049 | SMG1 | U32581 | 95.85 | 141.25 | 119.54 | | 95.85 | 224.41 | 108.10 | 0.48 |
| 213956_at | 9857 | CEP350 | AW299294 | 593.86 | 738.43 | 786.34 | | 593.86 | 905.12 | 436.09 | 0.48 |
| 202751_at | 24144 | TFIP11 | NM_012143 | 46.26 | 54.31 | 49.87 | | 46.26 | 103.54 | 49.90 | 0.48 |
| 214481_at | 8336 | HIST1H2AM | NM_003514 | 15.80 | 4.47 | 33.89 | | 15.80 | 28.52 | 13.75 | 0.48 |
| 204729_s_at | 6804 | STX1A | NM_004603 | 33.67 | 66.32 | 84.46 | | 33.67 | 103.82 | 50.06 | 0.48 |
| 206708_at | 3344 | FOXN2 | NM_002158 | 75.68 | 70.40 | 90.70 | | 75.68 | 168.35 | 81.27 | 0.48 |
| 216117_at | 27085 | MTBP | AK025114 | 10.22 | 38.67 | 36.82 | | 10.22 | 42.34 | 20.50 | 0.48 |
| 217012_at | | | AL080233 | 1.50 | 6.99 | 15.85 | | 1.50 | 18.26 | 8.86 | 0.49 |
| 208176_at | 26584 | DUX1 | NM_012146 | 22.57 | 24.42 | 5.20 | | 22.57 | 35.70 | 17.33 | 0.49 |
| 201451_x_at | 6009 | RHEB | D78132 | 10.51 | 14.12 | 30.45 | | 10.51 | 75.64 | 36.72 | 0.49 |
| 221278_at | 3218 | HOXB8 | NM_024016 | 4.08 | 2.57 | 15.70 | | 4.08 | 17.14 | 8.34 | 0.49 |
| 202599_s_at | 8204 | NRIP1 | NM_003489 | 645.40 | 663.59 | 609.51 | | 645.40 | 1479.20 | 719.89 | 0.49 |
| 209043_at | 9061 | PAPSS1 | AF033026 | 467.40 | 584.83 | 371.60 | | 467.40 | 830.78 | 405.25 | 0.49 |
| 216596_at | 26082 | DKFZP434L187 | AL117445 | 9.06 | 7.00 | 7.87 | | 9.06 | 26.69 | 13.03 | 0.49 |
| 216557_x_at | 2537 /// | IFI6 /// | U92706 | 6.06 | 24.01 | 25.75 | | 6.06 | 44.38 | 21.68 | 0.49 |
| | | IGHA1 | | | | | | | | | |
| | | /// | | | | | | | | | |
| | | IGHG1 | | | | | | | | | |
| | | /// I | | | | | | | | | |
| 201899_s_at | 7319 | UBE2A | NM_003336 | 1836.90 | 1907.49 | 1727.04 | | 1836.90 | 3167.95 | 1549.17 | 0.49 |
| 201213_at | | | AI090331 | 116.62 | 133.61 | 110.86 | | 116.62 | 194.86 | 95.57 | 0.49 |
| 220510_at | 57127 | RHBG | NM_020407 | 11.17 | 8.74 | 9.79 | | 11.17 | 16.85 | 8.27 | 0.49 |
| 217599_s_at | 29969 | MDFIC | BE910600 | 118.63 | 81.89 | 90.10 | | 118.63 | 574.23 | 281.77 | 0.49 |
| 218962_s_at | 64418 | TMEM168 | NM_022484 | 643.50 | 629.20 | 654.83 | | 643.50 | 1087.93 | 534.50 | 0.49 |
| 205038_at | 10320 | IKZF1 | BG540504 | 317.07 | 308.68 | 163.79 | | 317.07 | 1541.16 | 757.29 | 0.49 |
| 207490_at | 80086 | TUBA4 | NM_025019 | 48.59 | 62.15 | 32.35 | | 48.59 | 76.47 | 37.58 | 0.49 |
| 217663_at | | | AW264320 | 23.76 | 36.95 | 47.22 | | 23.76 | 55.26 | 27.17 | 0.49 |
| 209555_s_at | 948 | CD36 | BE968792 | 273.65 | 331.19 | 282.13 | | 273.65 | 423.74 | 208.36 | 0.49 |
| 216453_at | 57336 | ZNF287 | AL359578 | 10.13 | 10.64 | 25.14 | | 10.13 | 19.72 | 9.71 | 0.49 |
| 204695_at | 993 | CDC25A | AI343459 | 351.24 | 493.20 | 225.60 | | 351.24 | 879.42 | 433.11 | 0.49 |
| 204789_at | 752 | FMNL1 | NM_005892 | 468.33 | 458.86 | 410.11 | | 468.33 | 886.29 | 437.39 | 0.49 |
| 216877_at | 401014 | DKFZp686O1327 | U80770 | 25.52 | 55.39 | 53.03 | | 25.52 | 66.72 | 32.93 | 0.49 |
| 221439_at | 10741 | RBBP9 | NM_006606 | 6.43 | 7.52 | 8.89 | | 6.43 | 12.53 | 6.19 | 0.49 |
| 205894_at | 415 | ARSE | NM_000047 | 17.55 | 11.97 | 7.02 | | 17.55 | 35.06 | 17.32 | 0.49 |
| 211085_s_at | 6789 | STK4 | Z25430 | 152.17 | 142.55 | 158.54 | | 152.17 | 232.05 | 114.64 | 0.49 |
| 208086_s_at | 1756 | DMD | M92650 | 8.43 | 4.87 | 7.26 | | 8.43 | 27.15 | 13.42 | 0.49 |
| 211646_at | | | L14453 | 2.65 | 1.09 | 0.45 | | 2.65 | 16.03 | 7.95 | 0.50 |
| 206737_at | 7481 | WNT11 | NM_004626 | 4.49 | 3.92 | 4.48 | | 4.49 | 17.09 | 8.49 | 0.50 |
| 218324_s_at | 65244 | SPATS2 | NM_023071 | 50.62 | 101.66 | 79.55 | | 50.62 | 93.85 | 46.64 | 0.50 |
| 208110_x_at | 81857 | MED25 | NM_030973 | 6.75 | 7.90 | 6.33 | | 6.75 | 12.53 | 6.25 | 0.50 |
| 219790_s_at | 4883 | NPR3 | NM_000908 | 7.82 | 11.53 | 3.49 | | 7.82 | 17.26 | 8.61 | 0.50 |
| 215059_at | 79625 | C4orf31 | AA053967 | 1.62 | 2.22 | 1.96 | | 1.62 | 13.63 | 6.82 | 0.50 |
| 200914_x_at | 3895 | KTN1 | NM_004986 | 1049.11 | 752.35 | 1272.78 | | 1049.11 | 1823.45 | 912.58 | 0.50 |
| 212027_at | 58517 | RBM25 | AI925305 | 860.97 | 770.64 | 725.45 | | 860.97 | 1467.98 | 734.70 | 0.50 |
| 215685_s_at | 1746 | DLX2 | BE792224 | 26.96 | 20.58 | 44.08 | | 26.96 | 54.20 | 27.13 | 0.50 |
| 204461_x_at | 5810 | RAD1 | NM_002853 | 782.00 | 679.22 | 754.59 | | 782.00 | 1292.64 | 647.02 | 0.50 |
| 203487_s_at | 25852 | ARMC8 | NM_015396 | 414.33 | 380.06 | 354.70 | | 414.33 | 676.69 | 338.88 | 0.50 |
| 202976_s_at | 22836 | RHOBTB3 | NM_014899 | 276.77 | 258.23 | 187.81 | | 276.77 | 471.05 | 236.38 | 0.50 |
| 204235_s_at | 51454 | GULP1 | AF200715 | 22.41 | 28.98 | 8.00 | | 22.41 | 62.21 | 31.22 | 0.50 |
| 208502_s_at | 5307 | PITX1 | NM_002653 | 34.58 | 27.12 | 28.72 | | 34.58 | 72.79 | 36.57 | 0.50 |
| 213406_at | 26118 | WSB1 | AA521269 | 66.97 | 43.60 | 68.43 | | 66.97 | 103.27 | 51.89 | 0.50 |
| 204080_at | 114034 | TOE1 | NM_025077 | 456.78 | 527.99 | 410.73 | | 456.78 | 812.13 | 408.13 | 0.50 |
| 212766_s_at | 81875 | ISG20L2 | AW294587 | 1031.22 | 1130.36 | 940.51 | | 1031.22 | 2546.09 | 1281.83 | 0.50 |
| 221458_at | 3355 | HTR1F | NM_000866 | 14.83 | 9.25 | 15.93 | | 14.83 | 28.25 | 14.24 | 0.50 |
| 217005_at | 3949 | LDLR | M28219 | 3.22 | 6.70 | 6.22 | | 3.22 | 19.65 | 9.93 | 0.51 |
| 204268_at | 6273 | S100A2 | NM_005978 | 46.22 | 20.03 | 31.12 | | 46.22 | 70.24 | 35.50 | 0.51 |
| 210111_s_at | 23008 | KIAA0265 | AF277175 | 773.86 | 707.52 | 768.96 | | 773.86 | 1300.30 | 658.08 | 0.51 |
| 217643_x_at | 440295 | LOC440295 | AA443771 | 37.82 | 45.53 | 46.48 | | 37.82 | 57.83 | 29.31 | 0.51 |
| 220388_at | 80307 | FER1L4 | NM_024777 | 9.67 | 10.23 | 8.71 | | 9.67 | 23.53 | 11.94 | 0.51 |
| 205596_s_at | 64750 | SMURF2 | AY014180 | 752.88 | 766.27 | 1137.30 | | 752.88 | 2313.63 | 1174.48 | 0.51 |
| 207675_x_at | 9048 | ARTN | NM_003976 | 73.79 | 48.25 | 51.77 | | 73.79 | 111.22 | 56.49 | 0.51 |
| 215474_at | 286053 | NSMCE2 | AK022094 | 9.63 | 60.47 | 84.58 | | 9.63 | 60.80 | 30.89 | 0.51 |
| 221177_at | 117153 | MIA2 | NM_025043 | 3.56 | 3.87 | 18.59 | | 3.56 | 21.10 | 10.73 | 0.51 |
| 221367_at | 4342 | MOS | NM_005372 | 17.57 | 13.01 | 26.89 | | 17.57 | 27.29 | 13.88 | 0.51 |
| 217280_x_at | 727729 | LOC727729 | AF061785 | 8.02 | 7.00 | 10.31 | | 8.02 | 18.62 | 9.47 | 0.51 |
| 205386_s_at | 4193 | MDM2 | NM_002392 | 50.74 | 45.02 | 48.74 | | 50.74 | 113.69 | 57.97 | 0.51 |
| 218874_s_at | 79969 | C6orf134 | NM_024909 | 27.90 | 17.85 | 33.20 | | 27.90 | 55.88 | 28.50 | 0.51 |
| 216454_at | 55621 | TRMT1 | AL390133 | 19.82 | 25.00 | 32.11 | | 19.82 | 30.54 | 15.61 | 0.51 |
| 206805_at | 10371 | SEMA3A | NM_006080 | 20.97 | 15.88 | 24.74 | | 20.97 | 86.67 | 44.30 | 0.51 |
| 221731_x_at | 1462 | CSPG2 | BF218922 | 259.21 | 481.92 | 388.87 | | 259.21 | 618.55 | 316.16 | 0.51 |
| 204256_at | 79071 | ELOVL6 | NM_024090 | 8.47 | 2.53 | 8.35 | | 8.47 | 15.05 | 7.70 | 0.51 |
| 209572_s_at | 8726 | EED | AF080227 | 1103.80 | 1447.37 | 1270.60 | | 1103.80 | 2735.45 | 1399.15 | 0.51 |
| 210170_at | 27295 | PDLIM3 | BC001017 | 6.07 | 1.30 | 6.38 | | 6.07 | 13.08 | 6.69 | 0.51 |
| 212182_at | 11163 | NUDT4 | AB007956 | 36.36 | 47.89 | 35.39 | | 36.36 | 55.54 | 28.45 | 0.51 |
| | // | /// | | | | | | | | | |
| | | NUDT4P1 | | | | | | | | | |
| 203947_at | 1479 | CSTF3 | NM_001326 | 1509.35 | 1518.13 | 884.81 | | 1509.35 | 2596.15 | 1331.39 | 0.51 |
| 210929_s_at | 197 | AHSG | AF130057 | 8.10 | 7.87 | 16.73 | | 8.10 | 27.44 | 14.08 | 0.51 |
| 220674_at | 79978 | FLJ22814 | NM_024916 | 2.92 | 4.52 | 8.35 | | 2.92 | 13.62 | 7.00 | 0.51 |
| 204688_at | 8910 | SGCE | NM_003919 | 19.25 | 29.06 | 27.11 | | 19.25 | 30.48 | 15.67 | 0.51 |
| 205438_at | 11099 | PTPN21 | NM_007039 | 3.20 | 5.14 | 2.34 | | 3.20 | 36.57 | 18.80 | 0.51 |
| 203910_at | 9411 | ARHGAP29 | NM_004815 | 11.30 | 21.65 | 25.04 | | 11.30 | 18.86 | 9.72 | 0.52 |
| 204763_s_at | 2775 | GNAO1 | NM_020988 | 5.44 | 15.02 | 23.25 | | 5.44 | 10.86 | 5.60 | 0.52 |
| 218333_at | 51009 | DERL2 | NM_016041 | 361.25 | 381.83 | 491.98 | | 361.25 | 697.53 | 359.57 | 0.52 |
| 219823_at | 79727 | LIN28 | NM_024674 | 9.11 | 12.82 | 37.37 | | 9.11 | 19.35 | 9.98 | 0.52 |
| 220375_s_at | | | NM_024752 | 95.23 | 90.24 | 76.40 | | 95.23 | 163.20 | 84.23 | 0.52 |
| 212696_s_at | 6047 | RNF4 | BF968633 | 1606.31 | 1349.15 | 1241.86 | | 1606.31 | 2520.53 | 1301.46 | 0.52 |
| 214857_at | 79946 | C10orf95 | AL050035 | 58.45 | 78.22 | 47.86 | | 58.45 | 179.88 | 93.26 | 0.52 |
| 215591_at | 23314 | SATB2 | AK025127 | 281.19 | 495.35 | 379.61 | | 281.19 | 543.96 | 282.09 | 0.52 |
| 211556_at | 10982 | MAPRE2 | AB016823 | 3.61 | 1.65 | 1.96 | | 3.61 | 17.01 | 8.82 | 0.52 |
| 214751_at | 90333 | ZNF468 | BE541042 | 267.41 | 308.40 | 303.11 | | 267.41 | 610.48 | 316.98 | 0.52 |
| 207290_at | 5362 | PLXNA2 | NM_025179 | 3.00 | 2.04 | 5.63 | | 3.00 | 21.53 | 11.19 | 0.52 |
| 202011_at | 7082 | TJP1 | NM_003257 | 31.56 | 14.89 | 16.59 | | 31.56 | 59.10 | 30.72 | 0.52 |
| 214237_x_at | 5074 | PAWR | AI760470 | 19.62 | 42.67 | 35.14 | | 19.62 | 44.00 | 22.88 | 0.52 |
| 216319_at | | | AK022686 | 7.52 | 1.90 | 7.57 | | 7.52 | 29.75 | 15.50 | 0.52 |
| 216110_x_at | | | AU147017 | 46.58 | 109.60 | 84.39 | | 46.58 | 135.38 | 70.62 | 0.52 |
| 217604_at | | | AI086530 | 90.69 | 91.95 | 104.00 | | 90.69 | 210.65 | 109.92 | 0.52 |
| 216469_at | 727867 | LOC727867 | AL163202 | 1.33 | 5.73 | 11.38 | | 1.33 | 25.81 | 13.48 | 0.52 |
| 207369_at | | | Z97632 | 12.09 | 24.32 | 26.20 | | 12.09 | 25.45 | 13.29 | 0.52 |
| 213907_at | 9521 | EEF1E1 | N32257 | 32.45 | 38.59 | 42.42 | | 32.45 | 50.17 | 26.23 | 0.52 |
| 222042_x_at | 399664 | RKHD1 | BF056534 | 11.91 | 8.76 | 12.25 | | 11.91 | 23.85 | 12.47 | 0.52 |
| 208605_s_at | 4914 | NTRK1 | NM_002529 | 3.05 | 6.12 | 1.55 | | 3.05 | 74.43 | 38.94 | 0.52 |
| 214661_s_at | 8602 | C4orf9 | R06783 | 831.88 | 560.56 | 779.27 | | 831.88 | 1279.38 | 669.46 | 0.52 |
| 221038_at | | | NM_018610 | 29.15 | 46.72 | 39.31 | | 29.15 | 64.99 | 34.09 | 0.52 |
| 205030_at | 2173 | FABP7 | NM_001446 | 7.56 | 9.22 | 19.31 | | 7.56 | 27.41 | 14.38 | 0.52 |
| 210673_x_at | 7080 | TITF1 | D50740 | 12.40 | 10.73 | 8.65 | | 12.40 | 19.09 | 10.02 | 0.52 |
| 209764_at | | | AL022312 | 24.66 | 24.31 | 17.34 | | 24.66 | 41.41 | 21.79 | 0.53 |
| 204481_at | 7862 | BRPF1 | NM_004634 | 624.18 | 645.33 | 571.52 | | 624.18 | 995.56 | 524.54 | 0.53 |
| 204244_s_at | 10926 | DBF4 | NM_006716 | 777.92 | 754.74 | 1146.76 | | 777.92 | 1236.63 | 651.74 | 0.53 |
| 212804_s_at | 26130 | GAPVD1 | AI797397 | 625.29 | 624.61 | 590.20 | | 625.29 | 1050.10 | 553.71 | 0.53 |
| 215213_at | 53371 | NUP54 | AU146949 | 10.37 | 28.70 | 26.00 | | 10.37 | 26.43 | 13.94 | 0.53 |
| 207043_s_at | 6536 | SLC6A9 | NM_006934 | 25.77 | 30.65 | 45.00 | | 25.77 | 39.89 | 21.05 | 0.53 |
| 206087_x_at | 3077 | HFE | NM_000410 | 23.96 | 30.90 | 25.87 | | 23.96 | 49.91 | 26.35 | 0.53 |
| 204885_s_at | 10232 | MSLN | NM_005823 | 5.85 | 4.68 | 9.92 | | 5.85 | 15.39 | 8.13 | 0.53 |
| 208843_s_at | 26003 | GORASP2 | BC001408 | 1166.36 | 913.23 | 1102.22 | | 1166.36 | 2140.69 | 1130.79 | 0.53 |
| 213515_x_at | 3047 /// | HBG1 | AI133353 | 7.55 | 17.08 | 17.43 | | 7.55 | 14.75 | 7.81 | 0.53 |
| | | /// | | | | | | | | | |
| | | HBG2 | | | | | | | | | |
| 219698_s_at | 64863 | METTL4 | NM_022840 | 229.77 | 268.51 | 217.64 | | 229.77 | 357.49 | 189.32 | 0.53 |
| 212646_at | 23180 | RFTN1 | D42043 | 598.16 | 797.78 | 1154.46 | | 598.16 | 2992.35 | 1590.21 | 0.53 |
| 201519_at | 9868 | TOMM70A | NM_014820 | 1139.68 | 1130.48 | 1445.11 | | 1139.68 | 2462.65 | 1309.62 | 0.53 |
| 203185_at | 9770 | RASSF2 | NM_014737 | 918.83 | 1236.44 | 624.67 | | 918.83 | 1444.26 | 768.21 | 0.53 |
| 206083_at | 575 | BAI1 | NM_001702 | 5.80 | 15.93 | 12.27 | | 5.80 | 15.98 | 8.50 | 0.53 |
| 210050_at | 7167 | TPI1 | M10036 | 37.52 | 52.62 | 69.45 | | 37.52 | 60.98 | 32.47 | 0.53 |
| 212606_at | 23001 | WDFY3 | AL536319 | 641.82 | 875.31 | 829.18 | | 641.82 | 1007.42 | 536.39 | 0.53 |
| 201542_at | 56681 | SAR1A | AY008268 | 723.66 | 797.87 | 823.10 | | 723.66 | 1172.98 | 625.20 | 0.53 |
| 216162_at | 55206 | SBNO1 | AK024128 | 5.34 | 3.04 | 22.00 | | 5.34 | 38.42 | 20.52 | 0.53 |
| 213351_s_at | 23023 | TMCC1 | AB018322 | 165.36 | 179.59 | 174.97 | | 165.36 | 319.85 | 171.06 | 0.53 |
| 213567_at | | | BF431965 | 551.09 | 504.28 | 556.12 | | 551.09 | 1373.65 | 735.42 | 0.54 |
| 213409_s_at | 6009 | RHEB | BF593727 | 67.27 | 60.90 | 98.52 | | 67.27 | 125.95 | 67.43 | 0.54 |
| 205217_at | 1678 | TIMM8A | NM_004085 | 126.84 | 90.15 | 96.93 | | 126.84 | 194.50 | 104.30 | 0.54 |
| 219053_s_at | 55048 | VPS37C | NM_017966 | 1198.05 | 1276.55 | 1540.24 | | 1198.05 | 2476.83 | 1329.01 | 0.54 |
| 213637_at | | | BE503392 | 438.11 | 436.35 | 477.85 | | 438.11 | 937.19 | 503.07 | 0.54 |
| 218707_at | 55311 | ZNF444 | NM_018337 | 91.95 | 95.72 | 117.76 | | 91.95 | 179.84 | 96.70 | 0.54 |
| 204010_s_at | 3845 | KRAS | NM_004985 | 33.72 | 33.88 | 43.86 | | 33.72 | 50.86 | 27.38 | 0.54 |
| 213165_at | 9857 | CEP350 | AI041204 | 1539.30 | 1494.91 | 1673.92 | | 1539.30 | 2332.14 | 1255.70 | 0.54 |
| 213560_at | 4616 | GADD45B | AV658684 | 11.79 | 35.24 | 58.52 | | 11.79 | 107.99 | 58.20 | 0.54 |
| 206235_at | 3981 | LIG4 | NM_002312 | 117.38 | 160.36 | 118.74 | | 117.38 | 222.17 | 119.80 | 0.54 |
| AFFX-r2-Bs- | | | AFFX-r2- | 2.24 | 8.15 | 3.04 | | 2.24 | 18.75 | 10.11 | 0.54 |
| lys-3_at | | | Bs-lys-3 | | | | | | | | |
| 205825_at | 5122 | PCSK1 | NM_000439 | 7.09 | 9.22 | 8.50 | | 7.09 | 17.48 | 9.43 | 0.54 |
| 208135_at | 6928 | TCF2 | NM_006481 | 5.83 | 9.21 | 10.04 | | 5.83 | 17.97 | 9.70 | 0.54 |
| 202445_s_at | 4853 | NOTCH2 | NM_024408 | 92.07 | 118.35 | 84.60 | | 92.07 | 181.68 | 98.05 | 0.54 |
| 209945_s_at | 2932 | GSK3B | BC000251 | 490.15 | 495.20 | 559.85 | | 490.15 | 803.29 | 433.67 | 0.54 |
| 203347_s_at | 22823 | MTF2 | NM_007358 | 599.35 | 632.99 | 579.21 | | 599.35 | 1402.04 | 757.01 | 0.54 |
| 209422_at | 51230 | PHF20 | AY027523 | 1019.85 | 1234.74 | 1533.52 | | 1019.85 | 2319.53 | 1254.73 | 0.54 |
| 219146_at | 79736 | C17orf42 | NM_024683 | 542.17 | 645.81 | 600.70 | | 542.17 | 959.75 | 519.55 | 0.54 |
| 202435_s_at | 1545 | CYP1B1 | AU154504 | 624.04 | 857.21 | 318.42 | | 624.04 | 1683.84 | 912.77 | 0.54 |
| 201997_s_at | 23013 | SPEN | NM_015001 | 1294.84 | 1381.87 | 1278.71 | | 1294.84 | 2117.06 | 1147.92 | 0.54 |
| 207935_s_at | 3860 | KRT13 | NM_002274 | 3.38 | 1.85 | 6.36 | | 3.38 | 11.03 | 5.99 | 0.54 |
| 209957_s_at | 4878 | NPPA | M30262 | 23.43 | 34.34 | 31.17 | | 23.43 | 79.99 | 43.45 | 0.54 |
| 212176_at | 25957 | C6orf111 | AA902326 | 1542.05 | 1485.49 | 1514.14 | | 1542.05 | 2351.72 | 1277.42 | 0.54 |
| 214957_at | 81569 | ACTL8 | BF594459 | 6.95 | 8.16 | 27.85 | | 6.95 | 16.82 | 9.15 | 0.54 |
| 212090_at | 2907 | GRINA | AL571424 | 908.66 | 1500.60 | 1270.25 | | 908.66 | 1519.22 | 826.93 | 0.54 |
| 207476_at | 50618 | ITSN2 | NM_018512 | 7.50 | 4.27 | 8.31 | | 7.50 | 18.44 | 10.04 | 0.54 |
| 212435_at | 51592 | TRIM33 | AA205593 | 565.79 | 554.14 | 538.25 | | 565.79 | 878.07 | 478.06 | 0.54 |
| 218170_at | 51015 | ISOC1 | NM_016048 | 609.14 | 797.72 | 811.27 | | 609.14 | 1734.46 | 944.32 | 0.54 |
| 221917_s_at | 2926 | GRSF1 | BF058465 | 46.94 | 42.56 | 48.07 | | 46.94 | 72.49 | 39.50 | 0.54 |
| 216884_at | 5782 | PTPN12 | S69182 | 7.44 | 12.68 | 36.09 | | 7.44 | 91.33 | 49.77 | 0.54 |
| 218757_s_at | 65109 | UPF3B | NM_023010 | 646.34 | 759.28 | 895.07 | | 646.34 | 1205.29 | 657.07 | 0.55 |
| 203346_s_at | 22823 | MTF2 | AF072814 | 1463.65 | 1632.74 | 1331.98 | | 1463.65 | 2687.47 | 1465.67 | 0.55 |
| 210495_x_at | 2335 | FN1 | AF130095 | 17.03 | 59.51 | 49.75 | | 17.03 | 99.64 | 54.35 | 0.55 |
| 214744_s_at | 9349 | RPL23 | AK021960 | 73.88 | 116.48 | 94.19 | | 73.88 | 161.75 | 88.25 | 0.55 |
| 221870_at | 30846 | EHD2 | AI417917 | 21.04 | 29.86 | 12.01 | | 21.04 | 129.40 | 70.60 | 0.55 |
| 204073_s_at | 745 | C11orf9 | NM_013279 | 32.85 | 39.30 | 32.69 | | 32.85 | 51.60 | 28.16 | 0.55 |
| 206386_at | 6906 | SERPINA7 | NM_000354 | 5.41 | 19.84 | 21.03 | | 5.41 | 19.51 | 10.65 | 0.55 |
| 212888_at | 23405 | DICER1 | BG109746 | 532.23 | 608.67 | 501.68 | | 532.23 | 798.74 | 436.21 | 0.55 |
| 211168_s_at | 5976 | UPF1 | D86988 | 503.32 | 554.73 | 450.32 | | 503.32 | 925.90 | 506.53 | 0.55 |
| 210542_s_at | 28232 | SLCO3A1 | BC000585 | 57.56 | 64.09 | 50.14 | | 57.56 | 122.36 | 66.97 | 0.55 |
| 208298_at | 7813 | EVI5 | NM_005665 | 7.93 | 3.06 | 2.90 | | 7.93 | 12.68 | 6.95 | 0.55 |
| 44822_s_at | 54531 | MIER2 | AW003889 | 192.34 | 196.07 | 136.93 | | 192.34 | 295.84 | 162.46 | 0.55 |
| 204686_at | 3667 | IRS1 | NM_005544 | 41.93 | 50.71 | 16.99 | | 41.93 | 131.26 | 72.11 | 0.55 |
| 211678_s_at | 55905 | ZNF313 | AF090934 | 1296.20 | 1152.15 | 1341.12 | | 1296.20 | 2414.30 | 1326.73 | 0.55 |
| 219567_s_at | 64789 | C1orf176 | NM_022774 | 936.37 | 1313.36 | 1059.32 | | 936.37 | 1463.09 | 804.25 | 0.55 |
| 210155_at | 4653 | MYOC | D88214 | 48.84 | 49.97 | 42.84 | | 48.84 | 74.98 | 41.22 | 0.55 |
| 219448_at | 54968 | TMEM70 | BC002748 | 65.56 | 57.68 | 70.05 | | 65.56 | 136.50 | 75.08 | 0.55 |
| 213457_at | 9258 | MFHAS1 | BF739959 | 620.68 | 643.62 | 571.84 | | 620.68 | 1174.55 | 646.13 | 0.55 |
| 213544_at | 3622 | ING2 | AI186701 | 22.40 | 14.76 | 35.39 | | 22.40 | 64.74 | 35.62 | 0.55 |
| 219624_at | 9530 | BAG4 | NM_004874 | 130.48 | 128.96 | 142.46 | | 130.48 | 202.16 | 111.28 | 0.55 |
| 214718_at | 57798 | GATAD1 | AK026142 | 421.81 | 467.90 | 544.32 | | 421.81 | 788.14 | 434.36 | 0.55 |
| 218783_at | 25896 | INTS7 | AL133049 | 307.52 | 316.47 | 258.88 | | 307.52 | 474.68 | 261.64 | 0.55 |
| 203575_at | 1459 | CSNK2A2 | NM_001896 | 402.45 | 415.51 | 340.72 | | 402.45 | 803.90 | 443.29 | 0.55 |
| 206164_at | 9635 | CLCA2 | NM_006536 | 5.90 | 7.51 | 3.32 | | 5.90 | 11.06 | 6.10 | 0.55 |
| 202073_at | 10133 | OPTN | AV757675 | 173.40 | 450.81 | 407.47 | | 173.40 | 441.30 | 243.39 | 0.55 |
| 215128_at | | | AV704232 | 116.39 | 162.95 | 132.27 | | 116.39 | 285.39 | 157.51 | 0.55 |
| 208287_at | 10255 | HCG9 | NM_005844 | 11.72 | 6.94 | 23.96 | | 11.72 | 22.89 | 12.63 | 0.55 |
| 207815_at | 5197 | PF4V1 | NM_002620 | 3.99 | 18.84 | 17.84 | | 3.99 | 23.36 | 12.91 | 0.55 |
| 201794_s_at | 9887 | SMG7 | NM_014837 | 858.91 | 791.45 | 990.44 | | 858.91 | 1352.73 | 748.10 | 0.55 |
| 215457_at | 10552 | ARPC1A | AF070647 | 23.57 | 4.00 | 31.40 | | 23.57 | 39.62 | 21.94 | 0.55 |
| 213734_at | | | BG260658 | 374.34 | 370.20 | 554.06 | | 374.34 | 962.96 | 533.48 | 0.55 |
| 204909_at | 1656 | DDX6 | NM_004397 | 63.64 | 81.28 | 63.34 | | 63.64 | 100.97 | 55.96 | 0.55 |
| 202565_s_at | 6840 | SVIL | NM_003174 | 141.61 | 96.43 | 148.47 | | 141.61 | 357.23 | 198.04 | 0.55 |
| 220651_s_at | 55388 | MCM10 | AB042719 | 261.54 | 280.33 | 216.37 | | 261.54 | 412.86 | 228.91 | 0.55 |
| 213500_at | 9276 | COPB2 | AI307760 | 217.20 | 244.58 | 156.09 | | 217.20 | 330.78 | 183.41 | 0.55 |
| 209558_s_at | 728014 / | HIP1R | AB013384 | 24.53 | 51.29 | 43.98 | | 24.53 | 73.28 | 40.67 | 0.56 |
| | | /// | | | | | | | | | |
| | | LOC728014 | | | | | | | | | |
| 216504_s_at | 64116 | SLC39A8 | AL049963 | 48.40 | 78.75 | 62.89 | | 48.40 | 166.93 | 92.66 | 0.56 |
| 206208_at | 762 | CA4 | NM_000717 | 5.51 | 24.52 | 18.78 | | 5.51 | 10.62 | 5.90 | 0.56 |
| 216142_at | | | AL137403 | 44.77 | 60.59 | 56.74 | | 44.77 | 82.88 | 46.06 | 0.56 |
| 209120_at | 7026 | NR2F2 | AL037401 | 29.67 | 56.19 | 56.29 | | 29.67 | 106.88 | 59.45 | 0.56 |
| 202230_s_at | 10523 | CHERP | NM_006387 | 1955.62 | 1567.98 | 1532.63 | | 1955.62 | 3126.88 | 1739.84 | 0.56 |
| 205335_s_at | 6728 | SRP19 | NM_003135 | 2107.55 | 2076.69 | 2037.09 | | 2107.55 | 3826.84 | 2132.52 | 0.56 |
| 204533_at | 3627 | CXCL10 | NM_001565 | 12.53 | 3433.73 | 4050.06 | | 12.53 | 1996.92 | 1112.92 | 0.56 |
| 219719_at | 51751 | HIGD1B | NM_016438 | 7.60 | 10.61 | 8.46 | | 7.60 | 15.40 | 8.59 | 0.56 |
| 201110_s_at | 7057 | THBS1 | NM_003246 | 2.69 | 49.97 | 64.93 | | 2.69 | 644.40 | 359.39 | 0.56 |
| 217400_at | 392454 | LOC392454 | AL034410 | 12.30 | 4.15 | 9.35 | | 12.30 | 19.04 | 10.64 | 0.56 |
| 202295_s_at | 1512 | CTSH | NM_004390 | 3602.55 | 5815.13 | 4593.58 | | 3602.55 | 6007.85 | 3361.13 | 0.56 |
| 222107_x_at | 11178 | LZTS1 | BE312985 | 8.73 | 37.20 | 35.80 | | 8.73 | 30.31 | 16.96 | 0.56 |
| 214133_at | 4588 | MUC6 | AI611214 | 16.68 | 36.64 | 51.79 | | 16.68 | 64.05 | 35.85 | 0.56 |
| 216725_at | 166614 | DCAMKL2 | AL359602 | 5.30 | 5.23 | 3.99 | | 5.30 | 10.41 | 5.83 | 0.56 |
| 210383_at | 6323 | SCN1A | AF225985 | 5.32 | 9.15 | 8.31 | | 5.32 | 12.37 | 6.93 | 0.56 |
| 214860_at | 84679 | SLC9A7 | AL022165 | 5.63 | 8.81 | 3.45 | | 5.63 | 12.57 | 7.04 | 0.56 |
| 204812_at | 9183 | ZW10 | NM_004724 | 383.66 | 380.50 | 389.53 | | 383.66 | 588.00 | 329.62 | 0.56 |
| 205709_s_at | 1040 | CDS1 | U65887 | 125.58 | 113.83 | 107.86 | | 125.58 | 209.70 | 117.59 | 0.56 |
| 210419_at | 8538 | BARX2 | AF031924 | 9.57 | 14.74 | 16.75 | | 9.57 | 19.18 | 10.77 | 0.56 |
| 220886_at | 55879 | GABRQ | NM_018558 | 16.38 | 2.86 | 6.70 | | 16.38 | 28.47 | 16.00 | 0.56 |
| 218956_s_at | 26024 | PTCD1 | NM_015545 | 270.00 | 270.28 | 268.69 | | 270.00 | 575.45 | 323.52 | 0.56 |
| 201667_at | 2697 | GJA1 | NM_000165 | 2.13 | 20.51 | 15.90 | | 2.13 | 102.85 | 57.84 | 0.56 |
| 203358_s_at | 2146 | EZH2 | NM_004456 | 1011.21 | 1048.92 | 1116.89 | | 1011.21 | 2182.16 | 1227.15 | 0.56 |
| 217828_at | 79811 | SLTM | NM_024755 | 1018.28 | 888.35 | 884.24 | | 1018.28 | 1653.10 | 930.70 | 0.56 |
| 203066_at | 51363 | GALNAC4S- | NM_014863 | 11.67 | 6.84 | 24.21 | | 11.67 | 24.13 | 13.60 | 0.56 |
| | | 6ST | | | | | | | | | |
| 208016_s_at | 185 | AGTR1 | NM_004835 | 6.06 | 7.07 | 15.78 | | 6.06 | 10.77 | 6.07 | 0.56 |
| 209705_at | | | BG033764 | 615.53 | 648.43 | 718.05 | | 615.53 | 1163.32 | 655.88 | 0.56 |
| 221982_x_at | 8270 | LAGE3 | AA034498 | 12.66 | 7.30 | 6.93 | | 12.66 | 21.49 | 12.13 | 0.56 |
| 213076_at | 80271 | ITPKC | D38169 | 72.56 | 81.20 | 98.58 | | 72.56 | 121.95 | 68.86 | 0.56 |
| 220939_s_at | 54878 | DPP8 | NM_017743 | 1150.90 | 1296.89 | 1132.00 | | 1150.90 | 1771.65 | 1000.55 | 0.56 |
| 220121_at | 55180 | LINS1 | NM_018148 | 80.59 | 84.47 | 92.25 | | 80.59 | 143.84 | 81.29 | 0.57 |
| 215092_s_at | 10725 | NFAT5 | AJ005683 | 126.73 | 287.38 | 240.81 | | 126.73 | 611.89 | 345.88 | 0.57 |
| 201934_at | 80335 | TMEM113 | N92524 | 1911.34 | 1566.53 | 1812.90 | | 1911.34 | 4097.14 | 2317.02 | 0.57 |
| 203038_at | 5796 | PTPRK | NM_002844 | 534.39 | 648.97 | 742.03 | | 534.39 | 1149.32 | 650.04 | 0.57 |
| 216655_s_at | | | AF041811 | 4.20 | 5.23 | 3.73 | | 4.20 | 21.65 | 12.24 | 0.57 |
| 218447_at | 56942 | C16orf61 | NM_020188 | 815.60 | 958.90 | 934.89 | | 815.60 | 1911.90 | 1081.77 | 0.57 |
| 216336_x_at | 4499 | MT1M | AL031602 | 771.07 | 469.15 | 498.68 | | 771.07 | 1218.16 | 689.54 | 0.57 |
| 221169_s_at | 59340 | HRH4 | AF312230 | 6.60 | 13.81 | 16.80 | | 6.60 | 13.76 | 7.79 | 0.57 |
| 221168_at | 59336 | PRDM13 | NM_021620 | 26.10 | 33.88 | 25.79 | | 26.10 | 57.25 | 32.44 | 0.57 |
| 222047_s_at | 51593 | ARS2 | AI523895 | 3175.02 | 3036.78 | 2857.66 | | 3175.02 | 4881.85 | 2767.32 | 0.57 |
| 201558_at | 8480 | RAE1 | NM_003610 | 1308.92 | 1246.68 | 1264.18 | | 1308.92 | 2214.87 | 1256.22 | 0.57 |
| 216814_at | | | AL353587 | 6.18 | 15.59 | 30.87 | | 6.18 | 22.78 | 12.94 | 0.57 |
| 204176_at | 27252 | KLHL20 | AA808694 | 171.63 | 221.34 | 206.44 | | 171.63 | 258.85 | 147.07 | 0.57 |
| 37152_at | 5467 | PPARD | L07592 | 816.89 | 1283.54 | 1017.45 | | 816.89 | 1808.92 | 1027.89 | 0.57 |
| 209115_at | 9039 | UBE1C | AL117566 | 1143.90 | 1476.21 | 1511.58 | | 1143.90 | 2027.89 | 1152.50 | 0.57 |
| 201109_s_at | 7057 | THBS1 | AV726673 | 8.37 | 54.04 | 71.48 | | 8.37 | 585.69 | 332.94 | 0.57 |
| 213673_x_at | 29937 | NENF | AA156251 | 23.67 | 12.40 | 64.29 | | 23.67 | 56.48 | 32.14 | 0.57 |
| 202429_s_at | 5530 | PPP3CA | AL353950 | 2546.81 | 2925.70 | 2581.92 | | 2546.81 | 4408.70 | 2510.26 | 0.57 |
| 214743_at | 1523 | CUTL1 | BE046521 | 517.17 | 815.05 | 750.95 | | 517.17 | 1328.98 | 756.84 | 0.57 |
| 214175_x_at | 8572 | PDLIM4 | AI254547 | 111.09 | 132.61 | 106.52 | | 111.09 | 1380.21 | 786.14 | 0.57 |
| 203255_at | 80204 | FBXO11 | NM_018693 | 467.30 | 553.26 | 512.44 | | 467.30 | 1039.36 | 593.22 | 0.57 |
| 200995_at | 10527 | IPO7 | AI741392 | 898.97 | 728.95 | 761.91 | | 898.97 | 1456.93 | 831.65 | 0.57 |
| 201678_s_at | 56941 | C3orf37 | AI937543 | 294.64 | 327.53 | 346.71 | | 294.64 | 538.95 | 307.69 | 0.57 |
| 220203_at | 353500 | BMP8A | NM_024732 | 11.78 | 7.99 | 13.24 | | 11.78 | 24.62 | 14.06 | 0.57 |
| 203963_at | 771 | CA12 | NM_001218 | 30.62 | 37.85 | 27.85 | | 30.62 | 70.78 | 40.44 | 0.57 |
| 210068_s_at | 361 | AQP4 | U63622 | 2.55 | 3.55 | 6.04 | | 2.55 | 11.20 | 6.40 | 0.57 |
| 219801_at | 80778 | ZNF34 | NM_030580 | 105.12 | 90.95 | 82.85 | | 105.12 | 161.75 | 92.45 | 0.57 |
| 210500_at | | | BC001957 | 9.70 | 27.09 | 31.23 | | 9.70 | 26.52 | 15.17 | 0.57 |
| 206207_at | 1178 | CLC | NM_001828 | 4.34 | 5.02 | 2.67 | | 4.34 | 10.98 | 6.28 | 0.57 |
| 211221_at | 10474 | TADA3L | AL117487 | 14.41 | 28.17 | 41.59 | | 14.41 | 21.97 | 12.57 | 0.57 |
| 218888_s_at | 81831 | NETO2 | AI335263 | 620.42 | 694.17 | 411.07 | | 620.42 | 1162.49 | 665.59 | 0.57 |
| 205962_at | 5062 | PAK2 | NM_002577 | 5.10 | 6.52 | 4.52 | | 5.10 | 10.55 | 6.04 | 0.57 |
| 209005_at | 26234 | FBXL5 | AF157323 | 402.10 | 489.15 | 494.98 | | 402.10 | 611.07 | 350.09 | 0.57 |
| 221506_s_at | 30000 | TNPO2 | BG258639 | 523.21 | 581.43 | 517.79 | | 523.21 | 926.33 | 531.48 | 0.57 |
| 221654_s_at | 9960 | USP3 | AF077040 | 1046.89 | 1022.30 | 1404.66 | | 1046.89 | 2334.51 | 1339.48 | 0.57 |
| 216170_at | 1937 | EEF1G | AK025271 | 62.43 | 129.40 | 107.42 | | 62.43 | 158.64 | 91.09 | 0.57 |
| 205016_at | 7039 | TGFA | NM_003236 | 31.45 | 26.63 | 46.20 | | 31.45 | 72.23 | 41.52 | 0.57 |
| 218939_at | 3954 | LETM1 | NM_012318 | 61.30 | 48.56 | 97.44 | | 61.30 | 104.34 | 60.07 | 0.58 |
| 211071_s_at | 10962 | MLLT11 | BC006471 | 80.14 | 87.38 | 86.95 | | 80.14 | 159.72 | 92.03 | 0.58 |
| 205525_at | 800 | CALD1 | NM_018495 | 20.67 | 18.24 | 25.10 | | 20.67 | 36.63 | 21.13 | 0.58 |
| 204449_at | 5082 | PDCL | NM_005388 | 214.13 | 210.87 | 480.31 | | 214.13 | 345.82 | 199.64 | 0.58 |
| 218833_at | 51776 | ZAK | NM_016653 | 81.01 | 82.76 | 82.80 | | 81.01 | 134.45 | 77.67 | 0.58 |
| 203355_s_at | 23362 | PSD3 | NM_015310 | 507.92 | 624.18 | 948.42 | | 507.92 | 3142.40 | 1815.89 | 0.58 |
| 203481_at | 55719 | C10orf6 | AI655902 | 235.56 | 196.06 | 205.55 | | 235.56 | 392.47 | 227.09 | 0.58 |
| 215363_x_at | 2346 | FOLH1 | AW168915 | 19.13 | 19.05 | 11.89 | | 19.13 | 44.97 | 26.04 | 0.58 |
| 204620_s_at | 1462 | CSPG2 | NM_004385 | 272.63 | 479.87 | 383.43 | | 272.63 | 597.02 | 345.69 | 0.58 |
| 204926_at | 3624 | INHBA | NM_002192 | 15.48 | 7.96 | 1.44 | | 15.48 | 27.03 | 15.67 | 0.58 |
| 211339_s_at | 3702 | ITK | D13720 | 17.30 | 11.10 | 13.78 | | 17.30 | 27.76 | 16.10 | 0.58 |
| 213122_at | 85453 | TSPYL5 | AI096375 | 891.44 | 626.71 | 650.82 | | 891.44 | 1365.32 | 792.27 | 0.58 |
| 212412_at | 10611 | PDLIM5 | AV715767 | 1104.80 | 1587.77 | 1280.50 | | 1104.80 | 1993.22 | 1157.15 | 0.58 |
| 221974_at | 63968 | PWCR1 | AW770748 | 163.05 | 222.80 | 210.50 | | 163.05 | 267.26 | 155.19 | 0.58 |
| 215045_at | 11189 | TNRC4 | BC004145 | 6.94 | 7.96 | 11.89 | | 6.94 | 18.72 | 10.87 | 0.58 |
| 210047_at | 4891 | SLC11A2 | AF064484 | 25.04 | 12.54 | 34.35 | | 25.04 | 105.56 | 61.33 | 0.58 |
| 202457_s_at | 5530 | PPP3CA | AA911231 | 1878.67 | 2067.17 | 1969.82 | | 1878.67 | 3811.17 | 2214.15 | 0.58 |
| 218859_s_at | 51575 | ESF1 | NM_016649 | 405.27 | 316.59 | 411.38 | | 405.27 | 687.15 | 399.25 | 0.58 |
| 219792_at | 79814 | AGMAT | NM_024758 | 17.48 | 32.52 | 33.58 | | 17.48 | 48.63 | 28.26 | 0.58 |
| 218279_s_at | 8337 | HIST2H2AA3 | BC001629 | 29.81 | 72.00 | 54.01 | | 29.81 | 94.79 | 55.10 | 0.58 |
| 221569_at | 54806 | AHI1 | AL136797 | 258.34 | 338.49 | 282.42 | | 258.34 | 546.72 | 317.96 | 0.58 |
| 203673_at | 7038 | TG | NM_003235 | 38.30 | 31.98 | 10.90 | | 38.30 | 115.66 | 67.27 | 0.58 |
| 202655_at | 7873 | ARMET | NM_006010 | 1387.14 | 2019.70 | 1247.65 | | 1387.14 | 2130.75 | 1239.48 | 0.58 |
| 201566_x_at | 3398 /// | ID2 /// | NM_002166 | 383.92 | 461.04 | 156.05 | | 383.92 | 1336.35 | 777.63 | 0.58 |
| | | ID2B | | | | | | | | | |
| 215750_at | 85373 | KIAA1659 | AB051446 | 86.88 | 114.23 | 125.76 | | 86.88 | 132.43 | 77.08 | 0.58 |
| 202843_at | 4189 | DNAJB9 | NM_012328 | 56.63 | 74.98 | 149.09 | | 56.63 | 366.95 | 214.10 | 0.58 |
| 221729_at | 1290 | COL5A2 | AL575735 | 20.18 | 11.28 | 22.13 | | 20.18 | 307.15 | 179.24 | 0.58 |
| 217947_at | 54918 | CMTM6 | NM_017801 | 5067.56 | 5746.33 | 5613.70 | | 5067.56 | 7918.64 | 4621.34 | 0.58 |
| 222308_x_at | 9984 | THOC1 | AV700403 | 17.44 | 13.13 | 9.93 | | 17.44 | 34.68 | 20.25 | 0.58 |
| 218649_x_at | 9147 | SDCCAG1 | NM_004713 | 690.20 | 739.86 | 832.53 | | 690.20 | 1344.82 | 785.54 | 0.58 |
| 204204_at | 1318 | SLC31A2 | NM_001860 | 69.78 | 160.89 | 124.61 | | 69.78 | 269.65 | 157.60 | 0.58 |
| 220765_s_at | 55679 | LIMS2 | NM_017980 | 104.77 | 130.97 | 110.48 | | 104.77 | 454.49 | 265.64 | 0.58 |
| 208816_x_at | 304 | ANXA2P2 | M62898 | 1277.14 | 1657.98 | 1732.89 | | 1277.14 | 2493.57 | 1457.84 | 0.58 |
| 209704_at | | | AL523380 | 411.75 | 465.01 | 465.04 | | 411.75 | 939.24 | 549.57 | 0.59 |
| 213044_at | 6093 | ROCK1 | N22548 | 2153.60 | 2018.87 | 2398.84 | | 2153.60 | 3533.11 | 2068.97 | 0.59 |
| 206652_at | 9205 | ZMYM5 | NM_016384 | 107.50 | 145.22 | 182.96 | | 107.50 | 215.98 | 126.50 | 0.59 |
| 32032_at | 8220 | DGCR14 | L77566 | 491.02 | 466.07 | 592.26 | | 491.02 | 865.24 | 507.43 | 0.59 |
| 217464_at | | | L48784 | 27.50 | 40.30 | 21.12 | | 27.50 | 49.38 | 28.96 | 0.59 |
| 220520_s_at | 54830 | NUP62CL | NM_017681 | 3.38 | 2.63 | 10.86 | | 3.38 | 10.11 | 5.93 | 0.59 |
| 203476_at | 7162 | TPBG | NM_006670 | 1547.74 | 1859.00 | 1448.97 | | 1547.74 | 2461.87 | 1445.51 | 0.59 |
| 217992_s_at | 79180 | EFHD2 | AW664179 | 2087.89 | 2742.68 | 1521.53 | | 2087.89 | 4132.64 | 2427.90 | 0.59 |
| 216357_at | 6642 | SNX1 | AL050148 | 3.46 | 11.61 | 25.37 | | 3.46 | 13.89 | 8.16 | 0.59 |
| 207058_s_at | 5071 | PARK2 | NM_004562 | 20.26 | 30.54 | 37.07 | | 20.26 | 34.28 | 20.16 | 0.59 |
| 220962_s_at | 29943 | PADI1 | NM_013358 | 28.98 | 76.92 | 62.85 | | 28.98 | 94.95 | 55.88 | 0.59 |
| 204042_at | 10810 | WASF3 | AB020707 | 8.08 | 8.35 | 9.49 | | 8.08 | 16.34 | 9.62 | 0.59 |
| 219290_x_at | 27071 | DAPP1 | NM_014395 | 699.32 | 1441.05 | 1434.34 | | 699.32 | 1269.02 | 747.23 | 0.59 |
| 201054_at | 10949 | HNRPA0 | BE966599 | 2367.56 | 2412.44 | 1967.07 | | 2367.56 | 3839.74 | 2262.69 | 0.59 |
| 219902_at | 23743 | BHMT2 | NM_017614 | 9.25 | 12.98 | 8.28 | | 9.25 | 14.57 | 8.59 | 0.59 |
| 209161_at | 9128 | PRPF4 | AI184802 | 1263.94 | 1229.26 | 1127.79 | | 1263.94 | 2497.99 | 1475.02 | 0.59 |
| 205664_at | 22944 | KIN | NM_0123111 | 223.92 | 193.14 | 225.06 | | 223.92 | 341.81 | 201.90 | 0.59 |
| 219049_at | 55790 | ChGn | NM_018371 | 38.51 | 26.81 | 31.40 | | 38.51 | 197.61 | 116.77 | 0.59 |
| 212989_at | 259230 | TMEM23 | AI377497 | 31.60 | 40.87 | 45.39 | | 31.60 | 54.14 | 32.00 | 0.59 |
| 219747_at | 79625 | C4orf31 | NM_024574 | 44.69 | 44.20 | 55.00 | | 44.69 | 124.89 | 73.81 | 0.59 |
| 206442_at | 6406 | SEMG1 | NM_003007 | 78.87 | 70.34 | 69.75 | | 78.87 | 163.27 | 96.60 | 0.59 |
| 212522_at | 5151 | PDE8A | W73272 | 123.91 | 182.94 | 229.48 | | 123.91 | 584.71 | 346.00 | 0.59 |
| 207279_s_at | 10529 | NEBL | NM_016365 | 12.47 | 19.33 | 16.29 | | 12.47 | 38.60 | 22.85 | 0.59 |
| 203562_at | 9638 | FEZ1 | NM_005103 | 274.24 | 1270.93 | 1430.17 | | 274.24 | 3040.29 | 1800.35 | 0.59 |
| 218358_at | 8812 | CCNK | NM_024324 | 2082.26 | 2626.58 | 1781.27 | | 2082.26 | 3180.78 | 1883.74 | 0.59 |
| 203620_s_at | 9873 | FCHSD2 | NM_014824 | 351.87 | 398.56 | 367.25 | | 351.87 | 597.74 | 354.69 | 0.59 |
| 206028_s_at | 10461 | MERTK | NM_006343 | 39.27 | 43.70 | 41.43 | | 39.27 | 67.32 | 39.95 | 0.59 |
| 202660_at | 3709 | ITPR2 | AA834576 | 66.59 | 98.91 | 79.22 | | 66.59 | 131.94 | 78.37 | 0.59 |
| 218131_s_at | 54815 | GATAD2A | AK024670 | 568.29 | 661.47 | 600.94 | | 568.29 | 1127.27 | 670.07 | 0.59 |
| 205946_at | 7434 | VIPR2 | NM_003382 | 6.03 | 8.98 | 25.12 | | 6.03 | 13.15 | 7.81 | 0.59 |
| 222106_at | 23627 | PRND | AL133396 | 4.92 | 8.82 | 26.67 | | 4.92 | 20.22 | 12.03 | 0.59 |
| 208462_s_at | 10060 | ABCC9 | NM_005691 | 13.41 | 11.27 | 7.50 | | 13.41 | 22.00 | 13.09 | 0.60 |
| 205527_s_at | 50628 | GEMIN4 | NM_015487 | 175.68 | 183.32 | 140.97 | | 175.68 | 350.20 | 208.47 | 0.60 |
| 220476_s_at | 55924 | C1orf183 | NM_019099 | 24.14 | 12.49 | 25.01 | | 24.14 | 49.36 | 29.39 | 0.60 |
| 209296_at | 5495 | PPM1B | AF136972 | 1521.64 | 1397.15 | 1972.36 | | 1521.64 | 2494.04 | 1485.51 | 0.60 |
| 210335_at | 9182 | PAMC1 | AF056209 | 10.06 | 7.72 | 21.71 | | 10.06 | 19.88 | 11.84 | 0.60 |
| 212972_x_at | | | AL080130 | 16.33 | 30.31 | 39.88 | | 16.33 | 29.54 | 17.63 | 0.60 |
| 203898_at | 27297 | RCP9 | AU154853 | 153.56 | 181.76 | 191.90 | | 153.56 | 294.30 | 175.82 | 0.60 |
| 207812_s_at | 26003 | GORASP2 | NM_015530 | 1356.37 | 1316.83 | 1221.07 | | 1356.37 | 2195.98 | 1312.22 | 0.60 |
| 212643_at | 93487 | C14orf32 | AI671747 | 3791.63 | 3457.95 | 3260.05 | | 3791.63 | 6364.90 | 3803.72 | 0.60 |
| 204296_at | 1639 | DCTN1 | NM_021196 | 28.07 | 28.90 | 59.15 | | 28.07 | 62.64 | 37.49 | 0.60 |
| 203419_at | 9757 | MLL4 | NM_014727 | 306.37 | 458.27 | 260.17 | | 306.37 | 500.38 | 299.54 | 0.60 |
| 204655_at | 6352 | CCL5 | NM_002985 | 1774.54 | 2421.70 | 2736.70 | | 1774.54 | 13645.23 | 8170.20 | 0.60 |
| 205255_x_at | 6932 | TCF7 | NM_003202 | 279.46 | 339.40 | 216.17 | | 279.46 | 540.90 | 323.87 | 0.60 |
| 219743_at | 23493 | HEY2 | NM_012259 | 3.36 | 7.80 | 6.20 | | 3.36 | 11.12 | 6.67 | 0.60 |
| 219128_at | 54980 | C2orf42 | NM_017880 | 198.95 | 205.65 | 231.04 | | 198.95 | 327.95 | 196.67 | 0.60 |
| 211149_at | 7404 | UTY | AF000994 | 69.65 | 100.26 | 99.90 | | 69.65 | 131.54 | 78.95 | 0.60 |
| 212726_at | 5253 | PHF2 | AB014562 | 500.61 | 479.08 | 465.57 | | 500.61 | 793.54 | 477.15 | 0.60 |
| 216731_s_at | 389768 | LOC389768 | AK023690 | 3.28 | 4.84 | 2.50 | | 3.28 | 12.82 | 7.71 | 0.60 |
| 213275_x_at | 1508 | CTSB | W47179 | 658.11 | 663.52 | 549.73 | | 658.11 | 1041.07 | 626.45 | 0.60 |
| 220667_at | | | NM_018540 | 11.65 | 7.88 | 23.12 | | 11.65 | 24.81 | 14.95 | 0.60 |
| 217579_x_at | 64225 | ARL6IP2 | AW301806 | 210.96 | 165.28 | 403.77 | | 210.96 | 375.05 | 226.03 | 0.60 |
| 220895_at | 57663 | USP29 | NM_020903 | 10.72 | 7.70 | 6.64 | | 10.72 | 16.14 | 9.73 | 0.60 |
| 222270_at | 57223 | SMEK2 | BG540048 | 27.47 | 48.27 | 47.65 | | 27.47 | 70.22 | 42.33 | 0.60 |
| 213479_at | 4885 | NPTX2 | U26662 | 259.79 | 365.58 | 240.72 | | 259.79 | 1620.57 | 977.75 | 0.60 |
| 218984_at | 54517 | PUS7 | NM_019042 | 533.43 | 367.17 | 404.26 | | 533.43 | 1091.49 | 658.54 | 0.60 |
| 221680_s_at | 51513 | ETV7 | AF147782 | 25.25 | 47.37 | 37.06 | | 25.25 | 57.60 | 34.77 | 0.60 |
| 215454_x_at | 6440 | SFTPC | AI831055 | 8.18 | 5.80 | 6.03 | | 8.18 | 13.10 | 7.91 | 0.60 |
| 219441_s_at | 79705 | LRRK1 | NM_024652 | 123.60 | 100.35 | 116.42 | | 123.60 | 232.30 | 140.48 | 0.60 |
| 212846_at | 23076 | KIAA0179 | AA811192 | 1082.23 | 830.79 | 848.05 | | 1082.23 | 1756.32 | 1062.20 | 0.60 |
| 203345_s_at | 22823 | MTF2 | AI566096 | 2207.34 | 2366.26 | 2104.05 | | 2207.34 | 3740.22 | 2265.45 | 0.61 |
| 212083_at | 113419 | TEX261 | AV759552 | 836.12 | 751.67 | 794.43 | | 836.12 | 1282.77 | 777.36 | 0.61 |
| 221244_s_at | | | NM_031268 | 44.60 | 46.48 | 48.65 | | 44.60 | 67.31 | 40.81 | 0.61 |
| 206878_at | 1610 | DAO | NM_001917 | 20.21 | 38.40 | 31.06 | | 20.21 | 36.77 | 22.31 | 0.61 |
| 220323_at | 79935 | CNTD2 | NM_024877 | 54.39 | 87.58 | 77.82 | | 54.39 | 83.78 | 50.84 | 0.61 |
| 201594_s_at | 9989 | PPP4R1 | NM_005134 | 761.99 | 1024.43 | 802.41 | | 761.99 | 1481.98 | 899.34 | 0.61 |
| 203464_s_at | 22905 | EPN2 | NM_014964 | 57.63 | 91.85 | 72.60 | | 57.63 | 95.79 | 58.14 | 0.61 |
| 204493_at | 637 | BID | NM_001196 | 382.02 | 933.59 | 809.83 | | 382.02 | 2783.34 | 1690.01 | 0.61 |
| 207844_at | 3596 | IL13 | NM_002188 | 24.38 | 46.53 | 40.21 | | 24.38 | 45.47 | 27.61 | 0.61 |
| 219531_at | 55722 | CEP72 | NM_018140 | 167.53 | 173.60 | 200.58 | | 167.53 | 300.73 | 182.67 | 0.61 |
| 214706_at | 7752 | ZNF200 | AU149447 | 376.85 | 445.79 | 419.95 | | 376.85 | 651.74 | 395.92 | 0.61 |
| 220961_s_at | 9238 | TBRG4 | NM_030900 | 102.97 | 124.95 | 97.25 | | 102.97 | 224.01 | 136.09 | 0.61 |
| 216247_at | | | AF113008 | 40.65 | 59.63 | 25.48 | | 40.65 | 80.87 | 49.13 | 0.61 |
| 217757_at | 2 | A2M | NM_000014 | 71.81 | 69.95 | 64.21 | | 71.81 | 113.55 | 69.01 | 0.61 |
| 205150_s_at | 9865 | KIAA0644 | AV724192 | 14.91 | 17.40 | 14.12 | | 14.91 | 31.84 | 19.36 | 0.61 |
| 212940_at | 1291 | COL6A1 | BE350145 | 18.11 | 25.09 | 35.21 | | 18.11 | 52.97 | 32.20 | 0.61 |
| 207860_at | 9437 | NCR1 | NM_004829 | 3.17 | 3.24 | 1.89 | | 3.17 | 10.70 | 6.50 | 0.61 |
| 209459_s_at | 18 | ABAT | AF237813 | 140.40 | 75.56 | 98.52 | | 140.40 | 519.06 | 315.70 | 0.61 |
| 221552_at | 57406 | ABHD6 | BC001698 | 93.69 | 117.93 | 165.66 | | 93.69 | 263.25 | 160.35 | 0.61 |
| 212458_at | 200734 | SPRED2 | H97931 | 831.60 | 1203.64 | 1317.60 | | 831.60 | 4278.55 | 2607.09 | 0.61 |
| 211445_x_at | 389240 / | NACAP1 | AF315951 | 5704.31 | 6411.03 | 9891.98 | | 5704.31 | 9755.78 | 5944.85 | 0.61 |
| | | /// | | | | | | | | | |
| | | LOC389240 | | | | | | | | | |
| 200673_at | 9741 | LAPTM4A | NM_014713 | 1716.40 | 1531.08 | 1677.57 | | 1716.40 | 2675.01 | 1630.72 | 0.61 |
| 217897_at | 53826 | FXYD6 | NM_022003 | 69.25 | 36.73 | 48.36 | | 69.25 | 209.49 | 127.73 | 0.61 |
| 206239_s_at | 6690 | SPINK1 | NM_003122 | 9.50 | 14.67 | 21.22 | | 9.50 | 20.58 | 12.55 | 0.61 |
| 217574_at | 1006 | CDH8 | T65123 | 2.00 | 7.87 | 15.49 | | 2.00 | 32.22 | 19.66 | 0.61 |
| 214841_at | 149111 | CNIH3 | AF070524 | 35.88 | 5.37 | 41.10 | | 35.88 | 154.61 | 94.40 | 0.61 |
| 206712_at | 79774 | GRTP1 | NM_024719 | 5.53 | 6.30 | 11.56 | | 5.53 | 11.69 | 7.14 | 0.61 |
| 217618_x_at | 3364 | HUS1 | AW007988 | 133.90 | 130.20 | 136.67 | | 133.90 | 243.64 | 148.93 | 0.61 |
| 203840_at | 8548 | BLZF1 | NM_003666 | 173.30 | 194.33 | 166.51 | | 173.30 | 306.20 | 187.28 | 0.61 |
| 213037_x_at | 6780 | STAU1 | AJ132258 | 2281.36 | 1958.66 | 2554.56 | | 2281.36 | 3630.45 | 2220.53 | 0.61 |
| 218042_at | 51138 | COPS4 | NM_016129 | 895.30 | 791.04 | 896.08 | | 895.30 | 1469.97 | 899.50 | 0.61 |
| 205994_at | 2005 | ELK4 | NM_001973 | 42.83 | 27.01 | 42.98 | | 42.83 | 80.40 | 49.20 | 0.61 |
| 204653_at | 7020 | TFAP2A | BF343007 | 62.25 | 352.23 | 341.09 | | 62.25 | 1355.87 | 829.89 | 0.61 |
| 208780_x_at | 9218 | VAPA | AF154847 | 5331.27 | 5276.47 | 6669.87 | | 5331.27 | 9326.57 | 5712.03 | 0.61 |
| 215775_at | 7057 | THBS1 | BF084105 | 8.33 | 41.92 | 44.68 | | 8.33 | 45.12 | 27.64 | 0.61 |
| 207480_s_at | 4212 | MEIS2 | NM_020149 | 327.69 | 256.31 | 279.73 | | 327.69 | 1017.66 | 623.66 | 0.61 |
| 218236_s_at | 23683 | PRKD3 | NM_005813 | 533.29 | 623.45 | 771.24 | | 533.29 | 1030.95 | 633.16 | 0.61 |
| 207570_at | 6473 | SHOX | NM_000451 | 3.25 | 7.56 | 7.05 | | 3.25 | 26.59 | 16.33 | 0.61 |
| 211269_s_at | 3559 | IL2RA | K03122 | 6.10 | 3.35 | 6.99 | | 6.10 | 17.80 | 10.94 | 0.61 |
| 213732_at | 6929 | TCF3 | AI871234 | 18.93 | 21.71 | 22.02 | | 18.93 | 36.10 | 22.19 | 0.61 |
| 218819_at | 26512 | INTS6 | NM_012141 | 904.26 | 1274.85 | 1041.84 | | 904.26 | 1658.35 | 1019.46 | 0.61 |
| 202842_s_at | 4189 | DNAJB9 | AL080081 | 396.07 | 479.83 | 595.35 | | 396.07 | 1556.19 | 956.83 | 0.61 |
| 209430_at | 9044 | BTAF1 | AJ001017 | 540.74 | 557.37 | 604.34 | | 540.74 | 1010.70 | 621.56 | 0.61 |
| 217264_s_at | 6337 | SCNN1A | U81961 | 4.44 | 11.93 | 14.34 | | 4.44 | 18.99 | 11.69 | 0.62 |
| 216192_at | 2173 | FABP7 | AL512688 | 8.19 | 10.96 | 6.39 | | 8.19 | 38.90 | 23.95 | 0.62 |
| 209802_at | 7262 | PHLDA2 | BC005034 | 33.05 | 20.28 | 26.10 | | 33.05 | 61.38 | 37.81 | 0.62 |
| 202054_s_at | 224 | ALDH3A2 | NM_000382 | 155.79 | 156.50 | 145.58 | | 155.79 | 275.61 | 169.83 | 0.62 |
| 217371_s_at | 3600 | IL15 | Y09908 | 13.08 | 18.82 | 18.19 | | 13.08 | 32.69 | 20.15 | 0.62 |
| 218263_s_at | 58486 | ZBED5 | NM_021211 | 1325.33 | 1337.89 | 1414.79 | | 1325.33 | 2247.35 | 1388.28 | 0.62 |
| 216038_x_at | 1616 | DAXX | BE965715 | 335.63 | 285.30 | 302.23 | | 335.63 | 620.78 | 383.80 | 0.62 |
| 202720_at | 26136 | TES | NM_015641 | 993.94 | 889.84 | 1123.07 | | 993.94 | 1646.51 | 1018.15 | 0.62 |
| 204619_s_at | 1462 | CSPG2 | BF590263 | 27.86 | 9.59 | 43.59 | | 27.86 | 57.19 | 35.36 | 0.62 |
| 214314_s_at | 9669 | EIF5B | BE138647 | 329.67 | 289.82 | 284.70 | | 329.67 | 566.31 | 350.57 | 0.62 |
| 213352_at | 23023 | TMCC1 | AB018322 | 106.92 | 161.57 | 137.23 | | 106.92 | 214.45 | 132.80 | 0.62 |
| 219022_at | 64897 | C12orf43 | NM_022895 | 348.72 | 354.57 | 362.11 | | 348.72 | 596.55 | 369.52 | 0.62 |
| 208469_s_at | 80864 | EGFL8 | NM_030652 | 21.30 | 62.19 | 62.15 | | 21.30 | 53.36 | 33.05 | 0.62 |
| 217550_at | 22926 | ATF6 | AA576497 | 222.62 | 315.80 | 284.91 | | 222.62 | 404.33 | 250.54 | 0.62 |
| 204722_at | 55800 | SCN3B | AW007335 | 10.53 | 13.04 | 14.60 | | 10.53 | 31.26 | 19.42 | 0.62 |
| 220996_s_at | 81626 | C1orf14 | NM_030933 | 7.22 | 4.00 | 4.45 | | 7.22 | 12.89 | 8.01 | 0.62 |
| 206417_at | 1259 | CNGA1 | NM_000087 | 23.28 | 12.07 | 32.35 | | 23.28 | 46.24 | 28.75 | 0.62 |
| 204516_at | 6314 | ATXN7 | BG390306 | 360.88 | 448.85 | 406.68 | | 360.88 | 617.16 | 383.77 | 0.62 |
| 202255_s_at | 26037 | SIPA1L1 | NM_015556 | 70.22 | 83.38 | 76.14 | | 70.22 | 266.24 | 165.66 | 0.62 |
| 208948_s_at | 6780 | STAU1 | BC000830 | 1935.73 | 1827.41 | 2306.62 | | 1935.73 | 3498.32 | 2176.79 | 0.62 |
| 210135_s_at | 6474 | SHOX2 | AF022654 | 3460.15 | 4612.13 | 4402.03 | | 3460.15 | 8271.93 | 5147.53 | 0.62 |
| 216830_at | 730909 | LOC730909 | AC004460 | 3.44 | 7.04 | 7.39 | | 3.44 | 14.18 | 8.82 | 0.62 |
| 200028_s_at | 56910 | STARD7 | NM_020151 | 3322.02 | 3987.67 | 5578.30 | | 3322.02 | 5058.23 | 3148.26 | 0.62 |
| 205173_x_at | 965 | CD58 | NM_001779 | 1039.87 | 2046.09 | 2432.88 | | 1039.87 | 2577.48 | 1604.72 | 0.62 |
| 200765_x_at | 1495 | CTNNA1 | NM_001903 | 1333.37 | 1579.45 | 1452.13 | | 1333.37 | 2113.12 | 1315.90 | 0.62 |
| 222063_s_at | 1040 | CDS1 | AI991484 | 8.41 | 31.67 | 28.21 | | 8.41 | 18.70 | 11.65 | 0.62 |
| 203556_at | 22882 | ZHX2 | NM_014943 | 119.00 | 133.57 | 110.55 | | 119.00 | 615.44 | 383.72 | 0.62 |
| 204349_at | 9443 | CRSP9 | BC005250 | 180.17 | 139.89 | 326.86 | | 180.17 | 333.79 | 208.15 | 0.62 |
| 209065_at | 7381 | UQCRB | BC005230 | 481.62 | 422.25 | 445.20 | | 481.62 | 735.25 | 458.52 | 0.62 |
| 201352_at | 10730 | YME1L1 | NM_014263 | 1525.93 | 1264.93 | 1815.25 | | 1525.93 | 2877.65 | 1796.07 | 0.62 |
| 215011_at | 8420 | SNHG3 | AJ006835 | 60.04 | 75.78 | 38.54 | | 60.04 | 139.09 | 86.90 | 0.62 |
| 218050_at | 51569 | UFM1 | NM_016617 | 692.72 | 719.87 | 1187.22 | | 692.72 | 2208.74 | 1380.11 | 0.62 |
| 216132_at | 23245 | ASTN2 | AK021992 | 6.29 | 18.19 | 20.26 | | 6.29 | 10.28 | 6.43 | 0.63 |
| 209798_at | 4863 | NPAT | D83243 | 320.67 | 306.07 | 351.91 | | 320.67 | 498.47 | 312.15 | 0.63 |
| 221193_s_at | 54819 | ZCCHC10 | NM_017665 | 463.67 | 457.06 | 544.79 | | 463.67 | 1000.85 | 626.77 | 0.63 |
| 205103_at | 10485 | C1orf61 | NM_006364 | 73.74 | 102.20 | 65.12 | | 73.74 | 154.86 | 97.00 | 0.63 |
| 218677_at | 57402 | S100A14 | NM_020672 | 13.70 | 17.72 | 27.49 | | 13.70 | 36.21 | 22.69 | 0.63 |
| 204593_s_at | 54471 | SMCR7L | AA046752 | 621.68 | 593.59 | 607.69 | | 621.68 | 1025.19 | 642.44 | 0.63 |
| 220966_x_at | 81873 | ARPC5L | NM_030978 | 1914.80 | 2009.32 | 2351.08 | | 1914.80 | 2981.97 | 1868.80 | 0.63 |
| 219844_at | 55088 | C10orf118 | NM_018017 | 20.21 | 30.40 | 25.63 | | 20.21 | 32.29 | 20.25 | 0.63 |
| 205437_at | 10520 | ZNF211 | NM_006385 | 235.16 | 313.25 | 289.22 | | 235.16 | 466.66 | 292.72 | 0.63 |
| 216086_at | 22987 | SV2C | AB028977 | 29.52 | 16.62 | 29.61 | | 29.52 | 100.11 | 62.81 | 0.63 |
| 219540_at | 10308 | ZNF267 | NM_003414 | 514.71 | 544.69 | 753.59 | | 514.71 | 1470.33 | 922.84 | 0.63 |
| 201190_s_at | 5306 | PITPNA | H15647 | 429.23 | 572.34 | 461.82 | | 429.23 | 1791.54 | 1125.31 | 0.63 |
| 216113_at | 10152 | ABI2 | AF070566 | 17.41 | 10.75 | 15.16 | | 17.41 | 27.82 | 17.48 | 0.63 |
| 213359_at | 3184 | HNRPD | W74620 | 554.16 | 734.55 | 636.69 | | 554.16 | 1436.56 | 902.80 | 0.63 |
| 46323_at | 124583 | CANT1 | AL120741 | 792.96 | 791.03 | 798.35 | | 792.96 | 1373.99 | 863.60 | 0.63 |
| 202365_at | 84747 | MGC5139 | BC004815 | 914.38 | 872.52 | 937.04 | | 914.38 | 1446.69 | 909.35 | 0.63 |
| 215810_x_at | 667 | DST | AL049215 | 11.98 | 25.79 | 46.93 | | 11.98 | 60.62 | 38.13 | 0.63 |
| 45653_at | 253980 | KCTD13 | AW026481 | 289.71 | 276.32 | 362.53 | | 289.71 | 440.68 | 277.48 | 0.63 |
| 1405_i_at | 6352 | CCL5 | M21121 | 1821.38 | 2494.79 | 2775.85 | | 1821.38 | 13200.54 | 8314.36 | 0.63 |
| 202193_at | 3985 | LIMK2 | NM_005569 | 197.52 | 265.61 | 274.95 | | 197.52 | 417.67 | 263.27 | 0.63 |
| 203812_at | | | AB011538 | 26.86 | 58.74 | 62.88 | | 26.86 | 64.33 | 40.57 | 0.63 |
| 215086_at | 25998 | IBTK | AB037838 | 30.19 | 36.89 | 51.24 | | 30.19 | 51.15 | 32.27 | 0.63 |
| 213222_at | 23236 | PLCB1 | AL049593 | 80.40 | 73.11 | 121.00 | | 80.40 | 132.89 | 83.86 | 0.63 |
| 221741_s_at | 54915 | YTHDF1 | AL096828 | 1411.69 | 1390.49 | 1433.95 | | 1411.69 | 2711.22 | 1711.48 | 0.63 |
| 217921_at | | | BE543064 | 190.20 | 195.75 | 162.92 | | 190.20 | 389.90 | 246.42 | 0.63 |
| 210702_s_at | 5740 | PTGIS | D38145 | 6.39 | 5.00 | 1.54 | | 6.39 | 13.11 | 8.29 | 0.63 |
| 205854_at | 7289 | TULP3 | AK024246 | 167.54 | 248.46 | 286.62 | | 167.54 | 362.71 | 229.74 | 0.63 |
| 206670_s_at | 253782 / | GAD1 | NM_013445 | 4.24 | 5.43 | 4.17 | | 4.24 | 10.29 | 6.52 | 0.63 |
| | | /// | | | | | | | | | |
| | | LASS6 | | | | | | | | | |
| 204780_s_at | 355 | FAS | NM_000043 | 26.39 | 33.87 | 40.50 | | 26.39 | 53.76 | 34.12 | 0.63 |
| 210636_at | 5467 | PPARD | BC002715 | 26.93 | 69.25 | 56.95 | | 26.93 | 52.71 | 33.46 | 0.63 |
| 211028_s_at | 3795 | KHK | BC006233 | 18.42 | 19.93 | 16.58 | | 18.42 | 30.64 | 19.46 | 0.64 |
| 204781_s_at | 355 | FAS | NM_000043 | 16.62 | 35.30 | 29.61 | | 16.62 | 31.02 | 19.73 | 0.64 |
| 208841_s_at | 9908 | G3BP2 | AB014560 | 1452.94 | 1479.71 | 1338.12 | | 1452.94 | 2487.86 | 1582.42 | 0.64 |
| 205100_at | 9945 | GFPT2 | NM_005110 | 24.06 | 34.37 | 31.69 | | 24.06 | 190.76 | 121.38 | 0.64 |
| 217935_s_at | 55245 | C20orf44 | NM_018244 | 367.57 | 388.98 | 356.64 | | 367.57 | 623.00 | 396.43 | 0.64 |
| 218313_s_at | 51809 | GALNT7 | NM_017423 | 685.51 | 813.24 | 616.16 | | 685.51 | 1333.29 | 849.43 | 0.64 |
| 202723_s_at | 2308 | FOXO1A | AW117498 | 69.08 | 78.66 | 55.05 | | 69.08 | 162.03 | 103.24 | 0.64 |
| 212599_at | 26053 | AUTS2 | AK025298 | 19.64 | 20.60 | 25.17 | | 19.64 | 40.25 | 25.65 | 0.64 |
| 221208_s_at | 79684 | C11orf61 | NM_024631 | 132.94 | 188.70 | 173.23 | | 132.94 | 294.70 | 187.84 | 0.64 |
| 216874_at | 401014 | DKFZp686O1327 | U80770 | 66.91 | 175.68 | 200.55 | | 66.91 | 206.56 | 131.67 | 0.64 |
| 211210_x_at | 4068 | SH2D1A | AF100539 | 5.43 | 7.71 | 5.37 | | 5.43 | 19.02 | 12.14 | 0.64 |
| 202293_at | 10274 | STAG1 | NM_005862 | 309.76 | 346.82 | 356.80 | | 309.76 | 481.01 | 307.06 | 0.64 |
| 202271_at | 23219 | FBXO28 | AB007952 | 1350.05 | 1452.41 | 1483.51 | | 1350.05 | 2591.69 | 1654.61 | 0.64 |
| 216993_s_at | 1302 | COL11A2 | U32169 | 67.02 | 84.79 | 72.25 | | 67.02 | 106.06 | 67.73 | 0.64 |
| 204661_at | 1043 | CD52 | NM_001803 | 579.06 | 587.07 | 457.80 | | 579.06 | 972.91 | 621.71 | 0.64 |
| 203508_at | 7133 | TNFRSF1B | NM_001066 | 572.31 | 801.24 | 236.66 | | 572.31 | 1149.71 | 735.84 | 0.64 |
| 203341_at | 10153 | CEBPZ | NM_005760 | 1314.48 | 1239.38 | 1179.44 | | 1314.48 | 2203.97 | 1411.09 | 0.64 |
| 201983_s_at | 1956 | EGFR | NM_005228 | 12.28 | 24.96 | 27.80 | | 12.28 | 34.35 | 22.00 | 0.64 |
| 202987_at | 10758 | TRAF3IP2 | NM_015524 | 21.83 | 10.52 | 29.84 | | 21.83 | 80.46 | 51.59 | 0.64 |
| 214581_x_at | 27242 | TNFRSF21 | BE568134 | 179.96 | 215.92 | 135.04 | | 179.96 | 489.11 | 313.70 | 0.64 |
| 222048_at | 157 | ADRBK2 | AA536000 | 57.47 | 78.29 | 98.63 | | 57.47 | 97.18 | 62.44 | 0.64 |
| 218851_s_at | 55339 | WDR33 | NM_018383 | 69.73 | 77.84 | 112.19 | | 69.73 | 128.53 | 82.68 | 0.64 |
| 209033_s_at | 1859 | DYRK1A | D86550 | 2329.08 | 2284.34 | 3123.43 | | 2329.08 | 3691.16 | 2377.82 | 0.64 |
| 209031_at | 23705 | IGSF4 | AL519710 | 144.00 | 176.61 | 140.94 | | 144.00 | 430.57 | 277.60 | 0.64 |
| 217767_at | 653879 | LOC653879 | NM_000064 | 773.37 | 1495.52 | 1178.52 | | 773.37 | 1752.15 | 1131.92 | 0.65 |
| 212950_at | 221395 | GPR116 | BF941499 | 5.28 | 20.47 | 15.58 | | 5.28 | 20.02 | 12.93 | 0.65 |
| 221538_s_at | 5361 | PLXNA1 | T16388 | 186.40 | 295.26 | 224.93 | | 186.40 | 687.09 | 444.55 | 0.65 |
| 219377_at | 64762 | FAM59A | NM_022751 | 133.94 | 155.88 | 209.60 | | 133.94 | 218.04 | 141.17 | 0.65 |
| 205284_at | 9816 | KIAA0133 | NM_014777 | 305.13 | 369.25 | 283.02 | | 305.13 | 682.23 | 442.04 | 0.65 |
| 215289_at | 388567 | ZNF749 | BE892698 | 2.08 | 17.62 | 16.87 | | 2.08 | 18.09 | 11.73 | 0.65 |
| 213618_at | 116984 | CENTD1 | AB011152 | 62.37 | 97.06 | 112.50 | | 62.37 | 263.80 | 171.11 | 0.65 |
| 220623_s_at | 80705 | TSGA10 | NM_025244 | 30.14 | 38.70 | 12.04 | | 30.14 | 56.68 | 36.78 | 0.65 |
| 219848_s_at | 9668 | ZNF432 | NM_014650 | 194.30 | 141.73 | 254.84 | | 194.30 | 311.26 | 202.01 | 0.65 |
| 213278_at | 66036 | MTMR9 | AW014788 | 475.20 | 517.24 | 698.96 | | 475.20 | 898.98 | 583.99 | 0.65 |
| 204529_s_at | 9760 | TOX | AI961231 | 17.75 | 24.41 | 35.72 | | 17.75 | 60.72 | 39.55 | 0.65 |
| 210946_at | 8611 | PPAP2A | AF014403 | 97.48 | 130.98 | 203.41 | | 97.48 | 195.26 | 127.21 | 0.65 |
| 204136_at | 1294 | COL7A1 | NM_000094 | 60.72 | 55.28 | 78.62 | | 60.72 | 162.49 | 105.89 | 0.65 |
| 218691_s_at | 8572 | PDLIM4 | NM_003687 | 4.55 | 4.89 | 5.20 | | 4.55 | 151.08 | 98.47 | 0.65 |
| 217544_at | 729806 / | LOC729806 | AA768909 | 112.17 | 190.34 | 162.62 | | 112.17 | 274.56 | 178.99 | 0.65 |
| | | /// | | | | | | | | | |
| | | LOC730619 | | | | | | | | | |
| 213861_s_at | 25895 | FAM119B | N67741 | 215.39 | 202.33 | 158.00 | | 215.39 | 351.94 | 229.48 | 0.65 |
| 45297_at | 30846 | EHD2 | AI417917 | 95.74 | 87.00 | 71.45 | | 95.74 | 144.40 | 94.16 | 0.65 |
| 213692_s_at | 79053 | ALG8 | AA904259 | 3.89 | 3.57 | 4.70 | | 3.89 | 20.32 | 13.26 | 0.65 |
| 200069_at | 9733 | SART3 | AI656011 | 582.33 | 526.70 | 618.52 | | 582.33 | 944.87 | 616.77 | 0.65 |
| 215067_x_at | 7001 | PRDX2 | AU147942 | 198.01 | 368.59 | 300.37 | | 198.01 | 329.30 | 215.14 | 0.65 |
| 204230_s_at | 57030 | SLC17A7 | NM_020309 | 62.54 | 78.99 | 57.21 | | 62.54 | 135.06 | 88.27 | 0.65 |
| 222197_s_at | | | AF131813 | 4.96 | 8.77 | 15.30 | | 4.96 | 11.17 | 7.31 | 0.65 |
| 202220_at | 22889 | KIAA0907 | NM_014949 | 2158.94 | 3002.45 | 1957.96 | | 2158.94 | 3789.31 | 2483.99 | 0.66 |
| 220270_at | 56163 | RNF17 | NM_019038 | 7.77 | 3.66 | 5.55 | | 7.77 | 19.52 | 12.80 | 0.66 |
| 214471_x_at | 3972 | LHB | NM_000894 | 13.71 | 5.86 | 7.49 | | 13.71 | 57.04 | 37.39 | 0.66 |
| 208660_at | 1431 | CS | BC000105 | 2968.51 | 2552.89 | 2743.70 | | 2968.51 | 5865.14 | 3845.51 | 0.66 |
| 214138_at | 7633 | ZNF79 | AA284829 | 106.60 | 106.92 | 107.51 | | 106.60 | 171.74 | 112.61 | 0.66 |
| 215087_at | 56905 | C15orf39 | AL109730 | 95.46 | 110.41 | 121.80 | | 95.46 | 174.64 | 114.62 | 0.66 |
| 213585_s_at | 5134 | PDCD2 | AA764988 | 30.02 | 58.73 | 53.27 | | 30.02 | 59.62 | 39.17 | 0.66 |
| 823_at | 6376 | CX3CL1 | U84487 | 5.74 | 7.30 | 22.08 | | 5.74 | 48.30 | 31.74 | 0.66 |
| 220608_s_at | | | NM_014106 | 36.90 | 44.48 | 59.57 | | 36.90 | 90.25 | 59.31 | 0.66 |
| 205724_at | 5317 | PKP1 | NM_000299 | 22.49 | 12.92 | 9.37 | | 22.49 | 42.36 | 27.84 | 0.66 |
| 216693_x_at | 50810 | HDGFRP3 | AL133102 | 21.71 | 47.74 | 39.87 | | 21.71 | 52.67 | 34.62 | 0.66 |
| 214656_x_at | 4641 | MYO1C | BE790157 | 173.29 | 327.17 | 269.11 | | 173.29 | 412.61 | 271.33 | 0.66 |
| 215082_at | 55000 | TUG1 | BF973387 | 163.51 | 118.99 | 150.38 | | 163.51 | 286.97 | 188.74 | 0.66 |
| | // | /// | | | | | | | | | |
| | | ELOVL5 | | | | | | | | | |
| 211764_s_at | 7321 | UBE2D1 | BC005980 | 101.20 | 156.08 | 178.95 | | 101.20 | 238.38 | 156.79 | 0.66 |
| 221062_at | 9953 | HS3ST3B1 | NM_006041 | 10.40 | 8.33 | 4.04 | | 10.40 | 49.34 | 32.46 | 0.66 |
| 213876_x_at | 8233 | ZRSR2 | AW089584 | 288.13 | 421.13 | 376.38 | | 288.13 | 456.03 | 300.00 | 0.66 |
| 40016_g_at | 23227 | MAST4 | AB002301 | 52.75 | 54.34 | 65.26 | | 52.75 | 142.91 | 94.10 | 0.66 |
| 216691_at | | | AL137705 | 6.99 | 2.59 | 4.47 | | 6.99 | 13.48 | 8.88 | 0.66 |
| 212852_s_at | 6738 | TROVE2 | AL538601 | 2410.62 | 2740.92 | 2451.15 | | 2410.62 | 3727.41 | 2454.72 | 0.66 |
| 209598_at | 10687 | PNMA2 | AB020690 | 62.45 | 98.10 | 105.80 | | 62.45 | 128.01 | 84.35 | 0.66 |
| 209403_at | 414060 / | TBC1D3 | AL136860 | 356.98 | 312.13 | 314.68 | | 356.98 | 546.19 | 359.92 | 0.66 |
| | | /// | | | | | | | | | |
| | | TBC1D3C | | | | | | | | | |
| | | /// | | | | | | | | | |
| | | LOC6533 | | | | | | | | | |
| 214112_s_at | 541578 / | CXorf40A | AA543076 | 689.74 | 601.70 | 673.93 | | 689.74 | 1073.97 | 708.60 | 0.66 |
| | | /// | | | | | | | | | |
| | | CXorf40B | | | | | | | | | |
| 219500_at | 23529 | CLCF1 | NM_013246 | 35.07 | 29.74 | 13.52 | | 35.07 | 102.82 | 67.87 | 0.66 |
| 214372_x_at | 10595 | ERN2 | AI732416 | 109.55 | 201.02 | 223.42 | | 109.55 | 223.09 | 147.47 | 0.66 |
| 215614_at | 644974 | LOC644974 | AK022038 | 12.63 | 12.40 | 12.05 | | 12.63 | 50.87 | 33.63 | 0.66 |
| 201602_s_at | 4659 | PPP1R12A | NM_002480 | 215.44 | 287.20 | 298.78 | | 215.44 | 388.08 | 256.86 | 0.66 |
| 214866_at | 5329 | PLAUR | X74039 | 153.90 | 213.29 | 96.43 | | 153.90 | 329.94 | 218.47 | 0.66 |
| 201409_s_at | 5500 | PPP1CB | W67887 | 546.52 | 512.06 | 671.00 | | 546.52 | 836.89 | 555.22 | 0.66 |
| 204721_s_at | 9829 | DNAJC6 | NM_014787 | 4.28 | 3.77 | 4.86 | | 4.28 | 14.26 | 9.47 | 0.66 |
| 222234_s_at | 79007 | DBNDD1 | AK022644 | 6.22 | 12.23 | 19.51 | | 6.22 | 43.98 | 29.19 | 0.66 |
| 202127_at | 8899 | PRPF4B | AB011108 | 264.99 | 306.02 | 286.71 | | 264.99 | 622.74 | 413.31 | 0.66 |
| 213221_s_at | 23235 | SNF1LK2 | AB018324 | 110.53 | 156.13 | 99.85 | | 110.53 | 204.87 | 136.04 | 0.66 |
| 219194_at | 57715 | SEMA4G | NM_017893 | 28.58 | 53.46 | 52.64 | | 28.58 | 63.92 | 42.45 | 0.66 |
| 209593_s_at | 27348 | TOR1B | AF317129 | 483.60 | 692.12 | 572.37 | | 483.60 | 807.28 | 536.51 | 0.66 |
| 213704_at | 5876 | RABGGTB | AA129753 | 197.33 | 177.45 | 219.25 | | 197.33 | 366.85 | 243.81 | 0.66 |
| 208989_s_at | 22992 | FBXL11 | AF179221 | 162.99 | 267.67 | 236.95 | | 162.99 | 299.12 | 199.08 | 0.67 |
| 214260_at | 10920 | COPS8 | AI079287 | 69.54 | 97.49 | 105.14 | | 69.54 | 119.44 | 79.52 | 0.67 |
| 214688_at | 7091 | TLE4 | BF217301 | 41.94 | 49.52 | 47.01 | | 41.94 | 66.86 | 44.53 | 0.67 |
| 215978_x_at | 152719 | LOC152719 | AK021514 | 1806.07 | 2790.17 | 3474.09 | | 1806.07 | 2924.90 | 1949.14 | 0.67 |
| 204740_at | 10256 | CNKSR1 | NM_006314 | 20.78 | 24.14 | 26.97 | | 20.78 | 46.39 | 30.91 | 0.67 |
| 212761_at | 6934 | TCF7L2 | AI703074 | 172.85 | 151.25 | 269.32 | | 172.85 | 328.32 | 218.86 | 0.67 |
| 213128_s_at | 7337 | UBE3A | AA527499 | 533.93 | 525.26 | 656.50 | | 533.93 | 1469.59 | 979.70 | 0.67 |
| 202405_at | 7073 | TIAL1 | BF432532 | 98.64 | 140.10 | 130.96 | | 98.64 | 150.71 | 100.50 | 0.67 |
| 206836_at | 6531 | SLC6A3 | NM_001044 | 32.57 | 47.75 | 67.11 | | 32.57 | 65.59 | 43.80 | 0.67 |
| 204566_at | 8493 | PPM1D | NM_003620 | 639.99 | 481.26 | 854.60 | | 639.99 | 1094.05 | 730.95 | 0.67 |
| 213410_at | 26098 | C10orf137 | AL050102 | 443.56 | 429.34 | 435.58 | | 443.56 | 693.36 | 463.29 | 0.67 |
| 213229_at | 23405 | DICER1 | BF590131 | 589.34 | 796.84 | 751.13 | | 589.34 | 1257.70 | 840.42 | 0.67 |
| 202324_s_at | 64746 | ACBD3 | NM_022735 | 1564.09 | 1347.60 | 1960.89 | | 1564.09 | 2512.28 | 1680.94 | 0.67 |
| 201712_s_at | 5903 | RANBP2 | NM_006267 | 264.00 | 289.96 | 271.50 | | 264.00 | 431.95 | 289.25 | 0.67 |
| 205054_at | 4703 | NEB | NM_004543 | 93.61 | 109.65 | 94.01 | | 93.61 | 220.07 | 147.41 | 0.67 |
| 201038_s_at | 8125 | ANP32A | T67821 | 568.73 | 749.56 | 643.22 | | 568.73 | 941.00 | 630.35 | 0.67 |
| 218947_s_at | 55149 | PAPD1 | NM_018109 | 285.78 | 249.08 | 237.07 | | 285.78 | 490.12 | 328.33 | 0.67 |
| 212158_at | 6383 | SDC2 | AL577322 | 784.59 | 589.45 | 861.24 | | 784.59 | 1824.34 | 1222.69 | 0.67 |
| 217373_x_at | 4193 | MDM2 | AJ276888 | 111.98 | 170.16 | 130.59 | | 111.98 | 204.89 | 137.35 | 0.67 |
| 218566_s_at | 26973 | CHORDC1 | NM_012124 | 828.63 | 1258.04 | 1179.87 | | 828.63 | 2132.94 | 1430.73 | 0.67 |
| 210837_s_at | 5144 | PDE4D | AF012074 | 29.95 | 21.81 | 28.93 | | 29.95 | 53.52 | 35.92 | 0.67 |
| 219363_s_at | 51001 | MTERFD1 | NM_015942 | 1244.05 | 1473.56 | 1572.44 | | 1244.05 | 2732.43 | 1834.85 | 0.67 |
| 216273_at | 10794 | ZNF460 | X78931 | 21.22 | 23.52 | 22.80 | | 21.22 | 48.25 | 32.43 | 0.67 |
| 208200_at | 3552 | IL1A | NM_000575 | 55.13 | 110.54 | 99.32 | | 55.13 | 113.12 | 76.07 | 0.67 |
| 213810_s_at | | | AW007137 | 72.66 | 71.00 | 135.01 | | 72.66 | 124.79 | 83.95 | 0.67 |
| 201952_at | 214 | ALCAM | AA156721 | 2044.41 | 3613.90 | 3693.11 | | 2044.41 | 6244.65 | 4201.66 | 0.67 |
| 201984_s_at | 1956 | EGFR | NM_005228 | 24.71 | 35.65 | 15.96 | | 24.71 | 56.33 | 37.91 | 0.67 |
| 218053_at | 55660 | PRPF40A | NM_017892 | 3256.96 | 3735.16 | 3812.97 | | 3256.96 | 5228.97 | 3518.93 | 0.67 |
| 210965_x_at | 8621 | CDC2L5 | BC001274 | 78.95 | 110.34 | 108.81 | | 78.95 | 123.86 | 83.37 | 0.67 |
| 221077_at | 55130 | ARMC4 | NM_018076 | 34.21 | 60.53 | 49.11 | | 34.21 | 60.89 | 40.99 | 0.67 |
| 218496_at | 246243 | RNASEH1 | BG534527 | 640.72 | 655.75 | 653.07 | | 640.72 | 1224.76 | 824.85 | 0.67 |
| 222203_s_at | 57665 | RDH14 | AK023625 | 430.76 | 452.66 | 377.55 | | 430.76 | 713.33 | 480.74 | 0.67 |
| 216480_x_at | 8028 | MLLT10 | AF060927 | 32.27 | 39.01 | 44.30 | | 32.27 | 55.76 | 37.65 | 0.68 |
| 214024_s_at | 85359 | DGCR6L | AA631156 | 3.04 | 13.54 | 12.89 | | 3.04 | 26.19 | 17.69 | 0.68 |
| 50400_at | 196743 | PAOX | AI743990 | 27.46 | 33.83 | 16.08 | | 27.46 | 54.67 | 36.95 | 0.68 |
| 208581_x_at | 4501 | MT1X | NM_005952 | 876.45 | 500.40 | 601.35 | | 876.45 | 2203.33 | 1489.46 | 0.68 |
| 201496_x_at | 4629 | MYH11 | NM_022844 | 10.07 | 9.74 | 10.26 | | 10.07 | 33.15 | 22.42 | 0.68 |
| 217845_x_at | 25994 | HIGD1A | NM_014056 | 1832.29 | 1853.31 | 1904.64 | | 1832.29 | 3290.28 | 2225.17 | 0.68 |
| 210652_s_at | 22996 | C1orf34 | BC004399 | 3.79 | 25.91 | 40.94 | | 3.79 | 84.15 | 56.95 | 0.68 |
| 205737_at | 3785 | KCNQ2 | NM_004518 | 17.68 | 32.00 | 27.10 | | 17.68 | 27.84 | 18.85 | 0.68 |
| 46270_at | 51271 | UBAP1 | AL039447 | 342.24 | 380.31 | 413.58 | | 342.24 | 649.74 | 440.14 | 0.68 |
| 221649_s_at | 56342 | PPAN | BC000535 | 312.43 | 366.94 | 234.87 | | 312.43 | 675.36 | 457.55 | 0.68 |
| 203769_s_at | 412 | STS | NM_000351 | 10.57 | 15.22 | 10.18 | | 10.57 | 34.55 | 23.41 | 0.68 |
| 215134_at | 55361 | PI4KII | H84390 | 26.18 | 29.67 | 36.22 | | 26.18 | 52.67 | 35.71 | 0.68 |
| 218761_at | 54778 | RNF111 | NM_017610 | 706.35 | 648.47 | 898.79 | | 706.35 | 1126.75 | 763.98 | 0.68 |
| 219383_at | 79899 | FLJ14213 | NM_024841 | 15.95 | 20.81 | 9.16 | | 15.95 | 25.82 | 17.51 | 0.68 |
| 215373_x_at | 80047 | FLJ12151 | AK022213 | 286.84 | 486.56 | 443.36 | | 286.84 | 449.99 | 305.38 | 0.68 |
| 201889_at | 10447 | FAM3C | NM_014888 | 676.05 | 797.67 | 690.68 | | 676.05 | 1529.69 | 1038.27 | 0.68 |
| 213903_s_at | 9125 | RQCD1 | AI380850 | 10.49 | 13.73 | 63.23 | | 10.49 | 58.80 | 39.94 | 0.68 |
| 215877_at | 89919 | C14orf56 | AK024445 | 7.32 | 9.83 | 6.32 | | 7.32 | 15.64 | 10.62 | 0.68 |
| 200884_at | 1152 | CKB | NM_001823 | 228.03 | 304.10 | 238.74 | | 228.03 | 1445.47 | 982.33 | 0.68 |
| 218614_at | 55196 | C12orf35 | NM_018169 | 804.52 | 1513.93 | 1308.08 | | 804.52 | 3148.79 | 2140.21 | 0.68 |
| 211977_at | 57720 | GPR107 | AK024651 | 217.94 | 269.29 | 272.32 | | 217.94 | 386.14 | 262.55 | 0.68 |
| 202345_s_at | 2171 /// | FABP5 | NM_001444 | 4523.51 | 3366.88 | 3513.20 | | 4523.51 | 16384.06 | 11146.49 | 0.68 |
| | | /// | | | | | | | | | |
| | | LOC728641 | | | | | | | | | |
| | | /// | | | | | | | | | |
| | | LOC729 | | | | | | | | | |
| 218708_at | 29107 | NXT1 | NM_013248 | 778.31 | 1166.08 | 763.95 | | 778.31 | 1400.02 | 952.50 | 0.68 |
| 216989_at | 6677 | SPAM1 | L13779 | 13.11 | 19.24 | 28.62 | | 13.11 | 28.25 | 19.22 | 0.68 |
| 221919_at | 10076 | PTPRU | AW450929 | 251.98 | 301.98 | 195.53 | | 251.98 | 400.23 | 272.58 | 0.68 |
| 212668_at | 57154 | SMURF1 | AI654869 | 20.74 | 21.17 | 35.19 | | 20.74 | 36.29 | 24.72 | 0.68 |
| 206693_at | 3574 | IL7 | NM_000880 | 12.25 | 6.30 | 2.16 | | 12.25 | 22.86 | 15.57 | 0.68 |
| 206139_at | 5298 | PIK4CB | NM_002651 | 213.39 | 239.42 | 201.75 | | 213.39 | 425.61 | 290.40 | 0.68 |
| 205956_x_at | 29893 | PSMC3IP | NM_013290 | 316.31 | 400.51 | 344.65 | | 316.31 | 495.96 | 338.41 | 0.68 |
| 213181_s_at | 4337 | MOCS1 | AL583528 | 29.77 | 26.74 | 25.30 | | 29.77 | 67.62 | 46.15 | 0.68 |
| 217063_x_at | 6955 | TRA@ | X61079 | 2.72 | 9.23 | 18.29 | | 2.72 | 23.34 | 15.93 | 0.68 |
| 215986_at | 64430 | C14orf135 | AU146999 | 19.83 | 18.51 | 47.63 | | 19.83 | 53.83 | 36.75 | 0.68 |
| 33148_at | 51663 | ZFR | AI459274 | 115.02 | 141.65 | 152.28 | | 115.02 | 184.56 | 126.04 | 0.68 |
| 221677_s_at | 29980 | DONSON | AF232674 | 565.80 | 758.88 | 535.20 | | 565.80 | 891.79 | 609.29 | 0.68 |
| 202076_at | 329 | BIRC2 | NM_001166 | 1990.37 | 2073.34 | 2233.86 | | 1990.37 | 3604.44 | 2463.37 | 0.68 |
| 200071_at | 10285 | SMNDC1 | BF224259 | 908.25 | 733.38 | 1170.30 | | 908.25 | 1573.19 | 1075.36 | 0.68 |
| 208424_s_at | 57019 | CIAPIN1 | NM_020313 | 445.08 | 432.13 | 457.50 | | 445.08 | 682.47 | 466.70 | 0.68 |
| 220344_at | 56673 | C11orf16 | NM_020643 | 6.98 | 11.69 | 12.15 | | 6.98 | 10.82 | 7.40 | 0.68 |
| 209568_s_at | 23179 | RGL1 | AF186779 | 146.44 | 1099.56 | 1182.05 | | 146.44 | 2480.91 | 1697.81 | 0.68 |
| 202388_at | 5997 | RGS2 | NM_002923 | 446.40 | 463.76 | 501.12 | | 446.40 | 839.25 | 574.37 | 0.68 |
| 219047_s_at | 79759 | ZNF668 | NM_024706 | 101.56 | 111.58 | 135.23 | | 101.56 | 180.00 | 123.19 | 0.68 |
| 219062_s_at | 54877 | ZCCHC2 | BE676543 | 266.43 | 339.99 | 333.79 | | 266.43 | 747.35 | 511.64 | 0.68 |
| 222239_s_at | 26512 | INTS6 | AL117626 | 466.70 | 675.29 | 449.82 | | 466.70 | 796.00 | 545.04 | 0.68 |
| 214056_at | 4170 | MCL1 | BF981280 | 115.20 | 143.89 | 147.31 | | 115.20 | 224.16 | 153.55 | 0.68 |
| 214985_at | | | AF070571 | 4.89 | 8.75 | 7.84 | | 4.89 | 13.75 | 9.42 | 0.69 |
| 204973_at | 2705 | GJB1 | NM_000166 | 24.10 | 20.54 | 28.13 | | 24.10 | 50.42 | 34.59 | 0.69 |
| 221639_x_at | 3192 | HNRPU | AF068846 | 10.18 | 49.40 | 46.67 | | 10.18 | 42.15 | 28.97 | 0.69 |
| 222182_s_at | 4848 | CNOT2 | BG105204 | 1543.44 | 1189.76 | 1716.16 | | 1543.44 | 2422.67 | 1666.06 | 0.69 |
| 202228_s_at | 27020 | NPTN | NM_017455 | 2921.44 | 3196.32 | 2764.11 | | 2921.44 | 5025.08 | 3456.04 | 0.69 |
| 211966_at | 1284 | COL4A2 | AA909035 | 81.19 | 106.34 | 79.25 | | 81.19 | 182.30 | 125.38 | 0.69 |
| 217880_at | | | NM_001256 | 615.00 | 638.24 | 1089.08 | | 615.00 | 998.24 | 686.60 | 0.69 |
| 210346_s_at | 57396 | CLK4 | AF212224 | 841.00 | 1016.99 | 925.71 | | 841.00 | 1415.61 | 973.80 | 0.69 |
| 209004_s_at | 26234 | FBXL5 | AF142481 | 1612.23 | 2097.63 | 2522.88 | | 1612.23 | 2599.68 | 1789.14 | 0.69 |
| 220308_at | 25790 | CCDC19 | NM_012337 | 24.68 | 18.43 | 35.70 | | 24.68 | 49.55 | 34.11 | 0.69 |
| 215888_at | 23047 | APRIN | AK026889 | 179.53 | 309.06 | 287.54 | | 179.53 | 358.20 | 246.99 | 0.69 |
| 202205_at | 7408 | VASP | NM_003370 | 607.69 | 646.02 | 574.87 | | 607.69 | 1055.66 | 728.22 | 0.69 |
| 222118_at | 55839 | CENPN | AK023669 | 254.76 | 350.51 | 256.95 | | 254.76 | 449.58 | 310.14 | 0.69 |
| 219092_s_at | 64768 | IPPK | NM_022755 | 116.77 | 183.23 | 164.92 | | 116.77 | 231.85 | 159.96 | 0.69 |
| 205039_s_at | 10320 | IKZF1 | BG540504 | 962.44 | 752.19 | 433.36 | | 962.44 | 2080.00 | 1436.83 | 0.69 |
| 211129_x_at | 1896 | EDA | AF061192 | 19.57 | 12.86 | 17.66 | | 19.57 | 32.45 | 22.41 | 0.69 |
| 200784_s_at | 4035 | LRP1 | BF304759 | 9.51 | 13.00 | 17.38 | | 9.51 | 15.26 | 10.55 | 0.69 |
| 206043_s_at | 9914 | ATP2C2 | NM_014861 | 12.10 | 9.27 | 19.04 | | 12.10 | 24.72 | 17.09 | 0.69 |
| 213225_at | 5495 | PPM1B | AJ271832 | 140.21 | 161.08 | 170.43 | | 140.21 | 242.34 | 167.59 | 0.69 |
| 213701_at | 91298 | C12orf29 | AW299245 | 609.18 | 529.79 | 616.46 | | 609.18 | 1011.04 | 699.53 | 0.69 |
| 203161_s_at | 9025 | RNF8 | NM_003958 | 3.22 | 8.17 | 13.48 | | 3.22 | 17.84 | 12.35 | 0.69 |
| 208091_s_at | 81552 | ECOP | NM_030796 | 953.79 | 1353.88 | 1177.53 | | 953.79 | 2878.56 | 1992.10 | 0.69 |
| 221743_at | 10658 | CUGBP1 | AI472139 | 2387.50 | 2660.11 | 3511.98 | | 2387.50 | 4046.64 | 2802.07 | 0.69 |
| 209958_s_at | 27241 | PTHB1 | AF095771 | 18.21 | 51.40 | 44.45 | | 18.21 | 39.14 | 27.10 | 0.69 |
| 215546_at | 5711 | PSMD5 | AK001065 | 7.82 | 7.01 | 7.63 | | 7.82 | 12.89 | 8.93 | 0.69 |
| 204308_s_at | 9895 | KIAA0329 | NM_014844 | 140.43 | 215.94 | 201.49 | | 140.43 | 407.18 | 282.95 | 0.69 |
| 215763_at | | | AL080072 | 9.50 | 4.29 | 3.63 | | 9.50 | 29.38 | 20.42 | 0.70 |
| 208882_s_at | 51366 | EDD1 | U69567 | 945.38 | 867.08 | 1068.37 | | 945.38 | 1484.56 | 1032.01 | 0.70 |
| 217939_s_at | 54812 | AFTPH | NM_017657 | 1520.47 | 1655.27 | 1558.81 | | 1520.47 | 2550.11 | 1772.95 | 0.70 |
| 214308_s_at | 3081 | HGD | AI478172 | 50.39 | 66.35 | 71.66 | | 50.39 | 224.25 | 156.05 | 0.70 |
| 214901_at | 7554 | ZNF8 | AI671579 | 99.90 | 127.90 | 116.70 | | 99.90 | 190.94 | 132.89 | 0.70 |
| 215290_at | 645323 | LOC645323 | AI480014 | 8.32 | 4.20 | 11.61 | | 8.32 | 15.01 | 10.45 | 0.70 |
| 221192_x_at | 79157 | ET | NM_024311 | 119.53 | 210.87 | 172.13 | | 119.53 | 204.14 | 142.17 | 0.70 |
| 206497_at | 55744 | C7orf44 | NM_018224 | 32.91 | 51.29 | 47.19 | | 32.91 | 57.14 | 39.80 | 0.70 |
| 207201_s_at | 6580 | SLC22A1 | NM_003057 | 1.23 | 7.13 | 3.56 | | 1.23 | 19.78 | 13.79 | 0.70 |
| 205020_s_at | 10124 | ARL4A | NM_005738 | 410.69 | 351.50 | 947.75 | | 410.69 | 768.54 | 536.18 | 0.70 |
| 201837_s_at | 9913 | SUPT7L | AF197954 | 710.81 | 903.90 | 958.65 | | 710.81 | 1187.79 | 828.96 | 0.70 |
| 211449_at | 2956 | MSH6 | D89646 | 10.46 | 13.90 | 6.52 | | 10.46 | 27.64 | 19.30 | 0.70 |
| 202351_at | 3685 | ITGAV | AI093579 | 900.06 | 1130.88 | 1112.19 | | 900.06 | 2638.30 | 1842.00 | 0.70 |
| 212964_at | 23119 | HIC2 | AI912206 | 458.21 | 356.89 | 498.96 | | 458.21 | 718.97 | 502.28 | 0.70 |
| 219028_at | 28996 | HIPK2 | NM_022740 | 84.90 | 111.34 | 76.49 | | 84.90 | 186.97 | 130.77 | 0.70 |
| 209246_at | 10061 | ABCF2 | AF261091 | 39.27 | 106.79 | 95.59 | | 39.27 | 156.67 | 109.58 | 0.70 |
| 220117_at | 79750 | ZNF659 | NM_024697 | 5.62 | 3.51 | 5.54 | | 5.62 | 30.25 | 21.16 | 0.70 |
| 204831_at | 1024 | CDK8 | R59697 | 358.00 | 394.44 | 498.47 | | 358.00 | 652.13 | 456.51 | 0.70 |
| 218650_at | 54487 | DGCR8 | NM_022775 | 465.67 | 567.09 | 506.57 | | 465.67 | 709.52 | 497.41 | 0.70 |
| 200921_s_at | 694 | BTG1 | NM_001731 | 1503.67 | 1492.57 | 2660.36 | | 1503.67 | 12888.20 | 9037.04 | 0.70 |
| 202423_at | 7994 | MYST3 | NM_006766 | 1695.24 | 1817.39 | 2043.63 | | 1695.24 | 3022.12 | 2119.35 | 0.70 |
| 209838_at | 9318 | COPS2 | AA496247 | 177.77 | 178.21 | 177.15 | | 177.77 | 277.06 | 194.36 | 0.70 |
| 216737_at | | | AK024525 | 1.92 | 14.72 | 13.93 | | 1.92 | 10.46 | 7.34 | 0.70 |
| 219178_at | 79691 | QTRTD1 | NM_024638 | 202.35 | 195.44 | 155.50 | | 202.35 | 332.97 | 233.70 | 0.70 |
| 203693_s_at | 1871 | E2F3 | NM_001949 | 382.28 | 471.34 | 584.57 | | 382.28 | 989.90 | 694.99 | 0.70 |
| 214551_s_at | 924 | CD7 | NM_006137 | 5.34 | 7.67 | 3.32 | | 5.34 | 18.78 | 13.19 | 0.70 |
| 212802_s_at | 26130 | GAPVD1 | AK023841 | 758.21 | 838.56 | 763.35 | | 758.21 | 1151.03 | 808.51 | 0.70 |
| 220102_at | 668 | FOXL2 | NM_023067 | 124.84 | 101.03 | 111.54 | | 124.84 | 189.96 | 133.53 | 0.70 |
| 212170_at | 10137 | RBM12 | BF447705 | 184.11 | 193.75 | 206.49 | | 184.11 | 311.94 | 219.32 | 0.70 |
| 206096_at | 7584 | ZNF35 | AI809774 | 134.20 | 164.65 | 138.85 | | 134.20 | 240.65 | 169.40 | 0.70 |
| 221749_at | 253943 | YTHDF3 | AU157915 | 1970.64 | 2080.51 | 2522.96 | | 1970.64 | 3456.17 | 2434.35 | 0.70 |
| 221277_s_at | 83480 | PUS3 | NM_031307 | 449.46 | 418.04 | 399.97 | | 449.46 | 775.74 | 546.42 | 0.70 |
| 209639_s_at | 6002 | RGS12 | AF030111 | 17.65 | 47.93 | 47.52 | | 17.65 | 46.40 | 32.69 | 0.70 |
| 213168_at | 6670 | SP3 | AU145005 | 1641.39 | 1383.59 | 2069.14 | | 1641.39 | 2641.44 | 1861.45 | 0.70 |
| 211962_s_at | 677 | ZFP36L1 | BG250310 | 93.53 | 182.47 | 272.65 | | 93.53 | 384.50 | 271.07 | 0.70 |
| 216035_x_at | 6934 | TCF7L2 | AV721430 | 77.97 | 95.55 | 113.77 | | 77.97 | 133.66 | 94.25 | 0.71 |
| 202857_at | 10330 | TMEM4 | NM_014255 | 4091.43 | 4007.43 | 3615.54 | | 4091.43 | 7501.73 | 5291.66 | 0.71 |
| 204934_s_at | 3249 | HPN | NM_002151 | 28.10 | 35.60 | 32.97 | | 28.10 | 56.85 | 40.10 | 0.71 |
| 212195_at | 3572 | IL6ST | AL049265 | 877.45 | 1383.26 | 1757.03 | | 877.45 | 1832.20 | 1292.85 | 0.71 |
| 205799_s_at | 6519 | SLC3A1 | NM_000341 | 28.63 | 30.21 | 32.41 | | 28.63 | 49.28 | 34.78 | 0.71 |
| 222168_at | 220 | ALDH1A3 | AF198444 | 10.34 | 9.23 | 8.40 | | 10.34 | 20.97 | 14.81 | 0.71 |
| 202512_s_at | 9474 | ATG5 | AK001899 | 223.75 | 207.22 | 164.81 | | 223.75 | 339.40 | 239.64 | 0.71 |
| 217572_at | | | AA654586 | 4.01 | 3.97 | 2.78 | | 4.01 | 10.34 | 7.30 | 0.71 |
| 203444_s_at | 9219 | MTA2 | NM_004739 | 16.43 | 7.55 | 8.93 | | 16.43 | 34.45 | 24.33 | 0.71 |
| 221356_x_at | 22953 | P2RX2 | NM_016318 | 8.16 | 9.71 | 11.87 | | 8.16 | 24.96 | 17.63 | 0.71 |
| 218242_s_at | 51111 | SUV420H1 | AA056099 | 861.09 | 861.46 | 1109.93 | | 861.09 | 1498.11 | 1058.86 | 0.71 |
| 221143_at | 29935 | RPA4 | NM_013347 | 59.19 | 194.71 | 170.61 | | 59.19 | 184.47 | 130.39 | 0.71 |
| 219735_s_at | 29842 | TFCP2L1 | NM_014553 | 10.82 | 15.00 | 5.94 | | 10.82 | 27.36 | 19.35 | 0.71 |
| 219782_s_at | 51333 | ZNF771 | NM_016643 | 6.41 | 11.84 | 20.31 | | 6.41 | 16.28 | 11.51 | 0.71 |
| 214235_at | 1577 | CYP3A5 | X90579 | 22.16 | 26.09 | 8.78 | | 22.16 | 34.79 | 24.64 | 0.71 |
| 219001_s_at | 79269 | WDR32 | NM_024345 | 600.71 | 779.97 | 669.32 | | 600.71 | 1071.57 | 759.37 | 0.71 |
| 202550_s_at | 9217 | VAPB | AF160212 | 552.79 | 593.15 | 692.99 | | 552.79 | 1074.42 | 761.54 | 0.71 |
| 203078_at | 8453 | CUL2 | U83410 | 26.90 | 34.69 | 26.84 | | 26.90 | 57.44 | 40.75 | 0.71 |
| 209654_at | 23379 | KIAA0947 | BC004902 | 210.18 | 237.85 | 261.27 | | 210.18 | 424.64 | 301.58 | 0.71 |
| 204656_at | 6461 | SHB | AL138752 | 61.03 | 93.10 | 71.89 | | 61.03 | 177.86 | 126.33 | 0.71 |
| 213682_at | 10762 | NUP50 | AL036344 | 1702.89 | 2121.78 | 2154.63 | | 1702.89 | 2858.81 | 2030.64 | 0.71 |
| 211936_at | 3309 | HSPA5 | AF216292 | 8112.84 | 12515.14 | 10649.28 | | 8112.84 | 16578.74 | 11784.32 | 0.71 |
| 219738_s_at | 5101 | PCDH9 | NM_020403 | 3.58 | 3.80 | 7.62 | | 3.58 | 10.28 | 7.30 | 0.71 |
| 205579_at | 3269 | HRH1 | NM_000861 | 56.44 | 85.32 | 137.67 | | 56.44 | 1043.96 | 742.28 | 0.71 |
| 216583_x_at | | | AC004079 | 108.06 | 145.46 | 131.13 | | 108.06 | 180.75 | 128.54 | 0.71 |
| 208309_s_at | 10892 | MALT1 | NM_006785 | 114.35 | 157.84 | 144.03 | | 114.35 | 235.70 | 167.71 | 0.71 |
| 203549_s_at | 4023 | LPL | NM_000237 | 525.11 | 978.15 | 796.81 | | 525.11 | 4603.58 | 3276.34 | 0.71 |
| 209323_at | 5612 | PRKRIR | AF081567 | 1265.89 | 1106.19 | 1227.42 | | 1265.89 | 2034.55 | 1448.23 | 0.71 |
| 219361_s_at | 64782 | ISG20L1 | NM_022767 | 151.18 | 137.02 | 81.90 | | 151.18 | 613.16 | 436.66 | 0.71 |
| 36552_at | 26005 | DKFZP586P0123 | AL080220 | 231.70 | 255.33 | 285.84 | | 231.70 | 364.63 | 259.93 | 0.71 |
| 214113_s_at | 9939 | RBM8A | AI738479 | 1299.62 | 1551.86 | 1432.72 | | 1299.62 | 2710.69 | 1933.28 | 0.71 |
| 201851_at | 6455 | SH3GL1 | NM_003025 | 399.33 | 441.70 | 290.76 | | 399.33 | 816.35 | 582.39 | 0.71 |
| 210018_x_at | 10892 | MALT1 | AB026118 | 129.64 | 189.31 | 154.54 | | 129.64 | 270.14 | 192.90 | 0.71 |
| 201910_at | 10160 | FARP1 | BF213279 | 9.44 | 7.99 | 14.61 | | 9.44 | 19.00 | 13.57 | 0.71 |
| 219522_at | 24147 | FJX1 | NM_014344 | 36.44 | 37.15 | 34.57 | | 36.44 | 250.00 | 178.70 | 0.71 |
| 91617_at | 54487 | DGCR8 | AI028241 | 170.83 | 226.61 | 185.19 | | 170.83 | 494.02 | 353.18 | 0.71 |
| 213262_at | 26278 | SACS | AI932370 | 312.38 | 280.21 | 255.51 | | 312.38 | 518.33 | 370.62 | 0.72 |
| 211326_x_at | 3077 | HFE | AF150664 | 20.03 | 11.40 | 23.61 | | 20.03 | 42.62 | 30.48 | 0.72 |
| 204229_at | 57030 | SLC17A7 | H40895 | 33.62 | 46.71 | 54.86 | | 33.62 | 81.09 | 57.98 | 0.72 |
| 221047_s_at | 4139 | MARK1 | NM_018650 | 12.38 | 31.15 | 30.69 | | 12.38 | 61.50 | 43.97 | 0.72 |
| 217494_s_at | 11191 | PTENP1 | AF023139 | 8.49 | 4.41 | 5.65 | | 8.49 | 15.21 | 10.88 | 0.72 |
| 201920_at | 6574 | SLC20A1 | NM_005415 | 1024.95 | 1531.14 | 1552.34 | | 1024.95 | 2201.60 | 1575.45 | 0.72 |
| 214047_s_at | 8930 | MBD4 | AI913365 | 1055.41 | 968.06 | 1488.41 | | 1055.41 | 1818.60 | 1301.52 | 0.72 |
| 201713_s_at | 5903 | RANBP2 | D42063 | 1466.56 | 1607.08 | 1524.59 | | 1466.56 | 2371.08 | 1698.11 | 0.72 |
| 210917_at | 7525 | YES1 | AF119914 | 5.23 | 16.96 | 16.60 | | 5.23 | 16.98 | 12.16 | 0.72 |
| 219286_s_at | 64783 | RBM15 | NM_022768 | 1471.49 | 1661.22 | 1642.61 | | 1471.49 | 2490.75 | 1784.15 | 0.72 |
| 210054_at | 79441 | C4orf15 | BC003648 | 492.56 | 486.38 | 589.89 | | 492.56 | 773.58 | 555.05 | 0.72 |
| 211775_x_at | 84796 | MGC13053 | BC006134 | 8.06 | 4.13 | 4.33 | | 8.06 | 14.79 | 10.61 | 0.72 |
| 222132_s_at | 55750 | MULK | AJ278150 | 353.70 | 420.12 | 338.88 | | 353.70 | 574.29 | 412.25 | 0.72 |
| 203282_at | 2632 | GBE1 | NM_000158 | 2653.48 | 2221.58 | 2792.95 | | 2653.48 | 4128.11 | 2964.30 | 0.72 |
| 212792_at | 23333 | DPY19L1 | AB020684 | 311.57 | 616.44 | 484.75 | | 311.57 | 649.02 | 466.12 | 0.72 |
| 219976_at | 51361 | HOOK1 | NM_015888 | 12.46 | 4.87 | 15.96 | | 12.46 | 18.78 | 13.49 | 0.72 |
| 201143_s_at | 1965 | EIF2S1 | BC002513 | 882.84 | 961.20 | 822.82 | | 882.84 | 1622.30 | 1166.00 | 0.72 |
| 214815_at | 51592 | TRIM33 | AU136587 | 69.95 | 123.66 | 162.19 | | 69.95 | 138.11 | 99.33 | 0.72 |
| 211951_at | 9221 | NOLC1 | AI355279 | 1100.14 | 881.10 | 909.25 | | 1100.14 | 1895.54 | 1363.79 | 0.72 |
| 214039_s_at | 55353 | LAPTM4B | T15777 | 3554.81 | 3718.56 | 3599.88 | | 3554.81 | 5666.72 | 4079.12 | 0.72 |
| 217001_x_at | 3111 | HLA- | M29335 | 12.98 | 29.41 | 32.98 | | 12.98 | 30.75 | 22.14 | 0.72 |
| | | DOA | | | | | | | | | |
| 214925_s_at | 6709 | SPTAN1 | AK026484 | 31.00 | 47.45 | 38.05 | | 31.00 | 65.36 | 47.06 | 0.72 |
| 221963_x_at | 730051 | LOC730051 | BE999967 | 303.85 | 286.02 | 286.26 | | 303.85 | 497.31 | 358.30 | 0.72 |
| 218937_at | 54925 | ZNF434 | NM_017810 | 330.65 | 302.59 | 352.84 | | 330.65 | 515.16 | 371.54 | 0.72 |
| 216673_at | 50858 | TTTY1 | AF000990 | 18.16 | 10.80 | 20.74 | | 18.16 | 34.83 | 25.12 | 0.72 |
| 206437_at | 8698 | EDG6 | NM_003775 | 22.91 | 14.04 | 11.03 | | 22.91 | 39.28 | 28.34 | 0.72 |
| 212377_s_at | 4853 | NOTCH2 | AU158495 | 1356.47 | 1934.58 | 1641.78 | | 1356.47 | 2441.62 | 1761.72 | 0.72 |
| 215893_x_at | 2262 | GPC5 | AF339787 | 13.62 | 32.77 | 25.52 | | 13.62 | 24.25 | 17.51 | 0.72 |
| 200797_s_at | 4170 | MCL1 | AI275690 | 4187.99 | 4593.08 | 5116.10 | | 4187.99 | 7034.20 | 5078.87 | 0.72 |
| 202613_at | 1503 | CTPS | NM_001905 | 4727.13 | 5077.70 | 3843.93 | | 4727.13 | 7671.52 | 5542.36 | 0.72 |
| 217162_at | 7258 | TSPY1 | M94893 | 5.65 | 6.68 | 6.95 | | 5.65 | 22.86 | 16.52 | 0.72 |
| 213136_at | 5771 | PTPN2 | AI828880 | 1015.49 | 1222.19 | 1430.00 | | 1015.49 | 1976.07 | 1428.65 | 0.72 |
| 215344_at | | | AL137325 | 14.82 | 18.68 | 39.41 | | 14.82 | 40.00 | 28.92 | 0.72 |
| 211725_s_at | 637 | BID | BC005884 | 1031.27 | 2006.66 | 1877.88 | | 1031.27 | 4296.30 | 3107.50 | 0.72 |
| 212154_at | 6383 | SDC2 | AI380298 | 761.09 | 569.17 | 722.51 | | 761.09 | 1237.27 | 895.70 | 0.72 |
| 221732_at | 124583 | CANT1 | AK026161 | 784.24 | 872.51 | 784.81 | | 784.24 | 1386.89 | 1004.36 | 0.72 |
| 213009_s_at | 4591 | TRIM37 | AK022701 | 393.96 | 426.07 | 508.32 | | 393.96 | 711.22 | 515.24 | 0.72 |
| 203804_s_at | 51747 | CROP | NM_006107 | 1529.19 | 1398.67 | 1469.66 | | 1529.19 | 2319.21 | 1680.83 | 0.72 |
| 214807_at | | | AI278204 | 41.44 | 59.51 | 43.94 | | 41.44 | 64.50 | 46.78 | 0.73 |
| 206131_at | 1208 | CLPS | NM_001832 | 8.66 | 9.73 | 14.65 | | 8.66 | 19.30 | 13.99 | 0.73 |
| 218732_at | 51651 | PTRH2 | NM_016077 | 600.26 | 516.28 | 603.23 | | 600.26 | 1051.28 | 762.69 | 0.73 |
| 206355_at | 2774 | GNAL | NM_002071 | 2.18 | 23.95 | 25.49 | | 2.18 | 21.05 | 15.27 | 0.73 |
| 204405_x_at | 27292 | DIMT1L | NM_014473 | 1103.60 | 1057.30 | 1251.65 | | 1103.60 | 1751.53 | 1272.07 | 0.73 |
| 216490_x_at | 442175 | LOC442175 | AL133267 | 33.36 | 38.68 | 48.87 | | 33.36 | 67.58 | 49.14 | 0.73 |
| 204837_at | 66036 | MTMR9 | AL080178 | 804.57 | 1018.62 | 1031.17 | | 804.57 | 1419.60 | 1032.26 | 0.73 |
| 218428_s_at | 51455 | REV1 | NM_016316 | 612.12 | 614.11 | 781.15 | | 612.12 | 944.68 | 687.90 | 0.73 |
| 214379_at | 660 | BMX | AI954458 | 10.38 | 34.44 | 33.99 | | 10.38 | 30.97 | 22.56 | 0.73 |
| 213415_at | 1193 | CLIC2 | AI768628 | 69.43 | 85.26 | 77.93 | | 69.43 | 237.75 | 173.17 | 0.73 |
| 208212_s_at | 238 | ALK | NM_004304 | 8.04 | 6.48 | 16.11 | | 8.04 | 17.29 | 12.60 | 0.73 |
| 215776_at | 3645 | INSRR | J05046 | 5.44 | 6.62 | 3.48 | | 5.44 | 13.32 | 9.71 | 0.73 |
| 205352_at | 5274 | SERPINI1 | NM_005025 | 239.82 | 290.12 | 285.15 | | 239.82 | 514.65 | 375.34 | 0.73 |
| 217106_x_at | 27292 | DIMT1L | AF091078 | 1108.60 | 741.84 | 1180.24 | | 1108.60 | 1709.37 | 1247.10 | 0.73 |
| 215114_at | 26168 | SENP3 | AK000923 | 52.39 | 78.02 | 82.02 | | 52.39 | 96.97 | 70.76 | 0.73 |
| 204601_at | 9683 | N4BP1 | NM_014664 | 190.90 | 325.74 | 325.99 | | 190.90 | 510.97 | 372.89 | 0.73 |
| 208901_s_at | 7150 | TOP1 | J03250 | 2887.11 | 2813.70 | 2784.18 | | 2887.11 | 5107.20 | 3728.25 | 0.73 |
| 210869_s_at | 4162 | MCAM | M29277 | 5.06 | 11.12 | 12.03 | | 5.06 | 17.53 | 12.80 | 0.73 |
| 210557_x_at | 1435 | CSF1 | M76453 | 8.39 | 38.30 | 32.20 | | 8.39 | 25.45 | 18.59 | 0.73 |
| 50221_at | 7942 | TFEB | AI524138 | 406.72 | 626.55 | 588.76 | | 406.72 | 1084.38 | 792.07 | 0.73 |
| 207513_s_at | 7743 | ZNF189 | NM_003452 | 919.08 | 837.63 | 1302.04 | | 919.08 | 1908.19 | 1394.27 | 0.73 |
| 219449_s_at | 54968 | TMEM70 | BC002748 | 1845.91 | 1870.07 | 2171.13 | | 1845.91 | 3162.08 | 2314.62 | 0.73 |
| 218578_at | 79577 | CDC73 | NM_024529 | 464.99 | 597.79 | 711.52 | | 464.99 | 1145.89 | 839.74 | 0.73 |
| 207680_x_at | 5077 | PAX3 | NM_013942 | 3.57 | 4.64 | 3.77 | | 3.57 | 11.00 | 8.06 | 0.73 |
| 211111_at | | | AB016902 | 20.65 | 16.42 | 4.45 | | 20.65 | 45.93 | 33.68 | 0.73 |
| 203082_at | 9790 | BMS1L | NM_014753 | 305.56 | 413.45 | 320.62 | | 305.56 | 467.58 | 342.88 | 0.73 |
| 203743_s_at | 6996 | TDG | NM_003211 | 1324.87 | 1331.25 | 1322.57 | | 1324.87 | 2469.34 | 1810.83 | 0.73 |
| 31846_at | 29984 | RHOD | AW003733 | 156.72 | 372.47 | 357.99 | | 156.72 | 350.43 | 257.00 | 0.73 |
| 214701_s_at | 2335 | FN1 | AJ276395 | 20.32 | 25.69 | 34.88 | | 20.32 | 38.80 | 28.58 | 0.74 |
| 208256_at | 1943 | EFNA2 | NM_001405 | 10.68 | 5.10 | 16.23 | | 10.68 | 26.39 | 19.44 | 0.74 |
| 215053_at | 10847 | SRCAP | AK023808 | 14.98 | 12.91 | 40.94 | | 14.98 | 39.03 | 28.76 | 0.74 |
| 217987_at | 54529 | ASNSD1 | NM_019048 | 1197.44 | 1010.71 | 930.38 | | 1197.44 | 1821.81 | 1342.78 | 0.74 |
| 213030_s_at | 5362 | PLXNA2 | AI688418 | 22.80 | 15.54 | 30.87 | | 22.80 | 39.11 | 28.83 | 0.74 |
| 216213_at | 4750 | NEK1 | AF155113 | 10.93 | 6.14 | 17.00 | | 10.93 | 22.05 | 16.26 | 0.74 |
| 201991_s_at | 3799 | KIF5B | BF223224 | 1858.93 | 2197.39 | 2283.59 | | 1858.93 | 2886.88 | 2130.15 | 0.74 |
| 208217_at | 2570 | GABRR2 | NM_002043 | 21.07 | 28.02 | 26.58 | | 21.07 | 34.27 | 25.30 | 0.74 |
| 212185_x_at | 4502 | MT2A | NM_005953 | 1296.38 | 1024.92 | 882.29 | | 1296.38 | 4233.39 | 3131.46 | 0.74 |
| 212907_at | 7779 | SLC30A1 | AI972416 | 1730.18 | 2175.51 | 4455.87 | | 1730.18 | 3091.75 | 2287.08 | 0.74 |
| 201144_s_at | 1965 | EIF2S1 | NM_004094 | 2093.74 | 2177.70 | 2519.45 | | 2093.74 | 3878.47 | 2869.31 | 0.74 |
| 204651_at | 4899 | NRF1 | AW003022 | 288.61 | 297.04 | 357.99 | | 288.61 | 440.23 | 326.21 | 0.74 |
| 33579_i_at | 8484 | GALR3 | Z97630 | 12.57 | 9.11 | 17.95 | | 12.57 | 34.71 | 25.72 | 0.74 |
| 202981_x_at | 6477 | SIAH1 | NM_003031 | 551.81 | 654.49 | 1013.27 | | 551.81 | 1156.91 | 857.74 | 0.74 |
| 214155_s_at | 113251 | LARP4 | AI743740 | 117.96 | 107.11 | 117.80 | | 117.96 | 216.79 | 160.83 | 0.74 |
| 203590_at | 1783 | DYNC1LI2 | NM_006141 | 765.19 | 1027.22 | 832.83 | | 765.19 | 1271.94 | 943.71 | 0.74 |
| 205178_s_at | 5930 | RBBP6 | NM_006910 | 723.44 | 845.08 | 639.59 | | 723.44 | 1229.40 | 912.59 | 0.74 |
| 201506_at | 7045 | TGFB1 | NM_000358 | 5679.79 | 4780.41 | 5140.67 | | 5679.79 | 8538.91 | 6345.51 | 0.74 |
| 44146_at | 26205 | GMEB2 | AA045183 | 535.44 | 484.37 | 540.33 | | 535.44 | 873.18 | 648.95 | 0.74 |
| 204622_x_at | 4929 | NR4A2 | NM_006186 | 14.85 | 62.09 | 57.73 | | 14.85 | 114.14 | 84.90 | 0.74 |
| 221799_at | 54480 | CSGlcA-T | AB037823 | 232.43 | 369.80 | 285.95 | | 232.43 | 519.09 | 386.20 | 0.74 |
| 222103_at | 466 | ATF1 | AI434345 | 293.91 | 162.30 | 316.61 | | 293.91 | 533.76 | 397.77 | 0.75 |
| 213370_s_at | 51460 | SFMBT1 | BF057298 | 269.51 | 323.18 | 281.04 | | 269.51 | 453.99 | 338.46 | 0.75 |
| 204630_s_at | 9527 | GOSR1 | NM_004871 | 1480.16 | 1820.33 | 2140.78 | | 1480.16 | 3284.39 | 2449.91 | 0.75 |
| 216263_s_at | | | AK022215 | 44.11 | 51.56 | 49.28 | | 44.11 | 72.18 | 53.85 | 0.75 |
| 204977_at | 1662 | DDX10 | NM_004398 | 676.61 | 609.50 | 708.35 | | 676.61 | 1024.69 | 764.61 | 0.75 |
| 210953_at | 9819 | TSC22D2 | AF201292 | 20.81 | 6.33 | 11.43 | | 20.81 | 38.03 | 28.39 | 0.75 |
| 221549_at | 83743 | GRWD1 | AF337808 | 231.45 | 257.01 | 210.61 | | 231.45 | 452.18 | 337.65 | 0.75 |
| 213750_at | | | AA928506 | 871.91 | 936.70 | 874.32 | | 871.91 | 1862.02 | 1390.70 | 0.75 |
| 214712_at | 445329 | SULT1A4 | AK023827 | 11.17 | 40.47 | 32.39 | | 11.17 | 33.60 | 25.10 | 0.75 |
| 213596_at | 837 | CASP4 | AL050391 | 77.77 | 133.92 | 129.03 | | 77.77 | 174.35 | 130.31 | 0.75 |
| 202451_at | 2965 | GTF2H1 | BC000365 | 958.24 | 1030.06 | 1421.25 | | 958.24 | 1642.29 | 1230.38 | 0.75 |
| 201437_s_at | 1977 | EIF4E | NM_001968 | 556.50 | 418.04 | 503.01 | | 556.50 | 876.69 | 656.82 | 0.75 |
| 203529_at | 5537 | PPP6C | NM_016294 | 2385.22 | 2315.48 | 2481.33 | | 2385.22 | 3677.76 | 2756.60 | 0.75 |
| 212229_s_at | 23014 | FBXO21 | AK001699 | 322.06 | 365.74 | 269.55 | | 322.06 | 526.35 | 394.75 | 0.75 |

### Example 3

### Effect of Tetrahydro-Isoalpha Acid on Brachial Artery Flow Mediated Dilation

The purpose of this study was to evaluate the effects on brachial artery endothelial responsiveness (a measure of cardiovascular risk) of oral tetrahydro-isoalpha acid administration (100 mg).

Brachial artery endothelial responsiveness was evaluated in three healthy subjects by measuring Flow Mediated Vasodilation using a Sonosite MicroMaxx ultrasound machine according to the procedures as outlined in Corretti, M C., et al., Guidelines for the Ultrasound Assessment of Endothelial-Dependent Flow-Mediated Vasodilation of the Brachial Artery—A Report of the International Brachial Artery Reactive Task Force. J. Am. Coll. Cadiol. 2002; 39(2): 257-65. Briefly, ischemia was induced in the brachial artery using a blood pressure cuff inflated to 50 mm Hg above systolic pressure with baseline and post hyperemic flow rate measured by ultrasound. The tests were repeated between one and two hours following an oral dose of 1056 mg of tetrahydro-isoalpha acid.

Results: Subjects were noted to have a 40.2% increase in flow rate above that observed from baseline (calculated as the 100×(ratio of hyperemic flow rate−baseline low rate/baseline flow rate)) prior to tetrahydro-isoalpha acid administration. A 63.3% increase in flow rate was observed from baseline (calculated as the 100×(ratio of hyperemic flow rate−baseline low rate /baseline flow rate)) one to two hours after tetrahydro-isoalpha acid administration. This is an increase in flow rate relative to baseline of 57%. It was further noted that tetrahydro-isoalpha acid administration resulted in a 28.2% increase in peak post hyperemic flow rate as compared to the peak post hyperemic flow rate in the untreated individuals. The increase in flow rate may be attributed to an increase in vessel diameter insofar as blood pressure and heart rate remained relatively constant during the protocol.

1. A method for improving a cardiovascular risk factor in a subject in need, the method comprising treating the subject with a therapeutically effective amount of a substituted 1,3-cyclopentadione compound wherein said amount modulates the expression of cardiovascular risk factor associated marker gene expression.

2. The method of claim 1, wherein the substituted 1,3-cyclopentadione compound is selected from the group comprising (+)-(4R,5S)-3,4-dihydroxy-2-(3-methylbutanoyl)-5-(3-methylbutyl)-4-(4-methylpentanoyl)cyclopent-2-en-1-one, (−)-(4S,5S)-3,4-dihydroxy-2-(3-methylbutanoyl)-5-(3-methylbutyl)-4-(4-methylpentanoyl)cyclopent-2-en-1-one, (+)-(4R,5S)-3,4-dihydroxy-5-(3-methylbutyl)-4-(4-methylpentanoyl)-2-(3-methylpropanoyl)cyclopent-2-en-1-one, (−)-(4S,5S)-3,4-dihydroxy-5-(3-methylbutyl)-4-(4-methylpentanoyl)-2-(3-methylpropanoyl)cyclopent-2-en-1-one, (+)-(4R,5S)-3,4-dihydroxy-2-(2-methylbutanoyl)-5-(3-methylbutyl)-4-(4-methylpentanoyl)cyclopent-2-en-1-one and (+)-(4R,5S)-3,4-dihydroxy-5-(3-methylbutyl)-4-(4-methylpentanoyl)-2-petanoylcyclopent-2-en-1-one.

3. The method of claim 1, wherein the cardiovascular risk associated marker gene is selected from the group consisting of TNFα, MCP-1, VCAM-1, MMP-3, ICAM1 and SDF1.

4. The method of claim 1, further comprising lifestyle modification or pharmaceutical treatment.

5. The method of claim 1, wherein the lifestyle modification or pharmaceutical treatment is selected from the group consisting of blood pressure reduction, cholesterol level modulation, diabetes treatment, increased exercise, inflammation, obesity and weight reduction, prothrombotic factors treatment, reduction in serum homocysteine and lipoprotein (a), serum triglyceride reduction, smoking cessation, and stress reduction.

6. A method for promoting vascular elasticity in a subject in need, the method comprising treating the subject with a therapeutically effective amount of a substituted 1,3-cyclopentadione compound wherein said amount increases vascular elasticity or dilation.

7. The method of claim 6, wherein the substituted 1,3-cyclopentadione compound is selected from the group comprising (+)-(4R,5S)-3,4-dihydroxy-2-(3-methylbutanoyl)-5-(3-methylbutyl)-4-(4-methylpentanoypcyclopent-2-en-1-one, (−)-(4S,5S)-3,4-dihydroxy-2-(3 -methylbutanoyl)-5-(3-methylbutyl)-4-(4-methylpentanoypcyclopent-2-en-1-one (+)-(4R,5S)-3,4-dihydroxy-5-(3-methylbutyl)-4-(4-methylpentanoyl)-2-(3-methylpropanoyl)cyclopent-2-en-1-one, (−)-(4S,5S)-3,4-dihydroxy-5-(3-methylbutyl)-4-(4-methylpentanoyl)-2-(3-methylpropanoyl)cyclopent-2-en-1-one, (+)-(4R,5S)-3,4-dihydroxy-2-(2-methylbutanoyl)-5-(3-methylbutyl)-4-(4-methylpentanoyl)cyclopent-2-en-1-one and (+)-(4R,5S)-3,4-dihydroxy-5-(3-methylbutyl)-4-(4-methylpentanoyl)-2-petanoylcyclopent-2-en-1-one.

8. A composition promoting cardiovascular health in a subject in need, said composition comprising a therapeutically effective amount of a substituted 1,3-cyclopentadione compound wherein said amount (a) modulates the expression of cardiovascular risk associated marker gene expression; or (b) increases vascular elasticity or dilation.

9. The composition of claim 8, wherein the substituted 1,3-cyclopentadione compound is selected from the group comprising (+)-(4R,5S)-3,4-dihydroxy-2-(3-methylbutanoyl)-5-(3-methylbutyl)-4-(4-methylpentanoyl)cyclopent-2-en-1-one, (−)-(4S,5S)-3,4-dihydroxy-2-(3-methylbutanoyl)-5-(3-methylbutyl)-4-(4-methylpentanoyl)cyclopent-2-en-1-one, (+)-(4R,5S)-3,4-dihydroxy-5-(3-methylbutyl)-4-(4-methylpentanoyl)-2-(3-methylpropanoyl)cyclopent-2-en-1-one, (−)-(4S,5S)-3,4-dihydroxy-5-(3-methylbutyl)-4-(4-methylpentanoyl)-2-(3-methylpropanoyl)cyclopent-2-en-1-one, (+)-(4R,5S)-3,4-dihydroxy-2-(2-methylbutanoyl)-5-(3-methylbutyl)-4-(4-methylpentanoyl)cyclopent-2-en-1-one and (+)-(4R,5S)-3,4-dihydroxy-5-(3-methylbutyl)-4-(4-methylpentanoyl)-2-petanoylcyclopent-2-en-1-one.

10. The composition of claim 8, wherein the composition further comprises a pharmaceutically acceptable excipient.

11. The composition of claim 10 wherein the pharmaceutically acceptable excipient is selected from the group consisting of coatings, isotonic and absorption delaying agents, binders, adhesives, lubricants, disintergrants, coloring agents, flavoring agents, sweetening agents, absorbants, detergents, and emulsifying agents.

12. The composition of claim 8, wherein the composition further comprises one or more members selected from the group consisting of antioxidants, vitamins, minerals, proteins, fats, and carbohydrates.
